SEQUENCE LISTING (234 sequences)

SEQ ID NO: 1 - RNA, 20 nt, synthetic construct
  source 1..20: mol_type = other RNA
gcaggcaggacaaccattac

SEQ ID NO: 2 - RNA, 20 nt, synthetic construct
  source 1..20: mol_type = other RNA
caggcaggacaaccattact

SEQ ID NO: 3 - RNA, 20 nt, synthetic construct
  source 1..20: mol_type = other RNA
aggagtcagatgctgtttcg

SEQ ID NO: 4 - RNA, 20 nt, synthetic construct
  source 1..20: mol_type = other RNA
ctaaaacagggaagcgaaaa

SEQ ID NO: 5 - RNA, 20 nt, synthetic construct
  source 1..20: mol_type = other RNA
cgcttccctgttttagctgc

SEQ ID NO: 6 - RNA, 20 nt, synthetic construct
  source 1..20: mol_type = other RNA
tgtcgccagcagctaaaaca

SEQ ID NO: 7 - RNA, 20 nt, synthetic construct
  source 1..20: mol_type = other RNA
ctgtcgccagcagctaaaac

SEQ ID NO: 8 - RNA, 20 nt, synthetic construct
  source 1..20: mol_type = other RNA
gcgacagttcagccatcact

SEQ ID NO: 9 - RNA, 20 nt, synthetic construct
  source 1..20: mol_type = other RNA
acatgaactcatccaagtga

SEQ ID NO: 10 - RNA, 20 nt, synthetic construct
  source 1..20: mol_type = other RNA
gttcatgtattgctttgcgt

SEQ ID NO: 11 - RNA, 20 nt, synthetic construct
  source 1..20: mol_type = other RNA
gacattcatgtcttccttga

SEQ ID NO: 12 - RNA, 20 nt, synthetic construct
  source 1..20: mol_type = other RNA
tgatggtctccacactcttt

SEQ ID NO: 13 - RNA, 20 nt, synthetic construct
  source 1..20: mol_type = other RNA
aaagagtgtggagaccatca

SEQ ID NO: 14 - RNA, 20 nt, synthetic construct
  source 1..20: mol_type = other RNA
ccagagcatccaaaagagtg

SEQ ID NO: 15 - RNA, 20 nt, synthetic construct
  source 1..20: mol_type = other RNA
gataatggaactcttttctt

SEQ ID NO: 16 - RNA, 20 nt, synthetic construct
  source 1..20: mol_type = other RNA
cattcagatgtagcggataa

SEQ ID NO: 17 - RNA, 20 nt, synthetic construct
  source 1..20: mol_type = other RNA
tgcaggtcattcagatgtag

SEQ ID NO: 18 - RNA, 20 nt, synthetic construct
  source 1..20: mol_type = other RNA
cttcttttacatatgggtcc

SEQ ID NO: 19 - RNA, 20 nt, synthetic construct
  source 1..20: mol_type = other RNA
tgcatcgttttgggttctct

SEQ ID NO: 20 - RNA, 20 nt, synthetic construct
  source 1..20: mol_type = other RNA
tttcagctctgcatcgtttt

SEQ ID NO: 21 - RNA, 20 nt, synthetic construct
  source 1..20: mol_type = other RNA
ttttcagctctgcatcgttt

SEQ ID NO: 22 - RNA, 20 nt, synthetic construct
  source 1..20: mol_type = other RNA
tgagcactgaaagcatgatc

SEQ ID NO: 23 - RNA, 20 nt, synthetic construct
  source 1..20: mol_type = other RNA
tgaaagcatgatccgggacg

SEQ ID NO: 24 - RNA, 20 nt, synthetic construct
  source 1..20: mol_type = other RNA
catgatccgggacgtggagc

SEQ ID NO: 25 - RNA, 20 nt, synthetic construct
  source 1..20: mol_type = other RNA
cttcttggggagcgcctcct

SEQ ID NO: 26 - RNA, 20 nt, synthetic construct
  source 1..20: mol_type = other RNA
gaggcgctccccaagaagac

SEQ ID NO: 27 - RNA, 20 nt, synthetic construct
  source 1..20: mol_type = other RNA
aggcgctccccaagaagaca

SEQ ID NO: 28 - RNA, 20 nt, synthetic construct
  source 1..20: mol_type = other RNA
ggcgctccccaagaagacag

SEQ ID NO: 29 - RNA, 20 nt, synthetic construct
  source 1..20: mol_type = other RNA
gcgctccccaagaagacagg

SEQ ID NO: 30 - RNA, 20 nt, synthetic construct
  source 1..20: mol_type = other RNA
caagaagacaggggggcccc

SEQ ID NO: 31 - RNA, 20 nt, synthetic construct
  source 1..20: mol_type = other RNA
ggggcccccctgtcttcttg

SEQ ID NO: 32 - RNA, 20 nt, synthetic construct
  source 1..20: mol_type = other RNA
ctggggcccccctgtcttct

SEQ ID NO: 33 - RNA, 20 nt, synthetic construct
  source 1..20: mol_type = other RNA
aagcaccgcctggagccctg

SEQ ID NO: 34 - RNA, 20 nt, synthetic construct
  source 1..20: mol_type = other RNA
caagcaccgcctggagccct

SEQ ID NO: 35 - RNA, 20 nt, synthetic construct
  source 1..20: mol_type = other RNA
gctgaggaacaagcaccgcc

SEQ ID NO: 36 - RNA, 20 nt, synthetic construct
  source 1..20: mol_type = other RNA
cctcttctccttcctgatcg

SEQ ID NO: 37 - RNA, 20 nt, synthetic construct
  source 1..20: mol_type = other RNA
ttctccttcctgatcgtggc

SEQ ID NO: 38 - RNA, 20 nt, synthetic construct
  source 1..20: mol_type = other RNA
ggcgcctgccacgatcagga

SEQ ID NO: 39 - RNA, 20 nt, synthetic construct
  source 1..20: mol_type = other RNA
tggtggcgcctgccacgatc

SEQ ID NO: 40 - RNA, 20 nt, synthetic construct
  source 1..20: mol_type = other RNA
ctgctgcactttggagtgat

SEQ ID NO: 41 - RNA, 20 nt, synthetic construct
  source 1..20: mol_type = other RNA
cgatcactccaaagtgcagc

SEQ ID NO: 42 - RNA, 20 nt, synthetic construct
  source 1..20: mol_type = other RNA
ttggagtgatcggcccccag

SEQ ID NO: 43 - RNA, 20 nt, synthetic construct
  source 1..20: mol_type = other RNA
tggagtgatcggcccccaga

SEQ ID NO: 44 - RNA, 20 nt, synthetic construct
  source 1..20: mol_type = other RNA
aggcactcacctcttccctc

SEQ ID NO: 45 - RNA, 20 nt, synthetic construct
  source 1..20: mol_type = other RNA
tgattagagagaggtccctg

SEQ ID NO: 46 - RNA, 20 nt, synthetic construct
  source 1..20: mol_type = other RNA
ctgattagagagaggtccct

SEQ ID NO: 47 - RNA, 20 nt, synthetic construct
  source 1..20: mol_type = other RNA
gctgattagagagaggtccc

SEQ ID NO: 48 - RNA, 20 nt, synthetic construct
  source 1..20: mol_type = other RNA
cctctctctaatcagccctc

SEQ ID NO: 49 - RNA, 20 nt, synthetic construct
  source 1..20: mol_type = other RNA
ccagagggctgattagagag

SEQ ID NO: 50 - RNA, 20 nt, synthetic construct
  source 1..20: mol_type = other RNA
tctaatcagccctctggccc

SEQ ID NO: 51 - RNA, 20 nt, synthetic construct
  source 1..20: mol_type = other RNA
tactgactgcctgggccaga

SEQ ID NO: 52 - RNA, 20 nt, synthetic construct
  source 1..20: mol_type = other RNA
ttactgactgcctgggccag

SEQ ID NO: 53 - RNA, 20 nt, synthetic construct
  source 1..20: mol_type = other RNA
gagacacttactgactgcct

SEQ ID NO: 54 - RNA, 20 nt, synthetic construct
  source 1..20: mol_type = other RNA
ggagacacttactgactgcc

SEQ ID NO: 55 - RNA, 20 nt, synthetic construct
  source 1..20: mol_type = other RNA
gggctacaggcttgtcactc

SEQ ID NO: 56 - RNA, 20 nt, synthetic construct
  source 1..20: mol_type = other RNA
tgggctacaggcttgtcact

SEQ ID NO: 57 - RNA, 20 nt, synthetic construct
  source 1..20: mol_type = other RNA
ttacctacaacatgggctac

SEQ ID NO: 58 - RNA, 20 nt, synthetic construct
  source 1..20: mol_type = other RNA
agagctcttacctacaacat

SEQ ID NO: 59 - RNA, 20 nt, synthetic construct
  source 1..20: mol_type = other RNA
cagagctcttacctacaaca

SEQ ID NO: 60 - RNA, 20 nt, synthetic construct
  source 1..20: mol_type = other RNA
tccagcaaaccctcaagctg

SEQ ID NO: 61 - RNA, 20 nt, synthetic construct
  source 1..20: mol_type = other RNA
cagcaaaccctcaagctgag

SEQ ID NO: 62 - RNA, 20 nt, synthetic construct
  source 1..20: mol_type = other RNA
ggagctgcccctcagcttga

SEQ ID NO: 63 - RNA, 20 nt, synthetic construct
  source 1..20: mol_type = other RNA
tggagctgcccctcagcttg

SEQ ID NO: 64 - RNA, 20 nt, synthetic construct
  source 1..20: mol_type = other RNA
agctccagtggctgaaccgc

SEQ ID NO: 65 - RNA, 20 nt, synthetic construct
  source 1..20: mol_type = other RNA
gctccagtggctgaaccgcc

SEQ ID NO: 66 - RNA, 20 nt, synthetic construct
  source 1..20: mol_type = other RNA
tggcccggcggttcagccac

SEQ ID NO: 67 - RNA, 20 nt, synthetic construct
  source 1..20: mol_type = other RNA
ccgccgggccaatgccctcc

SEQ ID NO: 68 - RNA, 20 nt, synthetic construct
  source 1..20: mol_type = other RNA
ccaggagggcattggcccgg

SEQ ID NO: 69 - RNA, 20 nt, synthetic construct
  source 1..20: mol_type = other RNA
gccaatgccctcctggccaa

SEQ ID NO: 70 - RNA, 20 nt, synthetic construct
  source 1..20: mol_type = other RNA
gccattggccaggagggcat

SEQ ID NO: 71 - RNA, 20 nt, synthetic construct
  source 1..20: mol_type = other RNA
tgccctcctggccaatggcg

SEQ ID NO: 72 - RNA, 20 nt, synthetic construct
  source 1..20: mol_type = other RNA
gctccacgccattggccagg

SEQ ID NO: 73 - RNA, 20 nt, synthetic construct
  source 1..20: mol_type = other RNA
tcagctccacgccattggcc

SEQ ID NO: 74 - RNA, 20 nt, synthetic construct
  source 1..20: mol_type = other RNA
atctctcagctccacgccat

SEQ ID NO: 75 - RNA, 20 nt, synthetic construct
  source 1..20: mol_type = other RNA
ggagctgagagataaccagc

SEQ ID NO: 76 - RNA, 20 nt, synthetic construct
  source 1..20: mol_type = other RNA
gctgagagataaccagctgg

SEQ ID NO: 77 - RNA, 20 nt, synthetic construct
  source 1..20: mol_type = other RNA
ccagctggtggtgccatcag

SEQ ID NO: 78 - RNA, 20 nt, synthetic construct
  source 1..20: mol_type = other RNA
cagctggtggtgccatcaga

SEQ ID NO: 79 - RNA, 20 nt, synthetic construct
  source 1..20: mol_type = other RNA
atgaggtacaggccctctga

SEQ ID NO: 80 - RNA, 20 nt, synthetic construct
  source 1..20: mol_type = other RNA
cctgggagtagatgaggtac

SEQ ID NO: 81 - RNA, 20 nt, synthetic construct
  source 1..20: mol_type = other RNA
tactcccaggtcctcttcaa

SEQ ID NO: 82 - RNA, 20 nt, synthetic construct
  source 1..20: mol_type = other RNA
cttggcccttgaagaggacc

SEQ ID NO: 83 - RNA, 20 nt, synthetic construct
  source 1..20: mol_type = other RNA
ggcgatgcggctgatggtgt

SEQ ID NO: 84 - RNA, 20 nt, synthetic construct
  source 1..20: mol_type = other RNA
cggcgatgcggctgatggtg

SEQ ID NO: 85 - RNA, 20 nt, synthetic construct
  source 1..20: mol_type = other RNA
ggagacggcgatgcggctga

SEQ ID NO: 86 - RNA, 20 nt, synthetic construct
  source 1..20: mol_type = other RNA
tctggtaggagacggcgatg

SEQ ID NO: 87 - RNA, 20 nt, synthetic construct
  source 1..20: mol_type = other RNA
cgccgtctcctaccagacca

SEQ ID NO: 88 - RNA, 20 nt, synthetic construct
  source 1..20: mol_type = other RNA
gaggttgaccttggtctggt

SEQ ID NO: 89 - RNA, 20 nt, synthetic construct
  source 1..20: mol_type = other RNA
ggcagagaggaggttgacct

SEQ ID NO: 90 - RNA, 20 nt, synthetic construct
  source 1..20: mol_type = other RNA
ccatcaagagcccctgccag

SEQ ID NO: 91 - RNA, 20 nt, synthetic construct
  source 1..20: mol_type = other RNA
catcaagagcccctgccaga

SEQ ID NO: 92 - RNA, 20 nt, synthetic construct
  source 1..20: mol_type = other RNA
ctggggtctccctctggcag

SEQ ID NO: 93 - RNA, 20 nt, synthetic construct
  source 1..20: mol_type = other RNA
tctggggtctccctctggca

SEQ ID NO: 94 - RNA, 20 nt, synthetic construct
  source 1..20: mol_type = other RNA
gatgggctcataccagggct

SEQ ID NO: 95 - RNA, 20 nt, synthetic construct
  source 1..20: mol_type = other RNA
ctggtatgagcccatctatc

SEQ ID NO: 96 - RNA, 20 nt, synthetic construct
  source 1..20: mol_type = other RNA
agatagatgggctcatacca

SEQ ID NO: 97 - RNA, 20 nt, synthetic construct
  source 1..20: mol_type = other RNA
tggtatgagcccatctatct

SEQ ID NO: 98 - RNA, 20 nt, synthetic construct
  source 1..20: mol_type = other RNA
cagatagatgggctcatacc

SEQ ID NO: 99 - RNA, 20 nt, synthetic construct
  source 1..20: mol_type = other RNA
atgagcccatctatctggga

SEQ ID NO: 100 - RNA, 20 nt, synthetic construct
  source 1..20: mol_type = other RNA
tgagcccatctatctgggag

SEQ ID NO: 101 - RNA, 20 nt, synthetic construct
  source 1..20: mol_type = other RNA
aagacccctcccagatagat

SEQ ID NO: 102 - RNA, 20 nt, synthetic construct
  source 1..20: mol_type = other RNA
gtcggtcacccttctccagc

SEQ ID NO: 103 - RNA, 20 nt, synthetic construct
  source 1..20: mol_type = other RNA
gactcagcgctgagatcaat

SEQ ID NO: 104 - RNA, 20 nt, synthetic construct
  source 1..20: mol_type = other RNA
gattgatctcagcgctgagt

SEQ ID NO: 105 - RNA, 20 nt, synthetic construct
  source 1..20: mol_type = other RNA
tcggcaaagtcgagatagtc

SEQ ID NO: 106 - RNA, 20 nt, synthetic construct
  source 1..20: mol_type = other RNA
ctcggcaaagtcgagatagt

SEQ ID NO: 107 - RNA, 20 nt, synthetic construct
  source 1..20: mol_type = other RNA
tatctcgactttgccgagtc

SEQ ID NO: 108 - RNA, 20 nt, synthetic construct
  source 1..20: mol_type = other RNA
atctcgactttgccgagtct

SEQ ID NO: 109 - RNA, 20 nt, synthetic construct
  source 1..20: mol_type = other RNA
cgactttgccgagtctgggc

SEQ ID NO: 110 - RNA, 20 nt, synthetic construct
  source 1..20: mol_type = other RNA
gagtctgggcaggtctactt

SEQ ID NO: 111 - RNA, 20 nt, synthetic construct
  source 1..20: mol_type = other RNA
agtctgggcaggtctacttt

SEQ ID NO: 112 - RNA, 20 nt, synthetic construct
  source 1..20: mol_type = other RNA
aaagtagacctgcccagact

SEQ ID NO: 113 - RNA, 20 nt, synthetic construct
  source 1..20: mol_type = other RNA
ggatgttcgtcctcctcaca

SEQ ID NO: 114-115 - skipped (2 entries)

SEQ ID NO: 116 - RNA, 4140 nt, synthetic construct
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 117 - RNA, 4197 nt, synthetic construct
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 118 - protein, 1398 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 119 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
ccagagcatccaaaagagtggttttagagctagaaatagcaagttaaaataaggctagtccgttatcaacttgaaaaagtggcaccgagtcggtgctttt

SEQ ID NO: 120 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
agagctcttacctacaacatgttttagagctagaaatagcaagttaaaataaggctagtccgttatcaacttgaaaaagtggcaccgagtcggtgctttt

SEQ ID NO: 121 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
gatcacgtcggccgttggcggttttagagctagaaatagcaagttaaaataaggctagtccgttatcaacttgaaaaagtggcaccgagtcggtgctttt

SEQ ID NO: 122 - DNA, 534 bp, unidentified
  source 1..534: mol_type = unassigned DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 123 - DNA, 534 bp, synthetic construct
  source 1..534: mol_type = other DNA
atgcacagctcagcactgctctgttgcctggtcctcctgactggggtgagggccagcccaggccagggcacccagtctgagaacagctgcacccacttcccaggcatcctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgatctccctgggggagaacctgaataccctcaggctgaggctacggcgctgtcatcgactccttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 124 - protein, 178 aa, unidentified
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 125 - protein, 178 aa, synthetic construct
MHSSALLCCLVLLTGVRASPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENKSKAVEQVKNAFNKLQEKGIYKAMSEFDIFINYIEAYMTMKIRN

SEQ ID NO: 126 - DNA, 669 bp, unidentified
  source 1..669: mol_type = unassigned DNA
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaagcgacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 127 - DNA, 669 bp, synthetic construct
  source 1..669: mol_type = other DNA
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaatacacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatgaggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 128 - DNA, 669 bp, synthetic construct
  source 1..669: mol_type = other DNA
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaacatacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 129 - DNA, 669 bp, synthetic construct
  source 1..669: mol_type = other DNA
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggacatcacacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaac

SEQ ID NO: 130 - protein, 223 aa, unidentified
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 131 - protein, 223 aa, synthetic construct
MACLGFQRHKAQLNLATRTWPCTLLFFLLFIPVFCKAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 132 - protein, 223 aa, synthetic construct
MACLGFQRHKAQLNLATRTWPCTLLFFLLFIPVFCKAMHVAQPAVVLASSRGIASFVCEYASPGKHTEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 133 - protein, 223 aa, synthetic construct
MACLGFQRHKAQLNLATRTWPCTLLFFLLFIPVFCKAMHVAQPAVVLASSRGIASFVCEYASPGHHTEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 134 - RNA, 22 nt, synthetic construct
  source 1..22: mol_type = other RNA
gttttagagctatgctgttttg

SEQ ID NO: 135 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 136 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 137 - DNA, 672 bp, unidentified
  source 1..672: mol_type = unassigned DNA
atggcctgcttgggcttccaaaggcataaagcccagcttaatcttgctactcgcacgtggccctgcacattgctctttttcctcctgttcattcccgtgttttgcaaggcgatgcatgtggcacaacctgccgtcgttctggcatcatcaagaggtattgctagcttcgtttgtgagtacgcctcccctggaaaagcgacggaggtgcgcgtcactgtattgcggcaagccgacagccaagttactgaagtctgcgcggcaacgtatatgatgggcaatgagctgacattccttgacgattcaatctgcacgggaacaagtagtggtaaccaggtgaatctcactattcaaggtctgagagccatggacaccggcctctacatttgtaaggtggagctgatgtatcctcccccatattatctggggatcggaaatgggacacagatatatgttattgatcccgagccatgtcccgatagtgacttcctcttgtggatacttgccgctgtgagcagtggtttgtttttttattcattcctccttacggcagtatcactttcaaaaatgctcaagaagcgaagtcctttgacaactggcgtatatgtcaaaatgccaccaacagagcccgaatgtgagaaacagttccagccgtactttattcctataaactga

SEQ ID NO: 138 - DNA, 537 bp, unidentified
  source 1..537: mol_type = unassigned DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 139-140 - skipped (2 entries)

SEQ ID NO: 141 - DNA, 8028 bp, synthetic construct
  source 1..8028: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagtcttcttttttgaagacacttcggactgtagaactctgaacctcgagcaatttaaaagaaaaggggggattggggggtacagtgcaggggaaagaatagtagacataatagcaacagacatacaaactaaagaattacaaaaacaaattacaaaaattcaaaatttctgcgttgttgtcggtgctcgttctctgctcttcacgctactgaattcatcaccggttcttcgaaggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtctagacgccaccatgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggggatccgagggcagaggaagccttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaatctaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctccgccttcgccctcagacgagtcggatctctctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 142 - DNA, 7127 bp, synthetic construct
  source 1..7127: mol_type = other DNA
gcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcgaatgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccctgatcattgtcgatcctaccatccactcgacacacccgccagggccctgccaagcttccgagctctcgatatcaaaggaggtacccaacatggtcagcaagggcgaggaactgttcaccggggtggtgcccatcctggtcgagctggacggcgacgtaaacggccacaagttcagcgtgtccggcgagggcgagggcgatgccacctacggcaagctgaccctgaagttcatctgtaccaccggcaagctgcccgtgccctggcccaccctcgtgaccaccctgacctacggcgtgcaatgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcaactcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagaagttgtctcctcctgcactgactgactgatacaatcgatttctggatccgcaggcctctgctagaagttgtctcctcctgcactgactgactgatacaatcgatttctggatccgcaggcctctgctagcttgactgactgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaac

SEQ ID NO: 143 - protein, 7 aa, synthetic construct
PKKKRKV

SEQ ID NO: 144 - protein, 7 aa, synthetic construct
PKKKRRV

SEQ ID NO: 145 - protein, 16 aa, synthetic construct
KRPAATKKAGQAKKKK

SEQ ID NO: 146-199 - skipped (54 entries)

SEQ ID NO: 200 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 201 - RNA, 70 nt, synthetic construct
  source 1..70: mol_type = other RNA
gttttagagctagaaatagcaagttaaaataaggctagtccgttatcacgaaagggcaccgagtcggtgc

SEQ ID NO: 202 - RNA, 121 nt, synthetic construct
  source 1..121: mol_type = other RNA
gttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 203 - RNA, 77 nt, synthetic construct
  source 1..77: mol_type = other RNA
gttgtagctccctgaaaccgttgctacaataaggccgtcgaaagatgtgccgcaacgctctgccttctggcatcgtt

SEQ ID NO: 204 - DNA, 1170 bp, Homo sapiens
  source 1..1170: mol_type = genomic DNA
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 205 - DNA, 1170 bp, synthetic construct
  source 1..1170: mol_type = other DNA
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcaccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 206 - DNA, 1170 bp, synthetic construct
  source 1..1170: mol_type = other DNA
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggcttttgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 207 - DNA, 1170 bp, synthetic construct
  source 1..1170: mol_type = other DNA
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctccagagacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 208 - DNA, 1170 bp, synthetic construct
  source 1..1170: mol_type = other DNA
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcaccttagcgcccactgctccagagacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagc

SEQ ID NO: 209 - DNA, 715 bp, unidentified
  source 1..715: mol_type = unassigned DNA
atgtcgaagggagaagaactgttcacaggagtcgtcccgatcctggtcgaactggacggagacgtcaacggacacaagttctcggtcagaggagaaggagaaggagacgcaacaaacggaaagctgacactgaagttcatctgcacaacaggaaagctgccggtcccgtggccgacactggtcacaacactgacatacggagtccagtgcttttcgagatacccggaccacatgaagagacacgacttcttcaagtcggcaatgccggaaggatacgtccaggaaagaacaatctcgttcaaggacgacggaacatacaagacaagagcagaagtcaagttcgaaggagacacactggtcaacagaatcgaactgaagggaatcgacttcaaggaagacggaaacatcctgggacacaagctggaatacaacttcaactcgcacaacgtctacatcacagcagacaagcagaagaacggaatcaaggcaaacttcaagatcagacacaacgtcgaagacggatcggtccagctggcagaccactaccagcagaacacaccgatcggagacggaccggtcctgctgccggacaaccactacctgtcgacacagtcggtcctgtcgaaggacccgaacgaaaagagagaccacatggtcctgctggaattcgtcacagcagcaggaatcacacacggaatggacgaactgtacaagt

SEQ ID NO: 210 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 211 - protein, 390 aa, synthetic construct
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFHLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 212 - protein, 390 aa, synthetic construct
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFCLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 213 - protein, 390 aa, synthetic construct
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSRDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 214 - protein, 390 aa, synthetic construct
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFHLSAHCSRDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 215 - protein, 238 aa, unidentified
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 216 - DNA, 7894 bp, synthetic construct
  source 1..7894: mol_type = other DNA
tatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgctagaccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaggctagctaggtggatccgaataaggcctgcaacgacacacacacgaacaagcagagccgctggacgccgactgcgggacgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcagcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagatgccaccgatgcatgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctccgccttcgccctcagacgagtcggatctctctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtata

SEQ ID NO: 217 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
tcacagagggatatctctcagttttagagctagaaatagcaagttaaaataaggctagtccgttatcaacttgaaaaagtggcaccgagtcggtgctttt

SEQ ID NO: 218 - RNA, 4140 nt, synthetic construct
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 219 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 220 - protein, 1254 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLTGGGGSVDVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 221 - DNA, 3363 bp, synthetic construct
  source 1..3363: mol_type = other DNA
atggacggctccggcggcggctcccccaagaagaagcggaaggtggaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtag

SEQ ID NO: 222 - RNA, 3921 nt, synthetic construct
  source 1..3921: mol_type = other RNA
gggaagctcagaataaacgctcaactttggccggatctgccaccatggacggctccggcggcggctcccccaagaagaagcggaaggtggaggacaagcggcccgccgccaccaagaaggccggccaggccaagaagaagaagggcggctccggcggcggcgccgccttcaagcccaaccccatcaactacatcctgggcctggacatcggcatcgcctccgtgggctgggccatggtggagatcgacgaggaggagaaccccatccggctgatcgacctgggcgtgcgggtgttcgagcgggccgaggtgcccaagaccggcgactccctggccatggcccggcggctggcccggtccgtgcggcggctgacccggcggcgggcccaccggctgctgcgggcccggcggctgctgaagcgggagggcgtgctgcaggccgccgacttcgacgagaacggcctgatcaagtccctgcccaacaccccctggcagctgcgggccgccgccctggaccggaagctgacccccctggagtggtccgccgtgctgctgcacctgatcaagcaccggggctacctgtcccagcggaagaacgagggcgagaccgccgacaaggagctgggcgccctgctgaagggcgtggccaacaacgcccacgccctgcagaccggcgacttccggacccccgccgagctggccctgaacaagttcgagaaggagtccggccacatccggaaccagcggggcgactactcccacaccttctcccggaaggacctgcaggccgagctgatcctgctgttcgagaagcagaaggagttcggcaacccccacgtgtccggcggcctgaaggagggcatcgagaccctgctgatgacccagcggcccgccctgtccggcgacgccgtgcagaagatgctgggccactgcaccttcgagcccgccgagcccaaggccgccaagaacacctacaccgccgagcggttcatctggctgaccaagctgaacaacctgcggatcctggagcagggctccgagcggcccctgaccgacaccgagcgggccaccctgatggacgagccctaccggaagtccaagctgacctacgcccaggcccggaagctgctgggcctggaggacaccgccttcttcaagggcctgcggtacggcaaggacaacgccgaggcctccaccctgatggagatgaaggcctaccacgccatctcccgggccctggagaaggagggcctgaaggacaagaagtcccccctgaacctgtcctccgagctgcaggacgagatcggcaccgccttctccctgttcaagaccgacgaggacatcaccggccggctgaaggaccgggtgcagcccgagatcctggaggccctgctgaagcacatctccttcgacaagttcgtgcagatctccctgaaggccctgcggcggatcgtgcccctgatggagcagggcaagcggtacgacgaggcctgcgccgagatctacggcgaccactacggcaagaagaacaccgaggagaagatctacctgccccccatccccgccgacgagatccggaaccccgtggtgctgcgggccctgtcccaggcccggaaggtgatcaacggcgtggtgcggcggtacggctcccccgcccggatccacatcgagaccgcccgggaggtgggcaagtccttcaaggaccggaaggagatcgagaagcggcaggaggagaaccggaaggaccgggagaaggccgccgccaagttccgggagtacttccccaacttcgtgggcgagcccaagtccaaggacatcctgaagctgcggctgtacgagcagcagcacggcaagtgcctgtactccggcaaggagatcaacctggtgcggctgaacgagaagggctacgtggagatcgaccacgccctgcccttctcccggacctgggacgactccttcaacaacaaggtgctggtgctgggctccgagaaccagaacaagggcaaccagaccccctacgagtacttcaacggcaaggacaactcccgggagtggcaggagttcaaggcccgggtggagacctcccggttcccccggtccaagaagcagcggatcctgctgcagaagttcgacgaggacggcttcaaggagtgcaacctgaacgacacccggtacgtgaaccggttcctgtgccagttcgtggccgaccacatcctgctgaccggcaagggcaagcggcgggtgttcgcctccaacggccagatcaccaacctgctgcggggcttctggggcctgcggaaggtgcgggccgagaacgaccggcaccacgccctggacgccgtggtggtggcctgctccaccgtggccatgcagcagaagatcacccggttcgtgcggtacaaggagatgaacgccttcgacggcaagaccatcgacaaggagaccggcaaggtgctgcaccagaagacccacttcccccagccctgggagttcttcgcccaggaggtgatgatccgggtgttcggcaagcccgacggcaagcccgagttcgaggaggccgacacccccgagaagctgcggaccctgctggccgagaagctgtcctcccggcccgaggccgtgcacgagtacgtgacccccctgttcgtgtcccgggcccccaaccggaagatgtccggcgcccacaaggacaccctgcggtccgccaagcggttcgtgaagcacaacgagaagatctccgtgaagcgggtgtggctgaccgagatcaagctggccgacctggagaacatggtgaactacaagaacggccgggagatcgagctgtacgaggccctgaaggcccggctggaggcctacggcggcaacgccaagcaggccttcgaccccaaggacaaccccttctacaagaagggcggccagctggtgaaggccgtgcgggtggagaagacccaggagtccggcgtgctgctgaacaagaagaacgcctacaccatcgccgacaacggcgacatggtgcgggtggacgtgttctgcaaggtggacaagaagggcaagaaccagtacttcatcgtgcccatctacgcctggcaggtggccgagaacatcctgcccgacatcgactgcaagggctaccggatcgacgactcctacaccttctgcttctccctgcacaagtacgacctgatcgccttccagaaggacgagaagtccaaggtggagttcgcctactacatcaactgcgactcctccaacggccggttctacctggcctggcacgacaagggctccaaggagcagcagttccggatctccacccagaacctggtgctgatccagaagtaccaggtgaacgagctgggcaaggagatccggccctgccggctgaagaagcggccccccgtgcggtagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaagttaaaaaaaaaaaactgaaaaaaaaaaaatttaaaaaaaaaaaatctag

SEQ ID NO: 223 - protein, 1120 aa, synthetic construct
MDGSGGGSPKKKRKVEDKRPAATKKAGQAKKKKGGSGGGAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 224 - RNA, 589 nt, synthetic construct
  source 1..589: mol_type = other RNA
gggagacccaagctggctagctcccgcagtcggcgtccagcggctctgcttgttcgtgtgtgtgtcgttgcaggccttattcggatccgccaccatgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtgatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagtctag

SEQ ID NO: 225 - RNA, 291 nt, synthetic construct
  source 1..291: mol_type = other RNA
atgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtga

SEQ ID NO: 226 - protein, 107 aa, synthetic construct
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 227 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 228 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
ttctttgtagaacttgaagtgttttagagctagaaatagcaagttaaaataaggctagtccgttatcaacttgaaaaagtggcaccgagtcggtgctttt

SEQ ID NO: 229 - protein, 178 aa, synthetic construct
MHSSALLCCLVLLTGVRASPGQGTQSENSCTHFPGNLPNMLRALRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

SEQ ID NO: 230 - DNA, 537 bp, synthetic construct
  source 1..537: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagctctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggctaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 231 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 232 - protein, 160 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 233 - protein, 160 aa, synthetic construct
SPGQGTQSENSCTHFPGNLPNMLRELRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 234 - protein, 160 aa, synthetic construct
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

(19) United States
(12) Patent Application Publication
(10) Pub. No.: US 2026/0258104 A1
(43) Pub. Date: Sep. 3, 2026

(54) ENGINEERED T CELLS

(71) Applicant: INTELLIA THERAPEUTICS, INC., Cambridge, MA (US)

(72) Inventor: Christopher RUDULIER, Cambridge, MA (US)

(21) Appl. No.: 18/878,817

(22) PCT Filed: Jun. 29, 2023
(86) PCT No.: PCT/US2023/069448

§ 371 Date: Dec. 24, 2024

Related U.S. Application Data
(60) Provisional application No. 63/367,275, filed on Jun. 29, 2022.

(51) Int. Cl. C07K 14/495; A61K 40/11; A61K 40/31; A61K 40/41; C07K 14/525; C07K 14/54; C07K 14/57; C07K 14/705; C07K 16/28; C12N 5/0783

(52) U.S. Cl. CPC C07K 14/495; A61K 40/11; A61K 40/31; A61K 40/416; A61K 40/418; C07K 14/525; C07K 14/54; C07K 14/5428; C07K 14/57; C07K 14/70521; C07K 16/2803; C12N 5/0636; C12N 2510/00

(57) ABSTRACT

The present disclosure relates to T cells engineered to comprise a heterologous nucleic acid sequence encoding a dual mutant transforming growth factor beta 1 (dmTGFB1) under control of a promoter sequence. In certain embodiments, the cells further comprise a modification, e.g., knockdown, of an endogenous nucleic acid sequence encoding a TNFA; a modification, e.g., knockdown, of an endogenous nucleic acid sequence encoding an IFNG or an IL17A; and insertion of sequence(s) encoding a regulatory T cell promoting molecule, and compositions and uses thereof.

### RELATED APPLICATIONS

The instant application hereby incorporates by reference U.S. Provisional Application No. 63/367,275, filed Jun. 29, 2022, the entire contents of which are expressly incorporated herein by reference in their entirety.

### SEQUENCE LISTING

The instant application contains a Sequence Listing which has been submitted electronically in XML format and is hereby incorporated by reference in its entirety. Said XML copy, created on Jun. 28, 2023, is named 12793_0036-00304_SL_fhfgd and is 359,125 bytes in size.

### BACKGROUND

Adaptive immunity is a defense mechanism by which the body is able to eliminate foreign pathogens. T cells are immune cells that are capable of mediating this immune response. T cell receptors (TCRs) are protein complexes on the surface of T cells that are capable of recognizing antigens. T cell diversity is derived from rearrangements of TCR alpha and beta loci.

One feature of adaptive immunity is the ability to distinguish “self” from “non-self” antigens. Autoimmune and autoinflammatory disorders are characterized by pathogenic immune responses against “self” antigens. Some rearrangements TCR alpha and beta loci generate self-reactive T cells. Owen et al., Regulatory T Cell Development in the Thymus, J Immunol 203(8) (2019). Many self-reactive T cells are eliminated by clonal deletion in the thymus, but others can escape clonal deletion and elicit deleterious immune responses. Id. Specialized T cells called regulatory T cells (Tregs) are important for “self” tolerance. Id. Tregs are capable of suppressing excessive immune responses, autoimmune responses, and undesired immune responses, for example in graft versus host disease. Id. Dysregulation of Tregs, e.g., if the number of Tregs is insufficient or if Tregs are not functioning properly, may contribute to autoimmune responses. Id.

Current therapies for treating autoimmune disorders aim to suppress the adaptive immune process or the activation of immune cells. While these therapies can suppress deleterious immune responses, e.g., autoimmune responses, they can also suppress beneficial immune responses. Treg therapies have been used to suppress antigen-specific immune responses in different diseases, including graft-versus-host disease (GvHD), in which donor cells mediate an immune attack of host tissues following hematopoietic stem cell transplantation. Pierini et al., T Cells Expressing Chimeric Antigen Receptor Promoter Immune Tolerance, JCI Insight 2(20) (2017). However, there are still “major challenges to the clinical implementation of Treg-based therapies.” Id. Thus, there remains a need for effective T cell therapies, including Treg therapies, for suppressing immune response(s), including inflammation and autoimmunity.

### SUMMARY

The present disclosure provides a dual mutant transforming growth factor beta 1 (dmTGFB1) polypeptide, for example a human dmTGFB1, and nucleic acids encoding the same; and methods and uses thereof. The present disclosure provides T cells or a population of T cells engineered to comprise a heterologous nucleic acid encoding a dmTGFB under control of a promoter sequence. The present disclosure also provides T cells or a population of T cells expressing a dmTGFB under control of a promoter sequence, and compositions and uses thereof, e.g., for suppressing immune response(s), including inflammation and autoimmunity.

In some embodiments, the cell further comprises a modification of an endogenous nucleic acid sequence encoding an interferon-gamma (IFNG) wherein the modification knocks down expression of the IFNG. In some embodiments, the cell comprises a modification of an endogenous nucleic acid sequence encoding a tumor necrosis factor alpha (TNFA) wherein the modification knocks down expression of TNFA. In some embodiments, the cell further comprises a modification of an endogenous nucleic acid sequence encoding an interleukin-17a (IL17A) wherein the modification knocks down expression of the IL17A. In some embodiments, the further modification comprises modification of an endogenous nucleic acid sequence encoding a TNFA and a modification of an endogenous nucleic acid sequence encoding an IFNG. In some embodiments, the further modification comprises modification of an endogenous nucleic acid sequence encoding a TNFA and a modification of an endogenous nucleic acid sequence encoding an IL17A. In some embodiments, the further modification comprises modification of an endogenous nucleic acid sequence encoding a TNFA, a modification of an endogenous nucleic acid sequence encoding an IFNG, and a modification of an endogenous nucleic acid sequence encoding an IL17A.

In some embodiments, the cell further comprises a heterologous nucleic acid encoding a regulatory T cell promoting molecule.

The present disclosure provides T cells or a population of T cells engineered to comprise a heterologous nucleic acid encoding a regulatory T cell promoting molecule under control of a promoter sequence; a modification of an endogenous nucleic acid sequence encoding a tumor necrosis factor alpha (TNFA) wherein the modification knocks down expression of TNFA, and a modification of an endogenous nucleic acid sequence encoding an interleukin-17a (IL17A) wherein the modification knocks down expression of the IL17A. In certain embodiments, the T cells or population of T cells do not include a modification of an endogenous nucleic acid sequence encoding an interferon-gamma (IFNG) wherein the modification knocks down expression of the IFNG.

In some embodiments, the regulatory T cell promoting molecule is selected from interleukin-10 (IL10), cytotoxic T-lymphocyte associated protein 4 (CTLA4), indoleamine 2,3-dioxygenase 1 (IDO1), ectonucleoside triphosphate diphosphohydrolase 1 (ENTPD1), 5′-nucleotidase ecto (NT5E), interleulin-22 (IL-22), amphiregulin (AREG), interleukin-35 (IL35), GARP, CD274 molecule (CD274), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), BTB domain and CNC homolog 2 (BACH2), and interleukin 2 receptor subunit alpha (IL2RA, CD25). In some embodiments, the regulatory T cell promoting molecule is IL10 or CTLA4. In some embodiments, the regulatory T cell promoting molecules are IL10 and CTLA4.

In some embodiments, the T cells or population of T cells are engineered to comprise a heterologous nucleic acid encoding dmTGFB1 and IL10, each under control of a promoter sequence; a modification of an endogenous nucleic acid sequence encoding TNFA wherein the modification knocks down expression of TNFA; and a modification of an endogenous nucleic acid sequence encoding IFNG wherein the modification knocks down expression of the IFNG, or a modification of an endogenous nucleic acid sequence encoding IL17A wherein the modification knocks down expression of the IL17A. In some embodiments, the cell comprises modifications of an endogenous nucleic acid sequence encoding each an IFNG and an IL17A, wherein the modification knocks down expression of each of the IFNG and the IL17A.

In some embodiments, the T cells or population of T cells are engineered to comprise a heterologous nucleic acid encoding dmTGFB1 and CTLA4, each under control of a promoter sequence; and a modification of an endogenous nucleic acid sequence encoding TNFA wherein the modification knocks down expression of TNFA. In some embodiments, the T cells or population of T cells comprises a further modification of an endogenous nucleic acid sequence encoding IFNG wherein the modification knocks down expression of the IFNG. In some embodiments, the T cells or population of T cells comprises a further modification of an endogenous nucleic acid sequence encoding IL17A wherein the modification knocks down expression of the IL17A. In some embodiments, the T cells or population of T cells comprises a modification of an endogenous nucleic acid sequence encoding each IFNG and TNFA, wherein the modifications knock down expression of each the IFNG and TNFA. In some embodiments, the T cells or population of T cells comprises a modification of an endogenous nucleic acid sequence encoding each IL17A and TNFA, wherein the modifications knock down expression of each the IL17A and TNFA. In some embodiments, the T cells or population of T cells comprises modifications of an endogenous nucleic acid sequence encoding each a TNFA, an IFNG, and an IL17A wherein the modification knocks down expression of each of the TNFA, IFNG, and IL17A.

In some embodiments, the T cells or population of T cells are engineered to comprise heterologous nucleic acid sequences encoding dmTGFB1, IL10, and CTLA4, each under control of a promoter sequence; and a modification of an endogenous nucleic acid sequence encoding TNFA wherein the modification knocks down expression of TNFA. In some embodiments, the T cells or population of T cells comprises a further modification of an endogenous nucleic acid sequence encoding IFNG wherein the modification knocks down expression of the IFNG. In some embodiments, the T cells or population of T cells comprises a further modification of an endogenous nucleic acid sequence encoding IL17A wherein the modification knocks down expression of the IL17A. In some embodiments, the T cells or population of T cells comprises modifications of an endogenous nucleic acid sequence encoding each IFNG and IL17A, wherein the modifications knock down expression of each the IFNG and IL17A.

In some embodiments, the T cells or population of T cells are further engineered to comprise a modification, e.g., knockdown, of an endogenous nucleic acid sequence encoding an interleukin-2 (IL2), an interleukin 6 (IL6), a perforin 1 (PRF1), a granzyme A (GZMA), a granzyme B (GZMB), Fas ligand (FasL, NF superfamily, member 6), ryanodine receptor 2 (RYR2), and colony stimulating factor 2 (CSF2). In some embodiments, the T cells or population of T cells are further engineered to comprise a modification, e.g., knockdown, of an endogenous nucleic acid sequence encoding RYR2.

In some embodiments, the T cells or population of T cells are further engineered to comprise a modification, e.g., knockdown, of an endogenous nucleic acid sequence encoding an endogenous T cell receptor (TCR).

In some embodiments, the T cells or population of T cells are further engineered to comprise a heterologous coding sequence for a targeting receptor under control of a promoter sequence. In some embodiments, the targeting receptor comprises a chimeric antigen receptor (CAR) or a T cell receptor (TCR). In some embodiments, the targeting receptor is targeted to a ligand selected from mucosal vascular addressin cell adhesion molecule 1 (MADCAM1), tumor necrosis factor alpha (TNFA), CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), desmoglein 3 (DSG3), and MHC class I HLA-A (HLA-A*02). In some embodiments, the targeting receptor is targeted to mucosal vascular addressin cell adhesion molecule 1 (MADCAM1). In some embodiments, the targeting receptor is targeted to tumor necrosis factor alpha (TNFA).

In some embodiments, at least 30%, 35%, preferably at least 40%, 45%, 50%, 55%, 60%, 65%, 70%, 75%, 80%, 85%, 90%, or 95% of the population of T cells comprises an insertion of the sequence encoding a dmTGFB, e.g., as assessed by sequencing, e.g., NGS. In certain embodiments, further modifications are present.

In some embodiments, at least 30%, 35%, preferably at least 40%, 45%, 50%, 55%, 60%, 65%, 70%, 75%, 80%, 85%, 90%, or 95% of the population of T cells comprises an insertion of the sequence encoding a regulatory T cell promoting molecule, e.g., as assessed by sequencing, e.g., NGS.

In some embodiments, at least 50%, 55%, 60%, 65%, preferably at least 70%, 75%, 80%, 85%, 90%, or 95% of the population of T cells comprises a modification, e.g., knockdown, in an IFNG sequence, e.g., as assessed by sequencing, e.g., NGS. In some embodiments, at least 50%, 55%, 60%, 65%, preferably at least 70%, 75%, 80%, 85%, 90%, or 95% of the population of T cells comprises a modification, e.g., knockdown, in an TNFA sequence, e.g., as assessed by sequencing, e.g., NGS.

In some embodiments, at least 50%, 55%, 60%, 65%, preferably at least 70%, 75%, 80%, 85%, 90%, or 95% of the population of T cells comprises a modification, e.g., knockdown, in an IL17A sequence, e.g., as assessed by sequencing, e.g., NGS.

In some embodiments, at least 50%, 55%, 60%, 65%, preferably at least 70%, 75%, 80%, 85%, 90%, or 95% of the population of T cells comprises a modification, e.g., knockdown, in a TCR sequence, e.g., as assessed by sequencing, e.g., NGS. In some embodiments, at least 40%, 45%, 50%, 55%, 60%, 65%, 70%, 75%, 80%, 85%, 90%, or 95% of the population of T cells comprises an insertion of the sequence encoding a targeting receptor, e.g., a CAR, e.g., as assessed by sequencing, e.g., NGS.

The modifications described herein for knocking down expression of a gene may comprise one or more of an insertion, deletion, or substitution. The heterologous sequences described herein may be incorporated into expression construct(s). Multiple heterologous sequences may be incorporated into a single expression construction or into separate expression constructs. The heterologous sequences described herein may be incorporated into episomal expression construct(s). The heterologous sequences described herein may be inserted into the genome, e.g., an untargeted insertion or a targeted insertion. In some embodiments, the targeted insertion is into a site selected from a TCR gene locus, a TNF gene locus, an IFNG gene locus, IL17A gene locus, IL6 gene locus, IL2 gene locus, an adeno-associated virus integration site 1 (AAVS1) locus.

Pharmaceutical compositions and uses of the engineered T cells are also provided herein. In some embodiments, the engineered T cells and pharmaceutical compositions thereof may be administered to a subject in need of immunosuppression. In some embodiments, the engineered T cells and pharmaceutical compositions thereof may be useful in the treatment of an immune disorder or an autoimmune disease, e.g., ulcerative colitis, Crohn's disease, rheumatoid arthritis, psoriasis, multiple sclerosis, systemic lupus erythematosus, type 1 diabetes, and graft versus host disease (GvHD).

In some embodiments, the insertion of sequence(s) or the modification, e.g., knockdown, of sequence(s) described herein may be mediated by guide RNAs in combination with an RNA-guided DNA binding agent, e.g., Cas nuclease. In some embodiments, the insertion of sequence(s) or the knockdown of sequence(s) described herein may be mediated by another suitable gene editing system, e.g., zinc finger nuclease (ZFN) system or transcription activator-like effector nuclease (TALEN) system.

### BRIEF DESCRIPTION OF THE DRAWINGS

FIG. 1 shows total TGF-β1 quantified in culture supernatants.

FIG. 2 shows active TGF-β1 quantified in culture supernatants.

FIG. 3 shows recent suppression of autologous T cell proliferation by T cells overexpressing wild-type or mutant TGF-β1 as measured by CTV dilution.

FIG. 4 Shows the probability of survival over time after injection of NOG mice with engineered CD3+CD4+ cells.

FIG. 5 shows the percent suppression of cell proliferation by engineered T cells as measured by CTV dilution.

FIG. 6 shows production of total TGF-β1 (pg/ml) by transduced cells upon cell stimulation.

FIG. 7 shows production of active TGF-β1 (pg/ml) by transduced cells upon cell stimulation.

FIG. 8 shows IFN-γ production (pg/ml) of transduced cells upon cell stimulation.

FIG. 9 shows TNF-α production (pg/ml) of transduced cells upon cell stimulation.

FIG. 10 shows IL-17a production (pg/ml) of transduced cells upon cell stimulation.

FIG. 11 shows IL-2 production (pg/ml) of transduced cells upon cell stimulation.

FIG. 12 shows IL-10 production (pg/ml) of transduced cells upon cell stimulation.

FIG. 13 shows IL-13 production (pg/ml) of transduced cells upon cell stimulation.

FIG. 14 shows the percent of mice in each cohort surviving at each timepoint after injection with engineered suppressive T cells.

FIG. 15 shows the percent of mice in each cohort surviving at each timepoint after injection with engineered suppressive T cells.

FIG. 16 shows percent of initial body weight individual mice in treatment groups.

FIG. 17 shows colon length in cm.

FIG. 18 shows the percent of Tregs recovered from each tissue stained with Cell Trace Violet.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended embodiments.

The section headings used herein are for organizational purposes only and are not to be construed as limiting the desired subject matter in any way. In the event that any material incorporated by reference contradicts any term defined in this specification or any other express content of this specification, this specification controls.

### I. Definitions

Before describing the present teachings in detail, it is to be understood that the disclosure is not limited to specific compositions or process steps, as such may vary. It should be noted that, as used in this specification and the appended embodiments, the singular form “a”, “an” and “the” include plural references unless the context clearly dictates otherwise. Thus, for example, reference to “a conjugate” includes a plurality of conjugates and reference to “a cell” includes a plurality or population of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or,” unless the context clearly indicates otherwise.

The term “about”, when used before a list or range, modifies each member of the list or each endpoint of the range. The term “about” or “approximately” means an acceptable error for a particular value as determined by one of ordinary skill in the art, which depends in part on how the value is measured or determined. The term “about” is used herein to mean within the typical ranges of tolerances in the art. For example, “about” can be understood as about 2 standard deviations from the mean. In certain embodiments, about means+/−10%. In certain embodiments, about means+/−5%.

In some embodiments a population of cells refers to a population of at least 10\hat{ }3, 10\hat{ }4, 10\hat{ }5 or 10\hat{ }6 cells, preferably 10\hat{ }7, 2×10\hat{ }7, 5×10\hat{ }7, or 10\hat{ }8 cells.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20 nucleotide nucleic acid molecule” means that 17, 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

In the event of a conflict between a sequence in the application and an indicated accession number or position in an accession number, the sequence in the application predominates.

Unless stated otherwise, the following terms and phrases as used herein are intended to have the following meanings.

As used herein, “knockdown” refers to a decrease in expression of a particular gene product (e.g., full-length or wild-type mRNA, protein, or both), e.g., in a cell, population of cells, tissue, or organ, by gene editing. In some embodiments, gene editing can be assessed by sequence, e.g., next generation sequencing (NGS). Expression may be decreased by at least 70%, 75%, 80%, 85%, 90%, 95%, or to below the level of detection of the assay as compared to a suitable control, e.g., wherein the gene sequence has not been modified. Knockdown of a protein can be measured by detecting the amount of the protein from a tissue, cell population, or fluid of interest. Methods for measuring knockdown of mRNA are known and include sequencing of mRNA isolated from a tissue or cell population of interest. Flow cytometry analysis is a known method for measuring knockdown of protein expression. For secreted proteins, knockdown may be assessed in a fluid such as tissue culture media or blood, or serum or plasma derived therefrom. In some embodiments, “knockdown” may refer to some loss of expression of a particular gene product, for example a decrease in the amount of full-length, wild-type mRNA transcribed or translated into full-length protein, or a decrease in the amount of protein expressed by a population of cells. It is well understood what changes in an mRNA sequence would result in decreased expression of a wild-type or full-length protein. In some embodiments, “knockdown” may refer to some loss of expression of a particular gene product, for example, an IFNG or TNFA gene product in a body fluid or tissue culture media. A modification of an endogenous nucleic acid sequence, e.g., encoding IFNG or TNFA, may result in a knockdown.

As used herein, “T cell receptor” or “TCR” refers to a receptor in a T cell. In general, a TCR is a heterodimer receptor molecule that contains two TCR polypeptide chains, α and β. α and β chain TCR polypeptides can complex with various CD3 molecules and elicit immune response(s), including inflammation and autoimmunity, after antigen binding. As used herein, a knockdown of TCR refers to a knockdown of any TCR gene in part or in whole, e.g., deletion of part of the TRBC1 gene, alone or in combination with knockdown of other TCR gene(s) in part or in whole.

“TRAC” is used to refer to the T cell receptor α chain. A human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

“TRBC” is used to refer to the T-cell receptor β-chain, e.g., TRBC1 and TRBC2. “TRBC1” and “TRBC2” refer to two homologous genes encoding the T-cell receptor β-chain, which are the gene products of the TRBC1 or TRBC2 genes.

A human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1.

A human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

As used herein, an “immune response” refers to one or more immune system reaction(s), e.g., increased production or activity of immune system cells, such as, but not limited to T cells, B cells, natural killer cells, monocytes, neutrophils, eosinophils, basophils, mast cells, erythrocytes, dendritic cells, antigen presenting cells, macrophages, or phagocytes as compared to an unstimulated control immune system. Exposure of the immune system to an antigen, e.g., a foreign or self-antigen such as but not limited to a pathogen (microorganism, virus, prion, fungus, etc.), an allergen (dust, pollen, dust mite, etc.), a toxin (chemical, drug, etc.), or physiological changes (hypercholesterolemia, obesity, organ transplant, etc.), may cause an immune response. An immune response can also include a response in which donor cells mediate an immune attack of host tissues following hematopoietic stem cell transplantation in GvHD. The immune response may result in inflammation. The immune response may target, attack, remove, or neutralize the antigen, e.g., foreign or self. The immune response may or may not be desirable. The immune response may be acute or chronic. The immune response may damage the cell, tissue, or organ against which the immune response is mounted.

As used herein, an “autoimmune response” refers to one or more immune system reaction(s) to a self-antigen, e.g., produced by a subject's own cells, tissues, or organs. The autoimmune response may result in increased production or activity of immune system cells, such as, but not limited to T cells, B cells, natural killer cells, monocytes, neutrophils, eosinophils, basophils, mast cells, erythrocytes, dendritic cells, antigen presenting cells, macrophages, or phagocytes as compared to a suitable control, e.g., a healthy control. The autoimmune response may result in inflammation, e.g., prolonged inflammation, or lead to an autoimmune disease. The autoimmune response may target, attack, remove, or neutralize the self-antigen produced by the subject's own cells, tissues, or organs, which may lead to an autoimmune disease.

As used herein, “suppressing” an immune response(s) refers to decreasing or inhibiting the level of one or more immune system reaction(s), e.g., the production or activity of the immune system cells compared to a suitable control, e.g., not treated with or prior to treatment with the engineered T cell described herein. “Suppressing” an immune response(s) may refer to decreased production or activity of the immune system cells compared to a suitable control, e.g., not treated with or prior to treatment with the engineered T cell described herein. “Suppressing” an immune response may refer to increasing immune tolerance. For example, production or activity of the immune system cells may be measured by cell count, e.g., lymphocyte count or spleen cell count; cell activity, e.g., T cell assay; or gene or protein expression, e.g., biomarker expression; wherein the production or activity is decreased by 50%, 55%, 60%, 65%, 70%, 75%, 80%, 85%, 90%, 95%, or below the level of detection of the assay compared to a suitable control, e.g., not treated with or prior to treatment with the engineered T cell described herein.

As used herein, an “autoimmune disease” or “autoimmune disorder” refers to a condition characterized by pathological immune responses to a subject's own antigens, cells, tissues, or organs. Examples of autoimmune diseases and disorders include, but are not limited to: ulcerative colitis, Crohn's disease, rheumatoid arthritis, psoriasis, multiple sclerosis, systemic lupus erythematosus, and type 1 diabetes. In some embodiments, the engineered T cells have autologous or allogenic use.

As used herein, an “immune disorder” is understood as a disease or condition characterized by a pathological or undesired immune response in a subject. In certain embodiments, an immune disorder is an autoimmune disease. In certain embodiments, an immune disorder is GvHD. In certain embodiments, a subject with an immune disorder is in need of suppression of an immune response. In certain embodiments, a subject with an immune disorder is in need of an increase in immune tolerance.

A “T cell” plays a central role in the immune response following exposure to an antigen. T cells can be naturally occurring or non-natural, e.g., when T cells are formed by engineering, e.g., from a stem cell or by transdifferentiation, e.g., reprogramming a somatic cell. T cells can be distinguished from other lymphocytes by the presence of a T cell receptor on the cell surface. Included in this definition are conventional adaptive T cells, which include helper CD4+ T cells, cytotoxic CD8+ T cells, memory T cells, and regulatory CD4+ T cells, and innate-like T cells including natural killer T cells, mucosal associated invariant T cells, and gamma delta T cells. In some embodiments, T cells are CD4+. In some embodiments, T cells are CD3+/CD4+. In some embodiments, T cells are CD8+.

A “regulatory T cell” or “Treg” refers to a specialized T cell that plays a central role in suppressing excessive immune response(s), including inflammation and autoimmunity. Tregs can be naturally occurring or non-natural, e.g., when Tregs are formed by engineering, including by insertion of a sequence encoding a dmTGFB1 molecule. Tregs when formed by engineering can include further modifications e.g., by modifications, e.g., knockdowns, of endogenous nucleic acid sequences encoding IFNG, IL17A, and TNFA and insertion of at least one sequence(s) encoding a regulatory T cell promoting molecule. A naturally occurring Treg or natural Treg or nTreg, also sometimes referred to as a thymus Treg or tTreg, is a specialized T cell that typically develops in the thymus gland and functions by suppressing excessive immune response(s). In some embodiments, a cell such as a conventional T cell or population of conventional T cells, e.g., a population of T cells not enriched for the presence of nTreg cells, may be engineered by insertion of a sequence encoding a dmTGFB1 molecule, and optionally further modifying endogenous nucleic sequences encoding, e.g., TNFA and IFNG, e.g., knocking down nucleic sequences encoding TNFA and IFNG, and insertion of sequence(s) encoding a regulatory T cell promoting molecule into the cell to exhibit the phenotypic characteristics and suppressive functions of a regulatory T cell, and these may be referred to as transduced or “engineered” T cells. In some embodiments, an engineered T cell comprises insertion of a sequence encoding a dmTGFB1 molecule; a modification of an endogenous nucleic acid sequence encoding an IFNG and a modification of an endogenous nucleic acid sequence encoding a TNFA, and insertion of a heterologous regulatory T cell promoting molecule such as IL10 or CTLA4. The modification of an endogenous nucleic acid sequence, e.g., a modification knocks down expression of an endogenous gene, may comprise or consist of one or more indel or substitution mutations in the genomic sequence.

As used herein, a dmTGFB1 is a mutant TGFB1 based on a wild type TGFB1 from any species including, for example, human, mouse, rat, or cynomolgus TGFB1. In certain embodiments, the dmTGFB1 is human dmTGFB1, i.e., a mutant TGFB1 relative to a wild type human TGFB1. Coding and amino acid sequences for wild type TGFB1 are readily available in sequence databases, including NCBI, and exemplary sequences can be found under accession numbers NM_000660.7 and NP_000651.3 (human), NM_011577.2 and NP_035707.1 (mouse), NM_021578.2 and NP_067589.1 (rat), and XM_005589339.3 and XP 005589396.1 (cynomolgus). Each accession number is incorporated by reference in the version available as of the date of filing of the instant application. The ability to map mutations onto wild type sequences is well within the ability of those of skill in the art.

Transforming growth factor beta-1 (TGFB1) is synthesized as a large precursor molecule. TGFB1 preprotein contains a signal peptide of 29 amino acids that is proteolytically cleaved. TGFB1 is further cleaved after amino acid 278 to form latency-associated peptide (LAP) and active TGFB1. LAP dimerizes with interchain disulfide links at C223 and C225. TGFB1 can be secreted as an inactive small latent complex that consists of a mature TGF-β1 homodimer non-covalently associated with an LAP homodimer at LAP residues 153-L59. LAP shields the type II receptor binding sites in the mature TGFB1. Most cells secrete TGFB1 as a large latent complex (LLC) of TGF-β1/LAP covalently bound between C33 in the LAP chains and latent TGFB-binding protein (LTBP). LTBPs facilitate TGFB1 folding, secretion, and possibly targeting to the extracellular matrix. Activation of the LLC occurs via the N-terminal domain of LTBP binding to the extracellular matrix.

Camurati-Engelmann Disease (CED) results from domain-specific heterozygous mutations in the transforming growth factor-beta-1 gene (TGFB1; OMIM entry 190180, incorporated by reference in the version available on the date of the filing of the application) on chromosome 19q13. Mutations reported in Camurati-Engelmann Disease families include (from Janssen et al., 2006. J Med Genet. 43:1.):

| Exon | DNA mutation | Protein mutation |
|---|---|---|
| 1 | 28_36 duplication | L10-L12 duplication |
| 1 | 241T → C | Y81H |
| 2 | 463C → T | R156C |
| 4 | 652C → T | R218C |
| 4 | 653G → A | R218H |
| 4 | 664C → G | H222D |
| 4 | ? | C223S |
| 4 | 667T → C | C223R |
| 4 | 667T → G | C223G |
| 4 | 673T → C | C225R |

As shown in the table, the majority of pathogenic variants in individuals with CED result in single amino-acid substitutions in the carboxy terminus of TGFB1 latency-associated peptide (LAP). The substitutions are near the site of interchain disulfide bonds between the LAP homodimers. These pathogenic variants disrupt dimerization of LAP and binding to active TGFB1 (Walton et al., 2010. J Biol Chem. 285:17029-37), leading to increased active TGFB1 release from the cell. Walton et al. further demonstrated that the stability of the resultant large latent complex is dependent upon covalent dimerization of LAP, which is facilitated by key residues (F198, D199, V200, L208, F217, and L219) at the dimer interface. R218H mutated fibroblasts from individuals with CED showed increased active TGFB1 in the cell media compared to normal fibroblasts (Saito et al., 2001. J Biol Chem. 276:11469-72). In vitro analysis of R218C, H222D, and C225R mutated constructs also showed increased active TGF-B1 in the medium of transfected cells. In contrast, the Leu11_Leu13dup and Tyr81His pathogenic variants caused a decrease in the amount of TGFB1 secreted. However, in a luciferase reporter assay specific for TGFB1-induced transcriptional response, the mutated cells showed increased luciferase activity, suggesting intracellular activation of the receptor (Janssens et al., 2003. J Biol Chem. 278:7718-24).

As used herein, in certain embodiments, a dmTGFB1 is a TGFB1 comprising mutations at two, or more, amino acid positions relative to a wild type TGFB1 that reduce the stability of the resultant large latent complex that is dependent upon covalent dimerization of LAP relative to each TGFB1 single mutant alone. Mutations at two or more amino acid positions can include mutations at positions selected from F198, D199, V200, L208, F217, L219, R218, H222, C223, and C225 relative to a wild type human TGFB1.

In certain embodiments, a dmTGFB1 is a is a TGFB1 comprising mutations at two, or more, amino acid positions relative to a wild type TGFB1 at positions selected from F198, D199, V200, L208, F217, L219, R218, H222, C223, and C225 relative to a wild type human TGFB1 wherein when the dmTGFB1 is produced it is secreted from a cell in which TGFB1 is typically expressed. In some embodiments, the dmTGFB1 comprises mutations that are naturally occurring, e.g., in CED. In some embodiments, the dmTGFB1 comprises mutations that are non-naturally occurring, e.g., not naturally occurring in CED.

In certain embodiments, a human dmTGFB1 comprises mutations at 2, or more, amino acid positions selected from R218, H222, C223, and C225, relative to a wild type human TGFB1.

In certain embodiments, a human dmTGFB1 comprises 2, or more, mutations selected from R218C/H, H222D, C223S/R/G, and C225R, relative to a wild type human TGFB1.

In certain embodiments, a human dmTGFB1 comprises 2, or more, mutations selected from R218C/H and C225R, relative to a wild type human TGFB1.

The dmTGFB1 provided herein has more free, active dmTGFB1 than either of the single mutants alone. In certain embodiments, the increase in active dmTGFB1 is at least additive as compared to the activity of the single mutants alone. In certain embodiments, the increase in active dmTGFB1 is significantly more than additive as compared to the activity of the single mutants alone. Methods to determine levels of total TGFB1 and active TGFB1 are known in the art using commercially available kits (e.g., LEGEND MAX Total TGF-β1 ELISA kit (BioLegend, Cat. 436707), and a LEGEND MAX Free Active TGF-β1 ELISA kit) and as provided in the Examples below.

As used herein, “regulatory T cell promoting molecules” refer to molecules that promote the conversion of conventional T cells to regulatory T cells including immunosuppressive molecules and Treg transcription factors. Further, regulatory T cell promoting molecules refer also to molecules that endow conventional T cells with regulatory activity, including Treg-associated immunosuppressive molecules and transcription factors. Regulatory T cell promoting molecules can be used in conjunction with dmTGFB1 promote the conversion of conventional T cells to regulatory T cells or to that endow conventional T cells with regulatory activity. Examples of immunosuppressive molecules to be used in combination with dmTGFB1 include, but are not limited to, interleukin-10 (IL10), cytotoxic T-lymphocyte associated protein 4 (CTLA4), indoleamine 2,3-dioxygenase 1 (IDO1), ectonucleoside triphosphate diphosphohydrolase 1 (ENTPD1), 5′-nucleotidase ecto (NT5E), interleukin-22 (IL22), amphiregulin (AREG), interleukin-35 (IL35), leucine rich repeat containing 32 (GARP), CD274 molecule (CD274), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), BTB domain and CNC homolog 2 (BACH2), and interleukin 2 receptor subunit alpha (IL2RA, CD25). In some embodiments, regulatory T cell promoting molecules may be used in specific combinations, e.g., IL10 and CTLA4, ENTPD1 and NT5E, and IL22 and AREG. In particular, an IL10 and CTLA4 combination for use in conjunction with dmTGFB1 is provided herein. In some embodiments, the expression of immunosuppressive molecules may be promoted by the expression of transcription factors such as FoxP3, Helios, Eos, IRF4, Lef1, or BACH2.

In some embodiments, a conventional T cell may be engineered to modify, insert, or delete sequences in the genome, and the “engineered” T cell exhibits one or more phenotypic characteristics and suppressive functions of a natural regulatory T cell. For example, the “engineered” T cell exhibits suppressive activity in a mixed lymphocyte reaction assay as provided in Example 2.4 below, or preferably is capable of inhibiting graft versus host disease in the mouse model presented in Example 3.2 below, preferably in a statistically significant manner (see also, e.g., Parmar et al., Ex vivo fucosylation of third-party human regulatory T cells enhances anti-graft-versus-host disease potency in vivo, Blood 125(9) (2015)). In some embodiments, the “engineered” T cell is a conventional T cell that that has been modified with the insertion of coding sequences for regulatory T cell promoting molecules, and with modification, e.g., knockdown, of expression of pro-inflammatory cytokines, e.g., TNFA in combination with one or both of IFNG and IL17A. In some embodiments, the starting T cell population for engineering is not enriched for the presence of natural Tregs, e.g., the starting T cell population has less than 20% natural Tregs.

As used herein, a “pro-inflammatory” molecule, e.g., cytokine, increases an immune response as described herein, e.g., reduces the efficacy of a Treg in the mouse model of graft-versus-host disease presented in Example 3.2 in a dose responsive manner. Examples of pro-inflammatory molecules include, but are not limited to, IFNG, TNFA, IL17A, IL6, IL2, perforin 1 (PRF1), granzyme A (GZMA), granzyme B (GZMB), Fas ligand (FasL, NF superfamily, member 6), ryanodine receptor 2 (RYR2), and colony stimulating factor 2 (CSF2).

As used herein, “targeting receptor” refers to a receptor present on the surface of a cell, e.g., a T cell, to permit binding of the cell to a target site, e.g., a specific cell or tissue in an organism. Targeting receptors include, but are not limited to a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion of a protein, e.g., mucosal addressin cell adhesion molecule-1 (MADCAM-1), TNFA, CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), demoglein 3 (DSG3), and MHC class I HLA-A (HLA-A*02).

As used herein, a “chimeric antigen receptor” refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837, the corresponding portions of the contents of each of which are incorporated herein by reference). A reversed universal CAR that promotes binding of an immune cell to a target cell through an adaptor molecule (see, e.g., WO2019238722, the contents of which are incorporated herein in their entirety) is also contemplated. CARs can be targeted to any antigen to which an antibody can be developed and are typically directed to molecules displayed on the surface of a cell or tissue to be targeted. In some embodiments, the CAR is capable of targeting engineered T cells to the gastrointestinal tract, e.g., the CAR targets MAdCAM-1. In some embodiments, the CAR is capable of targeting engineered T cells to tissues comprising endothelial cells, e.g., the CAR targets VCAM-1, e.g., for suppressing immune responses in disorders such as Crohn's disease and multiple sclerosis. In some embodiments, the CAR is capable of targeting engineered T cells to endothelial cells, e.g., the CAR targets CEACAM6, e.g., for suppressing immune responses in disorders such as Crohn's disease. In some embodiments, the CAR is capable of targeting engineered T cells to pre-B cells, e.g., the CAR targets CD19, e.g., for suppressing immune responses in disorders such as multiple sclerosis and systemic lupus erythematosus. In some embodiments, the CAR is capable of targeting engineered T cells to B lymphocytes, e.g., the CAR targets CD20, e.g., for suppressing immune responses in disorders such as multiple sclerosis and systemic lupus erythematosus. In some embodiments, the CAR is capable of targeting engineered T cells to an inflammatory tissue, e.g., the CAR targets TNFA, e.g., for suppressing immune responses in disorders such as rheumatoid arthritis, inflammatory bowel disease, ulcerative colitis, or Crohn's disease. In some embodiments, the CAR is capable of targeting engineered T cells to an inflammatory tissue, e.g., the CAR targets TGF-b1 e.g., for suppressing immune responses in disorders such as inflammatory bowel disease, ulcerative colitis, or Crohn's disease. In some embodiments, the CAR is capable of targeting engineered T cells to a neurological tissue, e.g., the CAR targets MBP, MOG, or PLP1 e.g., for suppressing immune responses in disorders such as multiple sclerosis. In some embodiments, the CAR is capable of targeting engineered T cells to tissues comprising mature B lymphocytes, e.g., the CAR targets TNFRSF17, e.g., for suppressing immune responses in disorders such as systemic lupus erythematosus. In some embodiments, the CAR is capable of targeting engineered T cells to synovial tissue, e.g., the CAR targets citrullinated vimentin e.g., for suppressing immune responses in disorders such as rheumatoid arthritis. In some embodiments, the CAR targets dipeptidyl peptidase like 6 (DPP6), solute carrier family 2 member 2 (SCL2A2), glutamate decarboxylase (GAD2), demoglein 3 (DSG3), or MHC class I HLA-A (HLA-A*02). Additional CAR targets, e.g., inflammatory antigens, are known in the art. See, e.g., WO2020092057A1, the contents of which are incorporated herein in their entirety.

As used herein, “treatment” refers to any administration or application of a therapeutic for disease or disorder in a subject, and includes inhibiting the disease, arresting its development, relieving one or more symptoms of the disease, curing the disease, preventing one or more symptoms of the disease, or preventing reoccurrence of one or more symptoms of the disease. Treating an autoimmune or inflammatory response or disorder may comprise alleviating the inflammation associated with the specific disorder resulting in the alleviation of disease-specific symptoms. Treatment with the engineered T cells described herein may be used before, after, or in combination with additional therapeutic agents, e.g., anti-inflammatory agents, immunosuppressive agents, or biologics for treatment of autoimmune disorders, e.g., Remicade, Humira.

A “promoter” refers to a regulatory region that controls the expression of a gene to which the regulatory region is linked.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy, 2′ halide, or a 2′-O-(2-methoxyethyl) (2′-O-moe) substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Nucleic acid includes “unlocked nucleic acid” or UNA. RNA and DNA can have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

As used herein, “open reading frame” or “ORF” of a gene refers to a sequence consisting of a series of codons that specify the amino acid sequence of the protein that the gene codes for. The ORF generally begins with a start codon (e.g., ATG in DNA or AUG in RNA) and ends with a stop codon, e.g., TAA, TAG or TGA in DNA or UAA, UAG, or UGA in RNA.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” or “guide region” or “targeting sequence” or “spacer” or “spacer sequence” and the like refers to a sequence within a gRNA that is complementary to a target sequence and functions to direct a gRNA to a target sequence for binding or modification (e.g., cleavage) by an RNA-guided nickase. A guide sequence can be 20 nucleotides in length, e.g., in the case of Streptococcus pyogenes (i.e., Spy Cas9 (also referred to as SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. A guide sequence can be 20-25 nucleotides in length, e.g., in the case of Nme Cas9, e.g., 20-, 21-, 22-, 23-, 24- or 25-nucleotides in length. For example, a guide sequence of 24 nucleotides in length can be used with Nme Cas9, e.g., Nme2 Cas9.

In some embodiments, the target sequence is in a genomic locus or on a chromosome, for example, and is complementary to the guide sequence. In some embodiments, the degree of complementarity or identity between a guide sequence and its corresponding target sequence may be about 75%, 80%, 85%, 90%, 95%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the degree of complementarity or identity between a guide sequence and its corresponding target sequence is at least 80%, 85%, preferably 90%, or 95%, for example when, the guide sequence comprises a sequence 24 contiguous nucleotides. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, preferably 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides. Mismatch positions are known in the art as provided in, for example, PAM distal mismatches tend to be better tolerated than PAM proximal matches. Mismatch tolerances at other positions are known in the art (see, e.g., Sternberg et al., 2015, Nature: 527:110-113)

As used herein, a “target sequence” or “genomic target sequence” refers to a sequence of nucleic acid in a target gene genomic locus, in either the positive or the negative strand, that has complementarity to the guide sequence of the gRNA, i.e., that is sufficiently complementary to the guide sequence of the gRNA to permit specific binding of the guide to the target sequence. The interaction of the target sequence and the guide sequence directs an RNA-guided DNA binding agent to bind, and potentially nick or cleave (depending on the activity of the agent), within the target sequence. The specific length of the target sequence and the number of mismatches possible between the target sequence and the guide sequence depend, for example, on the identity of the Cas9 nuclease being directed by the gRNA. Target sequences for Cas proteins include both the positive and negative strands of genomic DNA (i.e., the sequence given and the sequence's reverse complement), as a nucleic acid substrate for a Cas protein is a double stranded nucleic acid. Accordingly, where a guide sequence is said to be “complementary to a target sequence,” it is to be understood that the guide sequence may direct an RNA-guided DNA binding agent (e.g., dCas9 or impaired Cas9) to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

Target sequences for RNA-guided DNA binding agents include both the positive and negative strands of genomic DNA (i.e., the sequence given and the sequence's reverse complement), as a nucleic acid substrate for an RNA-guided DNA-binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA-binding agent” means a polypeptide or complex of polypeptides having RNA and DNA binding activity, or a DNA-binding subunit of such a complex, wherein the DNA binding activity is sequence-specific and depends on the sequence of the RNA. The term RNA-guided DNA-binding agent also includes nucleic acids encoding such polypeptides. Exemplary RNA-guided DNA-binding agents include Cas cleavases/nickases. Exemplary RNA-guided DNA-binding agents may include inactivated forms thereof (“dCas DNA-binding agents”), e.g., if those agents are modified to permit DNA cleavage, e.g., via fusion with a FokI cleavase domain. “Cas nuclease”, as used herein, encompasses Cas cleavases and Cas nickases. Cas cleavases and Cas nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated), for example if those agents are modified to permit DNA cleavage, or with a C to T deaminase or A to G deaminase activity. Cas nickases include nucleases in which one of the RuvC or HNH domain of the Cas protein, such that only a single strand is cleaved by the nuclease. In some embodiments, the RNA-guided DNA-binding agent comprises a deaminase region and an RNA-guided DNA nickase, such as a Cas9 nickase. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g, K810A, K1003A, R1060A variants), and eSPCas9 (1.1) (e.g., K848A, K1003A, R1060A variants) proteins and modifications thereof. Cpf1 protein, Zetsche et al., Cell, 163:1-13 (2015), also contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). As used herein, delivery of an RNA-guided DNA-binding agent (e.g., a Cas nuclease, a Cas9 nuclease, or an S. pyogenes Cas9 nuclease or an Neisseria meningitidis Cas9 nuclease) includes delivery of the polypeptide or mRNA.

As used herein, the term “editor” or “base editor” refers to an agent comprising a polypeptide that is capable of making a modification to a base (e.g., A, T, C, G, or U) within a nucleic acid sequence (e.g., DNA or RNA). In some embodiments, the editor is capable of deaminating a base within a nucleic acid. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase domain. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an enzymatically inactive RNA-guided DNA-binding proteins fused to a cytidine deaminase domain.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with the target sequence and the agent binds to the target sequence; in cases where the agent is a cleavase or nickase, binding can be followed by cleaving or nicking.

As used herein, the term “uracil glycosylase inhibitor”, “uracil-DNA glycosylase inhibitor” or “UGI” refers to a protein that is capable of inhibiting a uracil-DNA glycosylase (UDG) base-excision repair enzyme (e.g., UniPROT ID: P14739;

(SEQ ID NO: 226)
MTNLSDIIEKETGKQLVIQESILMLPEEVEEVIGNKPESD
ILVHTAYDESTDENVMLLTSDAPEYKPWALVIQDSNGENK
IKMLSGGSKRTADGSEFESPKKKRKVE;
or
(SEQ ID NO: 227)
TNLSDIIEKETGKQLVIQESILMLPEEVEEVIGNKPESDI
LVHTAYDESTDENVMLLTSDAPEYKPWALVIQDSNGENKI
KML).

Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences or nucleic acid sequences encoding the amino acid sequences provided herein are also contemplated. In certain embodiments, the nucleotide sequence encoding the Cas9 amino acid sequence is not a naturally occurring Cas9 nucleotide sequence. Sequences with at least 95%, 96%, 97%, 98%, or 99% identity to any of the Cas9 amino acid sequences provided herein are also contemplated. In certain embodiments, the Cas9 amino acid sequence is not a naturally occurring Cas9 sequence.

Exemplary open reading frame for Spy Cas9
(SEQ ID NO: 218)
AUGGACAAGAAGUACUCCAUCGGCCUGGACAUCGGCACCAACUCCGUGGGCUGGGCCGUGAUCAC
CGACGAGUACAAGGUGCCCUCCAAGAAGUUCAAGGUGCUGGGCAACACCGACCGGCACUCCAUCA
AGAAGAACCUGAUCGGCGCCCUGCUGUUCGACUCCGGCGAGACCGCCGAGGCCACCCGGCUGAAG
CGGACCGCCCGGCGGCGGUACACCCGGCGGAAGAACCGGAUCUGCUACCUGCAGGAGAUCUUCUC
CAACGAGAUGGCCAAGGUGGACGACUCCUUCUUCCACCGGCUGGAGGAGUCCUUCCUGGUGGAGG
AGGACAAGAAGCACGAGCGGCACCCCAUCUUCGGCAACAUCGUGGACGAGGUGGCCUACCACGAG
AAGUACCCCACCAUCUACCACCUGCGGAAGAAGCUGGUGGACUCCACCGACAAGGCCGACCUGCG
GCUGAUCUACCUGGCCCUGGCCCACAUGAUCAAGUUCCGGGGCCACUUCCUGAUCGAGGGCGACC
UGAACCCCGACAACUCCGACGUGGACAAGCUGUUCAUCCAGCUGGUGCAGACCUACAACCAGCUG
UUCGAGGAGAACCCCAUCAACGCCUCCGGCGUGGACGCCAAGGCCAUCCUGUCCGCCCGGCUGUC
CAAGUCCCGGCGGCUGGAGAACCUGAUCGCCCAGCUGCCCGGCGAGAAGAAGAACGGCCUGUUCG
GCAACCUGAUCGCCCUGUCCCUGGGCCUGACCCCCAACUUCAAGUCCAACUUCGACCUGGCCGAG
GACGCCAAGCUGCAGCUGUCCAAGGACACCUACGACGACGACCUGGACAACCUGCUGGCCCAGAU
CGGCGACCAGUACGCCGACCUGUUCCUGGCCGCCAAGAACCUGUCCGACGCCAUCCUGCUGUCCG
ACAUCCUGCGGGUGAACACCGAGAUCACCAAGGCCCCCCUGUCCGCCUCCAUGAUCAAGCGGUAC
GACGAGCACCACCAGGACCUGACCCUGCUGAAGGCCCUGGUGCGGCAGCAGCUGCCCGAGAAGUA
CAAGGAGAUCUUCUUCGACCAGUCCAAGAACGGCUACGCCGGCUACAUCGACGGCGGCGCCUCCC
AGGAGGAGUUCUACAAGUUCAUCAAGCCCAUCCUGGAGAAGAUGGACGGCACCGAGGAGCUGCUG
GUGAAGCUGAACCGGGAGGACCUGCUGCGGAAGCAGCGGACCUUCGACAACGGCUCCAUCCCCCA
CCAGAUCCACCUGGGCGAGCUGCACGCCAUCCUGCGGCGGCAGGAGGACUUCUACCCCUUCCUGA
AGGACAACCGGGAGAAGAUCGAGAAGAUCCUGACCUUCCGGAUCCCCUACUACGUGGGCCCCCUG
GCCCGGGGCAACUCCCGGUUCGCCUGGAUGACCCGGAAGUCCGAGGAGACCAUCACCCCCUGGAA
CUUCGAGGAGGUGGUGGACAAGGGCGCCUCCGCCCAGUCCUUCAUCGAGCGGAUGACCAACUUCG
ACAAGAACCUGCCCAACGAGAAGGUGCUGCCCAAGCACUCCCUGCUGUACGAGUACUUCACCGUG
UACAACGAGCUGACCAAGGUGAAGUACGUGACCGAGGGCAUGCGGAAGCCCGCCUUCCUGUCCGG
CGAGCAGAAGAAGGCCAUCGUGGACCUGCUGUUCAAGACCAACCGGAAGGUGACCGUGAAGCAGC
UGAAGGAGGACUACUUCAAGAAGAUCGAGUGCUUCGACUCCGUGGAGAUCUCCGGCGUGGAGGAC
CGGUUCAACGCCUCCCUGGGCACCUACCACGACCUGCUGAAGAUCAUCAAGGACAAGGACUUCCU
GGACAACGAGGAGAACGAGGACAUCCUGGAGGACAUCGUGCUGACCCUGACCCUGUUCGAGGACC
GGGAGAUGAUCGAGGAGCGGCUGAAGACCUACGCCCACCUGUUCGACGACAAGGUGAUGAAGCAG
CUGAAGCGGCGGCGGUACACCGGCUGGGGCCGGCUGUCCCGGAAGCUGAUCAACGGCAUCCGGGA
CAAGCAGUCCGGCAAGACCAUCCUGGACUUCCUGAAGUCCGACGGCUUCGCCAACCGGAACUUCA
UGCAGCUGAUCCACGACGACUCCCUGACCUUCAAGGAGGACAUCCAGAAGGCCCAGGUGUCCGGC
CAGGGCGACUCCCUGCACGAGCACAUCGCCAACCUGGCCGGCUCCCCCGCCAUCAAGAAGGGCAU
CCUGCAGACCGUGAAGGUGGUGGACGAGCUGGUGAAGGUGAUGGGCCGGCACAAGCCCGAGAACA
UCGUGAUCGAGAUGGCCCGGGAGAACCAGACCACCCAGAAGGGCCAGAAGAACUCCCGGGAGCGG
AUGAAGCGGAUCGAGGAGGGCAUCAAGGAGCUGGGCUCCCAGAUCCUGAAGGAGCACCCCGUGGA
GAACACCCAGCUGCAGAACGAGAAGCUGUACCUGUACUACCUGCAGAACGGCCGGGACAUGUACG
UGGACCAGGAGCUGGACAUCAACCGGCUGUCCGACUACGACGUGGACCACAUCGUGCCCCAGUCC
UUCCUGAAGGACGACUCCAUCGACAACAAGGUGCUGACCCGGUCCGACAAGAACCGGGGCAAGUC
CGACAACGUGCCCUCCGAGGAGGUGGUGAAGAAGAUGAAGAACUACUGGCGGCAGCUGCUGAACG
CCAAGCUGAUCACCCAGCGGAAGUUCGACAACCUGACCAAGGCCGAGCGGGGCGGCCUGUCCGAG
CUGGACAAGGCCGGCUUCAUCAAGCGGCAGCUGGUGGAGACCCGGCAGAUCACCAAGCACGUGGC
CCAGAUCCUGGACUCCCGGAUGAACACCAAGUACGACGAGAACGACAAGCUGAUCCGGGAGGUGA
AGGUGAUCACCCUGAAGUCCAAGCUGGUGUCCGACUUCCGGAAGGACUUCCAGUUCUACAAGGUG
CGGGAGAUCAACAACUACCACCACGCCCACGACGCCUACCUGAACGCCGUGGUGGGCACCGCCCU
GAUCAAGAAGUACCCCAAGCUGGAGUCCGAGUUCGUGUACGGCGACUACAAGGUGUACGACGUGC
GGAAGAUGAUCGCCAAGUCCGAGCAGGAGAUCGGCAAGGCCACCGCCAAGUACUUCUUCUACUCC
AACAUCAUGAACUUCUUCAAGACCGAGAUCACCCUGGCCAACGGCGAGAUCCGGAAGCGGCCCCU
GAUCGAGACCAACGGCGAGACCGGCGAGAUCGUGUGGGACAAGGGCCGGGACUUCGCCACCGUGC
GGAAGGUGCUGUCCAUGCCCCAGGUGAACAUCGUGAAGAAGACCGAGGUGCAGACCGGCGGCUUC
UCCAAGGAGUCCAUCCUGCCCAAGCGGAACUCCGACAAGCUGAUCGCCCGGAAGAAGGACUGGGA
CCCCAAGAAGUACGGCGGCUUCGACUCCCCCACCGUGGCCUACUCCGUGCUGGUGGUGGCCAAGG
UGGAGAAGGGCAAGUCCAAGAAGCUGAAGUCCGUGAAGGAGCUGCUGGGCAUCACCAUCAUGGAG
CGGUCCUCCUUCGAGAAGAACCCCAUCGACUUCCUGGAGGCCAAGGGCUACAAGGAGGUGAAGAA
GGACCUGAUCAUCAAGCUGCCCAAGUACUCCCUGUUCGAGCUGGAGAACGGCCGGAAGCGGAUGC
UGGCCUCCGCCGGCGAGCUGCAGAAGGGCAACGAGCUGGCCCUGCCCUCCAAGUACGUGAACUUC
CUGUACCUGGCCUCCCACUACGAGAAGCUGAAGGGCUCCCCCGAGGACAACGAGCAGAAGCAGCU
GUUCGUGGAGCAGCACAAGCACUACCUGGACGAGAUCAUCGAGCAGAUCUCCGAGUUCUCCAAGC
GGGUGAUCCUGGCCGACGCCAACCUGGACAAGGUGCUGUCCGCCUACAACAAGCACCGGGACAAG
CCCAUCCGGGAGCAGGCCGAGAACAUCAUCCACCUGUUCACCCUGACCAACCUGGGCGCCCCCGC
CGCCUUCAAGUACUUCGACACCACCAUCGACCGGAAGCGGUACACCUCCACCAAGGAGGUGCUGG
ACGCCACCCUGAUCCACCAGUCCAUCACCGGCCUGUACGAGACCCGGAUCGACCUGUCCCAGCUG
GGCGGCGACGGCGGCGGCUCCCCCAAGAAGAAGCGGAAGGUGUGA
Exemplary amino acid sequence for Spy Cas9
(SEQ ID NO: 219)
MDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQS
FLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRIDLSQL
GGDGGGSPKKKRKV
Exemplary open reading frame for Spy Cas9
(SEQ ID NO: 116)
AUGGACAAGAAGUACAGCAUCGGACUGGACAUCGGAACAAACAGCGUCGGAUGGGCAGUCAUCAC
AGACGAAUACAAGGUCCCGAGCAAGAAGUUCAAGGUCCUGGGAAACACAGACAGACACAGCAUCA
AGAAGAACCUGAUCGGAGCACUGCUGUUCGACAGCGGAGAAACAGCAGAAGCAACAAGACUGAAG
AGAACAGCAAGAAGAAGAUACACAAGAAGAAAGAACAGAAUCUGCUACCUGCAGGAAAUCUUCAG
CAACGAAAUGGCAAAGGUCGACGACAGCUUCUUCCACAGACUGGAAGAAAGCUUCCUGGUCGAAG
AAGACAAGAAGCACGAAAGACACCCGAUCUUCGGAAACAUCGUCGACGAAGUCGCAUACCACGAA
AAGUACCCGACAAUCUACCACCUGAGAAAGAAGCUGGUCGACAGCACAGACAAGGCAGACCUGAG
ACUGAUCUACCUGGCACUGGCACACAUGAUCAAGUUCAGAGGACACUUCCUGAUCGAAGGAGACC
UGAACCCGGACAACAGCGACGUCGACAAGCUGUUCAUCCAGCUGGUCCAGACAUACAACCAGCUG
UUCGAAGAAAACCCGAUCAACGCAAGCGGAGUCGACGCAAAGGCAAUCCUGAGCGCAAGACUGAG
CAAGAGCAGAAGACUGGAAAACCUGAUCGCACAGCUGCCGGGAGAAAAGAAGAACGGACUGUUCG
GAAACCUGAUCGCACUGAGCCUGGGACUGACACCGAACUUCAAGAGCAACUUCGACCUGGCAGAA
GACGCAAAGCUGCAGCUGAGCAAGGACACAUACGACGACGACCUGGACAACCUGCUGGCACAGAU
CGGAGACCAGUACGCAGACCUGUUCCUGGCAGCAAAGAACCUGAGCGACGCAAUCCUGCUGAGCG
ACAUCCUGAGAGUCAACACAGAAAUCACAAAGGCACCGCUGAGCGCAAGCAUGAUCAAGAGAUAC
GACGAACACCACCAGGACCUGACACUGCUGAAGGCACUGGUCAGACAGCAGCUGCCGGAAAAGUA
CAAGGAAAUCUUCUUCGACCAGAGCAAGAACGGAUACGCAGGAUACAUCGACGGAGGAGCAAGCC
AGGAAGAAUUCUACAAGUUCAUCAAGCCGAUCCUGGAAAAGAUGGACGGAACAGAAGAACUGCUG
GUCAAGCUGAACAGAGAAGACCUGCUGAGAAAGCAGAGAACAUUCGACAACGGAAGCAUCCCGCA
CCAGAUCCACCUGGGAGAACUGCACGCAAUCCUGAGAAGACAGGAAGACUUCUACCCGUUCCUGA
AGGACAACAGAGAAAAGAUCGAAAAGAUCCUGACAUUCAGAAUCCCGUACUACGUCGGACCGCUG
GCAAGAGGAAACAGCAGAUUCGCAUGGAUGACAAGAAAGAGCGAAGAAACAAUCACACCGUGGAA
CUUCGAAGAAGUCGUCGACAAGGGAGCAAGCGCACAGAGCUUCAUCGAAAGAAUGACAAACUUCG
ACAAGAACCUGCCGAACGAAAAGGUCCUGCCGAAGCACAGCCUGCUGUACGAAUACUUCACAGUC
UACAACGAACUGACAAAGGUCAAGUACGUCACAGAAGGAAUGAGAAAGCCGGCAUUCCUGAGCGG
AGAACAGAAGAAGGCAAUCGUCGACCUGCUGUUCAAGACAAACAGAAAGGUCACAGUCAAGCAGC
UGAAGGAAGACUACUUCAAGAAGAUCGAAUGCUUCGACAGCGUCGAAAUCAGCGGAGUCGAAGAC
AGAUUCAACGCAAGCCUGGGAACAUACCACGACCUGCUGAAGAUCAUCAAGGACAAGGACUUCCU
GGACAACGAAGAAAACGAAGACAUCCUGGAAGACAUCGUCCUGACACUGACACUGUUCGAAGACA
GAGAAAUGAUCGAAGAAAGACUGAAGACAUACGCACACCUGUUCGACGACAAGGUCAUGAAGCAG
CUGAAGAGAAGAAGAUACACAGGAUGGGGAAGACUGAGCAGAAAGCUGAUCAACGGAAUCAGAGA
CAAGCAGAGCGGAAAGACAAUCCUGGACUUCCUGAAGAGCGACGGAUUCGCAAACAGAAACUUCA
UGCAGCUGAUCCACGACGACAGCCUGACAUUCAAGGAAGACAUCCAGAAGGCACAGGUCAGCGGA
CAGGGAGACAGCCUGCACGAACACAUCGCAAACCUGGCAGGAAGCCCGGCAAUCAAGAAGGGAAU
CCUGCAGACAGUCAAGGUCGUCGACGAACUGGUCAAGGUCAUGGGAAGACACAAGCCGGAAAACA
UCGUCAUCGAAAUGGCAAGAGAAAACCAGACAACACAGAAGGGACAGAAGAACAGCAGAGAAAGA
AUGAAGAGAAUCGAAGAAGGAAUCAAGGAACUGGGAAGCCAGAUCCUGAAGGAACACCCGGUCGA
AAACACACAGCUGCAGAACGAAAAGCUGUACCUGUACUACCUGCAGAACGGAAGAGACAUGUACG
UCGACCAGGAACUGGACAUCAACAGACUGAGCGACUACGACGUCGACCACAUCGUCCCGCAGAGC
UUCCUGAAGGACGACAGCAUCGACAACAAGGUCCUGACAAGAAGCGACAAGAACAGAGGAAAGAG
CGACAACGUCCCGAGCGAAGAAGUCGUCAAGAAGAUGAAGAACUACUGGAGACAGCUGCUGAACG
CAAAGCUGAUCACACAGAGAAAGUUCGACAACCUGACAAAGGCAGAGAGAGGAGGACUGAGCGAA
CUGGACAAGGCAGGAUUCAUCAAGAGACAGCUGGUCGAAACAAGACAGAUCACAAAGCACGUCGC
ACAGAUCCUGGACAGCAGAAUGAACACAAAGUACGACGAAAACGACAAGCUGAUCAGAGAAGUCA
AGGUCAUCACACUGAAGAGCAAGCUGGUCAGCGACUUCAGAAAGGACUUCCAGUUCUACAAGGUC
AGAGAAAUCAACAACUACCACCACGCACACGACGCAUACCUGAACGCAGUCGUCGGAACAGCACU
GAUCAAGAAGUACCCGAAGCUGGAAAGCGAAUUCGUCUACGGAGACUACAAGGUCUACGACGUCA
GAAAGAUGAUCGCAAAGAGCGAACAGGAAAUCGGAAAGGCAACAGCAAAGUACUUCUUCUACAGC
AACAUCAUGAACUUCUUCAAGACAGAAAUCACACUGGCAAACGGAGAAAUCAGAAAGAGACCGCU
GAUCGAAACAAACGGAGAAACAGGAGAAAUCGUCUGGGACAAGGGAAGAGACUUCGCAACAGUCA
GAAAGGUCCUGAGCAUGCCGCAGGUCAACAUCGUCAAGAAGACAGAAGUCCAGACAGGAGGAUUC
AGCAAGGAAAGCAUCCUGCCGAAGAGAAACAGCGACAAGCUGAUCGCAAGAAAGAAGGACUGGGA
CCCGAAGAAGUACGGAGGAUUCGACAGCCCGACAGUCGCAUACAGCGUCCUGGUCGUCGCAAAGG
UCGAAAAGGGAAAGAGCAAGAAGCUGAAGAGCGUCAAGGAACUGCUGGGAAUCACAAUCAUGGAA
AGAAGCAGCUUCGAAAAGAACCCGAUCGACUUCCUGGAAGCAAAGGGAUACAAGGAAGUCAAGAA
GGACCUGAUCAUCAAGCUGCCGAAGUACAGCCUGUUCGAACUGGAAAACGGAAGAAAGAGAAUGC
UGGCAAGCGCAGGAGAACUGCAGAAGGGAAACGAACUGGCACUGCCGAGCAAGUACGUCAACUUC
CUGUACCUGGCAAGCCACUACGAAAAGCUGAAGGGAAGCCCGGAAGACAACGAACAGAAGCAGCU
GUUCGUCGAACAGCACAAGCACUACCUGGACGAAAUCAUCGAACAGAUCAGCGAAUUCAGCAAGA
GAGUCAUCCUGGCAGACGCAAACCUGGACAAGGUCCUGAGCGCAUACAACAAGCACAGAGACAAG
CCGAUCAGAGAACAGGCAGAAAACAUCAUCCACCUGUUCACACUGACAAACCUGGGAGCACCGGC
AGCAUUCAAGUACUUCGACACAACAAUCGACAGAAAGAGAUACACAAGCACAAAGGAAGUCCUGG
ACGCAACACUGAUCCACCAGAGCAUCACAGGACUGUACGAAACAAGAAUCGACCUGAGCCAGCUG
GGAGGAGACGGAGGAGGAAGCCCGAAGAAGAAGAGAAAGGUCUAG
Exemplary open reading frame for Spy Cas9 with Hibit tag
(SEQ ID NO: 117)
AUGGACAAGAAGUACUCCAUCGGCCUGGACAUCGGCACCAACUCCGUGGGCUGGGCCGUGAUCAC
CGACGAGUACAAGGUGCCCUCCAAGAAGUUCAAGGUGCUGGGCAACACCGACCGGCACUCCAUCA
AGAAGAACCUGAUCGGCGCCCUGCUGUUCGACUCCGGCGAGACCGCCGAGGCCACCCGGCUGAAG
CGGACCGCCCGGCGGCGGUACACCCGGCGGAAGAACCGGAUCUGCUACCUGCAGGAGAUCUUCUC
CAACGAGAUGGCCAAGGUGGACGACUCCUUCUUCCACCGGCUGGAGGAGUCCUUCCUGGUGGAGG
AGGACAAGAAGCACGAGCGGCACCCCAUCUUCGGCAACAUCGUGGACGAGGUGGCCUACCACGAG
AAGUACCCCACCAUCUACCACCUGCGGAAGAAGCUGGUGGACUCCACCGACAAGGCCGACCUGCG
GCUGAUCUACCUGGCCCUGGCCCACAUGAUCAAGUUCCGGGGCCACUUCCUGAUCGAGGGCGACC
UGAACCCCGACAACUCCGACGUGGACAAGCUGUUCAUCCAGCUGGUGCAGACCUACAACCAGCUG
UUCGAGGAGAACCCCAUCAACGCCUCCGGCGUGGACGCCAAGGCCAUCCUGUCCGCCCGGCUGUC
CAAGUCCCGGCGGCUGGAGAACCUGAUCGCCCAGCUGCCCGGCGAGAAGAAGAACGGCCUGUUCG
GCAACCUGAUCGCCCUGUCCCUGGGCCUGACCCCCAACUUCAAGUCCAACUUCGACCUGGCCGAG
GACGCCAAGCUGCAGCUGUCCAAGGACACCUACGACGACGACCUGGACAACCUGCUGGCCCAGAU
CGGCGACCAGUACGCCGACCUGUUCCUGGCCGCCAAGAACCUGUCCGACGCCAUCCUGCUGUCCG
ACAUCCUGCGGGUGAACACCGAGAUCACCAAGGCCCCCCUGUCCGCCUCCAUGAUCAAGCGGUAC
GACGAGCACCACCAGGACCUGACCCUGCUGAAGGCCCUGGUGCGGCAGCAGCUGCCCGAGAAGUA
CAAGGAGAUCUUCUUCGACCAGUCCAAGAACGGCUACGCCGGCUACAUCGACGGCGGCGCCUCCC
AGGAGGAGUUCUACAAGUUCAUCAAGCCCAUCCUGGAGAAGAUGGACGGCACCGAGGAGCUGCUG
GUGAAGCUGAACCGGGAGGACCUGCUGCGGAAGCAGCGGACCUUCGACAACGGCUCCAUCCCCCA
CCAGAUCCACCUGGGCGAGCUGCACGCCAUCCUGCGGCGGCAGGAGGACUUCUACCCCUUCCUGA
AGGACAACCGGGAGAAGAUCGAGAAGAUCCUGACCUUCCGGAUCCCCUACUACGUGGGCCCCCUG
GCCCGGGGCAACUCCCGGUUCGCCUGGAUGACCCGGAAGUCCGAGGAGACCAUCACCCCCUGGAA
CUUCGAGGAGGUGGUGGACAAGGGCGCCUCCGCCCAGUCCUUCAUCGAGCGGAUGACCAACUUCG
ACAAGAACCUGCCCAACGAGAAGGUGCUGCCCAAGCACUCCCUGCUGUACGAGUACUUCACCGUG
UACAACGAGCUGACCAAGGUGAAGUACGUGACCGAGGGCAUGCGGAAGCCCGCCUUCCUGUCCGG
CGAGCAGAAGAAGGCCAUCGUGGACCUGCUGUUCAAGACCAACCGGAAGGUGACCGUGAAGCAGC
UGAAGGAGGACUACUUCAAGAAGAUCGAGUGCUUCGACUCCGUGGAGAUCUCCGGCGUGGAGGAC
CGGUUCAACGCCUCCCUGGGCACCUACCACGACCUGCUGAAGAUCAUCAAGGACAAGGACUUCCU
GGACAACGAGGAGAACGAGGACAUCCUGGAGGACAUCGUGCUGACCCUGACCCUGUUCGAGGACC
GGGAGAUGAUCGAGGAGCGGCUGAAGACCUACGCCCACCUGUUCGACGACAAGGUGAUGAAGCAG
CUGAAGCGGCGGCGGUACACCGGCUGGGGCCGGCUGUCCCGGAAGCUGAUCAACGGCAUCCGGGA
CAAGCAGUCCGGCAAGACCAUCCUGGACUUCCUGAAGUCCGACGGCUUCGCCAACCGGAACUUCA
UGCAGCUGAUCCACGACGACUCCCUGACCUUCAAGGAGGACAUCCAGAAGGCCCAGGUGUCCGGC
CAGGGCGACUCCCUGCACGAGCACAUCGCCAACCUGGCCGGCUCCCCCGCCAUCAAGAAGGGCAU
CCUGCAGACCGUGAAGGUGGUGGACGAGCUGGUGAAGGUGAUGGGCCGGCACAAGCCCGAGAACA
UCGUGAUCGAGAUGGCCCGGGAGAACCAGACCACCCAGAAGGGCCAGAAGAACUCCCGGGAGCGG
AUGAAGCGGAUCGAGGAGGGCAUCAAGGAGCUGGGCUCCCAGAUCCUGAAGGAGCACCCCGUGGA
GAACACCCAGCUGCAGAACGAGAAGCUGUACCUGUACUACCUGCAGAACGGCCGGGACAUGUACG
UGGACCAGGAGCUGGACAUCAACCGGCUGUCCGACUACGACGUGGACCACAUCGUGCCCCAGUCC
UUCCUGAAGGACGACUCCAUCGACAACAAGGUGCUGACCCGGUCCGACAAGAACCGGGGCAAGUC
CGACAACGUGCCCUCCGAGGAGGUGGUGAAGAAGAUGAAGAACUACUGGCGGCAGCUGCUGAACG
CCAAGCUGAUCACCCAGCGGAAGUUCGACAACCUGACCAAGGCCGAGCGGGGCGGCCUGUCCGAG
CUGGACAAGGCCGGCUUCAUCAAGCGGCAGCUGGUGGAGACCCGGCAGAUCACCAAGCACGUGGC
CCAGAUCCUGGACUCCCGGAUGAACACCAAGUACGACGAGAACGACAAGCUGAUCCGGGAGGUGA
AGGUGAUCACCCUGAAGUCCAAGCUGGUGUCCGACUUCCGGAAGGACUUCCAGUUCUACAAGGUG
CGGGAGAUCAACAACUACCACCACGCCCACGACGCCUACCUGAACGCCGUGGUGGGCACCGCCCU
GAUCAAGAAGUACCCCAAGCUGGAGUCCGAGUUCGUGUACGGCGACUACAAGGUGUACGACGUGC
GGAAGAUGAUCGCCAAGUCCGAGCAGGAGAUCGGCAAGGCCACCGCCAAGUACUUCUUCUACUCC
AACAUCAUGAACUUCUUCAAGACCGAGAUCACCCUGGCCAACGGCGAGAUCCGGAAGCGGCCCCU
GAUCGAGACCAACGGCGAGACCGGCGAGAUCGUGUGGGACAAGGGCCGGGACUUCGCCACCGUGC
GGAAGGUGCUGUCCAUGCCCCAGGUGAACAUCGUGAAGAAGACCGAGGUGCAGACCGGCGGCUUC
UCCAAGGAGUCCAUCCUGCCCAAGCGGAACUCCGACAAGCUGAUCGCCCGGAAGAAGGACUGGGA
CCCCAAGAAGUACGGCGGCUUCGACUCCCCCACCGUGGCCUACUCCGUGCUGGUGGUGGCCAAGG
UGGAGAAGGGCAAGUCCAAGAAGCUGAAGUCCGUGAAGGAGCUGCUGGGCAUCACCAUCAUGGAG
CGGUCCUCCUUCGAGAAGAACCCCAUCGACUUCCUGGAGGCCAAGGGCUACAAGGAGGUGAAGAA
GGACCUGAUCAUCAAGCUGCCCAAGUACUCCCUGUUCGAGCUGGAGAACGGCCGGAAGCGGAUGC
UGGCCUCCGCCGGCGAGCUGCAGAAGGGCAACGAGCUGGCCCUGCCCUCCAAGUACGUGAACUUC
CUGUACCUGGCCUCCCACUACGAGAAGCUGAAGGGCUCCCCCGAGGACAACGAGCAGAAGCAGCU
GUUCGUGGAGCAGCACAAGCACUACCUGGACGAGAUCAUCGAGCAGAUCUCCGAGUUCUCCAAGC
GGGUGAUCCUGGCCGACGCCAACCUGGACAAGGUGCUGUCCGCCUACAACAAGCACCGGGACAAG
CCCAUCCGGGAGCAGGCCGAGAACAUCAUCCACCUGUUCACCCUGACCAACCUGGGCGCCCCCGC
CGCCUUCAAGUACUUCGACACCACCAUCGACCGGAAGCGGUACACCUCCACCAAGGAGGUGCUGG
ACGCCACCCUGAUCCACCAGUCCAUCACCGGCCUGUACGAGACCCGGAUCGACCUGUCCCAGCUG
GGCGGCGACGGCGGCGGCUCCCCCAAGAAGAAGCGGAAGGUGUCCGAGUCCGCCACCCCCGAGUC
CGUGUCCGGCUGGCGGCUGUUCAAGAAGAUCUCCUGA
Exemplary amino acid sequence for Spy Cas9 with Hibit tag
(SEQ ID NO: 118)
MDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQS
FLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRIDLSQL
GGDGGGSPKKKRKVSESATPESVSGWRLFKKIS
Exemplary amino acid sequence for Spy Cas9 nickase
(SEQ ID NO: 220)
MDKKYSIGLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLTGGGGSVDVNTEITKAPLSAS
MIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLY
EYFTVYNELTKVKYVTEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDREMIEERLKTYAHLFDD
KVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRVILADANLDKVLSAYN
KHRDKPIREQAENIIHLFTLTNLGAPAAFKYFDTTIDRKRYTSTKEVLDATLIHQSITGLYETRI
DLSQLGGDGGGSPKKKRKV
Exemplary open reading frame for Nme2Cas9
(SEQ ID NO: 221)
ATGGACGGCTCCGGCGGCGGCTCCCCCAAGAAGAAGCGGAAGGTGGAGGACAAGCGGCCCGCCGC
CACCAAGAAGGCCGGCCAGGCCAAGAAGAAGAAGGGCGGCTCCGGCGGCGGCGCCGCCTTCAAGC
CCAACCCCATCAACTACATCCTGGGCCTGGACATCGGCATCGCCTCCGTGGGCTGGGCCATGGTG
GAGATCGACGAGGAGGAGAACCCCATCCGGCTGATCGACCTGGGCGTGCGGGTGTTCGAGCGGGC
CGAGGTGCCCAAGACCGGCGACTCCCTGGCCATGGCCCGGCGGCTGGCCCGGTCCGTGCGGCGGC
TGACCCGGCGGCGGGCCCACCGGCTGCTGCGGGCCCGGCGGCTGCTGAAGCGGGAGGGCGTGCTG
CAGGCCGCCGACTTCGACGAGAACGGCCTGATCAAGTCCCTGCCCAACACCCCCTGGCAGCTGCG
GGCCGCCGCCCTGGACCGGAAGCTGACCCCCCTGGAGTGGTCCGCCGTGCTGCTGCACCTGATCA
AGCACCGGGGCTACCTGTCCCAGCGGAAGAACGAGGGCGAGACCGCCGACAAGGAGCTGGGCGCC
CTGCTGAAGGGCGTGGCCAACAACGCCCACGCCCTGCAGACCGGCGACTTCCGGACCCCCGCCGA
GCTGGCCCTGAACAAGTTCGAGAAGGAGTCCGGCCACATCCGGAACCAGCGGGGCGACTACTCCC
ACACCTTCTCCCGGAAGGACCTGCAGGCCGAGCTGATCCTGCTGTTCGAGAAGCAGAAGGAGTTC
GGCAACCCCCACGTGTCCGGCGGCCTGAAGGAGGGCATCGAGACCCTGCTGATGACCCAGCGGCC
CGCCCTGTCCGGCGACGCCGTGCAGAAGATGCTGGGCCACTGCACCTTCGAGCCCGCCGAGCCCA
AGGCCGCCAAGAACACCTACACCGCCGAGCGGTTCATCTGGCTGACCAAGCTGAACAACCTGCGG
ATCCTGGAGCAGGGCTCCGAGCGGCCCCTGACCGACACCGAGCGGGCCACCCTGATGGACGAGCC
CTACCGGAAGTCCAAGCTGACCTACGCCCAGGCCCGGAAGCTGCTGGGCCTGGAGGACACCGCCT
TCTTCAAGGGCCTGCGGTACGGCAAGGACAACGCCGAGGCCTCCACCCTGATGGAGATGAAGGCC
TACCACGCCATCTCCCGGGCCCTGGAGAAGGAGGGCCTGAAGGACAAGAAGTCCCCCCTGAACCT
GTCCTCCGAGCTGCAGGACGAGATCGGCACCGCCTTCTCCCTGTTCAAGACCGACGAGGACATCA
CCGGCCGGCTGAAGGACCGGGTGCAGCCCGAGATCCTGGAGGCCCTGCTGAAGCACATCTCCTTC
GACAAGTTCGTGCAGATCTCCCTGAAGGCCCTGCGGCGGATCGTGCCCCTGATGGAGCAGGGCAA
GCGGTACGACGAGGCCTGCGCCGAGATCTACGGCGACCACTACGGCAAGAAGAACACCGAGGAGA
AGATCTACCTGCCCCCCATCCCCGCCGACGAGATCCGGAACCCCGTGGTGCTGCGGGCCCTGTCC
CAGGCCCGGAAGGTGATCAACGGCGTGGTGCGGCGGTACGGCTCCCCCGCCCGGATCCACATCGA
GACCGCCCGGGAGGTGGGCAAGTCCTTCAAGGACCGGAAGGAGATCGAGAAGCGGCAGGAGGAGA
ACCGGAAGGACCGGGAGAAGGCCGCCGCCAAGTTCCGGGAGTACTTCCCCAACTTCGTGGGCGAG
CCCAAGTCCAAGGACATCCTGAAGCTGCGGCTGTACGAGCAGCAGCACGGCAAGTGCCTGTACTC
CGGCAAGGAGATCAACCTGGTGCGGCTGAACGAGAAGGGCTACGTGGAGATCGACCACGCCCTGC
CCTTCTCCCGGACCTGGGACGACTCCTTCAACAACAAGGTGCTGGTGCTGGGCTCCGAGAACCAG
AACAAGGGCAACCAGACCCCCTACGAGTACTTCAACGGCAAGGACAACTCCCGGGAGTGGCAGGA
GTTCAAGGCCCGGGTGGAGACCTCCCGGTTCCCCCGGTCCAAGAAGCAGCGGATCCTGCTGCAGA
AGTTCGACGAGGACGGCTTCAAGGAGTGCAACCTGAACGACACCCGGTACGTGAACCGGTTCCTG
TGCCAGTTCGTGGCCGACCACATCCTGCTGACCGGCAAGGGCAAGCGGCGGGTGTTCGCCTCCAA
CGGCCAGATCACCAACCTGCTGCGGGGCTTCTGGGGCCTGCGGAAGGTGCGGGCCGAGAACGACC
GGCACCACGCCCTGGACGCCGTGGTGGTGGCCTGCTCCACCGTGGCCATGCAGCAGAAGATCACC
CGGTTCGTGCGGTACAAGGAGATGAACGCCTTCGACGGCAAGACCATCGACAAGGAGACCGGCAA
GGTGCTGCACCAGAAGACCCACTTCCCCCAGCCCTGGGAGTTCTTCGCCCAGGAGGTGATGATCC
GGGTGTTCGGCAAGCCCGACGGCAAGCCCGAGTTCGAGGAGGCCGACACCCCCGAGAAGCTGCGG
ACCCTGCTGGCCGAGAAGCTGTCCTCCCGGCCCGAGGCCGTGCACGAGTACGTGACCCCCCTGTT
CGTGTCCCGGGCCCCCAACCGGAAGATGTCCGGCGCCCACAAGGACACCCTGCGGTCCGCCAAGC
GGTTCGTGAAGCACAACGAGAAGATCTCCGTGAAGCGGGTGTGGCTGACCGAGATCAAGCTGGCC
GACCTGGAGAACATGGTGAACTACAAGAACGGCCGGGAGATCGAGCTGTACGAGGCCCTGAAGGC
CCGGCTGGAGGCCTACGGCGGCAACGCCAAGCAGGCCTTCGACCCCAAGGACAACCCCTTCTACA
AGAAGGGCGGCCAGCTGGTGAAGGCCGTGCGGGTGGAGAAGACCCAGGAGTCCGGCGTGCTGCTG
AACAAGAAGAACGCCTACACCATCGCCGACAACGGCGACATGGTGCGGGTGGACGTGTTCTGCAA
GGTGGACAAGAAGGGCAAGAACCAGTACTTCATCGTGCCCATCTACGCCTGGCAGGTGGCCGAGA
ACATCCTGCCCGACATCGACTGCAAGGGCTACCGGATCGACGACTCCTACACCTTCTGCTTCTCC
CTGCACAAGTACGACCTGATCGCCTTCCAGAAGGACGAGAAGTCCAAGGTGGAGTTCGCCTACTA
CATCAACTGCGACTCCTCCAACGGCCGGTTCTACCTGGCCTGGCACGACAAGGGCTCCAAGGAGC
AGCAGTTCCGGATCTCCACCCAGAACCTGGTGCTGATCCAGAAGTACCAGGTGAACGAGCTGGGC
AAGGAGATCCGGCCCTGCCGGCTGAAGAAGCGGCCCCCCGTGCGGTAG
Exemplary mRNA for Nme2Cas9 nickase
(SEQ ID NO: 222)
GGGAAGCUCAGAAUAAACGCUCAACUUUGGCCGGAUCUGCCACCAUGGACGGCUCCGGCGGCGGC
UCCCCCAAGAAGAAGCGGAAGGUGGAGGACAAGCGGCCCGCCGCCACCAAGAAGGCCGGCCAGGC
CAAGAAGAAGAAGGGCGGCUCCGGCGGCGGCGCCGCCUUCAAGCCCAACCCCAUCAACUACAUCC
UGGGCCUGGACAUCGGCAUCGCCUCCGUGGGCUGGGCCAUGGUGGAGAUCGACGAGGAGGAGAAC
CCCAUCCGGCUGAUCGACCUGGGCGUGCGGGUGUUCGAGCGGGCCGAGGUGCCCAAGACCGGCGA
CUCCCUGGCCAUGGCCCGGCGGCUGGCCCGGUCCGUGCGGCGGCUGACCCGGCGGCGGGCCCACC
GGCUGCUGCGGGCCCGGCGGCUGCUGAAGCGGGAGGGCGUGCUGCAGGCCGCCGACUUCGACGAG
AACGGCCUGAUCAAGUCCCUGCCCAACACCCCCUGGCAGCUGCGGGCCGCCGCCCUGGACCGGAA
GCUGACCCCCCUGGAGUGGUCCGCCGUGCUGCUGCACCUGAUCAAGCACCGGGGCUACCUGUCCC
AGCGGAAGAACGAGGGCGAGACCGCCGACAAGGAGCUGGGCGCCCUGCUGAAGGGCGUGGCCAAC
AACGCCCACGCCCUGCAGACCGGCGACUUCCGGACCCCCGCCGAGCUGGCCCUGAACAAGUUCGA
GAAGGAGUCCGGCCACAUCCGGAACCAGCGGGGCGACUACUCCCACACCUUCUCCCGGAAGGACC
UGCAGGCCGAGCUGAUCCUGCUGUUCGAGAAGCAGAAGGAGUUCGGCAACCCCCACGUGUCCGGC
GGCCUGAAGGAGGGCAUCGAGACCCUGCUGAUGACCCAGCGGCCCGCCCUGUCCGGCGACGCCGU
GCAGAAGAUGCUGGGCCACUGCACCUUCGAGCCCGCCGAGCCCAAGGCCGCCAAGAACACCUACA
CCGCCGAGCGGUUCAUCUGGCUGACCAAGCUGAACAACCUGCGGAUCCUGGAGCAGGGCUCCGAG
CGGCCCCUGACCGACACCGAGCGGGCCACCCUGAUGGACGAGCCCUACCGGAAGUCCAAGCUGAC
CUACGCCCAGGCCCGGAAGCUGCUGGGCCUGGAGGACACCGCCUUCUUCAAGGGCCUGCGGUACG
GCAAGGACAACGCCGAGGCCUCCACCCUGAUGGAGAUGAAGGCCUACCACGCCAUCUCCCGGGCC
CUGGAGAAGGAGGGCCUGAAGGACAAGAAGUCCCCCCUGAACCUGUCCUCCGAGCUGCAGGACGA
GAUCGGCACCGCCUUCUCCCUGUUCAAGACCGACGAGGACAUCACCGGCCGGCUGAAGGACCGGG
UGCAGCCCGAGAUCCUGGAGGCCCUGCUGAAGCACAUCUCCUUCGACAAGUUCGUGCAGAUCUCC
CUGAAGGCCCUGCGGCGGAUCGUGCCCCUGAUGGAGCAGGGCAAGCGGUACGACGAGGCCUGCGC
CGAGAUCUACGGCGACCACUACGGCAAGAAGAACACCGAGGAGAAGAUCUACCUGCCCCCCAUCC
CCGCCGACGAGAUCCGGAACCCCGUGGUGCUGCGGGCCCUGUCCCAGGCCCGGAAGGUGAUCAAC
GGCGUGGUGCGGCGGUACGGCUCCCCCGCCCGGAUCCACAUCGAGACCGCCCGGGAGGUGGGCAA
GUCCUUCAAGGACCGGAAGGAGAUCGAGAAGCGGCAGGAGGAGAACCGGAAGGACCGGGAGAAGG
CCGCCGCCAAGUUCCGGGAGUACUUCCCCAACUUCGUGGGCGAGCCCAAGUCCAAGGACAUCCUG
AAGCUGCGGCUGUACGAGCAGCAGCACGGCAAGUGCCUGUACUCCGGCAAGGAGAUCAACCUGGU
GCGGCUGAACGAGAAGGGCUACGUGGAGAUCGACCACGCCCUGCCCUUCUCCCGGACCUGGGACG
ACUCCUUCAACAACAAGGUGCUGGUGCUGGGCUCCGAGAACCAGAACAAGGGCAACCAGACCCCC
UACGAGUACUUCAACGGCAAGGACAACUCCCGGGAGUGGCAGGAGUUCAAGGCCCGGGUGGAGAC
CUCCCGGUUCCCCCGGUCCAAGAAGCAGCGGAUCCUGCUGCAGAAGUUCGACGAGGACGGCUUCA
AGGAGUGCAACCUGAACGACACCCGGUACGUGAACCGGUUCCUGUGCCAGUUCGUGGCCGACCAC
AUCCUGCUGACCGGCAAGGGCAAGCGGCGGGUGUUCGCCUCCAACGGCCAGAUCACCAACCUGCU
GCGGGGCUUCUGGGGCCUGCGGAAGGUGCGGGCCGAGAACGACCGGCACCACGCCCUGGACGCCG
UGGUGGUGGCCUGCUCCACCGUGGCCAUGCAGCAGAAGAUCACCCGGUUCGUGCGGUACAAGGAG
AUGAACGCCUUCGACGGCAAGACCAUCGACAAGGAGACCGGCAAGGUGCUGCACCAGAAGACCCA
CUUCCCCCAGCCCUGGGAGUUCUUCGCCCAGGAGGUGAUGAUCCGGGUGUUCGGCAAGCCCGACG
GCAAGCCCGAGUUCGAGGAGGCCGACACCCCCGAGAAGCUGCGGACCCUGCUGGCCGAGAAGCUG
UCCUCCCGGCCCGAGGCCGUGCACGAGUACGUGACCCCCCUGUUCGUGUCCCGGGCCCCCAACCG
GAAGAUGUCCGGCGCCCACAAGGACACCCUGCGGUCCGCCAAGCGGUUCGUGAAGCACAACGAGA
AGAUCUCCGUGAAGCGGGUGUGGCUGACCGAGAUCAAGCUGGCCGACCUGGAGAACAUGGUGAAC
UACAAGAACGGCCGGGAGAUCGAGCUGUACGAGGCCCUGAAGGCCCGGCUGGAGGCCUACGGCGG
CAACGCCAAGCAGGCCUUCGACCCCAAGGACAACCCCUUCUACAAGAAGGGCGGCCAGCUGGUGA
AGGCCGUGCGGGUGGAGAAGACCCAGGAGUCCGGCGUGCUGCUGAACAAGAAGAACGCCUACACC
AUCGCCGACAACGGCGACAUGGUGCGGGUGGACGUGUUCUGCAAGGUGGACAAGAAGGGCAAGAA
CCAGUACUUCAUCGUGCCCAUCUACGCCUGGCAGGUGGCCGAGAACAUCCUGCCCGACAUCGACU
GCAAGGGCUACCGGAUCGACGACUCCUACACCUUCUGCUUCUCCCUGCACAAGUACGACCUGAUC
GCCUUCCAGAAGGACGAGAAGUCCAAGGUGGAGUUCGCCUACUACAUCAACUGCGACUCCUCCAA
CGGCCGGUUCUACCUGGCCUGGCACGACAAGGGCUCCAAGGAGCAGCAGUUCCGGAUCUCCACCC
AGAACCUGGUGCUGAUCCAGAAGUACCAGGUGAACGAGCUGGGCAAGGAGAUCCGGCCCUGCCGG
CUGAAGAAGCGGCCCCCCGUGCGGUAGCUAGCACCAGCCUCAAGAACACCCGAAUGGAGUCUCUA
AGCUACAUAAUACCAACUUACACUUUACAAAAUGUUGUCCCCCAAAAUGUAGCCAUUCGUAUCUG
CUCCUAAUAAAAAGAAAGUUUCUUCACAUUCUCUCGAGAAAAAAAAAAAAUGGAAAAAAAAAAAA
CGGAAAAAAAAAAAAGGUAAAAAAAAAAAAUAUAAAAAAAAAAAACAUAAAAAAAAAAAACGAAA
AAAAAAAAACGUAAAAAAAAAAAACUCAAAAAAAAAAAGAUAAAAAAAAAAAACCUAAAAAAAAA
AAAUGUAAAAAAAAAAAAGGGAAAAAAAAAAAACGCAAAAAAAAAAAACACAAAAAAAAAAAAUG
CAAAAAAAAAAAAUCGAAAAAAAAAAAAUCUAAAAAAAAAAAACGAAAAAAAAAAAACCCAAAAA
AAAAAAAGACAAAAAAAAAAAAUAGAAAAAAAAAAAGUUAAAAAAAAAAAACUGAAAAAAAAAAA
AUUUAAAAAAAAAAAAUCUAG
Exemplary amino acid sequence for Nme2Cas9
(SEQ ID NO: 223)
MDGSGGGSPKKKRKVEDKRPAATKKAGQAKKKKGGSGGGAAFKPNPINYILGLDIGIASVGWAMV
EIDEEENPIRLIDLGVRVFERAEVPKTGDSLAMARRLARSVRRLTRRRAHRLLRARRLLKREGVL
QAADFDENGLIKSLPNTPWQLRAAALDRKLTPLEWSAVLLHLIKHRGYLSQRKNEGETADKELGA
LLKGVANNAHALQTGDFRTPAELALNKFEKESGHIRNQRGDYSHTFSRKDLQAELILLFEKQKEF
GNPHVSGGLKEGIETLLMTQRPALSGDAVQKMLGHCTFEPAEPKAAKNTYTAERFIWLTKLNNLR
ILEQGSERPLTDTERATLMDEPYRKSKLTYAQARKLLGLEDTAFFKGLRYGKDNAEASTLMEMKA
YHAISRALEKEGLKDKKSPLNLSSELQDEIGTAFSLFKTDEDITGRLKDRVQPEILEALLKHISF
DKFVQISLKALRRIVPLMEQGKRYDEACAEIYGDHYGKKNTEEKIYLPPIPADEIRNPVVLRALS
QARKVINGVVRRYGSPARIHIETAREVGKSFKDRKEIEKRQEENRKDREKAAAKFREYFPNFVGE
PKSKDILKLRLYEQQHGKCLYSGKEINLVRLNEKGYVEIDHALPFSRTWDDSFNNKVLVLGSENQ
NKGNQTPYEYFNGKDNSREWQEFKARVETSRFPRSKKQRILLQKFDEDGFKECNLNDTRYVNRFL
CQFVADHILLTGKGKRRVFASNGQITNLLRGFWGLRKVRAENDRHHALDAVVVACSTVAMQQKIT
RFVRYKEMNAFDGKTIDKETGKVLHQKTHFPQPWEFFAQEVMIRVFGKPDGKPEFEEADTPEKLR
TLLAEKLSSRPEAVHEYVTPLFVSRAPNRKMSGAHKDTLRSAKRFVKHNEKISVKRVWLTEIKLA
DLENMVNYKNGREIELYEALKARLEAYGGNAKQAFDPKDNPFYKKGGQLVKAVRVEKTQESGVLL
NKKNAYTIADNGDMVRVDVFCKVDKKGKNQYFIVPIYAWQVAENILPDIDCKGYRIDDSYTFCFS
LHKYDLIAFQKDEKSKVEFAYYINCDSSNGRFYLAWHDKGSKEQQFRISTQNLVLIQKYQVNELG
KEIRPCRLKKRPPVR
Exemplary mRNA encoding UGI
(SEQ ID NO: 224)
GGGAGACCCAAGCUGGCUAGCUCCCGCAGUCGGCGUCCAGCGGCUCUGCUUGUUCGUGUGUGUGU
CGUUGCAGGCCUUAUUCGGAUCCGCCACCAUGGGACCGAAGAAGAAGAGAAAGGUCGGAGGAGGA
AGCACAAACCUGUCGGACAUCAUCGAAAAGGAAACAGGAAAGCAGCUGGUCAUCCAGGAAUCGAU
CCUGAUGCUGCCGGAAGAAGUCGAAGAAGUCAUCGGAAACAAGCCGGAAUCGGACAUCCUGGUCC
ACACAGCAUACGACGAAUCGACAGACGAAAACGUCAUGCUGCUGACAUCGGACGCACCGGAAUAC
AAGCCGUGGGCACUGGUCAUCCAGGACUCGAACGGAGAAAACAAGAUCAAGAUGCUGUGAUAGUC
UAGACAUCACAUUUAAAAGCAUCUCAGCCUACCAUGAGAAUAAGAGAAAGAAAAUGAAGAUCAAU
AGCUUAUUCAUCUCUUUUUCUUUUUCGUUGGUGUAAAGCCAACACCCUGUCUAAAAAACAUAAAU
UUCUUUAAUCAUUUUGCCUCUUUUCUCUGUGCUUCAAUUAAUAAAAAAUGGAAAGAACCUCGAGU
CUAG
Open reading frame for UGI
(SEQ ID NO: 225)
AUGGGACCGAAGAAGAAGAGAAAGGUCGGAGGAGGAAGCACAAACCUGUCGGACAUCAUCGAAAA
GGAAACAGGAAAGCAGCUGGUCAUCCAGGAAUCGAUCCUGAUGCUGCCGGAAGAAGUCGAAGAAG
UCAUCGGAAACAAGCCGGAAUCGGACAUCCUGGUCCACACAGCAUACGACGAAUCGACAGACGAA
AACGUCAUGCUGCUGACAUCGGACGCACCGGAAUACAAGCCGUGGGCACUGGUCAUCCAGGACUC
GAACGGAGAAAACAAGAUCAAGAUGCUGUGA
Exemplary amino acid sequence for UGI
(SEQ ID NO: 226)
MTNLSDIIEKETGKQLVIQESILMLPEEVEEVIGNKPESDILVHTAYDESTDENVMLLTSDAPEY
KPWALVIQDSNGENKIKMLSGGSKRTADGSEFESPKKKRKVE
Exemplary amino acid sequence for UGI
(SEQ ID NO: 227)
TNLSDIIEKETGKQLVIQESILMLPEEVEEVIGNKPESDILVHTAYDESTDENVMLLTSDAPEYK
PWALVIQDSNGENKIKML

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA-binding agent such as Cas9 to a target sequence, and the guide RNA hybridizes with and the agent binds to the target sequence; in cases where the agent is a cleavase or nickase, binding can be followed by double-stranded DNA cleavage or single-stranded DNA cleavage.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, a first sequence is considered to be “X % complementary to” a second sequence if X % of the bases of the first sequence base pairs with the second sequence. For example, a first sequence 5′AAGA3′ is 100% complementary to a second sequence 3′TTCT5′, and the second sequence is 100% complementary to the first sequence. In some embodiments, a first sequence 5′AAGA3′ is 100% complementary to a second sequence 3′TTCTGTGA5′, whereas the second sequence is 50% complementary to the first sequence.

As used herein, “mRNA” is used herein to refer to a polynucleotide that is entirely or predominantly RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indel” refers to insertion/deletion mutations consisting of a number of nucleotides that are either inserted or deleted at the site of a double-stranded break (DSB) in a target nucleic acid. As used herein, when indel formation that results in an insertion, the insertion is a random insertion at the site of a double stranded break and is not directed by or based on a template sequence.

As used herein, a “target sequence” refers to a sequence of nucleic acid in a target gene that has complementarity to the guide sequence of the gRNA. The interaction of the target sequence and the guide sequence directs an RNA-guided DNA-binding agent to bind, and potentially nick or cleave (depending on the activity of the agent), within the target sequence.

As used herein, “polypeptide” refers to a wild-type or variant protein (e.g., mutant, fragment, fusion, or combinations thereof). A variant polypeptide may possess at least or about 5%, 10%, 15%, 20%, 30%, 35%, 40%, 45%, 50%, 55%, 60%, 65%, 70%, 75%, 80%, 85%, 90%, 95%, or 100% functional activity of the wild-type polypeptide. In some embodiments, the variant is at least 70%, 75%, 80%, 85%, 90%, 92%, 93%, 94%, 95%, 96%, 97%, 98%, or 99% identical to the sequence of the wild-type polypeptide. In some embodiments, a variant polypeptide may be a hyperactive variant. In certain instances, the variant possesses between about 80% and about 120%, 140%, 160%, 180%, 200%, 300%, 400%, 500%, or more of a functional activity of the wild-type polypeptide. As used herein, a “heterologous gene” refers to a gene that has been introduced as an exogenous source within a cell (e.g., inserted at a genomic locus such as a safe harbor locus including a TCR gene locus). That is, the introduced gene is heterologous with respect to its insertion site. A polypeptide expressed from such heterologous gene is referred to as a “heterologous polypeptide.” The heterologous gene can be naturally-occurring or engineered, and can be wild-type or a variant. The heterologous gene may include nucleotide sequences other than the sequence that encodes the heterologous polypeptide (e.g., an internal ribosomal entry site). The heterologous gene can be a gene that occurs naturally in the genome, as a wild-type or a variant (e.g., mutant). For example, although the cell contains the gene of interest (as a wild-type or as a variant), the same gene or variant thereof can be introduced as an exogenous source for, e.g., expression at a locus that is highly expressed. The heterologous gene can also be a gene that is not naturally occurring in the genome, or that expresses a heterologous polypeptide that does not naturally occur in the genome. “Heterologous gene”, “exogenous gene”, and “transgene” are used interchangeably. In some embodiments, the heterologous gene or transgene includes an exogenous nucleic acid sequence, e.g., a nucleic acid sequence is not endogenous to the recipient cell. In some embodiments, the heterologous gene or transgene includes an exogenous nucleic acid sequence, e.g., a nucleic acid sequence that does not naturally occur in the recipient cell. For example, a heterologous gene a heterologous gene may be heterologous with respect to its insertion site and with respect to its recipient cell.

A “safe harbor” locus is a locus within the genome wherein a gene may be inserted without significant deleterious effects on the cell. Non-limiting examples of safe harbor loci that are targeted by nuclease(s) for use herein include AAVS1 (PPP1 R12C), TCR, B2M, and any of the loci targeted for knockdown described herein, e.g., TNFA, IFNG, IL17A, and IL6 genomic loci. In some embodiments, insertions at a locus or loci targeted for knockdown such as a TRC gene, e.g., TRAC gene, is advantageous for allogenic cells. Other suitable safe harbor loci are known in the art.

### II. Compositions

### A. Engineered T Cells

Provided herein are T cells and populations of T cells engineered to comprise a modification comprising insertion into the cell of heterologous sequence encoding a dmTGFB1 that is under the control of a promoter.

In some embodiments, the T cells comprising heterologous sequence encoding a dmTGFB1 under the control of a promoter are further engineered to comprise an additional modification e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under the control of a promoter sequence, as well as compositions and uses thereof. In some embodiments, the regulatory T cell promoting molecule is selected from IL10, CTLA4, IDO1, ENTPD1, NT5E, IL22, AREG, IL35, GARP, CD274, FOXP3, IKZF2, EOS, IRF4, LEF1, BACH2, and IL2RA.

In some embodiments, the T cells or population of T cells comprising heterologous sequence encoding a dmTGFB1 molecule under the control of a promoter is further engineered to comprise a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding two or more regulatory T cell promoting molecules each under the control of a promoter sequence. For example, the engineered T cell comprises a first heterologous sequence encoding a first regulatory T cell promoting molecule that is under the control of a first promoter and a second heterologous sequence encoding a second regulatory T cell promoting molecule that is under the control of a second promoter. The first promoter and the second promoter may be the same promoter or different promoters. In certain embodiments, the heterologous sequence encoding the dmTGFB1 molecule is under the control of promotor sequence that controls expression of a regulatory T cell promoting molecule. In certain embodiments, the heterologous sequence encoding the dmTGFB1 molecule is not under the control of promotor sequence that controls expression of a regulatory T cell promoting molecule. In certain embodiments, the heterologous sequence encoding the dmTGFB1 molecule is under the control of promotor sequence that controls expression of a targeting receptor. In certain embodiments, the heterologous sequence encoding the dmTGFB1 molecule is not under the control of promotor sequence that controls expression of a targeting receptor.

In some embodiments, the T cells or population of T cells comprising heterologous sequence encoding a dmTGFB1 under the control of a promoter is further engineered to comprise a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding IL10 that is under the control of a promoter. In some embodiments, the T cell comprising heterologous sequence encoding a dmTGFB1 under the control of a promoter is further engineered to comprise a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding CTLA4 that is under the control of a promoter. In some embodiments, the T cell comprising heterologous sequence encoding a dmTGFB1 under the control of a promoter is further engineered to comprise a modification, e.g., knockdown, of an endogenous nucleic acid sequence encoding a TNFA; a modification, e.g., knockdown, of an endogenous nucleic acid sequence encoding an IFNG or an IL17A, insertion into the cell of heterologous sequence(s) encoding IL10 that is under the control of a promoter, and insertion into the cell of heterologous sequence(s) encoding CTLA4 that is under the control of a promoter.

In some embodiments, the T cells or population of T cells comprising heterologous sequence encoding a dmTGFB1 under the control of a promoter is further engineered to comprise a modification, e.g., knockdown, of an endogenous nucleic acid sequence encoding a TNFA, a modification, e.g., knockdown, of an endogenous nucleic acid sequence encoding an IFNG or an IL17A; and insertion into the cell of heterologous sequence(s) encoding a regulatory T cell promoting molecule that is under the control of a promoter, and exhibits at least one suppressive activity of a naturally occurring regulatory T cell (nTreg), e.g., suppression of an immune response or biomarker in an in vitro or in vivo assay, e.g., an animal model of GvHD.

In some embodiments, the heterologous sequence(s) encoding dmTGFB1 or the regulatory T cell promoting molecule is incorporated into an expression construct. In some embodiments, heterologous sequences encoding two or more molecules may be incorporated into two or more separate expression constructs. For example, a first heterologous sequence encoding a dmTGFB1 is provided in a first expression construct, and a second heterologous sequence encoding a regulatory T cell promoting molecule is provided in a second, separate expression construct. In some embodiments, the expression construct is an episomal expression construct. In some embodiments, the heterologous sequence(s) is inserted into the genome, e.g., a targeted or an untargeted insertion.

In certain embodiments, modification, e.g., knockdown, of an endogenous nucleic acid sequence encoding an IFNG or an IL17A includes modification of IFNG and IL17A.

In some embodiments, the sequence(s) encoding dmTGFB1 or the regulatory T cell promoting molecule may be inserted into a site selected from a TCR gene locus, e.g., TRAC locus; a TNF gene locus, an IFNG gene locus, a IL17A locus, a IL6 locus, an IL2 locus, or an adeno-associated virus integration site 1 (AAVS1) locus.

In some embodiments, the engineered T cells or population of T cells engineered to comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule.

In some embodiments, the population of engineered T cells comprise a modification of an insertion of a sequence encoding a dmTGFB1 generated e.g., by gene editing, and e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, further comprises a modification, e.g., knockdown, in a TNFA sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous TNFA sequence. In some embodiments, the expression of TNFA (full-length, wild-type protein or mRNA) is decreased by at least 50%, 55%, 60%, 65%, preferably at least 70%, 75%, 80%, 85%, 90%, 95%, or to below the limit of detection of the assay as compared to a suitable control, e.g., wherein the TNFA gene has not been modified as determined, e.g., by ELISA or flow cytometry. Assays for TNFA protein and mRNA expression, e.g., in the population of T cells, are known in the art. In certain embodiments, knockdown of TNFA results in a TNFA level of 2500 pg/ml or less as determined, for example, using a custom U-PLEX Biomarker kit (Meso Scale Diagnostics, Cat. K15067L-2), according to manufacturer's instructions.

In some embodiments, the population of engineered T cells comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, further comprises a modification, e.g., knockdown, in an IFNG sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous IFNG sequence. In some embodiments, the expression of IFNG (full-length, wild-type protein or mRNA) is decreased by at least 50%, 55%, 60%, 65%, preferably at least 70%, 75%, 80%, 85%, 90%, 95%, or to below the limit of detection of the assay as compared to a suitable control, e.g., wherein the IFNG gene has not been modified as determined, e.g., by ELISA or flow cytometry. Assays for IFNG protein and mRNA expression, e.g., in the population of T cells, are known in the art. In certain embodiments, knockdown of IFNG results in an IFNG level of 300,000 pg/ml or less as determined, for example, using a custom U-PLEX Biomarker kit (Meso Scale Diagnostics, Cat. K15067L-2), according to manufacturer's instructions.

In some embodiments, the population of engineered T cells comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, further comprises a modification, e.g., knockdown, in an IL17A sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous IL17A sequence. In some embodiments, the expression of IL17A (full-length, wild-type protein or mRNA) is decreased by at least 50%, 55%, 60%, 65%, preferably at least 70%, 75%, 80%, 85%, 90%, 95%, or to below the limit of detection of the assay as compared to a suitable control, e.g., wherein the IL17A gene has not been modified as determined, e.g., by ELISA or flow cytometry. Assays for IL17A protein and mRNA expression, e.g., in the population of T cells, are known in the art.

In some embodiments, the modification that knocks down expression of a gene, e.g., TNFA, IFNG, or IL17A, is one or more of an insertion, a deletion, or a substitution.

In some embodiments, the engineered T cells or population of T cells comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, further comprise an insertion of sequence(s) encoding a regulatory T cell promoting molecule, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a regulatory T cell promoting molecule. In some embodiments, the inserted regulatory T cell promoting molecule, e.g., IL10, results in statistically significantly increased expression of protein or mRNA as compared to a suitable control, e.g., wherein the regulatory T cell promoting molecule gene has not been inserted as determined, e.g., by ELISA or flow cytometry. In some embodiments, the engineered T cells comprise an insertion of sequence(s) encoding IL10 by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion or a sequence encoding IL10. In some embodiments, the inserted sequence(s) encoding IL10 results in statistically significantly increased expression of protein or mRNA as compared to a suitable control, e.g., wherein the regulatory T cell promoting molecule. Assays for IL10 protein and mRNA expression, e.g., in the population of T cells, are known in the art, e.g., ELISA and flow cytometry. In certain embodiments, the level of IL10 is at least 300 pg/ml as determined, for example, using a custom U-PLEX Biomarker kit (Meso Scale Diagnostics, Cat. K15067L-2), according to manufacturer's instructions.

In some embodiments, the engineered T cells or population of T cells comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, further comprise an insertion of sequence(s) encoding CTLA4 e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion or a sequence encoding CTLA4. In some embodiments, the inserted sequence(s) encoding CTLA4 results in statistically significantly increased expression of protein or mRNA as compared to a suitable control, e.g., wherein the regulatory T cell promoting molecule. Assays for CTLA4 protein and mRNA expression, e.g., in the population of T cells, are described herein and known in the art, e.g., ELISA and flow cytometry.

In some embodiments, a population of T cells comprises T cells engineered to comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, are further engineered to comprise a modification, e.g., knockdown, of an endogenous nucleic acid sequence encoding a TNFA; a modification, e.g., knockdown, of an endogenous nucleic acid sequence encoding an IFNG or an IL17A; and insertion of sequences encoding a regulatory T cell promoting molecule. In some embodiments, at least 40%, 45%, preferably at least 50%, 55%, 60%, 65%, 70%, 75%, 80%, 85%, 90%, 95%, or 100% (e.g., within the detection limits of the assay used) of the T cells in the population of T cells are engineered to comprise a heterologous regulatory T cell promoting molecule, e.g., as assessed by sequencing, e.g., NGS. In some embodiments, at least 50%, 55%, 60%, 65%, preferably at least 70%, 75%, preferably at least 80%, 85%, 90%, 95%, or 100% of the T cells in the population of T cells are engineered to comprise a modification, e.g., knockdown, of sequence(s) encoding TNFA, e.g., as assessed by sequencing, e.g., NGS. In some embodiments, at least 50%, 55%, 60%, 65%, preferably at least 70%, 75%, 80%, 85%, 90%, 95%, or 100% of the T cells in the population of T cells are engineered to comprise a modification, e.g., knockdown, of sequence(s) encoding IFNG, e.g., as assessed by sequencing, e.g., NGS. In some embodiments, at least 40%, 45%, preferably at least 50%, 55%, 60%, 65%, 70%, 75%, 80%, 85%, 90%, 95%, or 100% of the T cells in the population of T cells are engineered to comprise insertion of sequences encoding a regulatory T cell promoting molecule, e.g., as assessed by sequencing, e.g., NGS. In some embodiments, at least 30%, 35%, preferably at least 40%, 45%, 50%, 55%, 60%, 65%, 70%, 75%, 80%, 85%, 90%, 95%, or 100% of the T cells in the population of T cells are engineered to comprise insertion of sequence(s) encoding IL10, e.g., as assessed by sequencing, e.g., NGS. In some embodiments, at least 30%, 35%, preferably at least 40%, 45%, 50%, 55%, 60%, 65%, 70%, 75%, 80%, 85%, 90%, 95%, or 100% of the T cells in the population of T cells are engineered to comprise insertion of sequence(s) encoding CTLA4, e.g., as assessed by sequencing, e.g., NGS.

In some embodiments, the engineered T cells or population of T cells engineered to comprise a modification, e.g., an insertion of a sequence encoding a dmTGFB1, e.g., by gene editing, e.g., as assessed by sequencing, e.g., NGS, wherein at least 30%, 35%, preferably at least 40%, 45%, 50%, 55%, 60%, 65%, 70%, 75%, 80%, 85%, 90%, or 95% of cells comprise an insertion of a sequence encoding a dmTGFB1 molecule, further comprise a modification, e.g., knockdown, of an endogenous nucleic acid sequence encoding a TNFA; a modification, e.g., knockdown, of an endogenous nucleic acid sequence encoding an IFNG or an IL17A; and insertion of heterologous sequence(s) encoding a regulatory T cell promoting molecule under control of a promoter sequence, further comprise a modification of an endogenous nucleic acid sequence encoding an interleukin 6 (IL6), interleukin 2 (IL2), a perforin 1 (PRF1), a granzyme A (GZMA), a granzyme B (GZMB), Fas ligand (FasL, NF superfamily, member 6), ryanodine receptor 2 (RYR2), and colony stimulating factor 2 (CSF2) wherein the modification knocks down expression of the IL17A, the IL6, the IL2, the PRF1, the GZMA, the GZMB, the FASL, the RYR2, or the CSF2, respectively. In certain embodiments, the further modification of an endogenous nucleic acid sequence comprises modification of RYR2.

In certain embodiments, the modifications comprise modification of IFNG and IL17A.

In some embodiments, the T cells or population of T cells are engineered using a gene editing system, e.g., using an RNA-guided DNA binding agent. In some embodiments, the T cells are engineered using a CRISPR/Cas gene editing system. In some embodiments, the T cells are engineered using a CRISPR/Cas type II gene editing system, e.g., using Cpf1. In some embodiments, the T cells are engineered using a CRISPR/Cas9 gene editing system, e.g., using SpyCas9. Exemplary Cas9 sequences are provided herein.

In some embodiments, the T cells or population of T cells are engineered using guide RNAs that specifically target sites within the IFNG and TNFA genes to provide knockdown of the of IFNG and TNFA genes. Exemplary sequences are provided in Tables 1 and 2 for knockdown of IFNG and TNFA, respectively, as are genomic coordinates of the target of each listed guide sequence.

In some embodiments, the engineered T cells or population of T cells comprise IFNG and TNFA genes that are knocked down using a guide RNA disclosed herein with an RNA-guided DNA binding agent. In some embodiments, disclosed herein are T cells engineered by inducing a break (e.g., double-stranded break (DSB) or single-stranded break (nick)) within the IL-7A, IFNG, and TNFA genes of a T cell, e.g., using a guide RNA disclosed herein with an RNA-guided DNA-binding agent (e.g., a CRISPR/Cas system). The methods may be used in vitro or ex vivo, e.g., in the manufacture of cell products for suppressing immune response(s), including inflammation and autoimmunity. In some embodiments, the guide RNAs disclosed herein mediate a target-specific cutting by an RNA-guided DNA-binding agent (e.g., Cas nuclease) at a site described herein within an IFNG gene. In some embodiments, the guide RNAs disclosed herein mediate a target-specific cutting by an RNA-guided DNA-binding agent (e.g., Cas nuclease) at a site described herein within a TNFA gene. It will be appreciated that, in some embodiments, the guide RNAs comprise guide sequences that bind to, or are capable of binding to, said regions.

Engineered T cells or population of T cells comprising a genetic modification at genomic coordinates chosen from those listed in Table 1 are provided, e.g., cells comprising an indel or substitution mutation within any of the listed genomic ranges within IFNG. Engineered T cells comprising a genetic modification at genomic coordinates chosen from those listed in Table 2 are also provided, e.g., cells comprising an indel or substitution mutation within any of the listed genomic ranges within TNFA. In some embodiments, the engineered T cell will comprise a modification within a genomic coordinate region chosen from Table 1 and a modification with a genomic coordinate region chosen from Table 2.

In some embodiments, the guide RNAs disclosed herein comprise a guide sequence that is 95%, 90%, 85%, 80%, or 75% identical to a sequence selected from the group of sequences in Table 1 or Table 2. In some embodiments, the guide RNAs disclosed herein comprise a guide sequence that is 95%, 90%, 85%, 80%, or 75% identical to a sequence selected from the group of sequences in Table 1 or Table 2.

In some embodiments, the guide RNAs disclosed herein comprise a guide sequence having at least 15, 16, 17, 18, 19, or 20 contiguous nucleotides of a sequence selected from the group consisting of a sequence that is 95%, 90%, 85%, 80%, or 75% identical to a sequence selected from the group of sequences in Table 1. In some embodiments, the guide RNAs disclosed herein comprise a guide sequence at least 17, 18, 19, or 20 contiguous nucleotides of a sequence selected from the group of sequences in Table 1. In some embodiments, the guide RNAs disclosed herein comprise a guide sequence that is 95%, 90%, 85%, 80%, or 75% identical to a sequence selected from the group of sequences in Table 1. In some embodiments, the guide RNAs disclosed herein comprise a guide sequence that is 17, 18, 19, or 20 contiguous nucleotides of a sequence selected from the group of sequences in Table 1. In some embodiments, the guide RNAs disclosed herein comprise a guide sequence that is selected from the group of sequences in Table 1.

In some embodiments, the guide RNAs disclosed herein comprise a guide sequence having at least 15, 16, 17, 18, 19, or 20 contiguous nucleotides of a sequence selected from the group consisting of a sequence that is 95%, 90%, 85%, 80%, or 75% identical to a sequence selected from the group of sequences in Table 2. In some embodiments, the guide RNAs disclosed herein comprise a guide sequence at least 17, 18, 19, or 20 contiguous nucleotides of a sequence selected from the group of sequences in Table 2. In some embodiments, the guide RNAs disclosed herein comprise a guide sequence that is 95%, 90%, 85%, 80%, or 75% identical to a sequence selected from the group of sequences in Table 2. In some embodiments, the guide RNAs disclosed herein comprise a guide sequence that is 17, 18, 19, or 20 contiguous nucleotides of a sequence selected from the group of sequences in Table 2. In some embodiments, the guide RNAs disclosed herein comprise a guide sequence that is selected from the group of sequences in Table 2.

Genomic coordinates throughout are according to human reference genome hg38 unless otherwise noted.

In certain embodiments a guide RNA comprising a guide sequence targeting IFNG and a guide RNA comprising a guide sequence targeting TNFA are included.

TABLE 1
Human guide sequences and chromosomal coordinates for knockdown of
IFNG

| Exemplary Genomic | | | | SEQ |
|---|---|---|---|---|
| Coordinates (hg38) | Exon | Strand | Guide Sequence (5′ to 3′) | ID NO |
| chr12:68155336-68155356 | 4 | + | GCAGGCAGGACAACCAUUAC | 1 |
| chr12:68155337-68155357 | 4 | + | CAGGCAGGACAACCAUUACU | 2 |
| chr12:68155374-68155394 | 4 | − | AGGAGUCAGAUGCUGUUUCG | 3 |
| chr12:68155394-68155414 | 4 | − | CUAAAACAGGGAAGCGAAAA | 4 |
| chr12:68155398-68155418 | 4 | + | CGCUUCCCUGUUUUAGCUGC | 5 |
| chr12:68155406-68155426 | 4 | − | UGUCGCCAGCAGCUAAAACA | 6 |
| chr12:68155407-68155427 | 4 | − | CUGUCGCCAGCAGCUAAAAC | 7 |
| chr12:68155420-68155440 | 4 | + | GCGACAGUUCAGCCAUCACU | 8 |
| chr12:68155435-68155455 | 4 | − | ACAUGAACUCAUCCAAGUGA | 9 |
| chr12:68155447-68155467 | 4 | + | GUUCAUGUAUUGCUUUGCGU | 10 |
| chr12:68157972-68157992 | 3 | + | GACAUUCAUGUCUUCCUUGA | 11 |
| chr12:68157989-68158009 | 3 | + | UGAUGGUCUCCACACUCUUU | 12 |
| chr12:68157989-68158009 | 3 | − | AAAGAGUGUGGAGACCAUCA | 13 |
| chr12:68158001-68158021 | 3 | − | CCAGAGCAUCCAAAAGAGUG | 14 |
| chr12:68158215-68158235 | 2 | − | GAUAAUGGAACUCUUUUCUU | 15 |
| chr12:68158230-68158250 | 2 | − | CAUUCAGAUGUAGCGGAUAA | 16 |
| chr12:68158237-68158257 | 2 | − | UGCAGGUCAUUCAGAUGUAG | 17 |
| chr12:68159524-68159544 | 1 | + | CUUCUUUUACAUAUGGGUCC | 18 |
| chr12:68159559-68159579 | 1 | − | UGCAUCGUUUUGGGUUCUCU | 19 |
| chr12:68159568-68159588 | 1 | − | UUUCAGCUCUGCAUCGUUUU | 20 |
| chr12:68159569-68159589 | 1 | − | UUUUCAGCUCUGCAUCGUUU | 21 |

TABLE 2
Human guide sequences and chromosomal coordinates for knockdown of
TNFA

| Exemplary Genomic | | | | SEQ |
|---|---|---|---|---|
| Coordinates (hg38) | Exon | Strand | Guide sequence (5′ to 3′) | ID NO |
| chr6:31575742-31575762 | 1 | + | UGAGCACUGAAAGCAUGAUC | 22 |
| chr6:31575749-31575769 | 1 | + | UGAAAGCAUGAUCCGGGACG | 23 |
| chr6:31575755-31575775 | 1 | + | CAUGAUCCGGGACGUGGAGC | 24 |
| chr6:31575781-31575801 | 1 | − | CUUCUUGGGGAGCGCCUCCU | 25 |
| chr6:31575783-31575803 | 1 | + | GAGGCGCUCCCCAAGAAGAC | 26 |
| chr6:31575784-31575804 | 1 | + | AGGCGCUCCCCAAGAAGACA | 27 |
| chr6:31575785-31575805 | 1 | + | GGCGCUCCCCAAGAAGACAG | 28 |
| chr6:31575786-31575806 | 1 | + | GCGCUCCCCAAGAAGACAGG | 29 |
| chr6:31575794-31575814 | 1 | + | CAAGAAGACAGGGGGGCCCC | 30 |
| chr6:31575794-31575814 | 1 | − | GGGGCCCCCCUGUCUUCUUG | 31 |
| chr6:31575796-31575816 | 1 | − | CUGGGGCCCCCCUGUCUUCU | 32 |
| chr6:31575813-31575833 | 1 | − | AAGCACCGCCUGGAGCCCUG | 33 |
| chr6:31575814-31575834 | 1 | − | CAAGCACCGCCUGGAGCCCU | 34 |
| chr6:31575823-31575843 | 1 | − | GCUGAGGAACAAGCACCGCC | 35 |
| chr6:31575842-31575862 | 1 | + | CCUCUUCUCCUUCCUGAUCG | 36 |
| chr6:31575846-31575866 | 1 | + | UUCUCCUUCCUGAUCGUGGC | 37 |
| chr6:31575853-31575873 | 1 | − | GGCGCCUGCCACGAUCAGGA | 38 |
| chr6:31575857-31575877 | 1 | − | UGGUGGCGCCUGCCACGAUC | 39 |
| chr6:31575888-31575908 | 1 | + | CUGCUGCACUUUGGAGUGAU | 40 |
| chr6:31575890-31575910 | 1 | − | CGAUCACUCCAAAGUGCAGC | 41 |
| chr6:31575898-31575918 | 1 | + | UUGGAGUGAUCGGCCCCCAG | 42 |
| chr6:31575899-31575919 | 1 | + | UGGAGUGAUCGGCCCCCAGA | 43 |
| chr6:31575917-31575937 | 1 | − | AGGCACUCACCUCUUCCCUC | 44 |
| chr6:31576538-31576558 | 2 | − | UGAUUAGAGAGAGGUCCCUG | 45 |
| chr6:31576539-31576559 | 2 | − | CUGAUUAGAGAGAGGUCCCU | 46 |
| chr6:31576540-31576560 | 2 | − | GCUGAUUAGAGAGAGGUCCC | 47 |
| chr6:31576544-31576564 | 2 | + | CCUCUCUCUAAUCAGCCCUC | 48 |
| chr6:31576547-31576567 | 2 | − | CCAGAGGGCUGAUUAGAGAG | 49 |
| chr6:31576550-31576570 | 2 | + | UCUAAUCAGCCCUCUGGCCC | 50 |
| chr6:31576562-31576582 | 2 | − | UACUGACUGCCUGGGCCAGA | 51 |
| chr6:31576563-31576583 | 2 | − | UUACUGACUGCCUGGGCCAG | 52 |
| chr6:31576570-31576590 | 2 | − | GAGACACUUACUGACUGCCU | 53 |
| chr6:31576571-31576591 | 2 | − | GGAGACACUUACUGACUGCC | 54 |
| chr6:31576785-31576805 | 3 | − | GGGCUACAGGCUUGUCACUC | 55 |
| chr6:31576786-31576806 | 3 | − | UGGGCUACAGGCUUGUCACU | 56 |
| chr6:31576798-31576818 | 3 | − | UUACCUACAACAUGGGCUAC | 57 |
| chr6:31576805-31576825 | 3 | − | AGAGCUCUUACCUACAACAU | 58 |
| chr6:31576806-31576826 | 3 | − | CAGAGCUCUUACCUACAACA | 59 |
| chr6:31577110-31577130 | 4 | + | UCCAGCAAACCCUCAAGCUG | 60 |
| chr6:31577112-31577132 | 4 | + | CAGCAAACCCUCAAGCUGAG | 61 |
| chr6:31577122-31577142 | 4 | − | GGAGCUGCCCCUCAGCUUGA | 62 |
| chr6:31577123-31577143 | 4 | − | UGGAGCUGCCCCUCAGCUUG | 63 |
| chr6:31577136-31577156 | 4 | + | AGCUCCAGUGGCUGAACCGC | 64 |
| chr6:31577137-31577157 | 4 | + | GCUCCAGUGGCUGAACCGCC | 65 |
| chr6:31577143-31577163 | 4 | − | UGGCCCGGCGGUUCAGCCAC | 66 |
| chr6:31577152-31577172 | 4 | + | CCGCCGGGCCAAUGCCCUCC | 67 |
| chr6:31577155-31577175 | 4 | − | CCAGGAGGGCAUUGGCCCGG | 68 |
| chr6:31577159-31577179 | 4 | + | GCCAAUGCCCUCCUGGCCAA | 69 |
| chr6:31577163-31577183 | 4 | − | GCCAUUGGCCAGGAGGGCAU | 70 |
| chr6:31577164-31577184 | 4 | + | UGCCCUCCUGGCCAAUGGCG | 71 |
| chr6:31577170-31577190 | 4 | − | GCUCCACGCCAUUGGCCAGG | 72 |
| chr6:31577173-31577193 | 4 | − | UCAGCUCCACGCCAUUGGCC | 73 |
| chr6:31577178-31577198 | 4 | − | AUCUCUCAGCUCCACGCCAU | 74 |
| chr6:31577185-31577205 | 4 | + | GGAGCUGAGAGAUAACCAGC | 75 |
| chr6:31577188-31577208 | 4 | + | GCUGAGAGAUAACCAGCUGG | 76 |
| chr6:31577200-31577220 | 4 | + | CCAGCUGGUGGUGCCAUCAG | 77 |
| chr6:31577201-31577221 | 4 | + | CAGCUGGUGGUGCCAUCAGA | 78 |
| chr6:31577216-31577236 | 4 | − | AUGAGGUACAGGCCCUCUGA | 79 |
| chr6:31577227-31577247 | 4 | − | CCUGGGAGUAGAUGAGGUAC | 80 |
| chr6:31577237-31577257 | 4 | + | UACUCCCAGGUCCUCUUCAA | 81 |
| chr6:31577245-31577265 | 4 | − | CUUGGCCCUUGAAGAGGACC | 82 |
| chr6:31577295-31577315 | 4 | − | GGCGAUGCGGCUGAUGGUGU | 83 |
| chr6:31577296-31577316 | 4 | − | CGGCGAUGCGGCUGAUGGUG | 84 |
| chr6:31577301-31577321 | 4 | − | GGAGACGGCGAUGCGGCUGA | 85 |
| chr6:31577308-31577328 | 4 | − | UCUGGUAGGAGACGGCGAUG | 86 |
| chr6:31577311-31577331 | 4 | + | CGCCGUCUCCUACCAGACCA | 87 |
| chr6:31577322-31577342 | 4 | − | GAGGUUGACCUUGGUCUGGU | 88 |
| chr6:31577331-31577351 | 4 | − | GGCAGAGAGGAGGUUGACCU | 89 |
| chr6:31577349-31577369 | 4 | + | CCAUCAAGAGCCCCUGCCAG | 90 |
| chr6:31577350-31577370 | 4 | + | CAUCAAGAGCCCCUGCCAGA | 91 |
| chr6:31577362-31577382 | 4 | − | CUGGGGUCUCCCUCUGGCAG | 92 |
| chr6:31577363-31577383 | 4 | − | UCUGGGGUCUCCCUCUGGCA | 93 |
| chr6:31577397-31577417 | 4 | − | GAUGGGCUCAUACCAGGGCU | 94 |
| chr6:31577401-31577421 | 4 | + | CUGGUAUGAGCCCAUCUAUC | 95 |
| chr6:31577402-31577422 | 4 | − | AGAUAGAUGGGCUCAUACCA | 96 |
| chr6:31577402-31577422 | 4 | + | UGGUAUGAGCCCAUCUAUCU | 97 |
| chr6:31577403-31577423 | 4 | − | CAGAUAGAUGGGCUCAUACC | 98 |
| chr6:31577406-31577426 | 4 | 1 | AUGAGCCCAUCUAUCUGGGA | 99 |
| chr6:31577407-31577427 | 4 | + | UGAGCCCAUCUAUCUGGGAG | 100 |
| chr6:31577414-31577434 | 4 | − | AAGACCCCUCCCAGAUAGAU | 101 |
| chr6:31577437-31577457 | 4 | − | GUCGGUCACCCUUCUCCAGC | 102 |
| chr6:31577454-31577474 | 4 | + | GACUCAGCGCUGAGAUCAAU | 103 |
| chr6:31577455-31577475 | 4 | − | GAUUGAUCUCAGCGCUGAGU | 104 |
| chr6:31577480-31577500 | 4 | − | UCGGCAAAGUCGAGAUAGUC | 105 |
| chr6:31577481-31577501 | 4 | − | CUCGGCAAAGUCGAGAUAGU | 106 |
| chr6:31577483-31577503 | 4 | + | UAUCUCGACUUUGCCGAGUC | 107 |
| chr6:31577484-31577504 | 4 | + | AUCUCGACUUUGCCGAGUCU | 108 |
| chr6:31577488-31577508 | 4 | + | CGACUUUGCCGAGUCUGGGC | 109 |
| chr6:31577498-31577518 | 4 | + | GAGUCUGGGCAGGUCUACUU | 110 |
| chr6:31577499-31577519 | 4 | + | AGUCUGGGCAGGUCUACUUU | 111 |
| chr6:31577499-31577519 | 4 | − | AAAGUAGACCUGCCCAGACU | 112 |
| chr6:31577532-31577552 | 4 | − | GGAUGUUCGUCCUCCUCACA | 113 |

A non-limiting modified guide sequence for knockdown of TNFA is shown below (hg38 coordinates chr12: 68158001-68158021, G019757): mC*mC*mA*GAGCAUCCAAAAGAGUGGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 119), wherein m is a 2′-OMe modified nucleotide/nucleoside residue, * is indicative of a phosphorothioate linkage between the residues, a capital letter indicates a residue, preferably comprising a ribose sugar.

A non-limiting modified guide sequence for knockdown of IFNG is shown below (hg38 coordinates chr6: 31576805-31576825, G019753):

(SEQ ID NO: 120)
mA*mG*mA*GCUCUUACCUACAACAUGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmU*mU*mU
*mU.

A non-limiting modified guide sequence for knockdown of IL17A is shown below (hg38 coordinates chr6: 52189069-52189089):

(SEQ ID NO: 217)
mU*mC*mA*CAGAGGGAUAUCUCUCAGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmU*mU*mU
*mU.

An exemplary modified mock guide is shown below (hg38 coordinates chr1: 0-20):

(SEQ ID NO: 121)
mG*mA*mU*CACGUCGGCCGUUGGCGGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmU*mU*mU
*mU.

In some embodiments, disclosed herein are T cells engineered by introducing or inserting a heterologous dmTGFB1 nucleic acid within a genomic locus of a T cell or a population of T cells using a guide RNA with an RNA-guided DNA binding agent, and a construct (e.g., donor construct or template) comprising a heterologous dmTGFB1 nucleic acid, e.g., to make an engineered T cell. In some embodiments, disclosed herein are T cells engineered by expressing a heterologous dmTGFB1 from a genomic locus of a T cell or a population of T cells, e.g., using a guide RNA with an RNA-guided DNA-binding agent and a construct (e.g., donor) comprising a heterologous dmTGFB1 nucleic acid. In some embodiments, disclosed herein are T cells engineered by inducing a break (e.g., double-stranded break (DSB) or single-stranded break (nick)) within the genome of a T cell or a population of T cells for inserting the dmTGFB1 gene, e.g., using a guide RNA with an RNA-guided DNA-binding agent (e.g., a CRISPR/Cas system). Cells and cell populations made by the methods are also provided.

In some embodiments, disclosed herein are T cells engineered by introducing or inserting a heterologous dmTGFB1 nucleic acid within a genomic locus of a T cell, are further engineered by introducing or inserting a heterologous IL10 nucleic acid within a genomic locus of a T cell or a population of T cells using a guide RNA with an RNA-guided DNA binding agent, and a construct (e.g., donor construct or template) comprising a heterologous IL10 nucleic acid, e.g., to make an engineered T cell. In some embodiments, disclosed herein are T cells engineered by expressing a heterologous IL10 from a genomic locus of a T cell or a population of T cells, e.g., using a guide RNA with an RNA-guided DNA-binding agent and a construct (e.g., donor) comprising a heterologous IL10 nucleic acid. In some embodiments, disclosed herein are T cells engineered by inducing a break (e.g., double-stranded break (DSB) or single-stranded break (nick)) within the genome of a T cell or a population of T cells for inserting the IL10 gene, e.g., using a guide RNA with an RNA-guided DNA-binding agent (e.g., a CRISPR/Cas system). Cells and cell populations made by the methods are also provided.

In some embodiments, disclosed herein are T cells engineered by introducing or inserting a heterologous dmTGFB1 nucleic acid within a genomic locus of a T cell, are further engineered by introducing or inserting a heterologous CTLA4 nucleic acid within a genomic locus of a T cell or a population of T cells using a guide RNA with an RNA-guided DNA binding agent, and a construct (e.g., donor construct or template) comprising a heterologous CTLA4 nucleic acid, e.g., to make an engineered T cell. In some embodiments, disclosed herein are T cells engineered by expressing a heterologous CTLA4 from the genomic locus of a T cell or a population of T cells, e.g., using a guide RNA with an RNA-guided DNA-binding agent and a construct (e.g., donor) comprising a heterologous CTLA4 nucleic acid. In some embodiments, disclosed herein are T cells engineered by inducing a break (e.g., double-stranded break (DSB) or single-stranded break (nick)) within the genome of a T cell or a population of T cells for inserting the CTLA4 gene, e.g., using a guide RNA with an RNA-guided DNA-binding agent (e.g., a CRISPR/Cas system). Cells and cell populations made by the methods are also provided.

In some embodiments, disclosed herein are T cells engineered by introducing or inserting a heterologous dmTGFB1 nucleic acid within a genomic locus of a T cell, are further engineered by introducing or inserting a heterologous CTLA4 nucleic acid and a heterologous IL10 nucleic acid within a genomic locus of a T cell or a population of T cells using a guide RNA with an RNA-guided DNA binding agent, and one or more constructs (e.g., donor construct or template) comprising a heterologous CTLA4 nucleic acid and a heterologous IL10 nucleic acid, e.g., to make an engineered T cell. In some embodiments, disclosed herein are T cells engineered by expressing a heterologous CTLA4 and a heterologous IL10 from the genomic locus of a T cell or a population of T cells, e.g., using a guide RNA with an RNA-guided DNA-binding agent and one or more constructs (e.g., donor construct or template) comprising a heterologous CTLA4 nucleic acid and a heterologous IL10 nucleic acid. In some embodiments, disclosed herein are T cells engineered by inducing a break (e.g., double-stranded break (DSB) or single-stranded break (nick)) within the genome of a T cell or a population of T cells for inserting the CTLA4 gene and the IL10 gene, e.g., using a guide RNA with an RNA-guided DNA-binding agent (e.g., a CRISPR/Cas system). In some embodiments, the guide RNAs mediate a target-specific cutting by an RNA-guided DNA-binding agent (e.g., Cas nuclease) at a site described herein for insertion of sequence(s) encoding two or more regulatory T cell promoting molecule, e.g., IL10 and CTLA4. It will be appreciated that, in some embodiments, the guide RNAs comprise guide sequences that bind to, or are capable of binding to, said regions. Cells and cell populations made by the methods are also provided.

Exemplary nucleotide and polypeptide sequences of regulatory T cell promoting molecules are provided below. Methods for identifying alternate nucleotide sequences encoding polypeptide sequences, including alternate naturally occurring variants and non-human homologues, are known in the art. Exemplary nucleic acid sequences encoding dmTGFB1, IL10, and CTLA4 are provided below. Other suitable dmTGFB1, IL10, and CTLA4 sequences are known in the art or can be designed based on the disclosure provided herein. See, e.g., Gorby et al., Engineered IL-10 variants elicit potent immuno-modulatory activities at therapeutic low ligand doses, BioRxiv (2020); Saxton et al. Structure-based decoupling of the pro- and anti-inflammatory functions of interleukin-10, Science, (2021)371:eabc8433 (doi: 10.1126/science.abc8433), WO2021243057; and Xu et al., Affinity and cross-reactivity engineering of CTLA4-Ig to modulate T cell costimulation, J Immunol (2012), the contents, variants (in particular IL-10 variants), and sequences of which are hereby incorporated by reference. Methods for identifying alternate IL10 and CTLA4 sequences are also known in the art. See, e.g., id. For example, Gorby discloses the anti-inflammatory and pro-cytotoxicity activities of IL-10. “In addition to its anti-inflammatory activities, recent studies have shown that IL-10 can increase the cytotoxic function of CD8 T cells, augmenting their ability to target tumors and boosting the anti-cancer response (Oft, 2019). This seems paradoxical as IL-10 in the tumor microenvironment is linked to tumor evasion of the immune response, most likely due to IL-10's inhibitory effects on antigen presentation (Mannino et al., 2015; Yue et al., 1997). Despite this paradox, several studies have elegantly demonstrated that IL-10 can improve production of the CD8 effector molecules granzyme B and interferon gamma both in vitro and in vivo (Emmerich et al., 2012; Mumm et al., 2011; Mumm and Oft, 2013). Currently there are several clinical trials testing the anti-tumor properties of IL-10, with already initial promising results (Naing et al., 2019). In these trials high doses of PEGylated IL-10 (Pegilodekakin) were used, which resulted in prolonged IL-10 retention in the circulation to ensure efficacy, again highlighting that effective IL-10 in vivo responses need high concentrations and sustained levels of IL-10. Saxton et al., used yeast display-based directed evolution to engineer IL-10 variants. IL-10 is an immunoregulatory cytokine with both anti-inflammatory and immunostimulatory properties and is frequently dysregulated in disease. Mechanistically, IL-10 functions as a secreted homodimer that engages two copies of a heterodimeric receptor complex comprising the private receptor subunit, IL-10Rα, and the shared subunit, IL-10Rβ. The IL-10-dependent dimerization of IL-10Rα and IL-10Rβ in turn initiates activation of the transcription factor STAT3, which mediates the diverse biological effects of IL-10. Saxton et al. teach IL-10 variants with a range of IL-10Rβ binding strengths uncovered substantial differences in response thresholds across immune cell populations, providing a means of manipulating IL-10 cell type selectivity. A “super-10” variant (D25A/E96A) with enhanced affinity for IL-10Rβ, enabling assembly of the hexameric IL-10-IL-10Rα-IL-10Rβ complex was identified by Saxton et al. Further variants (e.g., D25K, D25A, N21A/R104A, and D25A/N21A/R104A based on amino acid numbering of SEQ ID NO: 231 displayed myeloid-biased activity by suppressing macrophage activation without stimulating inflammatory CD8+ T cells, thereby uncoupling the major opposing functions of IL-10. In certain embodiments, IL-10 variants, referred to herein as “suppressive IL-10 variants,” with impaired immunostimulatory properties, e.g. variants that retain myeloid-biased activity by suppressing macrophage activation and that display impaired stimulation of inflammatory CD8+ T cells, are used in the engineered T cells provided herein. In certain embodiments, suppressive IL-10 variants used in the engineered T cells provided herein include substitutions selected from D25K, D25A, D25A/E96A, N21A/R104A, and D25A/N21A/R104A, e.g., D25A/E96A. The results from Saxton et al. provide a mechanistic blueprint for tuning the pleiotropic actions of IL-10. Further suppressive IL-10 variants are provided in WO2021243057, which is incorporated herein by reference, which provides a number of IL-10 sequences, including polypeptides have an altered binding affinity for IL-10Rβ compared to binding affinity of a reference IL-10 polypeptide lacking the one or more amino acid substitution, and methods to characterize activity. Further variants, including suppressive IL-10 variants are disclosed and predicted to displayed myeloid-biased activity by suppressing macrophage activation without stimulating inflammatory CD8+ T cells, similar to the D25K, D25A, D25A/E96A, N21A/R104A, and D25A/N21A/R104A variants. Embodiments provided therein include suppressive IL-10 variants with one or more amino acid substitution is at a position corresponding to an amino acid residue selected from D25, H14, N18, R24, D28, E74, H90, N92, E96, T100, and R104, optionally including a further substitution at one or more amino acids selected from N21, M22, R32, and S93. In certain embodiments, D25 is substituted with an amino acid selected from K, A, N, H, I, K, or V. In certain embodiments, E96 is substituted with an amino acid selected from A, N, D, Q, H, K, or S. Exemplary combinations of substitutions can include a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. Exemplary substitutions include (a) D25A; (b) D25K; (c) E96A; (d) E96K; (e) D25A/E96A; (f) N21A/R104A; (g) N21A/D25A; (h) N21A/D25A/E96A; and (i) N21A/M22A/D25A. Sequences with at least 75%, 80%, 85%, 90%, 95%, 96%, 97%, 98%, or 99% identity to any of the nucleic acid sequences, amino acid sequences, or nucleic acid sequences encoding the amino acid sequences described herein, e.g., due to mutations or truncations, are also contemplated. In some embodiments, a nucleic acid sequence encoding any of the amino acid sequences provided herein is also provided.

Non-limiting exemplary nucleic acid sequences encoding TGFB1 are provided:

Wild-type TGFB1
(SEQ ID NO: 204)
ATGCCGCCCTCCGGGCTGCGGCTGCTGCTGCTGCTGCTACCGCTGCTGT
GGCTACTGGTGCTGACGCCTGGCCGGCCGGCCGCGGGACTATCCACCTGCAAGA
CTATCGACATGGAGCTGGTGAAGCGGAAGCGCATCGAGGCCATCCGCGGCCAGA
TCCTGTCCAAGCTGCGGCTCGCCAGCCCCCCGAGCCAGGGGGAGGTGCCGCCCG
GCCCGCTGCCCGAGGCCGTGCTCGCCCTGTACAACAGCACCCGCGACCGGGTGG
CCGGGGAGAGTGCAGAACCGGAGCCCGAGCCTGAGGCCGACTACTACGCCAAGG
AGGTCACCCGCGTGCTAATGGTGGAAACCCACAACGAAATCTATGACAAGTTCA
AGCAGAGTACACACAGCATATATATGTTCTTCAACACATCAGAGCTCCGAGAAG
CGGTACCTGAACCCGTGTTGCTCTCCCGGGCAGAGCTGCGTCTGCTGAGGCTCAA
GTTAAAAGTGGAGCAGCACGTGGAGCTGTACCAGAAATACAGCAACAATTCCTG
GCGATACCTCAGCAACCGGCTGCTGGCACCCAGCGACTCGCCAGAGTGGTTATCT
TTTGATGTCACCGGAGTTGTGCGGCAGTGGTTGAGCCGTGGAGGGGAAATTGAG
GGCTTTCGCCTTAGCGCCCACTGCTCCTGTGACAGCAGGGATAACACACTGCAAG
TGGACATCAACGGGTTCACTACCGGCCGCCGAGGTGACCTGGCCACCATTCATGG
CATGAACCGGCCTTTCCTGCTTCTCATGGCCACCCCGCTGGAGAGGGCCCAGCAT
CTGCAAAGCTCCCGGCACCGCCGAGCCCTGGACACCAACTATTGCTTCAGCTCCA
CGGAGAAGAACTGCTGCGTGCGGCAGCTGTACATTGACTTCCGCAAGGACCTCG
GCTGGAAGTGGATCCACGAGCCCAAGGGCTACCATGCCAACTTCTGCCTCGGGC
CCTGCCCCTACATTTGGAGCCTGGACACGCAGTACAGCAAGGTCCTGGCCCTGTA
CAACCAGCATAACCCGGGCGCCTCGGCGGCGCCGTGCTGCGTGCCGCAGGCGCT
GGAGCCGCTGCCCATCGTGTACTACGTGGGCCGCAAGCCCAAGGTGGAGCAGCT
GTCCAACATGATCGTGCGCTCCTGCAAGTGCAGC
Dual Mutant (dm) TGFB1 (R218 C, C225R)
(SEQ ID NO: 208)
ATGCCGCCCTCCGGGCTGCGGCTGCTGCTGCTGCTGCTACCGCTGCTGT
GGCTACTGGTGCTGACGCCTGGCCGGCCGGCCGCGGGACTATCCACCTGCAAGA
CTATCGACATGGAGCTGGTGAAGCGGAAGCGCATCGAGGCCATCCGCGGCCAGA
TCCTGTCCAAGCTGCGGCTCGCCAGCCCCCCGAGCCAGGGGGAGGTGCCGCCCG
GCCCGCTGCCCGAGGCCGTGCTCGCCCTGTACAACAGCACCCGCGACCGGGTGG
CCGGGGAGAGTGCAGAACCGGAGCCCGAGCCTGAGGCCGACTACTACGCCAAGG
AGGTCACCCGCGTGCTAATGGTGGAAACCCACAACGAAATCTATGACAAGTTCA
AGCAGAGTACACACAGCATATATATGTTCTTCAACACATCAGAGCTCCGAGAAG
CGGTACCTGAACCCGTGTTGCTCTCCCGGGCAGAGCTGCGTCTGCTGAGGCTCAA
GTTAAAAGTGGAGCAGCACGTGGAGCTGTACCAGAAATACAGCAACAATTCCTG
GCGATACCTCAGCAACCGGCTGCTGGCACCCAGCGACTCGCCAGAGTGGTTATCT
TTTGATGTCACCGGAGTTGTGCGGCAGTGGTTGAGCCGTGGAGGGGAAATTGAG
GGCTTTCACCTTAGCGCCCACTGCTCCAGAGACAGCAGGGATAACACACTGCAA
GTGGACATCAACGGGTTCACTACCGGCCGCCGAGGTGACCTGGCCACCATTCATG
GCATGAACCGGCCTTTCCTGCTTCTCATGGCCACCCCGCTGGAGAGGGCCCAGCA
TCTGCAAAGCTCCCGGCACCGCCGAGCCCTGGACACCAACTATTGCTTCAGCTCC
ACGGAGAAGAACTGCTGCGTGCGGCAGCTGTACATTGACTTCCGCAAGGACCTC
GGCTGGAAGTGGATCCACGAGCCCAAGGGCTACCATGCCAACTTCTGCCTCGGG
CCCTGCCCCTACATTTGGAGCCTGGACACGCAGTACAGCAAGGTCCTGGCCCTGT
ACAACCAGCATAACCCGGGCGCCTCGGCGGCGCCGTGCTGCGTGCCGCAGGCGC
TGGAGCCGCTGCCCATCGTGTACTACGTGGGCCGCAAGCCCAAGGTGGAGCAGC
TGTCCAACATGATCGTGCGCTCCTGCAAGTGCAGC
Non-limiting exemplary amino acid sequences of human TGFB1
Wild-type
(SEQ ID NO: 210)
MPPSGLRLLLLLLPLLWLLVLTPGRPAAGLSTCKTIDMELVKRKRIEAIRGQILSKLRL
ASPPSQGEVPPGPLPEAVLALYNSTRDRVAGESAEPEPEPEADYYAKEVTRVLMVET
HNEIYDKFKQSTHSIYMFFNTSELREAVPEPVLLSRAELRLLRLKLKVEQHVELYQKY
SNNSWRYLSNRLLAPSDSPEWLSFDVTGVVRQWLSRGGEIEGFRLSAHCSCDSRDNT
LQVDINGFTTGRRGDLATIHGMNRPFLLLMATPLERAQHLQSSRHRRALDTNYCFSS
TEKNCCVRQLYIDFRKDLGWKWIHEPKGYHANFCLGPCPYIWSLDTQYSKVLALYN
QHNPGASAAPCCVPQALEPLPIVYYVGRKPKVEQLSNMIVRSCKCS
dmTGFB1 R218C, C225R
(SEQ ID NO: 214)
MPPSGLRLLLLLLPLLWLLVLTPGRPAAGLSTCKTIDMELVKRKRIEAIRGQILSKLRL
ASPPSQGEVPPGPLPEAVLALYNSTRDRVAGESAEPEPEPEADYYAKEVTRVLMVET
HNEIYDKFKQSTHSIYMFFNTSELREAVPEPVLLSRAELRLLRLKLKVEQHVELYQKY
SNNSWRYLSNRLLAPSDSPEWLSFDVTGVVRQWLSRGGEIEGFHLSAHCSRDSRDNT
LQVDINGFTTGRRGDLATIHGMNRPFLLLMATPLERAQHLQSSRHRRALDTNYCFSS
TEKNCCVRQLYIDFRKDLGWKWIHEPKGYHANFCLGPCPYIWSLDTQYSKVLALYN
QHNPGASAAPCCVPQALEPLPIVYYVGRKPKVEQLSNMIVRSCKCS
Non-limiting exemplary nucleic acid sequences encoding
IL10 are provided:
Wild-type IL10:
(SEQ ID NO: 122)
ATGCACAGCTCAGCACTGCTCTGTTGCCTGGTCCTCCTGACTGGGGTGAGGGCCA
GCCCAGGCCAGGGCACCCAGTCTGAGAACAGCTGCACCCACTTCCCAGGCAACC
TGCCTAACATGCTTCGAGATCTCCGAGATGCCTTCAGCAGAGTGAAGACTTTCTT
TCAAATGAAGGATCAGCTGGACAACTTGTTGTTAAAGGAGTCCTTGCTGGAGGA
CTTTAAGGGTTACCTGGGTTGCCAAGCCTTGTCTGAGATGATCCAGTTTTACCTG
GAGGAGGTGATGCCCCAAGCTGAGAACCAAGACCCAGACATCAAGGCGCATGTG
AACTCCCTGGGGGAGAACCTGAAGACCCTCAGGCTGAGGCTACGGCGCTGTCAT
CGATTTCTTCCCTGTGAAAACAAGAGCAAGGCCGTGGAGCAGGTGAAGAATGCC
TTTAATAAGCTCCAAGAGAAAGGCATCTACAAAGCCATGAGTGAGTTTGACATCT
TCATCAACTACATAGAAGCCTACATGACAATGAAGATACGAAAC
High affinity IL10 (N36I, N110I, K117N, F129L):
(SEQ ID NO: 123)
ATGCACAGCTCAGCACTGCTCTGTTGCCTGGTCCTCCTGACTGGGGTGAGGGCCA
GCCCAGGCCAGGGCACCCAGTCTGAGAACAGCTGCACCCACTTCCCAGGCATCC
TGCCTAACATGCTTCGAGATCTCCGAGATGCCTTCAGCAGAGTGAAGACTTTCTT
TCAAATGAAGGATCAGCTGGACAACTTGTTGTTAAAGGAGTCCTTGCTGGAGGA
CTTTAAGGGTTACCTGGGTTGCCAAGCCTTGTCTGAGATGATCCAGTTTTACCTG
GAGGAGGTGATGCCCCAAGCTGAGAACCAAGACCCAGACATCAAGGCGCATGTG
atcTCCCTGGGGGAGAACCTGAATACCCTCAGGCTGAGGCTACGGCGCTGTCATCG
ActcCTTCCCTGTGAAAACAAGAGCAAGGCCGTGGAGCAGGTGAAGAATGCCTTT
AATAAGCTCCAAGAGAAAGGCATCTACAAAGCCATGAGTGAGTTTGACATCTTC
ATCAACTACATAGAAGCCTACATGACAATGAAGATACGAAAC
Non-limiting exemplary amino acid sequences of IL10 are
provided:
Wild-type IL10:
(SEQ ID NO: 124)
MHSSALLCCLVLLTGVRASPGQGTQSENSCTHFPGNLPNMLRDLRDAFSRVKTFFQ
MKDQLDNLLLKESLLEDFKGYLGCQALSEMIQFYLEEVMPQAENQDPDIKAHVNSL
GENLKTLRLRLRRCHRFLPCENKSKAVEQVKNAFNKLQEKGIYKAMSEFDIFINYIEA
YMTMKIRN
High affinity IL10 (N36I, N110I, K117N, F129L):
(SEQ ID NO: 125)
MHSSALLCCLVLLTGVRASPGQGTQSENSCTHFPGILPNMLRDLRDAFSRVKTFFQM
KDQLDNLLLKESLLEDFKGYLGCQALSEMIQFYLEEVMPQAENQDPDIKAHVISLGE
NLNTLRLRLRRCHRLLPCENKSKAVEQVKNAFNKLQEKGIYKAMSEFDIFINYIEAY
MTMKIRN
Mature wild-type IL10
(SEQ ID NO: 231)
SPGQGTQSENSCTHFPGNLPNMLRDLRDAFSRVKTFFQMKDQLDNLLLKESLLEDFK
GYLGCQALSEMIQFYLEEVMPQAENQDPDIKAHVNSLGENLKTLRLRLRRCHRFLPC
ENKSKAVEQVKNAFNKLQEKGIYKAMSEFDIFINYIEAYMTMKIRN
IL10 (D25K)
(SEQ ID NO: 232)
SPGQGTQSENSCTHFPGNLPNMLRKLRDAFSRVKTFFQMKDQLDNLLLKESLLEDFK
GYLGCQALSEMIQFYLEEVMPQAENQDPDIKAHVNSLGENLKTLRLRLRRCHRFLPC
ENKSKAVEQVKNAFNKLQEKGIYKAMSEFDIFINYIEAYMTMKIRN
IL10 (D25E)
(SEQ ID NO: 233)
SPGQGTQSENSCTHFPGNLPNMLRELRDAFSRVKTFFQMKDQLDNLLLKESLLEDFK
GYLGCQALSEMIQFYLEEVMPQAENQDPDIKAHVNSLGENLKTLRLRLRRCHRFLPC
ENKSKAVEQVKNAFNKLQEKGIYKAMSEFDIFINYIEAYMTMKIRN
IL10 (D25K/E96A)
(SEQ ID NO: 234)
SPGQGTQSENSCTHFPGNLPNMLRKLRDAFSRVKTFFQMKDQLDNLLLKESLLEDFK
GYLGCQALSEMIQFYLEEVMPQAENQDPDIKAHVNSLGANLKTLRLRLRRCHRFLPC
ENKSKAVEQVKNAFNKLQEKGIYKAMSEFDIFINYIEAYMTMKIRN

Non-limiting exemplary nucleic acid sequences encoding TGFB1 are provided:
Wild-type TGFB1
Dual mutant TGFB1 (R218X wherein X is H or C, C225R)

Non-limiting exemplary nucleic acid sequences encoding CTLA4 are provided:

Wild-type CTLA4:
(SEQ ID NO: 126)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAAGCGACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAAC
High affinity CTLA4 (belatacept; Binding domain: A29Y, L104E):
(SEQ ID NO: 127)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAATACACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATGAGGGGATCGGAAATGGGA
CACAGATATATGTTATTGATCCCGAGCCATGTCCCGATAGTGACTTCCTCTTGTG
GATACTTGCCGCTGTGAGCAGTGGTTTGTTTTTTTATTCATTCCTCCTTACGGCAG
TATCACTTTCAAAAATGCTCAAGAAGCGAAGTCCTTTGACAACTGGCGTATATGT
CAAAATGCCACCAACAGAGCCCGAATGTGAGAAACAGTTCCAGCCGTACTTTAT
TCCTATAAAC
High affinity CTLA4 (Binding domain: A29H):
(SEQ ID NO: 128)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGAAAACATACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAAC
High affinity CTLA4 (Binding domain: K28H, A29H):
(SEQ ID NO: 129)
ATGGCCTGCTTGGGCTTCCAAAGGCATAAAGCCCAGCTTAATCTTGCTACTCGCA
CGTGGCCCTGCACATTGCTCTTTTTCCTCCTGTTCATTCCCGTGTTTTGCAAGGCG
ATGCATGTGGCACAACCTGCCGTCGTTCTGGCATCATCAAGAGGTATTGCTAGCT
TCGTTTGTGAGTACGCCTCCCCTGGACATCACACGGAGGTGCGCGTCACTGTATT
GCGGCAAGCCGACAGCCAAGTTACTGAAGTCTGCGCGGCAACGTATATGATGGG
CAATGAGCTGACATTCCTTGACGATTCAATCTGCACGGGAACAAGTAGTGGTAAC
CAGGTGAATCTCACTATTCAAGGTCTGAGAGCCATGGACACCGGCCTCTACATTT
GTAAGGTGGAGCTGATGTATCCTCCCCCATATTATCTGGGGATCGGAAATGGGAC
ACAGATATATGTTATTGATCCCGAGCCATGTCCCGATAGTGACTTCCTCTTGTGG
ATACTTGCCGCTGTGAGCAGTGGTTTGTTTTTTTATTCATTCCTCCTTACGGCAGT
ATCACTTTCAAAAATGCTCAAGAAGCGAAGTCCTTTGACAACTGGCGTATATGTC
AAAATGCCACCAACAGAGCCCGAATGTGAGAAACAGTTCCAGCCGTACTTTATT
CCTATAAAC

Non-limiting exemplary amino acid sequences of CTLA4 are provided:

Wild-type CTLA4:
(SEQ ID NO: 130)
MACLGFQRHKAQLNLATRTWPCTLLFFLLFIPVFCKAMHVAQPAVVLASSRGIASFV
CEYASPGKATEVRVTVLRQADSQVTEVCAATYMMGNELTFLDDSICTGTSSGNQVN
LTIQGLRAMDTGLYICKVELMYPPPYYLGIGNGTQIYVIDPEPCPDSDFLLWILAAVSS
GLFFYSFLLTAVSLSKMLKKRSPLTTGVYVKMPPTEPECEKQFQPYFIPIN
High affinity CTLA4 (belatacept; Binding domain: A29Y, L104E):
(SEQ ID NO: 131)
MACLGFQRHKAQLNLATRTWPCTLLFFLLFIPVFCKAMHVAQPAVVLASSRGIASFV
CEYASPGKYTEVRVTVLRQADSQVTEVCAATYMMGNELTFLDDSICTGTSSGNQVN
LTIQGLRAMDTGLYICKVELMYPPPYYEGIGNGTQIYVIDPEPCPDSDFLLWILAAVSS
GLFFYSFLLTAVSLSKMLKKRSPLTTGVYVKMPPTEPECEKQFQPYFIPIN
High affinity CTLA4 (Binding domain: A29H):
(SEQ ID NO: 132)
MACLGFQRHKAQLNLATRTWPCTLLFFLLFIPVFCKAMHVAQPAVVLASSRGIASFV
CEYASPGKHTEVRVTVLRQADSQVTEVCAATYMMGNELTFLDDSICTGTSSGNQVN
LTIQGLRAMDTGLYICKVELMYPPPYYLGIGNGTQIYVIDPEPCPDSDFLLWILAAVSS
GLFFYSFLLTAVSLSKMLKKRSPLTTGVYVKMPPTEPECEKQFQPYFIPIN
High affinity CTLA4 (Binding domain: K28H, A29H):
(SEQ ID NO: 133)
MACLGFQRHKAQLNLATRTWPCTLLFFLLFIPVFCKAMHVAQPAVVLASSRGIASFV
CEYASPGHHTEVRVTVLRQADSQVTEVCAATYMMGNELTFLDDSICTGTSSGNQVN
LTIQGLRAMDTGLYICKVELMYPPPYYLGIGNGTQIYVIDPEPCPDSDFLLWILAAVSS
GLFFYSFLLTAVSLSKMLKKRSPLTTGVYVKMPPTEPECEKQFQPYFIPIN

In some embodiments, the engineered T cells or population of T cells comprising heterologous sequence encoding a dmTGFB1 under the control of a promoter, comprising a further modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, exhibits at least one suppressive activity of a naturally occurring regulatory T cell (nTreg), e.g., suppression of an immune response(s) or biomarker in an in vitro or in vivo assay, e.g., an animal model of GvHD. In some embodiments, the engineered T cells or population of T cells comprising a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding a regulatory T cell promoting molecule under control of a promoter sequence, exhibits improved suppressive activity as compared to a nTreg, e.g., increased suppression of an immune response or biomarker in an in vitro or in vivo assay, e.g., an animal model of GvHD. For example, in a mouse model of GvHD, mice receiving the engineered T cell comprising a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence, exhibit improved survival compared to a control, e.g., mice receiving PBMC. In certain embodiments, the engineered cells comprise modifications for knockdown of an endogenous nucleic acid sequence encoding each an IFNG and an IL17A. In certain embodiments, the regulatory T cell promoting molecule comprises IL10 or CTLA4. In certain embodiments, the regulatory T cell promoting molecule comprises IL10 and CTLA4.

In certain embodiments, the engineered T cells or population of T cells comprise two or more heterologous coding sequences, e.g., a dmTGFB1 and a regulatory T cell promoting molecule, under the control of a promoter. In certain embodiments, each heterologous coding sequence is under control of a separate promoter. In certain embodiments, two heterologous coding sequences are under control of the same promoter. In certain embodiments, two or more heterologous coding sequences are under control of the same promoter. In certain embodiments, when the engineered T cells or population of T cells comprise three or more heterologous coding sequences, e.g., a dmTGFB1 and a regulatory T cell promoting molecule, under the control of a promoter, each heterologous sequence is each independently under control of a separate promoter or under control of a promoter that controls expression of more than one heterologous coding sequence.

### B. Targeting Receptor

In some embodiments, the engineered T cells comprising a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding dmTGFB1 molecule and a regulatory T cell promoting molecule, each under control of a promoter sequence, further comprise insertion into the cell of heterologous sequence(s) encoding a targeting receptor. The sequence(s) encoding the targeting receptor is under the control of a promoter sequence, e.g., an endogenous promoter or a heterologous promoter. In certain embodiments, the engineered cells comprise modifications for knockdown of an endogenous nucleic acid sequence encoding each an IFNG and an IL17A.

In some embodiments, the engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding a regulatory T cell promoting molecule, under control of a promoter sequence, further comprise insertion into the cell of heterologous sequence(s) encoding a targeting receptor. The sequence(s) encoding the targeting receptor is under the control of a promoter sequence, e.g., an endogenous promoter or a heterologous promoter. In certain embodiments, the engineered T cells do not comprise a modification, e.g., knockdown, of an endogenous nucleic acid sequence encoding an IFNG.

In some embodiments, the targeting receptor is a chimeric antigen receptor (CAR), a T-cell receptor (TCR), or a receptor for a cell surface molecule operably linked through at least a transmembrane domain in an internal signaling domain capable of activating a T cell upon binding of the extracellular receptor portion. In some embodiments, the targeting receptor may be a receptor present on the surface of a cell, e.g., a T cell, to permit binding of the cell to a target site, e.g., a specific cell or tissue in an organism. The targeting receptor need not be an antigen receptor, e.g., the targeting receptor may be an RGD peptide that is capable of targeting an integrin. In some embodiments, the targeting receptor targets a molecule selected from the group consisting of MAdCAM-1, TNFA, CEACAM6, VCAM-1, citrullinated vimentin, myelin basic protein (MBP), MOG (myelin oligodendrocyte glycoprotein), proteolipid protein 1 (PLP1), CD19 molecule (CD19), CD20 molecule (CD20), TNFRSF17, dipeptidyl peptidase like 6 (DPP6), solute carrier family 2 member 2 (SCL2A2), glutamate decarboxylase (GAD2), demoglein 3 (DSG3), and MHC class I HLA-A (HLA-A*02). In some embodiments, the targeting receptor targets MAdCAM-1. In some embodiments, the targeting receptor targets TNFA.

In some embodiments, the engineered T cells comprise a modification, e.g., knockdown, of an endogenous nucleic acid sequence encoding an IFNG, a modification, e.g., knockdown, of an endogenous nucleic acid sequence encoding a TNFA insertion of sequence(s) encoding a regulatory T cell promoting molecule selected from IL10, CTLA4, IDO1, ENTPD1, NT5E, IL22, AREG, IL35, GARP, CD274, FOXP3, IKZF2, EOS, IRF4, LEF1, BACH2, and IL2RA; and insertion of sequence(s) encoding a targeting receptor, e.g., a CAR, e.g., a CAR targeting MAdCAM-1. Alternatively, the targeting receptor targets TNFA.

In some embodiments, the engineered T cells comprise a modification, e.g., knockdown, of an endogenous nucleic acid sequence encoding a TNFA; a modification, e.g., knockdown, of an endogenous nucleic acid sequence encoding an IFNG or an IL17A insertion of sequences encoding dmTGFB1 and IL10, and a targeting receptor, e.g., a CAR, e.g., a CAR targeting MAdCAM-1. Alternatively, the targeting receptor targets TNFA.

In some embodiments, the engineered T cells comprise a modification, e.g., knockdown, of an endogenous nucleic acid sequence encoding a TNFA; a modification, e.g., knockdown, of an endogenous nucleic acid sequence encoding an IFNG or an IL17A; insertion of sequences encoding dmTGFB1 and CTLA4, and a targeting receptor, e.g., a CAR, e.g., a CAR targeting MAdCAM-1. Alternatively, the targeting receptor targets TNFA.

In some embodiments, the engineered T cells comprise a modification, e.g., knockdown, of an endogenous nucleic acid sequence encoding a TNFA; a modification, e.g., knockdown, of an endogenous nucleic acid sequence encoding an IFNG or an IL17A; insertion of sequences encoding dmTGFB1, IL10, CTLA4, and a targeting receptor, e.g., a CAR, e.g., a CAR targeting MAdCAM-1. Alternatively, the targeting receptor targets TNFA.

In some embodiments, the engineered T cells comprise a modification, e.g., knockdown, of an endogenous nucleic acid sequence encoding a TNFA; a modification, e.g., knockdown, of an endogenous nucleic acid sequence encoding an IL17A insertion of sequences encoding IL10 and a targeting receptor, e.g., a CAR, e.g., a CAR targeting MAdCAM-1. Alternatively, the targeting receptor targets TNFA. In certain embodiments, the engineered T cells do not comprise a modification, e.g., knockdown, of an endogenous nucleic acid sequence encoding an IFNG.

In some embodiments, the engineered T cells comprise a modification, e.g., knockdown, of an endogenous nucleic acid sequence encoding a TNFA; a modification, e.g., knockdown, of an endogenous nucleic acid sequence encoding an IL17A; insertion of sequences encoding CTLA4 and a targeting receptor, e.g., a CAR, e.g., a CAR targeting MAdCAM-1. Alternatively, the targeting receptor targets TNFA. In certain embodiments, the engineered T cells do not comprise a modification, e.g., knockdown, of an endogenous nucleic acid sequence encoding an IFNG.

In some embodiments, the engineered T cells comprise a modification, e.g., knockdown, of an endogenous nucleic acid sequence encoding a TNFA; a modification, e.g., knockdown, of an endogenous nucleic acid sequence encoding an IL17A; insertion of sequences encoding IL10, CTLA4, and a targeting receptor, e.g., a CAR, e.g., a CAR targeting MAdCAM-1. Alternatively, the targeting receptor targets TNFA. In certain embodiments, the engineered T cells do not comprise a modification, e.g., knockdown, of an endogenous nucleic acid sequence encoding an IFNG.

In some embodiments, the sequence(s) encoding the targeting receptor is incorporated into an expression construct. In some embodiments, the expression construct comprising the sequence(s) encoding the targeting receptor further comprises sequence(s) encoding a regulatory T cell promoting molecule, e.g., the sequence(s) encoding the targeting receptor and the sequence(s) encoding the regulatory T cell promoting molecule are incorporated into the same expression construct. In some embodiments, the expression construct comprising the sequence(s) encoding the targeting receptor does not further comprise sequence(s) encoding a regulatory T cell promoting molecule, e.g., the sequence(s) encoding the regulatory T cell promoting molecule are incorporated into a separate expression construct. In some embodiments, the expression construct comprising the sequence(s) encoding the targeting receptor is an episomal expression construct. In some embodiments, the sequence(s) encoding the targeting receptor is inserted into the genome, e.g., a targeted or an untargeted insertion.

In some embodiments, the sequence(s) encoding the targeting receptor may be inserted into a site selected from a TCR gene locus, e.g., TRAC locus, a TNF gene locus, an IFNG gene locus, IL17A locus, a IL6 locus, an IL2 locus, or an adeno-associated virus integration site 1 (AAVS1) locus.

In some embodiments, the engineered T cells comprise an insertion of sequence(s) encoding a targeting receptor by gene editing, e.g., as assessed by sequencing, e.g., NGS.

In some embodiments, a population of T cells comprises T cells that are engineered to comprise a modification, e.g., knockdown, of an endogenous nucleic acid sequence encoding a TNFA a modification, e.g., knockdown, of an endogenous nucleic acid sequence encoding an IFNG or an IL17A; insertion of sequences encoding a regulatory T cell promoting molecule, and insertion of sequence(s) encoding a targeting receptor, e.g., a CAR. In some embodiments, at least 40%, 45%, preferably at least 50%, 55%, 60%, 65%, 70%, 75%, 80%, 85%, 90%, 95%, or 100% of the T cells in the population of T cells are engineered to comprise insertion of sequence(s) encoding the targeting receptor, e.g., as assessed by sequencing, e.g., NGS. It is understood that a T cell population can be enriched for a population of cells having a targeting receptor using selection methods known in the art.

In some embodiments, disclosed herein are T cells engineered by introducing or inserting a targeting receptor, e.g., a CAR, nucleic acid within a T cell, e.g., within a genomic locus of a T cell or a population of T cells using a guide RNA with an RNA-guided DNA binding agent, and a construct (e.g., donor construct or template) comprising a targeting receptor, e.g., a CAR, nucleic acid, e.g., to make an engineered T cell. In some embodiments, disclosed herein are T cells engineered by expressing a targeting receptor, e.g., a CAR, from the genomic locus of a T cell or a population of T cells, e.g., using a guide RNA with an RNA-guided DNA-binding agent and a construct (e.g., donor) comprising a targeting receptor, e.g., a CAR, nucleic acid. In some embodiments, disclosed herein are T cells engineered by inducing a break (e.g., double-stranded break (DSB) or single-stranded break (nick)) within the genome of a T cell or a population of T cells for inserting the targeting receptor, e.g., a CAR, e.g., using a guide RNA with an RNA-guided DNA-binding agent (e.g., a CRISPR/Cas system). Cells and cell populations made by the methods are also provided.

In some embodiments, the targeting receptor, e.g., a CAR, is capable of conferring target specificity to the engineered T cell comprising the targeting receptor, e.g., a CAR, e.g., to particular cells, tissues, or organs.

In some embodiments, the targeting receptor, e.g., a CAR, is capable of targeting engineered T cells to the gastrointestinal system, e.g., the targeting receptor is a CAR targeting MAdCAM-1, e.g., for suppressing immune responses in disorders such as inflammatory bowel disease, ulcerative colitis, or Crohn's disease.

In some embodiments, the targeting receptor, e.g., a CAR, is capable of targeting engineered T cells to an inflammatory tissue, e.g., the targeting receptor is a CAR targeting TNFA, e.g., for suppressing immune responses in disorders such as inflammatory bowel disease, ulcerative colitis, or Crohn's disease.

In some embodiments, the targeting receptor, e.g., a CAR, is capable of targeting engineered T cells to endothelial cells, e.g., the targeting receptor is a CAR targeting CEACAM6, e.g., for suppressing immune response(s), including inflammation, in disorders such as Crohn's disease.

In some embodiments, the targeting receptor, e.g., a CAR, is capable of targeting engineered T cells to tissues comprising endothelial cells, e.g., the targeting receptor is a CAR targeting VCAM-1, e.g., for suppressing immune responses in disorders such as Crohn's disease and multiple sclerosis.

In some embodiments, the CAR is capable of targeting engineered T cells to synovial tissue, e.g., the targeting receptor is a CAR targeting citrullinated vimentin e.g., for suppressing immune responses in disorders such as rheumatoid arthritis.

In some embodiments, the targeting receptor, e.g., a CAR, is capable of targeting engineered T cells to a neurological tissue, e.g., the targeting receptor is a CAR targeting MBP, MOG, or PLP1, e.g., for suppressing immune responses in disorders such as multiple sclerosis.

In some embodiments, the targeting receptor, e.g., a CAR, is capable of targeting engineered T cells to B cells, e.g., the targeting receptor is a CAR targeting CD19, e.g., for suppressing immune responses in disorders such as multiple sclerosis and systemic lupus erythematosus.

In some embodiments, the targeting receptor, e.g., a CAR, is capable of targeting engineered T cells to B cells, e.g., the targeting receptor is a CAR targeting CD20, e.g., for suppressing immune responses in disorders such as multiple sclerosis and systemic lupus erythematosus.

In some embodiments, the targeting receptor, e.g., a CAR, is capable of targeting engineered T cells to tissues comprising mature B lymphocytes, e.g., the targeting receptor is a CAR targeting TNFRSF17, e.g., for suppressing immune responses in disorders such as systemic lupus erythematosus.

In some embodiments, the targeting receptor, e.g., a CAR, targets SCL2A2. In some embodiments, the targeting receptor, e.g., a CAR, targets DPP6. In some embodiments, the targeting receptor, e.g., a CAR, targets GAD2. In some embodiments, the targeting receptor, e.g., a CAR, targets DSG3. In some embodiments, the targeting receptor, e.g., a CAR, targets MHC class I HLA-A (HLA-A*02).

Additional CAR targets, e.g., inflammatory antigens, are known in the art. See, e.g., WO2020092057A1, the contents of which are incorporated herein in their entirety. In some embodiments, the insertion can be assessed by detecting the amount of protein or mRNA in an engineered T cell, population of engineered T cells, tissue, body fluid of interest, or tissue culture media comprising the engineered T cells. In some embodiments, the insertion by gene editing can be assessed by sequence, e.g., next generation sequencing (NGS). Assays for protein and mRNA expression of the targeting receptor, e.g., a CAR, are described herein and known in the art.

In certain embodiments, the engineered T cells or population of T cells comprise two or more heterologous coding sequences, e.g., a dmTGFB1, a regulatory T cell promoting molecule, and a targeting receptor; under the control of a promoter. In certain embodiments, each heterologous coding sequence is under control of a separate promoter. In certain embodiments, two heterologous coding sequences are under control of the same promoter. In certain embodiments, two or more heterologous coding sequences are under control of the same promoter. In certain embodiments, when the engineered T cells or population of T cells comprise three or more heterologous coding sequences, e.g., a dmTGFB1, a regulatory T cell promoting molecule, and a targeting receptor; under the control of a promoter, each heterologous sequence is each independently under control of a separate promoter or under control of a promoter that controls expression of more than one heterologous coding sequence.

In some embodiments, the engineered T cells or population of T cells do not include a heterologous targeting receptor.

### C. T Cell Receptor (TCR)

In some embodiments, the engineered T cells or population of T cells comprising a modification, e.g., insertion into the cell of heterologous sequence(s) encoding a dmTGFB1 molecule under control of a promoter sequence, further comprising a modification, e.g., knockdown, of an endogenous nucleic acid sequence encoding TCR gene sequence(s).

In some embodiments, the engineered T cells or population of T cells comprising a modification comprising an insertion into the cell of heterologous sequence(s) encoding a dmTGFB1 molecule under control of a promoter sequence further comprise a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding a regulatory T cell promoting molecule under control of a promoter sequence; further comprise a modification, e.g., knockdown, of an endogenous nucleic acid sequence encoding TCR gene sequence(s).

In some embodiments, the engineered T cells or population of T cells comprising a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequences encoding a dmTGFB1 molecule and a regulatory T cell promoting molecule each under control of a promoter sequence, further comprising a modification, e.g., knockdown, of an endogenous nucleic acid sequence encoding TCR gene sequence(s).

In some embodiments, the engineered T cells or population of T cells comprising a modification comprising an insertion into the cell of heterologous sequence(s) encoding a dmTGFB1 molecule under control of a promoter sequence further comprise a modification, e.g., knockdown, of an endogenous nucleic acid sequence encoding a TNFA; a modification, e.g., knockdown, of an endogenous nucleic acid sequence encoding an IFNG or an IL17A; insertion into the cell of heterologous sequences encoding a dmTGFB1 molecule and a regulatory T cell promoting molecule each under control of a promoter sequence, insertion into the cell of heterologous sequence(s) encoding a targeting receptor; further comprises a modification, e.g., knockdown, of an endogenous nucleic acid sequence encoding TCR gene sequence(s).

In some embodiments, the engineered T cells or population of T cells comprising a modification comprising an insertion into the cell of heterologous sequence(s) encoding a regulatory T cell promoting molecule under control of a promoter sequence further comprise a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding a regulatory T cell promoting molecule under control of a promoter sequence; further comprise a modification, e.g., knockdown, of an endogenous nucleic acid sequence encoding TCR gene sequence(s). In certain embodiments, the engineered T cells or population of cells do not comprise a modification, e.g., knockdown, of an endogenous nucleic acid sequence encoding an IFNG.

In some embodiments, the engineered T cells or population of T cells comprising a modification, e.g., knockdown, of an endogenous nucleic acid sequence encoding a TNFA; a modification, e.g., knockdown, of an endogenous nucleic acid sequence encoding an IL17A; and insertion into the cell of heterologous sequences encoding a first regulatory T cell promoting molecule and a second regulatory T cell promoting molecule each under control of a promoter sequence, further comprising a modification, e.g., knockdown, of an endogenous nucleic acid sequence encoding TCR gene sequence(s). In certain embodiments, the engineered T cells or population of cells do not comprise a modification, e.g., knockdown, of an endogenous nucleic acid sequence encoding an IFNG.

In some embodiments, the engineered T cells or population of T cells comprising a modification comprising an insertion into the cell of heterologous sequence(s) encoding a regulatory T cell promoting molecule under control of a promoter sequence further comprise a modification, e.g., knockdown, of an endogenous nucleic acid sequence encoding a TNFA; a modification, e.g., knockdown, of an endogenous nucleic acid sequence encoding an IL17A; insertion into the cell of heterologous sequences encoding a first regulatory T cell promoting molecule and a second regulatory T cell promoting molecule each under control of a promoter sequence, insertion into the cell of heterologous sequence(s) encoding a targeting receptor; further comprises a modification, e.g., knockdown, of an endogenous nucleic acid sequence encoding TCR gene sequence(s). In certain embodiments, the engineered T cells or population of cells do not comprise a modification, e.g., knockdown, of an endogenous nucleic acid sequence encoding an IFNG.

Generally, a TCR is a heterodimer receptor molecule that contains two TCR polypeptide chains, α and β. Suitable α and β genomic sequences or loci to target for knockdown are known in the art. In some embodiments, the engineered T cells comprise a modification, e.g., knockdown, of a TCR α-chain gene sequence, e.g., TRAC. See, e.g., NCBI Gene ID: 28755; Ensembl: ENSG00000277734 (T-cell receptor Alpha Constant), US 2018/0362975, and WO2020081613.

In some embodiments, the engineered T cells or population of T cells comprise a modification comprising insertion of a sequence encoding a dmTGFB1.

In some embodiments, the engineered T cells or population of T cells comprising an insertion into the cell of heterologous sequence(s) encoding a dmTGFB1 molecule under control of a promoter sequence further comprise a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding a regulatory T cell promoting molecule selected from IL10, CTLA4, IDO1, ENTPD1, NT5E, IL22, AREG, IL35, GARP, CD274, FOXP3, IKZF2, EOS, IRF4, LEF1, BACH2, and IL2RA; and a modification, e.g., knockdown, of an endogenous nucleic acid sequence encoding TCR gene sequence(s).

In some embodiments, the engineered T cells or population of T cells comprising an insertion into the cell of heterologous sequence(s) encoding a dmTGFB1 molecule under control of a promoter sequence further comprise a modification, e.g., knockdown, of an endogenous nucleic acid sequence encoding a TNFA in combination with modification, e.g., knockdown, of an endogenous nucleic acid sequence encoding an IFNG or an IL17A; insertion of sequence(s) encoding a regulatory T cell promoting molecule selected from IL10, CTLA4, IDO1, ENTPD1, NT5E, IL22, AREG, IL35, GARP, CD274, FOXP3, IKZF2, EOS, IRF4, LEF1, BACH2, and; and a modification, e.g., knockdown, of an endogenous nucleic acid sequence encoding TCR gene sequence(s).

In some embodiments, the engineered T cells or population of T cells comprising an insertion into the cell of heterologous sequence(s) encoding a dmTGFB1 molecule under control of a promoter sequence further comprise a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding IL10 or CTLA4, and a modification, e.g., knockdown, of an endogenous nucleic acid sequence encoding TCR gene sequence(s).

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding dmTGFB1, IL10, and CTLA4, and a modification, e.g., knockdown, of an endogenous nucleic acid sequence encoding TCR gene sequence(s).

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding a dmTGFB1 molecule and a regulatory T cell promoting molecule, and a modification, e.g., knockdown, of an endogenous TCR gene sequence, e.g., TRAC gene sequence.

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding an IFNG or an IL17A
insertion of sequence(s) encoding a dmTGFB1 molecule; insertion of sequence(s) encoding a regulatory T cell promoting molecule selected from IL10, CTLA4, IDO1, ENTPD1, NT5E, IL22, AREG, IL35, GARP, CD274, FOXP3, IKZF2, EOS, IRF4, LEF1, BACH2, and IL2RA; and a modification, e.g., knockdown, of an endogenous TCR gene, e.g., a TRAC gene sequence.

In some embodiments, the engineered T cells or population of T cells comprise a modification, e.g., knockdown, of an endogenous nucleic acid sequence encoding a TNFA a modification, e.g., knockdown, of an endogenous nucleic acid sequence encoding an IFNG or an IL17A; insertion of a sequence encoding a dmTGFB1 molecule, insertion of sequence(s) encoding IL10 or CTLA4, and a modification, e.g., knockdown, of a TCR gene, e.g., a TRAC gene sequence.

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding an IFNG or an IL17A; insertion of a sequence encoding a dmTGFB1 molecule, insertion of sequence(s) encoding a regulatory T cell promoting molecule, and a modification, e.g., knockdown, of an endogenous TCR gene, e.g., a TRAC gene sequence.

In some embodiments, the engineered T cells or population of T cells comprising an insertion into the cell of heterologous sequence(s) encoding a regulatory T cell promoting molecule under control of a promoter sequence further comprise a modification, e.g., knockdown, of an endogenous nucleic acid sequence encoding a TNFA; a modification, e.g., knockdown, of an endogenous nucleic acid sequence encoding an IL17A; insertion of sequence(s) encoding IL10 or CTLA4, and a modification, e.g., knockdown, of an endogenous nucleic acid sequence encoding TCR gene sequence(s). In certain embodiments, the engineered T cells or population of cells do not comprise a modification, e.g., knockdown, of an endogenous nucleic acid sequence encoding an IFNG.

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding an IL17A; insertion of sequence(s) encoding IL10, and CTLA4, and a modification, e.g., knockdown, of an endogenous nucleic acid sequence encoding TCR gene sequence(s). In certain embodiments, the engineered T cells or population of cells do not comprise a modification, e.g., knockdown, of an endogenous nucleic acid sequence encoding an IFNG.

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding IL17A; insertion of sequence(s) encoding a first regulatory T cell promoting molecule and a second regulatory T cell promoting molecule, and a modification, e.g., knockdown, of an endogenous TCR gene sequence, e.g., TRAC gene sequence. In certain embodiments, the engineered T cells or population of cells do not comprise a modification, e.g., knockdown, of an endogenous nucleic acid sequence encoding an IFNG.

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding IL17A; insertion of sequence(s) encoding a first regulatory T cell promoting molecule; insertion of sequence(s) encoding a regulatory T cell promoting molecule, the regulatory T cell promoting molecules selected from IL10, CTLA4, IDO1, ENTPD1, NT5E, IL22, AREG, IL35, GARP, CD274, FOXP3, IKZF2, EOS, IRF4, LEF1, BACH2, and IL2RA; and a modification, e.g., knockdown, of an endogenous TCR gene, e.g., a TRAC gene sequence. In certain embodiments, the engineered T cells or population of cells do not comprise a modification, e.g., knockdown, of an endogenous nucleic acid sequence encoding an IFNG.

In some embodiments, the engineered T cells or population of T cells comprise a modification, e.g., knockdown, of an endogenous nucleic acid sequence encoding a TNFA a modification, e.g., knockdown, of an endogenous nucleic acid sequence encoding an IL17A; insertion of a sequence encoding a first regulatory T cell promoting molecule, insertion of sequence(s) encoding IL10 or CTLA4, and a modification, e.g., knockdown, of a TCR gene, e.g., a TRAC gene sequence. In certain embodiments, the engineered T cells or population of cells do not comprise a modification, e.g., knockdown, of an endogenous nucleic acid sequence encoding an IFNG.

In some embodiments, the engineered T cells or population of T cells comprise a modification, e.g., knockdown, of an endogenous nucleic acid sequence encoding a TNFA; a modification, e.g., knockdown, of an endogenous nucleic acid sequence encoding an IL17A; insertion of a sequence encoding a first regulatory T cell promoting molecule, insertion of a sequence encoding a second regulatory T cell promoting molecule, and a modification, e.g., knockdown, of an endogenous TCR gene, e.g., a TRAC gene sequence. In certain embodiments, the engineered T cells or population of cells do not comprise a modification, e.g., knockdown, of an endogenous nucleic acid sequence encoding an IFNG.

In any of these embodiments, the engineered T cells or population of T cells may further comprise insertion of sequence(s) encoding a targeting receptor as described herein, e.g., a CAR, e.g., a CAR targeting MAdCAM-1.

In any of these embodiments, the engineered T cells or population of T cells may further comprise insertion of sequence(s) encoding a targeting receptor as described herein, e.g., a CAR, e.g., a CAR targeting TNFA.

In some embodiments, the engineered T cells or population of T cells comprising an insertion into the cell of heterologous sequence(s) encoding a dmTGFB1 molecule under control of a promoter sequence further comprise a modification, e.g., knockdown, of a TRC gene sequence by gene editing, e.g., as assessed by sequencing, e.g., NGS, wherein at least 50%, 55%, 60%, 65%, preferably at least 70%, 75%, 80%, 85%, 90%, or 95% of cells comprise an insertion, deletion, or substitution in the endogenous TRC gene sequence. In some embodiments, TRC is decreased by at least 50%, 55%, 60%, 65%, preferably at least 70%, 75%, 80%, 85%, 90%, 95%, or to below the limit of detection of the assay as compared to a suitable control, e.g., wherein the TRC gene has not been modified. Assays for TRC protein and mRNA expression are known in the art.

In some embodiments, the engineered T cells or population of T cells comprise an insertion of sequence(s) encoding a targeting receptor by gene editing, e.g., as assessed by sequencing, e.g., NGS.

In some embodiments, a population of T cells comprises T cells that comprise an insertion into the cell of heterologous sequence(s) encoding a dmTGFB1 molecule under control of a promoter sequence are further engineered to comprise a modification, e.g., knockdown, of an endogenous nucleic acid sequence encoding a TNFA; a modification, e.g., knockdown, of an endogenous nucleic acid sequence encoding insertion of sequence(s) encoding a regulatory T cell promoting molecule, and a modification, e.g., knockdown, of at least one TCR gene sequence. In some embodiments, at least 50%, 55%, 60%, 65%, preferably at least 70%, 75%, 80%, 85%, 90%, 95%, or 100% of the T cells in the population of T cells are engineered to comprise a modification, e.g., knockdown, of at least one TCR gene sequence, e.g., as assessed by sequencing, e.g., NGS.

In some embodiments, a population of T cells comprises T cells that comprise an insertion into the cell of heterologous sequence(s) encoding a dmTGFB1 molecule under control of a promoter sequence are further engineered to comprise a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding a regulatory T cell promoting molecule, and a modification, e.g., knockdown, of at least one TCR gene sequence. In some embodiments, at least 50%, 55%, 60%, 65%, preferably at least 70%, 75%, 80%, 85%, 90%, 95%, or 100% of the T cells in the population of T cells are engineered to comprise a modification, e.g., knockdown, of at least one TCR gene sequence, e.g., as assessed by sequencing, e.g., NGS.

In some embodiments, guide RNAs that specifically target sites within the TCR genes, e.g., TRAC gene, are used to provide a modification, e.g., knockdown, of the TCR genes.

In some embodiments, the TCR gene is modified, e.g., knocked down, in a T cell using a guide RNA with an RNA-guided DNA binding agent. In some embodiments, disclosed herein are T cells engineered by inducing a break (e.g., double-stranded break (DSB) or single-stranded break (nick)) within the TCR genes of a T cell, e.g., using a guide RNA with an RNA-guided DNA-binding agent (e.g., a CRISPR/Cas system). The methods may be used in vitro or ex vivo, e.g., in the manufacture of cell products for suppressing immune response.

In some embodiments, the guide RNAs mediate a target-specific cutting by an RNA-guided DNA-binding agent (e.g., Cas nuclease) at a site described herein within a TCR gene. It will be appreciated that, in some embodiments, the guide RNAs comprise guide sequences that bind to, or are capable of binding to, said regions.

### D. Guide RNA

In any of the embodiments herein, the guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the sgRNA comprises one or more linkages between nucleotides that is not a phosphodiester linkage.

In each of the composition, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown herein, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA.” The sgRNA may comprise a crRNA (or a portion thereof) comprising a guide sequence shown herein covalently linked to a trRNA. The sgRNA may comprise 15, 16, 17, 18, 19, or 20 contiguous nucleotides of a guide sequence shown herein. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild-type trRNA. The length of the trRNA depends on the CRISPR/Cas system used. In some embodiments, the trRNA comprises or consists of 55, 60, 65, 70, 75, 80, 85, 90, 95, 100, or more than 100 nucleotides. In some embodiments, the trRNA may comprise certain secondary structures, such as, for example, one or more hairpin or stem-loop structures, or one or more bulge structures.

In some embodiments, the target sequence or region may be complementary to the guide sequence of the guide RNA. In some embodiments, the degree of complementarity or identity between a guide sequence of a guide RNA and its corresponding target sequence may be 75%, 80%, 85%, 90%, 95%, or 100%. In some embodiments, the target sequence and the guide sequence of the gRNA may be 100% complementary or identical. In other embodiments, the target sequence and the guide sequence of the gRNA may contain one mismatch. For example, the target sequence and the guide sequence of the gRNA may contain 1, 2, 3, 4, or 5 mismatches, where the total length of the guide sequence is about 20, or 20. In some embodiments, the target sequence and the guide sequence of the gRNA may contain 1-4 mismatches where the guide sequence is about 20, or 20 nucleotides. In some embodiments, the degree of complementarity or identity between a guide sequence and its corresponding target sequence is at least 80%, 85%, preferably 90%, or 95%, for example when, the guide sequence comprises a sequence 24 contiguous nucleotides. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, preferably 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides.

In any of the embodiments herein, each of the guide sequences herein may further comprise additional nucleotides to form a crRNA or guide RNA, e.g., with the following exemplary nucleotide sequence following the guide sequence at its 3′ end: GUUUUAGAGCUAUGCUGUUUUG (SEQ ID NO: 134) in 5′ to 3′ orientation. In the case of a sgRNA, the above guide sequences may further comprise additional nucleotides to form a sgRNA, e.g., with the following exemplary nucleotide sequence following the 3′ end of the guide sequence:

(SEQ ID NO: 135)
GUUUUAGAGCUAGAAAUAGCAAGUUAAAAUAAGGCUAGUCCGUUA
UCAACUUGAAAAAGUGGCACCGAGUCGGUGCUUUU;
(SEQ ID NO: 136)
GUUUUAGAGCUAGAAAUAGCAAGUUAAAAUAAGGCUAGUCCGUUA
UCAACUUGAAAAAGUGGCACCGAGUCGGUGC;
(SEQ ID NO: 200)
GUUUUAGAGCUAGAAAUAGCAAGUUAAAAUAAGGCUAGUCCGUUA
UCAACUUGGCACCGAGUCGGUGC;
(SEQ ID NO: 201)
GUUUUAGAGCUAGAAAUAGCAAGUUAAAAUAAGGCUAGUCCGUUA
UCACGAAAGGGCACCGAGUCGGUGC;
(SEQ ID NO: 202)
GUUGUAGCUCCCUUUCUCAUUUCGGAAACGAAAUGAGAACCGUUG
CUACAAUAAGGCCGUCUGAAAAGAUGUGCCGCAACGCUCUGCCCC
UUAAAGCUUCUGCUUUAAGGGGCAUCGUUUA;
(SEQ ID NO: 203)
GUUGUAGCUCCCUGAAACCGUUGCUACAAUAAGGCCGUCGAAAGA
UGUGCCGCAACGCUCUGCCUUCUGGCAUCGUU.

In any embodiments, the guide RNAs disclosed herein bind to a region upstream of a protospacer adjacent motif (PAM). As would be understood by those of skill in the art, the PAM sequence occurs on the strand opposite to the strand that contains the target sequence and varies with the CRISPR/Cas system. That is, the PAM sequence is on the complement strand of the target strand (the strand that contains the target sequence to which the guide RNA binds). In some embodiments, the PAM is selected from NGG, NNGRRT, NNGRR(N), NNAGAAW, NNNNG(A/C)TT, and NNNNRYAC, e.g., when the Cas system includes a SpyCas9. In other embodiments, PAM sequences include NCC, N4GAYW, N4GYTT, N4GTCT, NNNNCC(a), NNNNCAAA (wherein N is defined as any nucleotide, W is defined as either A or T, and R is defined as either A or G; and (a) is a preferred, but not required, A after the second C)), e.g., when the Cas system includes an NmeCas9.

In some embodiments, the guide RNA sequences provided herein are complementary to a sequence adjacent to a PAM sequence.

In some embodiments, the guide RNA sequence comprises a sequence that is complementary to a sequence within a genomic region selected from the tables herein according to coordinates in human reference genome hg38. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides from within a genomic region selected from the tables herein. In some embodiments, the guide RNA sequence comprises a sequence that is complementary to a sequence that comprises 5, 6, 7, 8, 9, 10, 11, 12, 13, 14, 15, 16, 17, 18, 19, 20, 21, 22, 23, 24, or 25 consecutive nucleotides spanning a genomic region selected from the tables herein.

The guide RNAs disclosed herein mediate a target-specific cutting resulting in a double-stranded break (DSB). The guide RNAs disclosed herein mediate a target-specific cutting resulting in a single-stranded break (SSB or nick).

E. Chemically Modified gRNA

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of the 3′ end or 5′ end of the oligonucleotide to provide exonuclease stability, e.g., with 2′ O-me, 2′ halide, or 2′ deoxy substituted ribose; or inverted abasic terminal nucleotide, or replacement of phosphodiester with phosphothioate.

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA. Certain gRNAs comprise at least one modified residue at or near the 5′ end and 3′ end of the RNA.

In some embodiments, the guide RNAs disclosed herein comprise one of the modification patterns disclosed in WO2018107028 the contents of which are hereby incorporated by reference in relevant part. In some embodiments, the guide RNAs disclosed herein comprise one of the structures/modification patterns disclosed in US20170114334, which are hereby incorporated by reference. In some embodiments, the guide RNAs disclosed herein comprise one of the structures/modification patterns disclosed in WO2017136794, WO2017004279, WO2019237069, US2018187186, US2019048338, WO2021119275, or WO2022125968, which are hereby incorporated by reference.

F. mRNAs Encoding RNA-Guided DNA-Binding Agents

In some embodiments, a cell or method comprises an mRNA comprising an open reading frame (ORF) encoding an RNA-guided DNA-binding agent, such as a Cas nuclease as described herein. Cas9 ORFs are provided herein and are known in the art. As one example, the Cas9 ORF can be codon optimized, such that coding sequence includes one or more alternative codons for one or more amino acids. An “alternative codon” as used herein refers to variations in codon usage for a given amino acid, and may or may not be a preferred or optimized codon (codon optimized) for a given expression system. Preferred codon usage, or codons that are well-tolerated in a given system of expression, is known in the art. The Cas9 coding sequences, Cas9 mRNAs, and Cas9 protein sequences of WO2013/176772, WO2014/065596, WO2016/106121, WO2019/067910 and WO2022/125968 are hereby incorporated by reference. In particular, the ORFs and Cas9 amino acid sequences of the table at paragraph [0449] WO2019/067910, and the Cas9 mRNAs and ORFs of paragraphs [2014]-[0234] of WO2019/067910 are hereby incorporated by reference.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, for example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. See, e.g., CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133). In Cap2, the riboses of the first and second cap-proximal nucleotides of the mRNA both comprise a 2′-methoxy. See, e.g., Katibah et al. (2014) Proc Natl Acad Sci USA 111(33): 12025-30; Abbas et al. (2017) Proc Natl Acad Sci USA 114(11): E2106-E2115.

In some embodiments, the mRNA further comprises a poly-adenylated (poly-A) tail. In some embodiments, the poly-A tail comprises 20, 30, 40, 50, 60, 70, 80, 90, or 100 adenines (SEQ ID NO: 147), optionally up to 300 adenines (SEQ ID NO: 148). In some embodiments, the poly-A tail comprises 95, 96, 97, 98, 99, or 100 adenine nucleotides (SEQ ID NO: 149).

### G. T Cells for Engineering

The engineered cells provided herein are prepared from a population of cells enriched for CD4+ T cells. Such cells can be readily obtained from fresh leukopak samples, commercially available from various sources including, e.g., StemCell Technologies. CD4+ T cells can be isolated using commercially available kits using routine methods, e.g., by negative selection using the human CD4+ T cell isolation kit. However, methods of preparation of CD4+ T cells from other sources are also known in the art. For example, multipotent cells such as hematopoietic stem cell (HSCs such as those isolated from bone marrow or cord blood), hematopoietic progenitor cells (e.g., lymphoid progenitor cell), or mesenchymal stem cells (MSC) can be used to obtain CD4+ T cells. Multipotent cells are capable of developing into more than one cell type, but are more limited than pluripotent cells in breadth of differentiation. The multipotent cells may be derived from established cell lines or isolated from human bone marrow or umbilical cords. For example, the HSCs may be isolated from a patient or a healthy donor following G-CSF-induced mobilization, plerixafor-induced mobilization, or a combination thereof. To isolate HSCs from the blood or bone marrow, the cells in the blood or bone marrow may be panned by antibodies that bind unwanted cells, such as antibodies to CD4 and CD8 (T cells), CD45 (B cells), GR-I (granulocytes), and Iad (differentiated antigen-presenting cells) (see, e.g., Inaba, et al. (1992) J Exp Med. 176:1693-1702). Methods to promote differentiation into CD4+ T cells are known in the art.

### III. Method of Delivery

The guide RNA, RNA-guided DNA binding agents (e.g., Cas nuclease), and nucleic acid sequences disclosed herein can be delivered to a cell or population of cells, in vitro or ex vivo, for the production of engineered T cells comprising insertion of a sequence encoding a dmTGFB1; optionally further comprising a modification, e.g., knockdown, of an endogenous nucleic acid sequence encoding a TNFA a modification, e.g., knockdown, of an endogenous nucleic acid sequence encoding an IFNG or an IL17A, insertion of sequence(s) encoding a regulatory T cell promoting molecule, e.g., IL10, CTLA4; and optionally further comprising insertion of sequence(s) encoding a targeting receptor, e.g., a CAR, and optionally further comprising a modification, e.g., knockdown, of TCR sequence(s), using various known and suitable methods available in the art. The guide RNA, RNA-guided DNA binding agents, and nucleic acid constructs can be delivered individually or together in any combination, using the same or different delivery methods as appropriate.

Conventional viral and non-viral based gene delivery methods can be used to introduce the guide RNA as well as the RNA-guided DNA-binding agent and donor construct in cells (e.g., mammalian cells) and target tissues. As further provided herein, non-viral vector delivery systems nucleic acids such as non-viral vectors, plasmid vectors, and, e.g., naked nucleic acid, and nucleic acid complexed with a delivery vehicle such as a liposome, lipid nanoparticle (LNP), or poloxamer. Viral vector delivery systems include DNA and RNA viruses.

Methods and compositions for non-viral delivery of nucleic acids include electroporation, lipofection, microinjection, biolistics, virosomes, liposomes, immunoliposomes, LNPs, polycation or lipid: nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

Various delivery systems (e.g., vectors, liposomes, LNPs) containing the guide RNAs, RNA-guided DNA binding agent, and donor construct, singly or in combination, can also be administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and well known to those of skill in the art.

In certain embodiments, the present disclosure provides DNA or RNA vectors encoding any of the compositions disclosed herein e.g., guide RNAs comprising any one or more of the guide sequences described herein, e.g., for modifying (e.g., knocking down) IFNG and TNFA or a donor construct comprising a sequence encoding a dmTGFB1 molecule, sequence(s) encoding a regulatory T cell promoting molecule, e.g., IL10, or a targeting receptor, e.g., a CAR, e.g., a MAdCAM-1 CAR. In some embodiments, the vector also comprises a sequence encoding an RNA-guided DNA binding agent. In certain embodiments, the invention comprises DNA or RNA vectors encoding any one or more of the compositions described herein, or in any combination. In some embodiments, the vectors further comprise, e.g., promoters, enhancers, and regulatory sequences. In some embodiments, the vector that comprises a guide RNA comprising any one or more of the guide sequences described herein also comprises one or more nucleotide sequence(s) encoding a crRNA, a trRNA, or a crRNA and trRNA, as disclosed herein.

In certain embodiments, the present disclosure provides DNA or RNA vectors encoding a regulatory T cell promoting molecules and a targeting receptor. Such vectors allow for selection of cells based on the presence of the receptor for cells that also contain a coding sequence for the regulatory T cell promoting molecule. Positive and negative selection methods based on the presence of cell surface molecules are known in the art.

In some embodiments, the vector comprises a nucleotide sequence encoding a guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35:2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA may be located on the same vector comprising the nucleotide sequence encoding an RNA-guided DNA-binding agent such as a Cas protein. In some embodiments, expression of the guide RNA and of the RNA-guided DNA-binding agent such as a Cas protein may be driven by their own corresponding promoters. In some embodiments, expression of the guide RNA may be driven by the same promoter that drives expression of the RNA-guided DNA-binding agent such as a Cas protein. In some embodiments, the guide RNA and the RNA-guided DNA-binding agent such as a Cas protein transcript may be contained within a single transcript. For example, the guide RNA may be within an untranslated region (UTR) of the RNA-guided DNA-binding agent such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the RNA-guided DNA-binding agent such as a Cas protein and the guide RNA from the same vector in close temporal proximity may facilitate more efficient formation of the CRISPR RNP complex.

In some embodiments, the nucleotide sequence encoding the guide RNA or RNA-guided DNA-binding agent may be located on the same vector comprising the construct that comprises the sequence encoding the dmTGFB1 molecule, the regulatory T cell promoting molecule, e.g., IL10, CTLA4; or targeting receptor, e.g., a CAR, e.g., a MAdCAM-1 CAR. In some embodiments, proximity of the construct comprising the sequence encoding the dmTGFB1 molecule, the regulatory T cell promoting molecule, e.g., IL10, CTLA4; or targeting receptor, e.g., a CAR, e.g., a MAdCAM-1 CAR and the guide RNA (or the RNA-guided DNA binding agent) on the same vector may facilitate more efficient insertion of the construct into a site of insertion created by the guide RNA/RNA-guided DNA binding agent.

In certain embodiments, DNA and RNA vectors can include more than one open reading frame for expression under a single promoter, either present in the vector or at the genomic insertion site. In such embodiments, a coding sequence for a self-cleaving peptide can be included between the open reading frames. The self-cleaving peptide may be, for example, a 2A peptide, for example, a P2A peptide, an E2A peptide, a F2A peptide, or a T2A peptide.

In some embodiments, the vector comprises one or more nucleotide sequence(s) encoding a sgRNA and an mRNA encoding an RNA-guided DNA binding agent, which can be a Cas protein, such as Cas9 or Cpf1. In some embodiments, the vector comprises one or more nucleotide sequence(s) encoding a crRNA, a trRNA, and an mRNA encoding an RNA-guided DNA binding agent, which can be a Cas protein, such as, Cas9 or Cpf1. In one embodiment, the Cas9 is from Streptococcus pyogenes (i.e., Spy Cas9). In some embodiments, the Cas nuclease is the Cas9 nuclease from Neisseria meningitidis (i.e., Nme Cas9, e.g., Nme1, Nme2, or Nme3 Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector comprises a donor construct comprising a sequence that encodes the dmTGFB1 molecule, the regulatory T cell promoting molecule, e.g., IL10, or targeting receptor, e.g., a CAR, e.g., MAdCAM-1, as disclosed herein. In some embodiments, in addition to the donor construct disclosed herein, the vector may further comprise nucleic acids that encode the guide RNAs described herein or nucleic acid encoding an RNA-guided DNA-binding agent (e.g., a Cas nuclease such as Cas9). In some embodiments, a nucleic acid encoding an RNA-guided DNA-binding agent are each or both on a separate vector from a vector that comprises the donor construct disclosed herein. In any of the embodiments, the vector may include other sequences that include, but are not limited to, promoters, enhancers, regulatory sequences, as described herein. In some embodiments, the promoter does not drive the expression of the regulatory T cell promoting molecule, e.g., IL10, or targeting receptor, e.g., a CAR, e.g., MAdCAM-1, of the donor construct. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease (e.g., Cas9). In some embodiments, the vector comprises one or more nucleotide sequence(s) encoding a crRNA, a trRNA, and an mRNA encoding an RNA-guided DNA nuclease, which can be a Cas nuclease, such as, Cas9. In some embodiments, the Cas9 is from Streptococcus pyogenes (i.e., Spy Cas9). In some embodiments, the Cas nuclease is the Cas9 nuclease from Neisseria meningitidis. In some embodiments, the nucleotide sequence encoding the crRNA, trRNA, or crRNA and trRNA (which may be a sgRNA) comprises or consists of a guide sequence flanked by all or a portion of a repeat sequence from a naturally occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild-type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a may be reduced. In some embodiments, viral genes (such as, e.g., integrase) that promote integration of the viral sequence into a genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a cell or population of cells along with the vector system described herein. In other embodiments, the virus may be helper-free. For example, the virus may be capable of amplifying and packaging the vectors without a helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector.

In some embodiments, “AAV” refers all serotypes, subtypes, and naturally occurring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV. In certain embodiments, the AAV is acceptable for use in ex vivo applications for human cells. In certain embodiments, the AAV is AAV6. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV).

In some embodiments, the lentivirus may be integrating. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or other vectors, which have smaller cloning capacity, it may be necessary to use more than one vector to deliver all the components of a vector system as disclosed herein. For example, one AAV vector may contain sequences encoding an RNA-guided DNA-binding agent such as a Cas protein (e.g., Cas9), while a second AAV vector may contain one or more guide sequences.

In some embodiments, the vector system may be capable of driving expression of one or more nuclease components in a cell. In some embodiments, the vector does not comprise a promoter that drives expression of one or more coding sequences once it is integrated in a cell (e.g., uses the cell's endogenous promoter such as when inserted at specific genomic loci of the cell, as exemplified herein). Suitable promoters to drive expression in different types of cells, e.g., CD4+ T cells, are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding an RNA-guided DNA-binding agent such as a Cas protein (e.g., Cas9) described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the vector may comprise any one or more of the constructs comprising a sequence encoding the dmTGFB1 molecule; the regulatory T cell promoting molecule, e.g., IL10, CTLA4; or targeting receptor, e.g., a CAR, e.g., a MAdCAM-1 CAR, as described herein. In some embodiments, the sequence of the dmTGFB1 molecule, the regulatory T cell promoting molecule, e.g., IL10, CTLA4; or targeting receptor, e.g., a CAR, e.g., a MAdCAM-1 CAR, may be operably linked to at least one transcriptional or translational control sequence. In some embodiments, the sequence of the dmTGFB1 molecule, the regulatory T cell promoting molecule, e.g., IL10, CTLA4; or targeting receptor, e.g., a CAR, e.g., a MAdCAM-1 CARmay be operably linked to at least one promoter. In some embodiments, the sequence of the dmTGFB1 molecule, the regulatory T cell promoting molecule, e.g., IL10, CTLA4; or targeting receptor, e.g., a CAR, e.g., a MAdCAM-1 CAR, is not linked to a promoter that drives the expression of the heterologous gene.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In certain embodiments, the promoter is acceptable for use in ex vivo applications in human cells.

In some embodiments, the promoter may be a tissue-specific promoter, e.g., a promoter specific for expression in a T cell.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters.

Non-limiting exemplary viral vector sequences are provided below:

CTLA4 insert (nucleotide sequence)
(SEQ ID NO: 137)
ATGGCCTGCTTGGGCTTCCAAAGGCATAAAGCCCAGCTTAATCTTGCTACTCGCACGTGGCCCTG
CACATTGCTCTTTTTCCTCCTGTTCATTCCCGTGTTTTGCAAGGCGATGCATGTGGCACAACCTG
CCGTCGTTCTGGCATCATCAAGAGGTATTGCTAGCTTCGTTTGTGAGTACGCCTCCCCTGGAAAA
GCGACGGAGGTGCGCGTCACTGTATTGCGGCAAGCCGACAGCCAAGTTACTGAAGTCTGCGCGGC
AACGTATATGATGGGCAATGAGCTGACATTCCTTGACGATTCAATCTGCACGGGAACAAGTAGTG
GTAACCAGGTGAATCTCACTATTCAAGGTCTGAGAGCCATGGACACCGGCCTCTACATTTGTAAG
GTGGAGCTGATGTATCCTCCCCCATATTATCTGGGGATCGGAAATGGGACACAGATATATGTTAT
TGATCCCGAGCCATGTCCCGATAGTGACTTCCTCTTGTGGATACTTGCCGCTGTGAGCAGTGGTT
TGTTTTTTTATTCATTCCTCCTTACGGCAGTATCACTTTCAAAAATGCTCAAGAAGCGAAGTCCT
TTGACAACTGGCGTATATGTCAAAATGCCACCAACAGAGCCCGAATGTGAGAAACAGTTCCAGCC
GTACTTTATTCCTATAAACTGA
CTLA4 insert (amino acid sequence)
(SEQ ID NO: 130)
MACLGFQRHKAQLNLATRTWPCTLLFFLLFIPVFCKAMHVAQPAVVLASSRGIASFVCEYASPGK
ATEVRVTVLRQADSQVTEVCAATYMMGNELTFLDDSICTGTSSGNQVNLTIQGLRAMDTGLYICK
VELMYPPPYYLGIGNGTQIYVIDPEPCPDSDFLLWILAAVSSGLFFYSFLLTAVSLSKMLKKRSP
LTTGVYVKMPPTEPECEKQFQPYFIPIN
IL10 insert (nucleotide sequence)
(SEQ ID NO: 138)
ATGCACAGCTCAGCACTGCTCTGTTGCCTGGTCCTCCTGACTGGGGTGAGGGCCAGCCCAGGCCA
GGGCACCCAGTCTGAGAACAGCTGCACCCACTTCCCAGGCAACCTGCCTAACATGCTTCGAGATC
TCCGAGATGCCTTCAGCAGAGTGAAGACTTTCTTTCAAATGAAGGATCAGCTGGACAACTTGTTG
TTAAAGGAGTCCTTGCTGGAGGACTTTAAGGGTTACCTGGGTTGCCAAGCCTTGTCTGAGATGAT
CCAGTTTTACCTGGAGGAGGTGATGCCCCAAGCTGAGAACCAAGACCCAGACATCAAGGCGCATG
TGAACTCCCTGGGGGAGAACCTGAAGACCCTCAGGCTGAGGCTACGGCGCTGTCATCGATTTCTT
CCCTGTGAAAACAAGAGCAAGGCCGTGGAGCAGGTGAAGAATGCCTTTAATAAGCTCCAAGAGAA
AGGCATCTACAAAGCCATGAGTGAGTTTGACATCTTCATCAACTACATAGAAGCCTACATGACAA
TGAAGATACGAAACTGA
IL10 insert (amino acid sequence)
(SEQ ID NO: 124)
MHSSALLCCLVLLTGVRASPGQGTQSENSCTHFPGNLPNMLRDLRDAFSRVKTFFQMKDQLDNLL
LKESLLEDFKGYLGCQALSEMIQFYLEEVMPQAENQDPDIKAHVNSLGENLKTLRLRLRRCHRFL
PCENKSKAVEQVKNAFNKLQEKGIYKAMSEFDIFINYIEAYMTMKIRN
Empty lentivector
(SEQ ID NO: 141)
ACGCGTGTAGTCTTATGCAATACTCTTGTAGTCTTGCAACATGGTAACGATGAGTTAGCAACATG
CCTTACAAGGAGAGAAAAAGCACCGTGCATGCCGATTGGTGGAAGTAAGGTGGTACGATCGTGCC
TTATTAGGAAGGCAACAGACGGGTCTGACATGGATTGGACGAACCACTGAATTGCCGCATTGCAG
AGATATTGTATTTAAGTGCCTAGCTCGATACATAAACGGGTCTCTCTGGTTAGACCAGATCTGAG
CCTGGGAGCTCTCTGGCTAACTAGGGAACCCACTGCTTAAGCCTCAATAAAGCTTGCCTTGAGTG
CTTCAAGTAGTGTGTGCCCGTCTGTTGTGTGACTCTGGTAACTAGAGATCCCTCAGACCCTTTTA
GTCAGTGTGGAAAATCTCTAGCAGTGGCGCCCGAACAGGGACTTGAAAGCGAAAGGGAAACCAGA
GGAGCTCTCTCGACGCAGGACTCGGCTTGCTGAAGCGCGCACGGCAAGAGGCGAGGGGCGGCGAC
TGGTGAGTACGCCAAAAATTTTGACTAGCGGAGGCTAGAAGGAGAGAGATGGGTGCGAGAGCGTC
AGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTG
GCCTGTTAGAAACATCAGAAGGCTGTAGACAAATACTGGGACAGCTACAACCATCCCTTCAGACA
GGATCAGAAGAACTTAGATCATTATATAATACAGTAGCAACCCTCTATTGTGTGCATCAAAGGAT
AGAGATAAAAGACACCAAGGAAGCTTTAGACAAGATAGAGGAAGAGCAAAACAAAAGTAAGACCA
CCGCACAGCAAGCGGCCACTGATCTTCAGACCTGGAGGAGGAGATATGAGGGACAATTGGAGAAG
TGAATTATATAAATATAAAGTAGTAAAAATTGAACCATTAGGAGTAGCACCCACCAAGGCAAAGA
GAAGAGTGGTGCAGAGAGAAAAAAGAGCAGTGGGAATAGGAGCTTTGTTCCTTGGGTTCTTGGGA
GCAGCAGGAAGCACTATGGGCGCAGCGTCAATGACGCTGACGGTACAGGCCAGACAATTATTGTC
TGGTATAGTGCAGCAGCAGAACAATTTGCTGAGGGCTATTGAGGCGCAACAGCATCTGTTGCAAC
TCACAGTCTGGGGCATCAAGCAGCTCCAGGCAAGAATCCTGGCTGTGGAAAGATACCTAAAGGAT
CAACAGCTCCTGGGGATTTGGGGTTGCTCTGGAAAACTCATTTGCACCACTGCTGTGCCTTGGAA
TGCTAGTTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAGCAAGAA
AAGAATGAACAAGAATTATTGGAATTAGATAAATGGGCAAGTTTGTGGAATTGGTTTAACATAAC
AAATTGGCTGTGGTATATAAAATTATTCATAATGATAGTAGGAGGCTTGGTAGGTTTAAGAATAG
TTTTTGCTGTACTTTCTATAGTGAATAGAGTTAGGCAGGGATATTCACCATTATCGTTTCAGACC
CACCTCCCAACCCCGAGGGGACCCGACAGGCCCGAAGGAATAGAAGAAGAAGGTGGAGAGAGAGA
CAGAGACAGATCCATTCGATTAGTGAACGGATCTCGACGGTATCGATGGCCGCCCCCTTCACCGA
GGGCCTATTTCCCATGATTCCTTCATATTTGCATATACGATACAAGGCTGTTAGAGAGATAATTG
GAATTAATTTGACTGTAAACACAAAGATATTAGTACAAAATACGTGACGTAGAAAGTAATAATTT
CTTGGGTAGTTTGCAGTTTTAAAATTATGTTTTAAAATGGACTATCATATGCTTACCGTAACTTG
AAAGTATTTCGATTTCTTGGCTTTATATATCTTGTGGAAAGGACGAAACACCGGAGTCTTCTTTT
TTGAAGACACTTCGGACTGTAGAACTCTGAACCTCGAGCAATTTAAAAGAAAAGGGGGGATTGGG
GGGTACAGTGCAGGGGAAAGAATAGTAGACATAATAGCAACAGACATACAAACTAAAGAATTACA
AAAACAAATTACAAAAATTCAAAATTTCTGCGTTGTTGTCGGTGCTCGTTCTCTGCTCTTCACGC
TACTGAATTCATCACCGGTTCTTCGAAGGCCTCCGCGCCGGGTTTTGGCGCCTCCCGCGGGCGCC
CCCCTCCTCACGGCGAGCGCTGCCACGTCAGACGAAGGGCGCAGCGAGCGTCCTGATCCTTCCGC
CCGGACGCTCAGGACAGCGGCCCGCTGCTCATAAGACTCGGCCTTAGAACCCCAGTATCAGCAGA
AGGACATTTTAGGACGGGACTTGGGTGACTCTAGGGCACTGGTTTTCTTTCCAGAGAGCGGAACA
GGCGAGGAAAAGTAGTCCCTTCTCGGCGATTCTGCGGAGGGATCTCCGTGGGGCGGTGAACGCCG
ATGATTATATAAGGACGCGCCGGGTGTGGCACAGCTAGTTCCGTCGCAGCCGGGATTTGGGTCGC
GGTTCTTGTTTGTGGATCGCTGTGATCGTCACTTGGTCTAGACGCCACCATGAGCGGGGGCGAGG
AGCTGTTCGCCGGCATCGTGCCCGTGCTGATCGAGCTGGACGGCGACGTGCACGGCCACAAGTTC
AGCGTGCGCGGCGAGGGCGAGGGCGACGCCGACTACGGCAAGCTGGAGATCAAGTTCATCTGCAC
CACCGGCAAGCTGCCCGTGCCCTGGCCCACCCTGGTGACCACCCTCTGCTACGGCATCCAGTGCT
TCGCCCGCTACCCCGAGCACATGAAGATGAACGACTTCTTCAAGAGCGCCATGCCCGAGGGCTAC
ATCCAGGAGCGCACCATCCAGTTCCAGGACGACGGCAAGTACAAGACCCGCGGCGAGGTGAAGTT
CGAGGGCGACACCCTGGTGAACCGCATCGAGCTGAAGGGCAAGGACTTCAAGGAGGACGGCAACA
TCCTGGGCCACAAGCTGGAGTACAGCTTCAACAGCCACAACGTGTACATCCGCCCCGACAAGGCC
AACAACGGCCTGGAGGCTAACTTCAAGACCCGCCACAACATCGAGGGCGGCGGCGTGCAGCTGGC
CGACCACTACCAGACCAACGTGCCCCTGGGCGACGGCCCCGTGCTGATCCCCATCAACCACTACC
TGAGCACTCAGACCAAGATCAGCAAGGACCGCAACGAGGCCCGCGACCACATGGTGCTCCTGGAG
TCCTTCAGCGCCTGCTGCCACACCCACGGCATGGACGAGCTGTACAGGGGATCCGAGGGCAGAGG
AAGCCTTCTAACATGCGGTGACGTGGAGGAGAATCCCGGCCCTTCCGGGATGACCGAGTACAAGC
CCACGGTGCGCCTCGCCACCCGCGACGACGTCCCCAGGGCCGTACGCACCCTCGCCGCCGCGTTC
GCCGACTACCCCGCCACGCGCCACACCGTCGATCCGGACCGCCACATCGAGCGGGTCACCGAGCT
GCAAGAACTCTTCCTCACGCGCGTCGGGCTCGACATCGGCAAGGTGTGGGTCGCGGACGACGGCG
CCGCGGTGGCGGTCTGGACCACGCCGGAGAGCGTCGAAGCGGGGGCGGTGTTCGCCGAGATCGGC
CCGCGCATGGCCGAGTTGAGCGGTTCCCGGCTGGCCGCGCAGCAACAGATGGAAGGCCTCCTGGC
GCCGCACCGGCCCAAGGAGCCCGCGTGGTTCCTGGCCACCGTCGGCGTCTCGCCCGACCACCAGG
GCAAGGGTCTGGGCAGCGCCGTCGTGCTCCCCGGAGTGGAGGCGGCCGAGCGCGCCGGGGTGCCC
GCCTTCCTGGAGACCTCCGCGCCCCGCAACCTCCCCTTCTACGAGCGGCTCGGCTTCACCGTCAC
CGCCGACGTCGAGGTGCCCGAAGGACCGCGCACCTGGTGCATGACCCGCAAGCCCGGTGCCTGAA
TCTAGGTCGACAATCAACCTCTGGATTACAAAATTTGTGAAAGATTGACTGGTATTCTTAACTAT
GTTGCTCCTTTTACGCTATGTGGATACGCTGCTTTAATGCCTTTGTATCATGCTATTGCTTCCCG
TATGGCTTTCATTTTCTCCTCCTTGTATAAATCCTGGTTGCTGTCTCTTTATGAGGAGTTGTGGC
CCGTTGTCAGGCAACGTGGCGTGGTGTGCACTGTGTTTGCTGACGCAACCCCCACTGGTTGGGGC
ATTGCCACCACCTGTCAGCTCCTTTCCGGGACTTTCGCTTTCCCCCTCCCTATTGCCACGGCGGA
ACTCATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTGTGTTGCCACCTGGATTCTG
CGCGGGACGTCCTTCTGCTACGTCCCTTCGGCCCTCAATCCAGCGGACCTTCCTTCCCGCGGCCT
GCTGCCGGCTCTGCGGCCTCTTCCGCGTCTCCGCCTTCGCCCTCAGACGAGTCGGATCTCTCTTT
GGGCCGCCTCCCCGCCTGGTACCTTTAAGACCAATGACTTACAAGGCAGCTGTAGATCTTAGCCA
CTTTTTAAAAGAAAAGGGGGGACTGGAAGGGCTAATTCACTCCCAACGAAGATAAGATCTGCTTT
TTGCTTGTACTGGGTCTCTCTGGTTAGACCAGATCTGAGCCTGGGAGCTCTCTGGCTAACTAGGG
AACCCACTGCTTAAGCCTCAATAAAGCTTGCCTTGAGTGCTTCAAGTAGTGTGTGCCCGTCTGTT
GTGTGACTCTGGTAACTAGAGATCCCTCAGACCCTTTTAGTCAGTGTGGAAAATCTCTAGCAGTA
GTAGTTCATGTCATCTTATTATTCAGTATTTATAACTTGCAAAGAAATGAATATCAGAGAGTGAG
AGGAACTTGTTTATTGCAGCTTATAATGGTTACAAATAAAGCAATAGCATCACAAATTTCACAAA
TAAAGCATTTTTTTCACTGCATTCTAGTTGTGGTTTGTCCAAACTCATCAATGTATCTTATCATG
TCTGGCTCTAGCTATCCCGCCCCTAACTCCGCCCATCCCGCCCCTAACTCCGCCCAGTTCCGCCC
ATTCTCCGCCCCATGGCTGACTAATTTTTTTTATTTATGCAGAGGCCGAGGCCGCCTCGGCCTCT
GAGCTATTCCAGAAGTAGTGAGGAGGCTTTTTTGGAGGCCTAGACTTTTGCAGAGACCAAATTCG
TAATCATGTCATAGCTGTTTCCTGTGTGAAATTGTTATCCGCTCACAATTCCACACAACATACGA
GCCGGAAGCATAAAGTGTAAAGCCTGGGGTGCCTAATGAGTGAGCTAACTCACATTAATTGCGTT
GCGCTCACTGCCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGA
ATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTACGGCTACACT
AGAAGAACAGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTT
ACCAATGCTTAATCAGTGAGGCACCTATCTCAGCGATCTGTCTATTTCGTTCATCCATAGTTGCC
TGACTCCCCGTCGTGTAGATAACTACGATACGGGAGGGCTTACCATCTGGCCCCAGTGCTGCAAT
GATACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACAGGCATCGT
GGTGTCACGCTCGTCGTTTGGTATGGCTTCATTCAGCTCCGGTTCCCAACGATCAAGGCGAGTTA
CATGATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACATAGCAGAACTTTA
AAAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGCG
TTTCTGGGTGAGCAAAAACAGGAAGGCAAAATGCCGCAAAAAAGGGAATAAGGGCGACACGGAAA
TGTTGAATACTCATACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCACATTTCCCC
GAAAAGTGCCACCTGACGTCTAAGAAACCATTATTATCATGACATTAACCTATAAAAATAGGCGT
ATCACGAGGCCCTTTCGTCTCGCGCGTTTCGGTGATGACGGTGAAAACCTCTGACACATGCAGCT
CCCGGAGACGGTCACAGCTTGTCTGTAAGCGGATGCCGGGAGCAGACAAGCCCGTCAGGGCGCGT
CAGCGGGTGTTGGCGGGTGTCGGGGCTGGCTTAACTATGCGGCATCAGAGCAGATTGTACTGAGA
GTGCACCATATGCGGTGTGAAATACCGCACAGATGCGTAAGGAGAAAATACCGCATCAGGCGCCA
TTCGCCATTCAGGCTGCGCAACTGTTGGGAAGGGCGATCGGTGCGGGCCTCTTCGCTATTACGCC
AGCTGGCGAAAGGGGGATGTGCTGCAAGGCGATTAAGTTGGGTAACGCCAGGGTTTTCCCAGTCA
CGACGTTGTAAAACGACGGCCAGTGCCAAGCTG
Empty lentivector
(SEQ ID NO: 142)
GCGATCGCAGTAATCAATTACGGGGTCATTAGTTCATAGCCCATATATGGAGTTCCGCGTTACAT
AACTTACGGTAAATGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTGG
CAGTACATCTACGTATTAGTCATCGCTATTACCATGGTGATGCGGTTTTGGCAGTACATCAATGG
GCGTGGATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGACGCAA
ATGGGCGGTAGGCGTGTACGGTGGGAGGTCTATATAAGCAGAGCTCGTTTAGTGAACCGGGGTCT
CTCTGGTTAGACCAGATCTGAGCCTGGGAGCTCTCTGGCTAACTAGGGAACCCACTGCTTAAGCC
TCAATAAAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACAGGGACC
TGAAAGCGAAAGGGAAACCAGAGCTCTCTCGACGCAGGACTCGGCTTGCTGAAGCGCGCACGGCA
AGAGGCGAGGGGCGGCGACTGGTGAGTACGCCAAAAATTTTGACTAGCGGAGGCTAGAAGGAGAG
AGATGGGTGCGAGAGCGTCAGTATTAAGCGGGGGAGAATTAGATCGCGATGGGAAAAAATTCGGT
TAAGGCCAGGGGGAAAGAAAAAATATAAATTAAAACATATAGTATGGGCAAGCAGGGAGCTAGAA
CGATTCGCAGTTAATCCTGGCCTGTTAGAAACATCAGAAGGCTGTAGACAAATACTGGGACAGCT
ACAACCATCCCTTCAGACAGGATCAGAAGAACTTAGATCATTATATAATACAGTAGCAACCCTCT
ATTGTGTGCATCAAAGGATAGAGATAAAAGACACCAAGGAAGCTTTAGACAAGATAGAGGAAGAG
CAAAACAAAAGTAAGACCACCGCACAGCAAGCGGCCGCTGATCTTCAGACCTGGAGGAGGAGATA
TGAGGGACAATTGGAGAAGTGAATTATATAAATATAAAGTAGTAAAAATTGAACCATTAGGAGTA
GCACCCACCAAGGCAAAGAGAAGAGTGGTGCAGAGAGAAAAAAGAGCAGTGGGAATAGGAGCTTT
GTTCCTTGGGTTCTTGGGAGCAGCAGGAAGCACTATGGGCGCAGCCTCAATGACGCTGACGGTAC
AGGCCAGACAATTATTGTCTGGTATAGTGCAGCAGCAGAACAATTTGCTGAGGGCTATTGAGGCG
CAACAGCATCTGTTGCAACTCACAGTCTGGGGCATCAAGCAGCTCCAGGCAAGAATCCTGGCTGT
GGAAAGATACCTAAAGGATCAACAGCTCCTGGGGATTTGGGGTTGCTCTGGAAAACTCATTTGCA
CCACTGCTGTGCCTTGGAATGCTAGTTGGAGTAATAAATCTCTGGAACAGATTTGGAATCACACG
ACCTGGATGGAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGGCAAGTTTGT
GGAATTGGTTTAACATAACAAATTGGCTGTGGTATATAAAATTATTCATAATGATAGTAGGAGGC
TTGGTAGGTTTAAGAATAGTTTTTGCTGTACTTTCTATAGTGAATAGAGTTAGGCAGGGATATTC
ACCATTATCGTTTCAGACCCACCTCCCAACCCCGAGGGGACCCGACAGGCCCGAAGGAATAGAAG
AAGAAGGTGGAGAGAGAGACAGAGACAGATCCATTCGATTAGTGAACGGATCTCGACGGTATCGG
TTAACTTTTAAAAGAAAAGGGGGGATTGGGGGGTACAGTGCAGGGGAAAGAATAGTAGACATAAT
AGCAACAGACATACAAACTAAAGAATTACAAAAACAAATTACAAAAATTCAAAATTTTGGCTCCC
GATCGTTGCGTTACACACACAATTACTGCTGATCGAGTGTAGCCTTCGAATGAAAGACCCCACCT
GTAGGTTTGGCAAGATAGCTGCAGTAACGCCATTTTGCAAGGCATGGAAAAATACCAAACCAAGA
ATAGAGAAGTTCAGATCAAGGGGGGTACATGAAAATAGCTAACGTTGGGCCAAACAGGATATCTG
CGGTGAGCAGTTTCGGCCCCGGCCCGGGGCCAAGAACAGATGGTCACCGCAGTTTCGGCCCCGGC
CCGAGGCCAAGAACAGATGGTCCCCAGATATGGCCCAACCCTCAGCAGTTTCTTAAGACCCATCA
GATGTTTCCAGGCTCCCCCAAGGACCTGAAATGACCCTGCGCCTTATTTGAATTAACCAATCAGC
CTGCTTCTCGCTTCTGTTCGCGCGCTTCTGCTTCCCGAGCTCTATAAAAGAGCTCACAACCCCTC
ACTCGGCGCGCCAGTCCTCCGATTGACTGAGTCGCCCTGATCATTGTCGATCCTACCATCCACTC
GACACACCCGCCAGGGCCCTGCCAAGCTTCCGAGCTCTCGATATCAAAGGAGGTACCCAACATGG
TCAGCAAGGGCGAGGAACTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGCAAGCTGACCCT
GAAGTTCATCTGTACCACCGGCAAGCTGCCCGTGCCCTGGCCCACCCTCGTGACCACCCTGACCT
ACGGCGTGCAATGCTTCAGCCGCTACCCCGACCACATGAAGCAGCACGACTTCTTCAAGTCCGCC
ATGCCCGAAGGCTACGTCCAGGAGCGCACCATCTTCTTCAAGGACGACGGCAACTACAAGACCCG
CGCCGAGGTGAAGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATCGAGGACGG
CAGCGTGCAACTCGCCGACCACTACCAGCAGAACACCCCCATCGGCGACGGCCCCGTGCTGCTGC
CCGACAACCACTACCTGAGCACCCAGTCCGCCCTGAGCAAAGACCCCAACGAGAAGCGCGATCAC
ATGGTCCTGCTGGAGTTCGTGACCGCCGCCGGGATCACTCTCGGCATGGACGAGCTGTACAAGTA
GAAGTTGTCTCCTCCTGCACTGACTGACTGATACAATCGATTTCTGGATCCGCAGGCCTCTGCTA
GAAGTTGTCTCCTCCTGCACTGACTGACTGATACAATCGATTTCTGGATCCGCAGGCCTCTGCTA
GCTTGACTGACTGAGTCGACAATCAACCTCTGGATTACAAAATTTGTGAAAGATTGACTGGTATT
CTTAACTATGTTGCTCCTTTTACGCTATGTGGATACGCTGCTTTAATGCCTTTGTATCATGCTAT
TGCTTCCCGTATGGCTTTCATTTTCTCCTCCTTGTATAAATCCTGGTTGCTGTCTCTTTATGAGG
AGTTGTGGCCCGTTGTCAGGCAACGTGGCGTGGTGTGCACTGTGTTTGCTGACGCAACCCCCACT
GGTTGGGGCATTGCCACCACCTGTCAGCTCCTTTCCGGGACTTTCGCTTTCCCCCTCCCTATTGC
CACGGCGGAACTCATCGCCGCCTGCCTTGCCCGCTGCTGGACAGGGGCTCGGCTGTTGGGCACTG
ACAATTCCGTGGTGTTGTCGGGGAAGCTGACGTCCTTTCCATGGCTGCTCGCCTGTGTTGCCACC
TGGATTCTGCGCGGGACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCCCTCAGACGAGTCGGA
TCTCCCTTTGGGCCGCCTCCCCGCCTGGAATTCGAGCTCGGTACCTTTAAGACCAATGACTTACA
AGGCAGCTGTAGATCTTAGCCACTTTTTAAAAGAAAAGGGGGGACTGGAAGGGCTAATTCACTCC
CAACGAAGACAAGATCTGCTTTTTGCTTGTACTGGGTCTCTCTGGTTAGACCAGATCTGAGCCTG
GGAGCTCTCTGGCTAACTAGGGAACCCACTGCTTAAGCCTCAATAAAGCTTGCCTTGAGTGCTTC
AAGTAGTGTGTGCCCGTCTGTTGTGTGACTCTGGTAACTAGAGATCCCTCAGACCCTTTTAGTCA
GTGTGGAAAATCTCTAGCAGTCCTGGCCAACGTGAGCACCGTGCTGACCTCCAAATATCGTTAAG
CTGGAGCCTGGGAGCCGGCCTGGCCCTCCGCCCCCCCCACCCCCGCAGCCCACCCCTGGTCTTTG
AATAAAGTCTGAGTGAGTGGCCGACAGTGCCCGTGGAGTTCTCGTGACCTGAGGTGCAGGGCCGG
CGCTAGGGACACGTCCGTGCACGTGCCGAGGCCCCCTGTGCAGCTGCAAGGGACAGGCCTAGCCC
TGCAGGCCTAACTCCGCCCATCCCGCCCCTAACTCCGCCCAGTTCCGCCCATTCTCCGCCTCATG
GCTGACTAATTTTTTTTATTTATGCAGAGGCCGAGGCCGCCTCGGCCTCTGAGCTATTCCAGAAG
TAGTGAGGACGCTTTTTTGGAGGCCGAGGCTTTTGCAAAGATCGAACAAGAGACAGGACCTGCAG
GTTAATTAAATTTAAATCATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTCTTGAG
TCCAACCCGGTAAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGAGTTGGTAGCTCTTGA
TCCGGCAAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTTTAAAT
TAAAAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCATTTAAAT
GGCCGGCCTGGCGCGCCGTTTAAACCTAGATATTGATAGTCTGATCGGTCAACGTATAATCGAGT
CCTAGCTTTTGCAAACATCTATCAAGAGACAGGATCAGCAGGAGGCTTTCGCATGAGTATTCAAC
ATTTCCGTGTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCGCGAGTGGGTTACATCGAACTGGA
TCTCAACAGCGGTAAGATCCTTGAGAGTTTTCGCCCCGAAGAACGCTTTCCAATGATGAGCACTT
TTAAAGTTCTGCTATGTGGCGCGGTATTATCCCGTATTGACGCCGGGCAAGAGCAACTCGGTCGC
CGCATACACTATTCTCAGAATGACTTGGTTGAGTATTCACCAGTCACAGAAAAGCATCTTACGGA
TGGCATGACAGTAAGAGAATTATGCAGTGCTGCCATAACCATGAGTGATAACACTGCGGCCAACT
TACTTCTGACAACGATTGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGAGCGTGACAC
CACGATGCCTGTAGCAATGGCAACAACCTTGCGTAAACTATTAACTGGCGAACTACTTACTCTAG
CTTCCCGGCAACAGTTGATAGACTGGATGGAGGCGGATAAAGTTGCAGGACCACTTCTGCGCTCG
GCCCTTCCGGCTGGCTGGTTTATTGCTGATAAATCTGGAGCCGGTGAGCGTGGGTCTCGCGGTAT
CATTGCAGCACTGGGGCCAGATGGTAAGCCCTCCCGTATCGTAGTTATCTACACGACGGGGAGTC
AGGCAACTATGGATGAACGAAATAGACAGATCGCTGAGATAGGTGCCTCACTGATTAAGCATTGG
TAACCGATTCTAGGTGCATTGGCGCAGAAAAAAATGCCTGATGCGACGCTGCGCGTCTTATACTC
CCACATATGCCAGATTCAGCAACGGATACGGCTTCCCCAACTTGCCCACTTCCATACGTGTCCTC
CTTACCAGAAATTTATCCTTAAGATCCCGAATCGTTTAAAC

Other sequences are provided throughout the specifications and in the table below; and the Sequence Listing filed herewith which forms part of the specifications.

### Table of Sequences

In the following table, the terms “mA,” “mC,” “mU,” or “mG” are used to denote a nucleotide that has been modified with 2′-O-Me.

In the following table, each “N” is used to independently denote any nucleotide (e.g., A, U, T, C, G). In certain embodiments, the nucleotide is an unmodified RNA nucleotide residue, i.e., a ribose sugar and a phosphodiester backbone.

In the following table, a “*” is used to denote a PS modification. In this application, the terms A*, C*, U*, or G* may be used to denote a nucleotide that is linked to the next (e.g., 3′) nucleotide with a PS bond.

It is understood that if a DNA sequence (comprising Ts) is referenced with respect to an RNA, then Ts should be replaced with Us (which may be modified or unmodified depending on the context), and vice versa.

In the following table, single amino acid letter code is used to provide peptide sequences.

| SEQ | | |
|---|---|---|
| ID | Description | DNA sequence |
| 204 | Nucleic acid | ATGCCGCCCTCCGGGCTGCGGCTGCTGCTGCTGCTGCTACCGCTGCT |
| | sequence | GTGGCTACTGGTGCTGACGCCTGGCCGGCCGGCCGCGGGACTATCCA |
| | encoding | CCTGCAAGACTATCGACATGGAGCTGGTGAAGCGGAAGCGCATCGAG |
| | TGFB1 | GCCATCCGCGGCCAGATCCTGTCCAAGCTGCGGCTCGCCAGCCCCCC |
| | | GAGCCAGGGGGAGGTGCCGCCCGGCCCGCTGCCCGAGGCCGTGCTCG |
| | | CCCTGTACAACAGCACCCGCGACCGGGTGGCCGGGGAGAGTGCAGAA |
| | | CCGGAGCCCGAGCCTGAGGCCGACTACTACGCCAAGGAGGTCACCCG |
| | | CGTGCTAATGGTGGAAACCCACAACGAAATCTATGACAAGTTCAAGC |
| | | AGAGTACACACAGCATATATATGTTCTTCAACACATCAGAGCTCCGA |
| | | GAAGCGGTACCTGAACCCGTGTTGCTCTCCCGGGCAGAGCTGCGTCT |
| | | GCTGAGGCTCAAGTTAAAAGTGGAGCAGCACGTGGAGCTGTACCAGA |
| | | AATACAGCAACAATTCCTGGCGATACCTCAGCAACCGGCTGCTGGCA |
| | | CCCAGCGACTCGCCAGAGTGGTTATCTTTTGATGTCACCGGAGTIGT |
| | | GCGGCAGTGGTTGAGCCGTGGAGGGGAAATTGAGGGCTTTCGCCTTA |
| | | GCGCCCACTGCTCCTGTGACAGCAGGGATAACACACTGCAAGTGGAC |
| | | ATCAACGGGTTCACTACCGGCCGCCGAGGTGACCTGGCCACCATTCA |
| | | TGGCATGAACCGGCCTTTCCTGCTTCTCATGGCCACCCCGCTGGAGA |
| | | GGGCCCAGCATCTGCAAAGCTCCCGGCACCGCCGAGCCCTGGACACC |
| | | AACTATTGCTTCAGCTCCACGGAGAAGAACTGCTGCGTGCGGCAGCT |
| | | GTACATTGACTTCCGCAAGGACCTCGGCTGGAAGTGGATCCACGAGC |
| | | CCAAGGGCTACCATGCCAACTTCTGCCTCGGGCCCTGCCCCTACATT |
| | | TGGAGCCTGGACACGCAGTACAGCAAGGTCCTGGCCCTGTACAACCA |
| | | GCATAACCCGGGCGCCTCGGCGGCGCCGTGCTGCGTGCCGCAGGCGC |
| | | TGGAGCCGCTGCCCATCGTGTACTACGTGGGCCGCAAGCCCAAGGTG |
| | | GAGCAGCTGTCCAACATGATCGTGCGCTCCTGCAAGTGCAGC |
| 205 | Nucleic acid | ATGCCGCCCTCCGGGCTGCGGCTGCTGCTGCTGCTGCTACCGCTGCT |
| | sequence | GTGGCTACTGGTGCTGACGCCTGGCCGGCCGGCCGCGGGACTATCCA |
| | encoding | CCTGCAAGACTATCGACATGGAGCTGGTGAAGCGGAAGCGCATCGAG |
| | TGFB1 | GCCATCCGCGGCCAGATCCTGTCCAAGCTGCGGCTCGCCAGCCCCCC |
| | R218H | GAGCCAGGGGGAGGTGCCGCCCGGCCCGCTGCCCGAGGCCGTGCTCG |
| | | CCCTGTACAACAGCACCCGCGACCGGGTGGCCGGGGAGAGTGCAGAA |
| | | CCGGAGCCCGAGCCTGAGGCCGACTACTACGCCAAGGAGGTCACCCG |
| | | CGTGCTAATGGTGGAAACCCACAACGAAATCTATGACAAGTTCAAGC |
| | | AGAGTACACACAGCATATATATGTTCTTCAACACATCAGAGCTCCGA |
| | | GAAGCGGTACCTGAACCCGTGTTGCTCTCCCGGGCAGAGCTGCGTCT |
| | | GCTGAGGCTCAAGTTAAAAGTGGAGCAGCACGTGGAGCTGTACCAGA |
| | | AATACAGCAACAATTCCTGGCGATACCTCAGCAACCGGCTGCTGGCA |
| | | CCCAGCGACTCGCCAGAGTGGTTATCTTTTGATGTCACCGGAGTTGT |
| | | GCGGCAGTGGTTGAGCCGTGGAGGGGAAATTGAGGGCTTTCACCTTA |
| | | GCGCCCACTGCTCCTGTGACAGCAGGGATAACACACTGCAAGTGGAC |
| | | ATCAACGGGTTCACTACCGGCCGCCGAGGTGACCTGGCCACCATTCA |
| | | TGGCATGAACCGGCCTTTCCTGCTTCTCATGGCCACCCCGCTGGAGA |
| | | GGGCCCAGCATCTGCAAAGCTCCCGGCACCGCCGAGCCCTGGACACC |
| | | AACTATTGCTTCAGCTCCACGGAGAAGAACTGCTGCGTGCGGCAGCT |
| | | GTACATTGACTTCCGCAAGGACCTCGGCTGGAAGTGGATCCACGAGC |
| | | CCAAGGGCTACCATGCCAACTTCTGCCTCGGGCCCTGCCCCTACATT |
| | | TGGAGCCTGGACACGCAGTACAGCAAGGTCCTGGCCCTGTACAACCA |
| | | GCATAACCCGGGCGCCTCGGCGGCGCCGTGCTGCGTGCCGCAGGCGC |
| | | TGGAGCCGCTGCCCATCGTGTACTACGTGGGCCGCAAGCCCAAGGTG |
| | | GAGCAGCTGTCCAACATGATCGTGCGCTCCTGCAAGTGCAGC |
| 206 | Nucleic acid | ATGCCGCCCTCCGGGCTGCGGCTGCTGCTGCTGCTGCTACCGCTGCT |
| | sequence | GTGGCTACTGGTGCTGACGCCTGGCCGGCCGGCCGCGGGACTATCCA |
| | encoding | CCTGCAAGACTATCGACATGGAGCTGGTGAAGCGGAAGCGCATCGAG |
| | TGFB1 | GCCATCCGCGGCCAGATCCTGTCCAAGCTGCGGCTCGCCAGCCCCCC |
| | R218C | GAGCCAGGGGGAGGTGCCGCCCGGCCCGCTGCCCGAGGCCGTGCTCG |
| | | CCCTGTACAACAGCACCCGCGACCGGGTGGCCGGGGAGAGTGCAGAA |
| | | CCGGAGCCCGAGCCTGAGGCCGACTACTACGCCAAGGAGGTCACCCG |
| | | CGTGCTAATGGTGGAAACCCACAACGAAATCTATGACAAGTTCAAGC |
| | | AGAGTACACACAGCATATATATGTTCTTCAACACATCAGAGCTCCGA |
| | | GAAGCGGTACCTGAACCCGTGTTGCTCTCCCGGGCAGAGCTGCGTCT |
| | | GCTGAGGCTCAAGTTAAAAGTGGAGCAGCACGTGGAGCTGTACCAGA |
| | | AATACAGCAACAATTCCTGGCGATACCTCAGCAACCGGCTGCTGGCA |
| | | CCCAGCGACTCGCCAGAGTGGTTATCTTTTGATGTCACCGGAGTTGT |
| | | GCGGCAGTGGTTGAGCCGTGGAGGGGAAATTGAGGGCTTTTGCCTTA |
| | | GCGCCCACTGCTCCTGTGACAGCAGGGATAACACACTGCAAGTGGAC |
| | | ATCAACGGGTTCACTACCGGCCGCCGAGGTGACCTGGCCACCATTCA |
| | | TGGCATGAACCGGCCTTTCCTGCTTCTCATGGCCACCCCGCTGGAGA |
| | | GGGCCCAGCATCTGCAAAGCTCCCGGCACCGCCGAGCCCTGGACACC |
| | | AACTATTGCTTCAGCTCCACGGAGAAGAACTGCTGCGTGCGGCAGCT |
| | | GTACATTGACTTCCGCAAGGACCTCGGCTGGAAGTGGATCCACGAGC |
| | | CCAAGGGCTACCATGCCAACTTCTGCCTCGGGCCCTGCCCCTACATT |
| | | TGGAGCCTGGACACGCAGTACAGCAAGGTCCTGGCCCTGTACAACCA |
| | | GCATAACCCGGGCGCCTCGGCGGCGCCGTGCTGCGTGCCGCAGGCGC |
| | | TGGAGCCGCTGCCCATCGTGTACTACGTGGGCCGCAAGCCCAAGGTG |
| | | GAGCAGCTGTCCAACATGATCGTGCGCTCCTGCAAGTGCAGC |
| 207 | Nucleic acid | ATGCCGCCCTCCGGGCTGCGGCTGCTGCTGCTGCTGCTACCGCTGCT |
| | sequence | GTGGCTACTGGTGCTGACGCCTGGCCGGCCGGCCGCGGGACTATCCA |
| | encoding | CCTGCAAGACTATCGACATGGAGCTGGTGAAGCGGAAGCGCATCGAG |
| | TGFB1 | GCCATCCGCGGCCAGATCCTGTCCAAGCTGCGGCTCGCCAGCCCCCC |
| | C225R | GAGCCAGGGGGAGGTGCCGCCCGGCCCGCTGCCCGAGGCCGTGCTCG |
| | | CCCTGTACAACAGCACCCGCGACCGGGTGGCCGGGGAGAGTGCAGAA |
| | | CCGGAGCCCGAGCCTGAGGCCGACTACTACGCCAAGGAGGTCACCCG |
| | | CGTGCTAATGGTGGAAACCCACAACGAAATCTATGACAAGTTCAAGC |
| | | AGAGTACACACAGCATATATATGTTCTTCAACACATCAGAGCTCCGA |
| | | GAAGCGGTACCTGAACCCGTGTTGCTCTCCCGGGCAGAGCTGCGTCT |
| | | GCTGAGGCTCAAGTTAAAAGTGGAGCAGCACGTGGAGCTGTACCAGA |
| | | AATACAGCAACAATTCCTGGCGATACCTCAGCAACCGGCTGCTGGCA |
| | | CCCAGCGACTCGCCAGAGTGGTTATCTTTTGATGTCACCGGAGTTGT |
| | | GCGGCAGTGGTTGAGCCGTGGAGGGGAAATTGAGGGCTTTCGCCTTA |
| | | GCGCCCACTGCTCCAGAGACAGCAGGGATAACACACTGCAAGTGGAC |
| | | ATCAACGGGTTCACTACCGGCCGCCGAGGTGACCTGGCCACCATTCA |
| | | TGGCATGAACCGGCCTTTCCTGCTTCTCATGGCCACCCCGCTGGAGA |
| | | GGGCCCAGCATCTGCAAAGCTCCCGGCACCGCCGAGCCCTGGACACC |
| | | AACTATTGCTTCAGCTCCACGGAGAAGAACTGCTGCGTGCGGCAGCT |
| | | GTACATTGACTTCCGCAAGGACCTCGGCTGGAAGTGGATCCACGAGC |
| | | CCAAGGGCTACCATGCCAACTTCTGCCTCGGGCCCTGCCCCTACATT |
| | | TGGAGCCTGGACACGCAGTACAGCAAGGTCCTGGCCCTGTACAACCA |
| | | GCATAACCCGGGCGCCTCGGCGGCGCCGTGCTGCGTGCCGCAGGCGC |
| | | TGGAGCCGCTGCCCATCGTGTACTACGTGGGCCGCAAGCCCAAGGTG |
| | | GAGCAGCTGTCCAACATGATCGTGCGCTCCTGCAAGTGCAGC |
| 208 | Nucleic acid | ATGCCGCCCTCCGGGCTGCGGCTGCTGCTGCTGCTGCTACCGCTGCT |
| | sequence | GTGGCTACTGGTGCTGACGCCTGGCCGGCCGGCCGCGGGACTATCCA |
| | encoding | CCTGCAAGACTATCGACATGGAGCTGGTGAAGCGGAAGCGCATCGAG |
| | TGFB1 | GCCATCCGCGGCCAGATCCTGTCCAAGCTGCGGCTCGCCAGCCCCCC |
| | R218H | GAGCCAGGGGGAGGTGCCGCCCGGCCCGCTGCCCGAGGCCGTGCTCG |
| | C225R | CCCTGTACAACAGCACCCGCGACCGGGTGGCCGGGGAGAGTGCAGAA |
| | | CCGGAGCCCGAGCCTGAGGCCGACTACTACGCCAAGGAGGTCACCCG |
| | | CGTGCTAATGGTGGAAACCCACAACGAAATCTATGACAAGTTCAAGC |
| | | AGAGTACACACAGCATATATATGTTCTTCAACACATCAGAGCTCCGA |
| | | GAAGCGGTACCTGAACCCGTGTTGCTCTCCCGGGCAGAGCTGCGTCT |
| | | GCTGAGGCTCAAGTTAAAAGTGGAGCAGCACGTGGAGCTGTACCAGA |
| | | AATACAGCAACAATTCCTGGCGATACCTCAGCAACCGGCTGCTGGCA |
| | | CCCAGCGACTCGCCAGAGTGGTTATCTTTTGATGTCACCGGAGTTGT |
| | | GCGGCAGTGGTTGAGCCGTGGAGGGGAAATTGAGGGCTTTCACCTTA |
| | | GCGCCCACTGCTCCAGAGACAGCAGGGATAACACACTGCAAGTGGAC |
| | | ATCAACGGGTTCACTACCGGCCGCCGAGGTGACCTGGCCACCATTCA |
| | | TGGCATGAACCGGCCTTTCCTGCTTCTCATGGCCACCCCGCTGGAGA |
| | | GGGCCCAGCATCTGCAAAGCTCCCGGCACCGCCGAGCCCTGGACACC |
| | | AACTATTGCTTCAGCTCCACGGAGAAGAACTGCTGCGTGCGGCAGCT |
| | | GTACATTGACTTCCGCAAGGACCTCGGCTGGAAGTGGATCCACGAGC |
| | | CCAAGGGCTACCATGCCAACTTCTGCCTCGGGCCCTGCCCCTACATT |
| | | TGGAGCCTGGACACGCAGTACAGCAAGGTCCTGGCCCTGTACAACCA |
| | | GCATAACCCGGGCGCCTCGGCGGCGCCGTGCTGCGTGCCGCAGGCGC |
| | | TGGAGCCGCTGCCCATCGTGTACTACGTGGGCCGCAAGCCCAAGGTG |
| | | GAGCAGCTGTCCAACATGATCGTGCGCTCCTGCAAGTGCAGC |
| 209 | Nucleic acid | ATGTCGAAGGGAGAAGAACTGTTCACAGGAGTCGTCCCGATCCTGGT |
| | sequence | CGAACTGGACGGAGACGTCAACGGACACAAGTTCTCGGTCAGAGGAG |
| | encoding | AAGGAGAAGGAGACGCAACAAACGGAAAGCTGACACTGAAGTTCATC |
| | GFP | TGCACAACAGGAAAGCTGCCGGTCCCGTGGCCGACACTGGTCACAAC |
| | | ACTGACATACGGAGTCCAGTGCTTTTCGAGATACCCGGACCACATGA |
| | | AGAGACACGACTTCTTCAAGTCGGCAATGCCGGAAGGATACGTCCAG |
| | | GAAAGAACAATCTCGTTCAAGGACGACGGAACATACAAGACAAGAGC |
| | | AGAAGTCAAGTTCGAAGGAGACACACTGGTCAACAGAATCGAACTGA |
| | | AGGGAATCGACTTCAAGGAAGACGGAAACATCCTGGGACACAAGCTG |
| | | GAATACAACTTCAACTCGCACAACGTCTACATCACAGCAGACAAGCA |
| | | GAAGAACGGAATCAAGGCAAACTTCAAGATCAGACACAACGTCGAAG |
| | | ACGGATCGGTCCAGCTGGCAGACCACTACCAGCAGAACACACCGATC |
| | | GGAGACGGACCGGTCCTGCTGCCGGACAACCACTACCTGTCGACACA |
| | | GTCGGTCCTGTCGAAGGACCCGAACGAAAAGAGAGACCACATGGTCC |
| | | TGCTGGAATTCGTCACAGCAGCAGGAATCACACACGGAATGGACGAA |
| | | CTGTACAAGT |
| 210 | Amino acid | MPPSGLRLLLLLLPLLWLLVLTPGRPAAGLSTCKTIDMELVKRKRIE |
| | sequence for | AIRGQILSKLRLASPPSQGEVPPGPLPEAVLALYNSTRDRVAGESAE |
| | TGFB1 | PEPEPEADYYAKEVTRVLMVETHNEIYDKFKQSTHSIYMFENTSELR |
| | | EAVPEPVLLSRAELRLLRLKLKVEQHVELYQKYSNNSWRYLSNRLLA |
| | | PSDSPEWLSFDVTGVVRQWLSRGGEIEGFRLSAHCSCDSRDNTLQVD |
| | | INGFTTGRRGDLATIHGMNRPFLLLMATPLERAQHLQSSRHRRALDT |
| | | NYCFSSTEKNCCVRQLYIDFRKDLGWKWIHEPKGYHANFCLGPCPYI |
| | | WSLDTQYSKVLALYNQHNPGASAAPCCVPQALEPLPIVYYVGRKPKV |
| | | EQLSNMIVRSCKCS |
| 211 | Amino acid | MPPSGLRLLLLLLPLLWLLVLTPGRPAAGLSTCKTIDMELVKRKRIE |
| | sequence for | AIRGQILSKLRLASPPSQGEVPPGPLPEAVLALYNSTRDRVAGESAE |
| | TGFB1 | PEPEPEADYYAKEVTRVLMVETHNEIYDKFKQSTHSIYMFENTSELR |
| | R218H | EAVPEPVLLSRAELRLLRLKLKVEQHVELYQKYSNNSWRYLSNRLLA |
| | | PSDSPEWLSFDVTGVVRQWLSRGGEIEGFHLSAHCSCDSRDNTLQVD |
| | | INGFTTGRRGDLATIHGMNRPFLLLMATPLERAQHLQSSRHRRALDT |
| | | NYCFSSTEKNCCVRQLYIDFRKDLGWKWIHEPKGYHANFCLGPCPYI |
| | | WSLDTQYSKVLALYNQHNPGASAAPCCVPQALEPLPIVYYVGRKPKV |
| | | EQLSNMIVRSCKCS |
| 212 | Amino acid | MPPSGLRLLLLLLPLLWLLVLTPGRPAAGLSTCKTIDMELVKRKRIE |
| | sequence for | AIRGQILSKLRLASPPSQGEVPPGPLPEAVLALYNSTRDRVAGESAE |
| | TGFB1 | PEPEPEADYYAKEVTRVLMVETHNEIYDKFKQSTHSIYMFENTSELR |
| | R218C | EAVPEPVLLSRAELRLLRLKLKVEQHVELYQKYSNNSWRYLSNRLLA |
| | | PSDSPEWLSFDVTGVVRQWLSRGGEIEGFCLSAHCSCDSRDNTLQVD |
| | | INGFTTGRRGDLATIHGMNRPFLLLMATPLERAQHLQSSRHRRALDT |
| | | NYCFSSTEKNCCVRQLYIDERKDLGWKWIHEPKGYHANFCLGPCPYI |
| | | WSLDTQYSKVLALYNQHNPGASAAPCCVPQALEPLPIVYYVGRKPKV |
| | | EQLSNMIVRSCKCS |
| 213 | Amino acid | MPPSGLRLLLLLLPLLWLLVLTPGRPAAGLSTCKTIDMELVKRKRIE |
| | sequence for | AIRGQILSKLRLASPPSQGEVPPGPLPEAVLALYNSTRDRVAGESAE |
| | TGFB1 | PEPEPEADYYAKEVTRVLMVETHNEIYDKFKQSTHSIYMFENTSELR |
| | C225R | EAVPEPVLLSRAELRLLRLKLKVEQHVELYQKYSNNSWRYLSNRLLA |
| | | PSDSPEWLSFDVTGVVRQWLSRGGEIEGFRLSAHCSRDSRDNTLQVD |
| | | INGFTTGRRGDLATIHGMNRPFLLLMATPLERAQHLQSSRHRRALDT |
| | | NYCFSSTEKNCCVRQLYIDFRKDLGWKWIHEPKGYHANFCLGPCPYI |
| | | WSLDTQYSKVLALYNQHNPGASAAPCCVPQALEPLPIVYYVGRKPKV |
| | | EQLSNMIVRSCKCS |
| 214 | Amino acid | MPPSGLRLLLLLLPLLWLLVLTPGRPAAGLSTCKTIDMELVKRKRIE |
| | sequence for | AIRGQILSKLRLASPPSQGEVPPGPLPEAVLALYNSTRDRVAGESAE |
| | TGFB1 | PEPEPEADYYAKEVTRVLMVETHNEIYDKFKQSTHSIYMFENTSELR |
| | R218H | EAVPEPVLLSRAELRLLRLKLKVEQHVELYQKYSNNSWRYLSNRLLA |
| | C225R | PSDSPEWLSFDVTGVVRQWLSRGGEIEGFHLSAHCSRDSRDNTLQVD |
| | | INGFTTGRRGDLATIHGMNRPFLLLMATPLERAQHLQSSRHRRALDT |
| | | NYCFSSTEKNCCVRQLYIDFRKDLGWKWIHEPKGYHANFCLGPCPYI |
| | | WSLDTQYSKVLALYNQHNPGASAAPCCVPQALEPLPIVYYVGRKPKV |
| | | EQLSNMIVRSCKCS |
| 215 | Amino acid | MSKGEELFTGVVPILVELDGDVNGHKFSVRGEGEGDATNGKLTLKFI |
| | sequence for | CTTGKLPVPWPTLVTTLTYGVQCFSRYPDHMKRHDFFKSAMPEGYVQ |
| | GFP | ERTISFKDDGTYKTRAEVKFEGDTLVNRIELKGIDFKEDGNILGHKL |
| | | EYNFNSHNVYITADKQKNGIKANFKIRHNVEDGSVQLADHYQQNTPI |
| | | GDGPVLLPDNHYLSTQSVLSKDPNEKRDHMVLLEFVTAAGITHGMDE |
| | | LYK |
| 216 | Lentivirus | TATGAGTAAACTTGGTCTGACAGTTACCAATGCTTAATCAGTGAGGC |
| | vector | ACCTATCTCAGCGATCTGTCTATTTCGTTCATCCATAGTTGCCTGAC |
| | | TCCCCGTCGTGTAGATAACTACGATACGGGAGGGCTTACCATCTGGC |
| | | CCCAGTGCTGCAATGATACCGCGAGACCCACGCTCACCGGCTCCAGA |
| | | TTTATCAGCAATAAACCAGCCAGCCGGAAGGGCCGAGCGCAGAAGTG |
| | | GTCCTGCAACTTTATCCGCCTCCATCCAGTCTATTAATTGTTGCCGG |
| | | GAAGCTAGAGTAAGTAGTTCGCCAGTTAATAGTTTGCGCAACGTTGT |
| | | TGCCATTGCTACAGGCATCGTGGTGTCACGCTCGTCGTTTGGTATGG |
| | | CTTCATTCAGCTCCGGTTCCCAACGATCAAGGCGAGTTACATGATCC |
| | | CCCATGTTGTGCAAAAAAGCGGTTAGCTCCTTCGGTCCTCCGATCGT |
| | | TGTCAGAAGTAAGTTGGCCGCAGTGTTATCACTCATGGTTATGGCAG |
| | | CACTGCATAATTCTCTTACTGTCATGCCATCCGTAAGATGCTTTTCT |
| | | GTGACTGGTGAGTACTCAACCAAGTCATTCTGAGAATAGTGTATGCG |
| | | GCGACCGAGTTGCTCTTGCCCGGCGTCAATACGGGATAATACCGCGC |
| | | CACATAGCAGAACTTTAAAAGTGCTCATCATTGGAAAACGTTCTTCG |
| | | GGGCGAAAACTCTCAAGGATCTTACCGCTGTTGAGATCCAGTTCGAT |
| | | GTAACCCACTCGTGCACCCAACTGATCTTCAGCATCTTTTACTTTCA |
| | | CCAGCGTTTCTGGGTGAGCAAAAACAGGAAGGCAAAATGCCGCAAAA |
| | | AAGGGAATAAGGGCGACACGGAAATGTTGAATACTCATACTCTTCCT |
| | | TTTTCAATATTATTGAAGCATTTATCAGGGTTATTGTCTCATGAGCG |
| | | GATACATATTTGAATGTATTTAGAAAAATAAACAAATAGGGGTTCCG |
| | | CGCACATTTCCCCGAAAAGTGCCACCTGACGTCTAAGAAACCATTAT |
| | | TATCATGACATTAACCTATAAAAATAGGCGTATCACGAGGCCCTTTC |
| | | GTCTCGCGCGTTTCGGTGATGACGGTGAAAACCTCTGACACATGCAG |
| | | CTCCCGGAGACGGTCACAGCTTGTCTGTAAGCGGATGCCGGGAGCAG |
| | | ACAAGCCCGTCAGGGCGCGTCAGCGGGTGTTGGCGGGTGTCGGGGCT |
| | | GGCTTAACTATGCGGCATCAGAGCAGATTGTACTGAGAGTGCACCAT |
| | | ATGCGGTGTGAAATACCGCACAGATGCGTAAGGAGAAAATACCGCAT |
| | | CAGGCGCCATTCGCCATTCAGGCTGCGCAACTGTTGGGAAGGGCGAT |
| | | CGGTGCGGGCCTCTTCGCTATTACGCCAGCTGGCGAAAGGGGGATGT |
| | | GCTGCAAGGCGATTAAGTTGGGTAACGCCAGGGTTTTCCCAGTCACG |
| | | ACGTTGTAAAACGACGGCCAGTGCCAAGCTGACGCGTGTAGTCTTAT |
| | | GCAATACTCTTGTAGTCTTGCAACATGGTAACGATGAGTTAGCAACA |
| | | TGCCTTACAAGGAGAGAAAAAGCACCGTGCATGCCGATTGGTGGAAG |
| | | TAAGGTGGTACGATCGTGCCTTATTAGGAAGGCAACAGACGGGTCTG |
| | | ACATGGATTGGACGAACCACTGAATTGCCGCATTGCAGAGATATTGT |
| | | ATTTAAGTGCCTAGCTCGATACATAAACGGGTCTCTCTGGTTAGACC |
| | | AGATCTGAGCCTGGGAGCTCTCTGGCTAACTAGGGAACCCACTGCTT |
| | | AAGCCTCAATAAAGCTTGCCTTGAGTGCTTCAAGTAGTGTGTGCCCG |
| | | TCTGTTGTGTGACTCTGGTAACTAGAGATCCCTCAGACCCTTTTAGT |
| | | CAGTGTGGAAAATCTCTAGCAGTGGCGCCCGAACAGGGACTTGAAAG |
| | | CGAAAGGGAAACCAGAGGAGCTCTCTCGACGCAGGACTCGGCTTGCT |
| | | GAAGCGCGCACGGCAAGAGGCGAGGGGCGGCGACTGGTGAGTACGCC |
| | | AAAAATTTTGACTAGCGGAGGCTAGAAGGAGAGAGATGGGTGCGAGA |
| | | GCGTCAGTATTAAGCGGGGGAGAATTAGATCGCGATGGGAAAAAATT |
| | | CGGTTAAGGCCAGGGGGAAAGAAAAAATATAAATTAAAACATATAGT |
| | | ATGGGCAAGCAGGGAGCTAGAACGATTCGCAGTTAATCCTGGCCTGT |
| | | TAGAAACATCAGAAGGCTGTAGACAAATACTGGGACAGCTACAACCA |
| | | TCCCTTCAGACAGGATCAGAAGAACTTAGATCATTATATAATACAGT |
| | | AGCAACCCTCTATTGTGTGCATCAAAGGATAGAGATAAAAGACACCA |
| | | AGGAAGCTTTAGACAAGATAGAGGAAGAGCAAAACAAAAGTAAGACC |
| | | ACCGCACAGCAAGCGGCCACTGATCTTCAGACCTGGAGGAGGAGATA |
| | | TGAGGGACAATTGGAGAAGTGAATTATATAAATATAAAGTAGTAAAA |
| | | ATTGAACCATTAGGAGTAGCACCCACCAAGGCAAAGAGAAGAGTGGT |
| | | GCAGAGAGAAAAAAGAGCAGTGGGAATAGGAGCTTTGTTCCTTGGGT |
| | | TCTTGGGAGCAGCAGGAAGCACTATGGGCGCAGCGTCAATGACGCTG |
| | | ACGGTACAGGCCAGACAATTATTGTCTGGTATAGTGCAGCAGCAGAA |
| | | CAATTTGCTGAGGGCTATTGAGGCGCAACAGCATCTGTTGCAACTCA |
| | | CAGTCTGGGGCATCAAGCAGCTCCAGGCAAGAATCCTGGCTGTGGAA |
| | | AGATACCTAAAGGATCAACAGCTCCTGGGGATTTGGGGTTGCTCTGG |
| | | AAAACTCATTTGCACCACTGCTGTGCCTTGGAATGCTAGTTGGAGTA |
| | | ATAAATCTCTGGAACAGATTTGGAATCACACGACCTGGATGGAGTGG |
| | | GACAGAGAAATTAACAATTACACAAGCTTAATACACTCCTTAATTGA |
| | | AGAATCGCAAAACCAGCAAGAAAAGAATGAACAAGAATTATTGGAAT |
| | | TAGATAAATGGGCAAGTTTGTGGAATTGGTTTAACATAACAAATTGG |
| | | CTGTGGTATATAAAATTATTCATAATGATAGTAGGAGGCTTGGTAGG |
| | | TTTAAGAATAGTTTTTGCTGTACTTTCTATAGTGAATAGAGTTAGGC |
| | | AGGGATATTCACCATTATCGTTTCAGACCCACCTCCCAACCCCGAGG |
| | | GGACCCGACAGGCCCGAAGGAATAGAAGAAGAAGGTGGAGAGAGAGA |
| | | CAGAGACAGATCCATTCGATTAGTGAACGGATCTCGACGGTATCGAT |
| | | TTAAAAGAAAAGGGGGGATTGGGGGGTACAGTGCAGGGGAAAGAATA |
| | | GTAGACATAATAGCAACAGACATACAAACTAAAGAATTACAAAAACA |
| | | AATTACAAAAATTCAAAATTTTCGGGTTTATTACAGGGACAGCAGAG |
| | | ATCCAGTTTGCTAGACCATAGAGCCCACCGCATCCCCAGCATGCCTG |
| | | CTATTGTCTTCCCAATCCTCCCCCTTGCTGTCCTGCCCCACCCCACC |
| | | CCCCAGAATAGAATGACACCTACTCAGACAATGCGATGCAATTTCCT |
| | | CATTTTATTAGGAAAGGACAGTGGGAGTGGCACCTTCCAGGGTCAAG |
| | | GAAGGCACGGGGGAGGGGCAAACAACAGATGGCTGGCAACTAGAAGG |
| | | CACAGGCTAGCTAGGTGGATCCGAATAAGGCCTGCAACGACACACAC |
| | | ACGAACAAGCAGAGCCGCTGGACGCCGACTGCGGGACGAAAGGCCCG |
| | | GAGATGAGGAAGAGGAGAACAGCGCGGCAGACGTGCGCTTTTGAAGC |
| | | GTGCAGAATGCCGGGCCTCCGGAGGACCTTCGGGCGCCCGCCCCGCC |
| | | CCTGAGCCCGCCCCTGAGCCCGCCCCCGGACCCACCCCTTCCCAGCC |
| | | TCTGAGCCCAGAAAGCGAAGGAGCAAAGCTGCTATTGGCCGCTGCCC |
| | | CAAAGGCCTACCCGCTTCCATTGCTCAGCGGTGCTGTCCATCTGCAC |
| | | GAGACTAGTGAGACGTGCTACTTCCATTTGTCACGTCCTGCACGACG |
| | | CGAGCTGCGGGGCGGGGGGGAACTTCCTGACTAGGGGAGGAGTAGAA |
| | | GGTGGCGCGAAGGGGCCACCAAAGAACGGAGCCGGTTGGCGCCTACC |
| | | GGTGGATGTGGAATGTGTGCGAGGCCAGAGGCCACTTGTGTAGCGCC |
| | | AAGTGCCCAGCGGGGCTGCTAAAGCGCATGCTCCAGACTGCCTTGGG |
| | | AAAAGCGCCTCCCCTACCCATCGATGGCTCCGGTGCCCGTCAGTGGG |
| | | CAGAGCGCACATCGCCCACAGTCCCCGAGAAGTTGGGGGGAGGGGTC |
| | | GGCAATTGAACCGGTGCCTAGAGAAGGTGGCGCGGGGTAAACTGGGA |
| | | AAGTGATGTCGTGTACTGGCTCCGCCTTTTTCCCGAGGGTGGGGGAG |
| | | AACCGTATATAAGTGCAGTAGTCGCCGTGAACGTTCTTTTTCGCAAC |
| | | GGGTTTGCCGCCAGAACACAGGTAAGTGCCGTGTGTGGTTCCCGCGG |
| | | GCCTGGCCTCTTTACGGGTTATGGCCCTTGCGTGCCTTGAATTACTT |
| | | CCACCTGGCTGCAGTACGTGATTCTTGATCCCGAGCTTCGGGTTGGA |
| | | AGTGGGTGGGAGAGTTCGAGGCCTTGCGCTTAAGGAGCCCCTTCGCC |
| | | TCGTGCTTGAGTTGAGGCCTGGCCTGGGCGCTGGGGCCGCCGCGTGC |
| | | GAATCTGGTGGCACCTTCGCGCCTGTCTCGCTGCTTTCGATAAGTCT |
| | | CTAGCCATTTAAAATTTTTGATGACCTGCTGCGACGCTTTTTTTCTG |
| | | GCAAGATAGTCTTGTAAATGCGGGCCAAGATCTGCACACTGGTATTT |
| | | CGGTTTTTGGGGCCGCGGGCGGCGACGGGGCCCGTGCGTCCCAGCGC |
| | | ACATGTTCGGCGAGGCGGGGCCTGCGAGCGCGGCCACCGAGAATCGG |
| | | ACGGGGGTAGTCTCAAGCTGGCCGGCCTGCTCTGGTGCCTGGTCTCG |
| | | CGCCGCCGTGTATCGCCCCGCCCTGGGCGGCAAGGCTGGCCCGGTCG |
| | | GCACCAGTTGCGTGAGCGGAAAGATGGCCGCTTCCCGGCCCTGCTGC |
| | | AGGGAGCTCAAAATGGAGGACGCAGCGCTCGGGAGAGCGGGCGGGTG |
| | | AGTCACCCACACAAAGGAAAAGGGCCTTTCCGTCCTCAGCCGTCGCT |
| | | TCATGTGACTCCACGGAGTACCGGGCGCCGTCCAGGCACCTCGATTA |
| | | GTTCTCGAGCTTTTGGAGTACGTCGTCTTTAGGTTGGGGGGAGGGGT |
| | | TTTATGCGATGGAGTTTCCCCACACTGAGTGGGTGGAGACTGAAGTT |
| | | AGGCCAGCTTGGCACTTGATGTAATTCTCCTTGGAATTTGCCCTTTT |
| | | TGAGTTTGGATCTTGGTTCATTCTCAAGCCTCAGACAGTGGTTCAAA |
| | | GTTTTTTTCTTCCATTTCAGGTGTCGTGATCTAGATGCCACCGATGC |
| | | ATGTCGACAATCAACCTCTGGATTACAAAATTTGTGAAAGATTGACT |
| | | GGTATTCTTAACTATGTTGCTCCTTTTACGCTATGTGGATACGCTGC |
| | | TTTAATGCCTTTGTATCATGCTATTGCTTCCCGTATGGCTTTCATTT |
| | | TCTCCTCCTTGTATAAATCCTGGTTGCTGTCTCTTTATGAGGAGTTG |
| | | TGGCCCGTTGTCAGGCAACGTGGCGTGGTGTGCACTGTGTTTGCTGA |
| | | CGCAACCCCCACTGGTTGGGGCATTGCCACCACCTGTCAGCTCCTTT |
| | | CCGGGACTTTCGCTTTCCCCCTCCCTATTGCCACGGCGGAACTCATC |
| | | GCCGCCTGCCTTGCCCGCTGCTGGACAGGGGCTCGGCTGTTGGGCAC |
| | | TGACAATTCCGTGGTGTTGTCGGGGAAATCATCGTCCTTTCCTTGGC |
| | | TGCTCGCCTGTGTTGCCACCTGGATTCTGCGCGGGACGTCCTTCTGC |
| | | TACGTCCCTTCGGCCCTCAATCCAGCGGACCTTCCTTCCCGCGGCCT |
| | | GCTGCCGGCTCTGCGGCCTCTTCCGCGTCTCCGCCTTCGCCCTCAGA |
| | | CGAGTCGGATCTCTCTTTGGGCCGCCTCCCCGCCTGGTACCTTTAAG |
| | | ACCAATGACTTACAAGGCAGCTGTAGATCTTAGCCACTTTTTAAAAG |
| | | AAAAGGGGGGACTGGAAGGGCTAATTCACTCCCAACGAAGATAAGAT |
| | | CTGCTTTTTGCTTGTACTGGGTCTCTCTGGTTAGACCAGATCTGAGC |
| | | CTGGGAGCTCTCTGGCTAACTAGGGAACCCACTGCTTAAGCCTCAAT |
| | | AAAGCTTGCCTTGAGTGCTTCAAGTAGTGTGTGCCCGTCTGTTGTGT |
| | | GACTCTGGTAACTAGAGATCCCTCAGACCCTTTTAGTCAGTGTGGAA |
| | | AATCTCTAGCAGTAGTAGTTCATGTCATCTTATTATTCAGTATTTAT |
| | | AACTTGCAAAGAAATGAATATCAGAGAGTGAGAGGAACTTGTTTATT |
| | | GCAGCTTATAATGGTTACAAATAAAGCAATAGCATCACAAATTTCAC |
| | | AAATAAAGCATTTTTTTCACTGCATTCTAGTTGTGGTTTGTCCAAAC |
| | | TCATCAATGTATCTTATCATGTCTGGCTCTAGCTATCCCGCCCCTAA |
| | | CTCCGCCCATCCCGCCCCTAACTCCGCCCAGTTCCGCCCATTCTCCG |
| | | CCCCATGGCTGACTAATTTTTTTTATTTATGCAGAGGCCGAGGCCGC |
| | | CTCGGCCTCTGAGCTATTCCAGAAGTAGTGAGGAGGCTTTTTTGGAG |
| | | GCCTAGACTTTTGCAGAGACCAAATTCGTAATCATGTCATAGCTGTT |
| | | TCCTGTGTGAAATTGTTATCCGCTCACAATTCCACACAACATACGAG |
| | | CCGGAAGCATAAAGTGTAAAGCCTGGGGTGCCTAATGAGTGAGCTAA |
| | | CTCACATTAATTGCGTTGCGCTCACTGCCCGCTTTCCAGTCGGGAAA |
| | | CCTGTCGTGCCAGCTGCATTAATGAATCGGCCAACGCGCGGGGAGAG |
| | | GCGGTTTGCGTATTGGGCGCTCTTCCGCTTCCTCGCTCACTGACTCG |
| | | CTGCGCTCGGTCGTTCGGCTGCGGCGAGCGGTATCAGCTCACTCAAA |
| | | GGCGGTAATACGGTTATCCACAGAATCAGGGGATAACGCAGGAAAGA |
| | | ACATGTGAGCAAAAGGCCAGCAAAAGGCCAGGAACCGTAAAAAGGCC |
| | | GCGTTGCTGGCGTTTTTCCATAGGCTCCGCCCCCCTGACGAGCATCA |
| | | CAAAAATCGACGCTCAAGTCAGAGGTGGCGAAACCCGACAGGACTAT |
| | | AAAGATACCAGGCGTTTCCCCCTGGAAGCTCCCTCGTGCGCTCTCCT |
| | | GTTCCGACCCTGCCGCTTACCGGATACCTGTCCGCCTTTCTCCCTTC |
| | | GGGAAGCGTGGCGCTTTCTCATAGCTCACGCTGTAGGTATCTCAGTT |
| | | CGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTC |
| | | CAACCCGGTAAGACACGACTTATCGCCACTGGCAGCAGCCACTGGTA |
| | | ACAGGATTAGCAGAGCGAGGTATGTAGGCGGTGCTACAGAGTTCTTG |
| | | AAGTGGTGGCCTAACTACGGCTACACTAGAAGAACAGTATTTGGTAT |
| | | CTGCGCTCTGCTGAAGCCAGTTACCTTCGGAAAAAGAGTTGGTAGCT |
| | | CTTGATCCGGCAAACAAACCACCGCTGGTAGCGGTGGTTTTTTTGTT |
| | | TGCAAGCAGCAGATTACGCGCAGAAAAAAAGGATCTCAAGAAGATCC |
| | | TTTGATCTTTTCTACGGGGTCTGACGCTCAGTGGAACGAAAACTCAC |
| | | GTTAAGGGATTTTGGTCATGAGATTATCAAAAAGGATCTTCACCTAG |
| | | ATCCTTTTAAATTAAAAATGAAGTTTTAAATCAATCTAAAGTATA |
| 119 | G019753 | mC*mC*mA*GAGCAUCCAAAAGAGUGGUUUUAGAmGmCmUmAmGmAm |
| | | AmAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUmGm |
| | | CmU*mU*mU*mU |
| 120 | G019757 | mA*mG*mA*GCUCUUACCUACAACAUGUUUUAGAmGmCmUmAmGmAm |
| | | AmAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUmGm |
| | | CmU*mU*mU*mU |
| 217 | G027259 | mU*mC*mA*CAGAGGGAUAUCUCUCAGUUUUAGAmGmCmUmAmGmAm |
| | | AmAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUmGm |
| | | CmU*mU*mU*mU |
| 218 | Open reading | AUGGACAAGAAGUACUCCAUCGGCCUGGACAUCGGCACCAACUCCGU |
| | frame for | GGGCUGGGCCGUGAUCACCGACGAGUACAAGGUGCCCUCCAAGAAGU |
| | SpCas9 | UCAAGGUGCUGGGCAACACCGACCGGCACUCCAUCAAGAAGAACCUG |
| | | AUCGGCGCCCUGCUGUUCGACUCCGGCGAGACCGCCGAGGCCACCCG |
| | | GCUGAAGCGGACCGCCCGGCGGCGGUACACCCGGCGGAAGAACCGGA |
| | | UCUGCUACCUGCAGGAGAUCUUCUCCAACGAGAUGGCCAAGGUGGAC |
| | | GACUCCUUCUUCCACCGGCUGGAGGAGUCCUUCCUGGUGGAGGAGGA |
| | | CAAGAAGCACGAGCGGCACCCCAUCUUCGGCAACAUCGUGGACGAGG |
| | | UGGCCUACCACGAGAAGUACCCCACCAUCUACCACCUGCGGAAGAAG |
| | | CUGGUGGACUCCACCGACAAGGCCGACCUGCGGCUGAUCUACCUGGC |
| | | CCUGGCCCACAUGAUCAAGUUCCGGGGCCACUUCCUGAUCGAGGGCG |
| | | ACCUGAACCCCGACAACUCCGACGUGGACAAGCUGUUCAUCCAGCUG |
| | | GUGCAGACCUACAACCAGCUGUUCGAGGAGAACCCCAUCAACGCCUC |
| | | CGGCGUGGACGCCAAGGCCAUCCUGUCCGCCCGGCUGUCCAAGUCCC |
| | | GGCGGCUGGAGAACCUGAUCGCCCAGCUGCCCGGCGAGAAGAAGAAC |
| | | GGCCUGUUCGGCAACCUGAUCGCCCUGUCCCUGGGCCUGACCCCCAA |
| | | CUUCAAGUCCAACUUCGACCUGGCCGAGGACGCCAAGCUGCAGCUGU |
| | | CCAAGGACACCUACGACGACGACCUGGACAACCUGCUGGCCCAGAUC |
| | | GGCGACCAGUACGCCGACCUGUUCCUGGCCGCCAAGAACCUGUCCGA |
| | | CGCCAUCCUGCUGUCCGACAUCCUGCGGGUGAACACCGAGAUCACCA |
| | | AGGCCCCCCUGUCCGCCUCCAUGAUCAAGCGGUACGACGAGCACCAC |
| | | CAGGACCUGACCCUGCUGAAGGCCCUGGUGCGGCAGCAGCUGCCCGA |
| | | GAAGUACAAGGAGAUCUUCUUCGACCAGUCCAAGAACGGCUACGCCG |
| | | GCUACAUCGACGGCGGCGCCUCCCAGGAGGAGUUCUACAAGUUCAUC |
| | | AAGCCCAUCCUGGAGAAGAUGGACGGCACCGAGGAGCUGCUGGUGAA |
| | | GCUGAACCGGGAGGACCUGCUGCGGAAGCAGCGGACCUUCGACAACG |
| | | GCUCCAUCCCCCACCAGAUCCACCUGGGCGAGCUGCACGCCAUCCUG |
| | | CGGCGGCAGGAGGACUUCUACCCCUUCCUGAAGGACAACCGGGAGAA |
| | | GAUCGAGAAGAUCCUGACCUUCCGGAUCCCCUACUACGUGGGCCCCC |
| | | UGGCCCGGGGCAACUCCCGGUUCGCCUGGAUGACCCGGAAGUCCGAG |
| | | GAGACCAUCACCCCCUGGAACUUCGAGGAGGUGGUGGACAAGGGCGC |
| | | CUCCGCCCAGUCCUUCAUCGAGCGGAUGACCAACUUCGACAAGAACC |
| | | UGCCCAACGAGAAGGUGCUGCCCAAGCACUCCCUGCUGUACGAGUAC |
| | | UUCACCGUGUACAACGAGCUGACCAAGGUGAAGUACGUGACCGAGGG |
| | | CAUGCGGAAGCCCGCCUUCCUGUCCGGCGAGCAGAAGAAGGCCAUCG |
| | | UGGACCUGCUGUUCAAGACCAACCGGAAGGUGACCGUGAAGCAGCUG |
| | | AAGGAGGACUACUUCAAGAAGAUCGAGUGCUUCGACUCCGUGGAGAU |
| | | CUCCGGCGUGGAGGACCGGUUCAACGCCUCCCUGGGCACCUACCACG |
| | | ACCUGCUGAAGAUCAUCAAGGACAAGGACUUCCUGGACAACGAGGAG |
| | | AACGAGGACAUCCUGGAGGACAUCGUGCUGACCCUGACCCUGUUCGA |
| | | GGACCGGGAGAUGAUCGAGGAGCGGCUGAAGACCUACGCCCACCUGU |
| | | UCGACGACAAGGUGAUGAAGCAGCUGAAGCGGCGGCGGUACACCGGC |
| | | UGGGGCCGGCUGUCCCGGAAGCUGAUCAACGGCAUCCGGGACAAGCA |
| | | GUCCGGCAAGACCAUCCUGGACUUCCUGAAGUCCGACGGCUUCGCCA |
| | | ACCGGAACUUCAUGCAGCUGAUCCACGACGACUCCCUGACCUUCAAG |
| | | GAGGACAUCCAGAAGGCCCAGGUGUCCGGCCAGGGCGACUCCCUGCA |
| | | CGAGCACAUCGCCAACCUGGCCGGCUCCCCCGCCAUCAAGAAGGGCA |
| | | UCCUGCAGACCGUGAAGGUGGUGGACGAGCUGGUGAAGGUGAUGGGC |
| | | CGGCACAAGCCCGAGAACAUCGUGAUCGAGAUGGCCCGGGAGAACCA |
| | | GACCACCCAGAAGGGCCAGAAGAACUCCCGGGAGCGGAUGAAGCGGA |
| | | UCGAGGAGGGCAUCAAGGAGCUGGGCUCCCAGAUCCUGAAGGAGCAC |
| | | CCCGUGGAGAACACCCAGCUGCAGAACGAGAAGCUGUACCUGUACUA |
| | | CCUGCAGAACGGCCGGGACAUGUACGUGGACCAGGAGCUGGACAUCA |
| | | ACCGGCUGUCCGACUACGACGUGGACCACAUCGUGCCCCAGUCCUUC |
| | | CUGAAGGACGACUCCAUCGACAACAAGGUGCUGACCCGGUCCGACAA |
| | | GAACCGGGGCAAGUCCGACAACGUGCCCUCCGAGGAGGUGGUGAAGA |
| | | AGAUGAAGAACUACUGGCGGCAGCUGCUGAACGCCAAGCUGAUCACC |
| | | CAGCGGAAGUUCGACAACCUGACCAAGGCCGAGCGGGGCGGCCUGUC |
| | | CGAGCUGGACAAGGCCGGCUUCAUCAAGCGGCAGCUGGUGGAGACCC |
| | | GGCAGAUCACCAAGCACGUGGCCCAGAUCCUGGACUCCCGGAUGAAC |
| | | ACCAAGUACGACGAGAACGACAAGCUGAUCCGGGAGGUGAAGGUGAU |
| | | CACCCUGAAGUCCAAGCUGGUGUCCGACUUCCGGAAGGACUUCCAGU |
| | | UCUACAAGGUGCGGGAGAUCAACAACUACCACCACGCCCACGACGCC |
| | | UACCUGAACGCCGUGGUGGGCACCGCCCUGAUCAAGAAGUACCCCAA |
| | | GCUGGAGUCCGAGUUCGUGUACGGCGACUACAAGGUGUACGACGUGC |
| | | GGAAGAUGAUCGCCAAGUCCGAGCAGGAGAUCGGCAAGGCCACCGCC |
| | | AAGUACUUCUUCUACUCCAACAUCAUGAACUUCUUCAAGACCGAGAU |
| | | CACCCUGGCCAACGGCGAGAUCCGGAAGCGGCCCCUGAUCGAGACCA |
| | | ACGGCGAGACCGGCGAGAUCGUGUGGGACAAGGGCCGGGACUUCGCC |
| | | ACCGUGCGGAAGGUGCUGUCCAUGCCCCAGGUGAACAUCGUGAAGAA |
| | | GACCGAGGUGCAGACCGGCGGCUUCUCCAAGGAGUCCAUCCUGCCCA |
| | | AGCGGAACUCCGACAAGCUGAUCGCCCGGAAGAAGGACUGGGACCCC |
| | | AAGAAGUACGGCGGCUUCGACUCCCCCACCGUGGCCUACUCCGUGCU |
| | | GGUGGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCGAG |
| | | AAGAACCCCAUCGACUUCCUGGAGGCCAAGGGCUACAAGGAGGUGAA |
| | | GAAGGACCUGAUCAUCAAGCUGCCCAAGUACUCCCUGUUCGAGCUGG |
| | | AGAACGGCCGGAAGCGGAUGCUGGCCUCCGCCGGCGAGCUGCAGAAG |
| | | GGCAACGAGCUGGCCCUGCCCUCCAAGUACGUGAACUUCCUGUACCU |
| | | GGCCUCCCACUACGAGAAGCUGAAGGGCUCCCCCGAGGACAACGAGC |
| | | AGAAGCAGCUGUUCGUGGAGCAGCACAAGCACUACCUGGACGAGAUC |
| | | AUCGAGCAGAUCUCCGAGUUCUCCAAGCGGGUGAUCCUGGCCGACGC |
| | | CAACCUGGACAAGGUGCUGUCCGCCUACAACAAGCACCGGGACAAGC |
| | | CCAUCCGGGAGCAGGCCGAGAACAUCAUCCACCUGUUCACCCUGACC |
| | | AACCUGGGCGCCCCCGCCGCCUUCAAGUACUUCGACACCACCAUCGA |
| | | CCGGAAGCGGUACACCUCCACCAAGGAGGUGCUGGACGCCACCCUGA |
| | | UCCACCAGUCCAUCACCGGCCUGUACGAGACCCGGAUCGACCUGUCC |
| | | CAGCUGGGCGGCGACGGCGGCGGCUCCCCCAAGAAGAAGCGGAAGGU |
| | | GUGA |
| 219 | Amino acid | MDKKYSIGLDIGTNSVGWAVITDEYKVPSKKFKVLGNTDRHSIKKNL |
| | sequence for | IGALLFDSGETAEATRLKRTARRRYTRRKNRICYLQEIFSNEMAKVD |
| | SpCas9 | DSFFHRLEESFLVEEDKKHERHPIFGNIVDEVAYHEKYPTIYHLRKK |
| | | LVDSTDKADLRLIYLALAHMIKFRGHFLIEGDLNPDNSDVDKLFIQL |
| | | VQTYNQLFEENPINASGVDAKAILSARLSKSRRLENLIAQLPGEKKN |
| | | GLFGNLIALSLGLTPNFKSNFDLAEDAKLQLSKDTYDDDLDNLLAQI |
| | | GDQYADLFLAAKNLSDAILLSDILRVNTEITKAPLSASMIKRYDEHH |
| | | QDLTLLKALVRQQLPEKYKEIFFDQSKNGYAGYIDGGASQEEFYKFI |
| | | KPILEKMDGTEELLVKLNREDLLRKQRTFDNGSIPHQIHLGELHAIL |
| | | RRQEDFYPFLKDNREKIEKILTFRIPYYVGPLARGNSRFAWMTRKSE |
| | | ETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQL |
| | | KEDYFKKIECFDSVEISGVEDRFNASLGTYHDLLKIIKDKDELDNEE |
| | | NEDILEDIVLTLTLFEDREMIEERLKTYAHLFDDKVMKQLKRRRYTG |
| | | WGRLSRKLINGIRDKQSGKTILDFLKSDGFANRNEMQLIHDDSLTFK |
| | | EDIQKAQVSGQGDSLHEHIANLAGSPAIKKGILQTVKVVDELVKVMG |
| | | RHKPENIVIEMARENQTTQKGQKNSRERMKRIEEGIKELGSQILKEH |
| | | PVENTQLQNEKLYLYYLQNGRDMYVDQELDINRLSDYDVDHIVPQSF |
| | | LKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMN |
| | | TKYDENDKLIREVKVITLKSKLVSDFRKDFQFYKVREINNYHHAHDA |
| | | YLNAVVGTALIKKYPKLESEFVYGDYKVYDVRKMIAKSEQEIGKATA |
| | | KYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDP |
| | | KKYGGFDSPTVAYSVLVVAKVEKGKSKKLKSVKELLGITIMERSSFE |
| | | KNPIDFLEAKGYKEVKKDLIIKLPKYSLFELENGRKRMLASAGELQK |
| | | GNELALPSKYVNFLYLASHYEKLKGSPEDNEQKQLFVEQHKHYLDEI |
| | | IEQISEFSKRVILADANLDKVLSAYNKHRDKPIREQAENIIHLFTLT |
| | | NLGAPAAFKYFDTTIDRKRYTSTKEVLDATLIHQSITGLYETRIDLS |
| | | QLGGDGGGSPKKKRKV |
| 228 | G021925 | mU*mU*mC*UUUGUAGAACUUGAAGUGUUUUAGAmGmCmUmAmGmAm |
| | | AmAmUmAmGmCAAGUUAAAAUAAGGCUAGUCCGUUAUCAmAmCmUmU |
| | | mGmAmAmAmAmAmGmUmGmGmCmAmCmCmGmAmGmUmCmGmGmUmGm |
| | | CmU*mU*mU*mU |
| 229 | Amino acid | MHSSALLCCLVLLTGVRASPGQGTQSENSCTHFPGNLPNMLRALRDA |
| | sequence for | FSRVKTFFQMKDQLDNLLLKESLLEDFKGYLGCQALSEMIQFYLEEV |
| | IL10 | MPQAENQDPDIKAHVNSLGANLKTLRLRLRRCHRFLPCENKSKAVEQ |
| | D25/E96A | VKNAFNKLQEKGIYKAMSEFDIFINYIEAYMTMKIRN |
| 230 | Nucleic acid | ATGCACAGCTCAGCACTGCTCTGTTGCCTGGTCCTCCTGACTGGGGT |
| | sequence for | GAGGGCCAGCCCAGGCCAGGGCACCCAGTCTGAGAACAGCTGCACCC |
| | IL10 | ACTTCCCAGGCAACCTGCCTAACATGCTTCGAGCTCTCCGAGATGCC |
| | D25/E96A | TTCAGCAGAGTGAAGACTTTCTTTcaaatgaaggatcagctggacaA |
| | | CTTGTTGTTAAAGGAGTCCTTGCTGGAGGACTTTAAGGGTTACCTGG |
| | | GTTGCCAAGCCTTGTCTGAGATGATCCAGTTTTACCTGGAGGAGGTG |
| | | ATGCCCCAAGCTGAGAACCAAGACCCAGACATCAAGGCGCATGTGAA |
| | | CTCCCTGGGGGCTAACCTGAAGACCCTCAGGCTGAGGCTACGGCGCT |
| | | GTCATCGATTTCTTCCCTGTGAAAACAAGAGCAAGGCCGTGGAGCAG |
| | | GTGAAGAATGCCTTTAATAAGCTCCAAGAGAAAGGCATCTACAAAGC |
| | | CATGAGTGAGTTTGACATCTTCATCAACTACATAGAAGCCTACATGA |
| | | CAATGAAGATACGAAACTGA |

The vector comprising: a guide RNA, RNA-binding DNA binding agent, or donor construct comprising a sequence encoding the dmTGFB1 molecule, the regulatory T cell promoting molecule, e.g., IL10, CTLA4; or targeting receptor, e.g., a CAR, individually or in any combination, may be delivered by liposome, a nanoparticle, an exosome, or a microvesicle. The vector may also be delivered by a lipid nanoparticle (LNP). One or more guide RNA, RNA-binding DNA binding agent (e.g., mRNA), or donor construct comprising a sequence encoding a heterologous protein, individually or in any combination, may be delivered by LNP, liposome, a nanoparticle, an exosome, or a microvesicle. One or more guide RNA, RNA-binding DNA binding agent (e.g., mRNA), or donor construct comprising a sequence encoding a heterologous protein, individually or in any combination, may be delivered by LNP. In some embodiments, one or more guide RNA and an RNA-guided DNA-binding agent (e.g., mRNA) are delivered by LNP. A donor construct may be delivered by viral vector.

Lipid nanoparticles (LNPs) are a well-known means for delivery of nucleotide and protein cargo, and may be used for delivery of any of the guide RNAs, RNA-guided DNA binding agent, or donor construct disclosed herein.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”-lamellar phase lipid bilayers that, in some embodiments, are substantially spherical and, in more particular embodiments, can comprise an aqueous core, e.g., comprising a substantial portion of RNA molecules), a dispersed phase in an emulsion, micelles, or an internal phase in a suspension (see, e.g., WO2017173054, the contents of which are hereby incorporated by reference in their entirety). Any LNP known to those of skill in the art to be capable of delivering nucleotides to subjects may be utilized. Exemplary LNP formulations and methods for delivery of agents to T cell for modification are provided, for example, in WO2021222287.

In some embodiments, provided herein is a method for delivering any of the guide RNAs described herein or donor construct disclosed herein, alone or in combination, to a cell or a population of cells or a subject, wherein any one or more of the components is associated with an LNP. In some embodiments, the method further comprises an RNA-guided DNA-binding agent (e.g., Cas9 or a sequence encoding Cas9).

In some embodiments, provided herein is a composition comprising any of the guide RNAs described herein or donor construct disclosed herein, alone or in combination, with an LNP. In some embodiments, the composition further comprises an RNA-guided DNA-binding agent (e.g., Cas9 or a sequence encoding Cas9).

In some embodiments, the LNPs comprise cationic lipids. In some embodiments, the LNPs comprise (9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino) propoxy) carbonyl)oxy)methyl) propyl (9Z,12Z)-octadeca-9,12-dienoate) or another ionizable lipid. See, e.g., lipids of WO2019067992, WO2017173054, WO2015095340, and WO2014136086, as well as references provided therein. In some embodiments, the LNPs comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, LNPs associated with the construct disclosed herein are for use in preparing a cell-based medicament for suppressing immune response. Methods for preparation of cell-based therapeutics and reagents for use in cell based therapeutics are known in the art.

In some embodiments, any of the guide RNAs described herein, RNA-guided DNA binding agents, or donor construct disclosed herein, alone or in combination, whether naked or as part of a vector, is formulated in or administered via a lipid nanoparticle; see e.g., WO2019067992, WO2017173054, or WO2021222287 the contents of which are hereby incorporated by reference in their entirety.

In some embodiments, an LNP composition comprising: an RNA component and a lipid component, wherein the lipid component comprises an amine lipid such as a biodegradable, ionizable lipid. In some instances, the lipid component comprises biodegradable, ionizable lipid, cholesterol, DSPC (distearoylphosphatidylcholine), and PEG-DMG (1,2-dimyristoyl-rac-glycero-3-methylpolyoxyethylene glycol 2000 (PEG2k-DMG)). In certain embodiments, the lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy) butanoyl)oxy)-2-((((3-(diethylamino) propoxy) carbonyl)oxy)methyl)propyl (9Z,12Z)-octadeca-9,12-dienoate), cholesterol, DSPC (distearoylphosphatidylcholine), and PEG2k-DMG (1,2-dimyristoyl-rac-glycero-3-methylpolyoxyethylene glycol 2000 (PEG2k-DMG)). In certain embodiments, the components are present in a 50 Lipid A: 38 cholesterol: 9 DSPC: 3 PEG-DMG molar ratio, respectively. In certain embodiments, the components are present in a 35 Lipid A: 47.5 cholesterol: 15 DSPC: 2.5 PEG-DMG molar ratio, respectively. The lipid nucleic acid assemblies may be formulated with a lipid amine to RNA phosphate (N:P) molar ratio of about 6, and a ratio of gRNA to mRNA of 2:1, 1:1, or 1:2 by weight.

It will be apparent that a guide RNA, an RNA-guided DNA-binding agent (e.g., Cas nuclease or a nucleic acid encoding a Cas nuclease), and a donor construct comprising a sequence encoding the dmTGFB1 molecule; the regulatory T cell promoting molecule, e.g., IL10, or targeting receptor, e.g., a CAR can be delivered using the same or different systems. For example, the guide RNA, Cas nuclease, and construct can be carried by the same vector (e.g., AAV). Alternatively, the Cas nuclease (as a protein or mRNA) or gRNA can be carried by a plasmid or LNP, while the donor construct can be carried by a vector such as AAV.

The different delivery systems can be delivered simultaneously or in any sequential order. In some embodiments, the donor construct, guide RNA, and Cas nuclease can be delivered simultaneously, e.g., in one vector, two vectors, individual vectors, one LNP, two LNPs, individual LNPs, or a combination thereof. In some embodiments, the donor construct can be delivered as a vector or associated with a LNP, prior to (e.g., about 1, 2, 3, 4, 5, 6, 7, 8, 9, 10, 11, 12, 13, 14, or more days) delivering the guide RNA or Cas nuclease, as a vector or associated with a LNP singly or together or as a ribonucleoprotein (RNP). As a further example, the guide RNA and Cas nuclease, as a vector or associated with a LNP singly or together or as a ribonucleoprotein (RNP), can be delivered prior to (e.g., about 1, 2, 3, 4, 5, 6, 7, 8, 9, 10, 11, 12, 13, 14, or more days) delivering the construct, as a vector or associated with a LNP.

### IV. Method of Engineering T Cells

The disclosure provides methods of engineering T cells to comprise a modification by insertion into the cell of heterologous sequence encoding dmTGFB1 under control of a promoter sequence. The disclosure provides methods of engineering T cells further comprising a modification e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding a regulatory T cell promoting molecule under control of a promoter sequence. The disclosure provides methods of engineering T cells to comprise a modification by insertion into the cell of heterologous sequence encoding dmTGFB1 under control of a promoter sequence. The disclosure provides methods of engineering T cells further comprising a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding IL10 under control of a promoter sequence. The disclosure provides methods of engineering T cells to comprise a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding CTLA4 under control of a promoter sequence. The disclosure provides methods of engineering T cells to comprise a modification by insertion into the cell of heterologous sequence encoding dmTGFB1 under control of a promoter sequence. The disclosure provides methods of engineering T cells further comprising a modification, e.g., knockdown, of an endogenous nucleic acid sequence encoding a TNFA; a modification, e.g., knockdown, of an endogenous nucleic acid sequence encoding an IFNG or IL17A; and insertion into the cell of heterologous sequences encoding IL10 and CTLA4, each under control of a promoter sequence. In certain embodiments, the engineered T cells comprise a modification in an endogenous nucleic acid sequence encoding each an IFNG and an IL17A.

In some embodiments, the methods comprise engineering T cells to comprise a modification by insertion into the cell of heterologous sequence encoding dmTGFB1 under control of a promoter sequence, a modification, e.g., knockdown, of an endogenous nucleic acid sequence encoding a TNFA; a modification, e.g., knockdown, of an endogenous nucleic acid sequence encoding an IFNG or an IL17A; insertion into the cell of heterologous sequence(s) encoding a regulatory T cell promoting molecule, e.g., IL10 or CTLA4, and further comprise a modification, e.g., knockdown, of TCR sequence(s). In certain embodiments, the engineered T cells comprise a modification in an endogenous nucleic acid sequence encoding each an IFNG and an IL17A.

In some embodiments, the methods comprise engineering T cells to comprise a modification by insertion into the cell of heterologous sequence encoding dmTGFB1 under control of a promoter sequence, a modification, e.g., knockdown, of an endogenous nucleic acid sequence encoding a TNFA; a modification, e.g., knockdown, of an endogenous nucleic acid sequence encoding an IFNG or an IL17A; insertion into the cell of heterologous sequence(s) encoding a regulatory T cell promoting molecule, e.g., IL10 or CTLA4, and further comprise insertion into the cell of heterologous sequence(s) encoding a targeting receptor, e.g., a CAR. In certain embodiments, the engineered T cells comprise a modification in an endogenous nucleic acid sequence encoding each an IFNG and an IL17A.

In some embodiments, the methods comprise engineering T cells to comprise a modification by insertion into the cell of heterologous sequence encoding dmTGFB1 under control of a promoter sequence, a modification, e.g., knockdown, of an endogenous nucleic acid sequence encoding a TNFA; a modification, e.g., knockdown, of an endogenous nucleic acid sequence encoding an IFNG or an IL17A; insertion into the cell of heterologous sequence(s) encoding a regulatory T cell promoting molecule, e.g., IL10 or CTLA4, a modification, e.g., knockdown, of TCR sequence(s), and insertion into the cell of heterologous sequence(s) encoding a targeting receptor, e.g., a CAR.

In some embodiments, the methods comprise modification by insertion into the cell of heterologous sequence encoding dmTGFB1 under control of a promoter sequence, a modification, e.g., knockdown, of an endogenous nucleic acid sequence encoding a TNFA; the modification, e.g., knockdown, of an endogenous nucleic acid sequence encoding an IFNG or an IL17A; insertion into the cell of heterologous sequence(s) encoding a regulatory T cell promoting molecule, optional knockdown of a TCR gene and optional insertion into the cell of a targeting receptor, e.g., a CAR, are engineered using the CRISPR/Cas system and the guide RNAs disclosed herein. In certain embodiments, the engineered T cells comprise a modification in an endogenous nucleic acid sequence encoding each an IFNG and an IL17A.

The disclosure provides methods of engineering T cells to comprise a modification by insertion into the cell of heterologous sequence encoding a regulatory T cell promoting molecule under control of a promoter sequence, further comprising a modification, e.g., knockdown, of an endogenous nucleic acid sequence encoding a TNFA; and a modification, e.g., knockdown, of an endogenous nucleic acid sequence encoding an IL17A. The disclosure provides methods of engineering T cells to comprise a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding IL10 under control of a promoter sequence. The disclosure provides methods of engineering T cells to comprise a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding CTLA4 under control of a promoter sequence. The disclosure provides methods of engineering T cells further comprising a modification, e.g., knockdown, of an endogenous nucleic acid sequence encoding an IL17A, a modification, e.g., knockdown, of an endogenous nucleic acid sequence encoding a TNFA, and insertion into the cell of heterologous sequences encoding IL10 and CTLA4, each under control of a promoter sequence. In certain embodiments, the methods do not comprise a modification e.g., knockdown, of an endogenous nucleic acid sequence encoding an IFNG.

In some embodiments, the methods comprise engineering T cells to comprise a modification, e.g., knockdown, of an endogenous nucleic acid sequence encoding a TNFA; a modification, e.g., knockdown, of an endogenous nucleic acid sequence encoding an IL17A; insertion into the cell of heterologous sequence(s) encoding a regulatory T cell promoting molecule, e.g., IL10 or CTLA4, and further comprise a modification, e.g., knockdown, of TCR sequence(s). In certain embodiments, the methods do not comprise a modification e.g., knockdown, of an endogenous nucleic acid sequence encoding an IFNG.

In some embodiments, the methods comprise engineering T cells to comprise a modification, e.g., knockdown, of an endogenous nucleic acid sequence encoding a TNFA; a modification, e.g., knockdown, of an endogenous nucleic acid sequence encoding an IL17A; insertion into the cell of heterologous sequence(s) encoding a regulatory T cell promoting molecule, e.g., IL10 or CTLA4, and further comprise insertion into the cell of heterologous sequence(s) encoding a targeting receptor, e.g., a CAR. In certain embodiments, the methods do not comprise a modification e.g., knockdown, of an endogenous nucleic acid sequence encoding an IFNG.

In some embodiments, the methods comprise engineering T cells to comprise a modification, e.g., knockdown, of an endogenous nucleic acid sequence encoding a TNFA; a modification, e.g., knockdown, of an endogenous nucleic acid sequence encoding an IL17A; insertion into the cell of heterologous sequence(s) encoding a regulatory T cell promoting molecule, e.g., IL10 or CTLA4, a modification, e.g., knockdown, of TCR sequence(s), and insertion into the cell of heterologous sequence(s) encoding a targeting receptor, e.g., a CAR. In certain embodiments, the methods do not comprise a modification e.g., knockdown, of an endogenous nucleic acid sequence encoding an IFNG.

In some embodiments, the methods comprise modification, e.g., knockdown, of an endogenous nucleic acid sequence encoding a TNFA; the modification, e.g., knockdown, of an endogenous nucleic acid sequence encoding an IL17A; insertion into the cell of heterologous sequence(s) encoding a regulatory T cell promoting molecule, optional knockdown of a TCR gene and optional insertion into the cell of a targeting receptor, e.g., a CAR, are engineered using the CRISPR/Cas system and the guide RNAs disclosed herein. In certain embodiments, the methods do not comprise a modification e.g., knockdown, of an endogenous nucleic acid sequence encoding an IFNG.

In these embodiments, the regulatory T cell promoting molecule to be inserted may be provided via a donor construct. The regulatory T cell promoting molecule provided via a donor construct may be selected from IL10, CTLA4, IDO1, ENTPD1, NT5E, IL22, AREG, IL35, GARP, CD274, FOXP3, IKZF2, EOS, IRF4, LEF1, BACH2, and IL2RA; and a modification, e.g., knockdown, of TCR gene sequence(s).

In these embodiments, the targeting receptor to be inserted may be provided via a donor construct. In some embodiments, the targeting receptor may be a chimeric antigen receptor (CAR), a T-cell receptor (TCR), or a receptor for a cell surface molecule operably linked through at least a transmembrane domain in an internal signaling domain capable of activating a T cell upon binding of the extracellular receptor portion. In some embodiments, the targeting receptor may be a receptor present on the surface of a cell, e.g., a T cell, to permit binding of the cell to a target site, e.g., a specific cell or tissue in an organism. In some of these embodiments, the targeting receptor is a CAR capable of targeting MAdCAM-1.

Suitable gene editing systems for engineering the T cells to comprise insertions and modifications, e.g., knockdowns, are disclosed herein and known in the art. In some embodiments, the gene editing systems include but are not limited to the CRISPR/Cas system; zinc finger nuclease (ZFN) system; transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence, such as a CRISPR/Cas9 system. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, TALEs of genome targeting Nature Biotech. 29:135-136 (2011)). The restriction enzymes can be introduced into cells, for use in gene editing or for genome editing in situ, a technique known as genome editing with engineered nucleases. Such methods and compositions for use therein are known in the art. See, e.g., WO2019147805, WO2014040370, WO2018073393, the contents of which are hereby incorporated in their entireties.

Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

RNA interference (RNAi) is a biological process in which RNA molecules inhibit gene expression or translation, by neutralizing targeted mRNA molecules. Small interfering RNAs (siRNAs) are central to RNA interference. RNAs are the direct products of genes, and these small RNAs (typically each strand being 19-23 nucleotides in length forming a duplex of 19-21 nucleotides) can direct the RNA induced silencing (RISC) complex to degrade messenger RNA (mRNA) molecules and thus decrease their activity by preventing translation, via post-transcriptional gene silencing. Short hairpin RNAs (shRNAs) are siRNAs that are a single RNA strand wherein the strands forming the duplex region have a hairpin structure, often generated by transcription from an expression vector. RNAi can also be accomplished by longer RNA duplex structures referred to as Dicer substrate molecules, which are cleaved by the enzyme Dicer before being loaded into RISC to promote target mRNA cleavage. Such methods and compositions for use are known in the art. In the compositions and methods provided herein, it is preferred that the RNA molecule to promote RNA interference is provided as an expression vector for durability, see, e.g., WO2018208837, the contents of which are hereby incorporated in their entireties. In some embodiments, RNAi is used with an expression vector.

It will be appreciated that the present disclosure contemplates methods of insertion performed with or without the guide RNAs disclosed herein (e.g., using a ZFN system to cause a break in a target DNA sequence, creating a site for insertion of the construct). For methods that use guide RNAs disclosed herein, the methods include the use of the CRISPR/Cas system to modify, e.g., knockdown, a nucleic acid sequence encoding TNFA, IFNG, or TCR. It will also be appreciated that the present disclosure contemplates methods of modifying, e.g., knocking down, TNFA, IFNG, or TCR, which can be performed without the guide RNAs disclosed herein (e.g., using a ZFN system to cause a break in a target DNA sequence, creating a site for insertion of the construct).

In some embodiments, the donor construct comprising the sequence for insertion, e.g., a sequence encoding a dmTGFB1 or a regulatory T cell promoting molecule, e.g., IL10, or CTLA4, is inserted at a genomic locus for a sequence that is targeted for modification, e.g., knockdown, e.g., a TCR gene.

In some embodiments, a CRISPR/Cas system (e.g., a guide RNA and RNA-guided DNA binding agent) can be used to create a site of insertion at a desired locus within a genome, at which site a donor construct comprising a sequence encoding dmTGFB1; a regulatory T cell promoting molecule, e.g., IL10, CTLA4; or targeting receptor, e.g., a CAR, e.g., a MAdCAM-1 CAR, disclosed herein can be inserted to express dmTGFB1; a regulatory T cell promoting molecule, e.g., IL10, CTLA4, or a CAR, e.g., a MAdCAM-1 CAR. The targeting receptor, e.g., a CAR, e.g., a MAdCAM-1 CAR; or inserted sequence, dmTGFB1or a regulatory T cell promoting molecule, e.g., IL10, or CTLA4, may be heterologous with respect to its insertion site or locus, for example a safe harbor locus or a TCR locus from which dmTGFB1; a regulatory T cell promoting molecule, e.g., IL10, CTLA4; or targeting receptor, e.g., a CAR, e.g., MAdCAM-1 CAR, is not normally expressed, as described herein. In some embodiments, a guide RNA described herein can be used according to the present methods with an RNA-guided DNA-binding agent (e.g., Cas nuclease) to create a site of insertion, at which site a donor construct comprising a sequence encoding dmTGFB1; a regulatory T cell promoting molecule, e.g., IL10, CTLA4; or targeting receptor, e.g., a CAR, e.g., a MAdCAM-1 CAR can be inserted to express dmTGFB1; a regulatory T cell promoting molecule, e.g., IL10, CTLA4; or a CAR, e.g., a MAdCAM-1 CAR. The guide RNAs for insertion of dmTGFB1; a regulatory T cell promoting molecule, e.g., IL10, CTLA4; or targeting receptor, e.g., a CAR, e.g., a MAdCAM-1 CAR, into specific genomic loci, are exemplified and described herein. In alternative embodiments, the targeting receptor is a TNFA targeting receptor.

In some embodiments, CD4+ T cells are engineered by transduction (e.g., using viral or non-viral delivery) with a gRNA (e.g., gRNA targeting IFNG, TNFA, IL17A, or TCR for knockdown), an RNA-guided DNA-binding agent (e.g., Cas nuclease), a donor construct. In some embodiments, the engineered T cells are: 1) transduced with a gRNA targeting a nucleic acid sequence encoding a pro-inflammatory cytokine, e.g., IFNG or TNFA, an RNA guided DNA binding agent (e.g., Cas nuclease), and 2) transduced with a donor construct comprising nucleic acid sequence(s) encoding dmTGFB1; a regulatory T cell promoting molecule, e.g., IL10 or CTLA4; and a targeting receptor, e.g., a CAR, e.g., a MAdCAM-1 CAR; or a TNFA targeting receptor. In certain embodiments, the engineered cells are selected for expression of the targeting receptor.

In some embodiments, CD4+ T cells are engineered by transduction with a gRNA (e.g., gRNA targeting IFNG, TNFA, IL17A, or TCR for knockdown), an RNA-guided DNA-binding agent (e.g., Cas nuclease) and a donor construct. In some embodiments, the engineered T cells are: 1) transduced with a donor construct comprising nucleic acid sequence(s) encoding dmTGFB1, a regulatory T cell promoting molecule, e.g., IL10 or CTLA4, and a targeting receptor, e.g., a CAR, e.g., a MAdCAM-1 CAR; or a TNFA targeting receptor, and 2) transduced with a gRNA targeting a nucleic acid sequence encoding a pro-inflammatory cytokine, e.g., IFNG or TNFA, an RNA guided DNA binding agent (e.g., Cas nuclease). In certain embodiments, the engineered cells are selected for expression of the targeting receptor.

In some embodiments, CD4+ T cells are engineered by transduction with a gRNA (e.g., gRNA targeting IFNG, TNFA, or TCR for knockdown), an RNA-guided DNA-binding agent (e.g., Cas nuclease), a donor construct. In some embodiments, the engineered T cells are: 1) transduced with a donor construct comprising nucleic acid sequence(s) encoding dmTGFB1; a regulatory T cell promoting molecule, e.g., IL10 or CTLA4; and a targeting receptor, e.g., a CAR, e.g., a MAdCAM-1 CAR; or a TNFA targeting receptor, and 2) transduced with a gRNA targeting a nucleic acid sequence encoding a pro-inflammatory cytokine, e.g., IFNG, IL17A, or TNFA; and an RNA guided DNA binding agent (e.g., Cas nuclease).

As described herein, the donor construct comprising a sequence encoding dmTGFB1; a regulatory T cell promoting molecule, e.g., IL10, CTLA4; or a targeting receptor e.g., a CAR, guide RNA (e.g., gRNA targeting IFNG, IL17A, TNFA, or TCR for knockdown), and RNA-guided DNA-binding agent can be delivered using any suitable delivery system and method known in the art. In some embodiments, the guide RNA and Cas nuclease are associated with an LNP and delivered to the cell or the population of cells prior to delivering the donor construction comprising a sequence encoding dmTGFB1; a regulatory T cell promoting molecule, e.g., IL10, CTLA4; or a targeting receptor, e.g., a CAR. In some embodiments, the guide RNA and Cas nuclease are associated with an LNP and delivered to the cell or the population of cells after delivering the donor construction comprising a sequence encoding dmTGFB1; a regulatory T cell promoting molecule, e.g., IL10, CTLA4, or a targeting receptor, e.g., a CAR.

In some embodiments, administration of the gRNAs, donor construct, and RNA-guided DNA binding agents described herein to a naturally occurring T cell is capable of converting the naturally occurring T cell, e.g., a CD4+ T cell, to a cell that exhibits the characteristics, e.g., immune response suppressive characteristics, of a regulatory T cell.

gRNAs, donor constructs, and RNA-guided DNA binding agents for modifying, e.g., knocking down, IFNG, TNFA, IL17A, or TCR gene expression or inserting a sequence encoding dmTGFB1; a regulatory T cell promoting molecule, e.g., IL10, CTLA4, or a targeting receptor, e.g., a CAR, e.g., a MAdCAM-1 CAR, or a TNFA targeting receptor, may be introduced to a conventional T cell or population of conventional T cells to generate the engineered T cells or population of T cells described herein.

Methods of using various RNA-guided DNA-binding agents, e.g., a nuclease, such as a Cas nuclease, e.g., Cas9, are also well known in the art. While the use of a CRISPR/Cas system is exemplified herein, it will be appreciated that suitable variations to the system can also be used. It will be appreciated that, depending on the context, the RNA-guided DNA-binding agent can be provided as a nucleic acid (e.g., DNA or mRNA), such as the mRNAs encoding an RNA-guided DNA-binding agent provided above, or as a protein. In some embodiments, the present method can be practiced in a cell that already comprises or expresses an RNA-guided DNA-binding agent.

In some embodiments, the RNA-guided DNA-binding agent, such as a Cas9 nuclease, has cleavase activity, which can also be referred to as double-strand endonuclease activity. In some embodiments, the RNA-guided DNA-binding agent, such as a Cas9 nuclease, has nickase activity, which can also be referred to as single-strand endonuclease activity. In some embodiments, the RNA-guided DNA-binding agent comprises a Cas nuclease. Examples of Cas nucleases include those of the type II CRISPR systems of S. pyogenes, S. aureus, Neisseria meningitidis, and other prokaryotes (see, e.g., the list in the next paragraph), and variant or mutant (e.g., engineered, non-naturally occurring, naturally occurring, or other variant) versions thereof. See, e.g., US20160312198; US20160312199.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA-binding agent is called a ribonucleoprotein complex (RNP). In some embodiments, the RNA-guided DNA-binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild-type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild-type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22: 163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)). Exemplary amino acid substitutions in the HNH or HNH-like nuclease domain or RuvC or RuvC-like domains for N. meningitidis include Nme2Cas9D16A (HNH nickase) and Nme2Cas9H588A (RuvC nickase).

In some embodiments, a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-5 NLS(s). In some embodiments, the RNA-guided DNA-binding agent may be fused with 2, 3, or 4 NLS(s). In some embodiments, the RNA-guided DNA-binding agent may be fused with two NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the NLS is not linked to the C-terminus. It may also be inserted within the RNA-guided DNA-binding agent sequence. In other embodiments, the RNA-guided DNA-binding agent may be fused with more than one NLS. In certain circumstances, at least the two NLSs are the same (e.g., two SV40 NLSs). In certain embodiments, at least two different NLSs are present the RNA-guided DNA binding agent. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 143) or PKKKRRV (SEQ ID NO: 144). In some embodiments, the NLS may be a bipartite sequence, such as the NLS of nucleoplasmin, KRPAATKKAGQAKKKK (SEQ ID NO: 145). In a specific embodiment, a single PKKKRKV (SEQ ID NO: 143) NLS may be linked at the C-terminus of the RNA-guided DNA-binding agent. One or more linkers are optionally included at the fusion site.

### V. Method of Treatment

The disclosure provides methods for suppressing an immune response in a subject, comprising administering engineered T cells comprising an insertion into the cell of a heterologous sequence encoding a dmTGFB1 under control of a promoter sequence; a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding a regulatory T cell promoting molecule under control of a promoter sequence. The disclosure provides methods for suppressing an immune response in a subject, comprising administering engineered T cells comprising a modification, e.g., knockdown, of an endogenous nucleic acid sequence encoding a TNFA; a modification, e.g., knockdown, of an endogenous nucleic acid sequence encoding an IFNG or an IL17A; and insertion into the cell of a heterologous sequence encoding IL10 under control of a promoter sequence. The disclosure provides methods for suppressing an immune response in a subject, comprising administering engineered T cells comprising an insertion into the cell of a heterologous sequence encoding a dmTGFB1 under control of a promoter sequence; a modification, e.g., knockdown, of an endogenous nucleic acid sequence encoding a TNFA; a modification, e.g., knockdown, of an endogenous nucleic acid sequence encoding an IFNG or an IL17A; and insertion into the cell of a heterologous sequence encoding CTLA4 under control of a promoter sequence. The disclosure provides methods for suppressing an immune response in a subject, comprising administering engineered T cells comprising an insertion into the cell of a heterologous sequence encoding a dmTGFB1 under control of a promoter sequence; a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequences encoding IL10 and CTLA4, each under control of a promoter sequence.

The disclosure provides methods for treating an autoimmune disorder in a subject, comprising administering engineered T cells comprising an insertion into the cell of a heterologous sequence encoding dmTGFB1 under control of a promoter sequence; a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding a regulatory T cell promoting molecule under control of a promoter sequence. The disclosure provides methods for treating an autoimmune disorder in a subject, comprising administering engineered T cells comprising an insertion into the cell of a heterologous sequence encoding dmTGFB1 under control of a promoter sequence; a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence encoding IL10 under control of a promoter sequence. The disclosure provides methods for treating an autoimmune disorder in a subject, comprising administering engineered T cells comprising an insertion into the cell of a heterologous sequence encoding dmTGFB1 under control of a promoter sequence; a modification, e.g., knockdown, of an endogenous nucleic acid sequence encoding an IFNG, a modification, e.g., knockdown, of an endogenous nucleic acid sequence encoding a TNFA, and insertion into the cell of heterologous sequence(s) encoding CTLA4 under control of a promoter sequence. The disclosure provides methods for treating an autoimmune disorder in a subject, comprising administering engineered T cells comprising an insertion into the cell of a heterologous sequence encoding dmTGFB1 under control of a promoter sequence; a modification, e.g., knockdown, of an endogenous nucleic acid sequence encoding an IFNG, a modification, e.g., knockdown, of an endogenous nucleic acid sequence encoding a TNFA, and insertion into the cell of heterologous sequences encoding IL10 and CTLA4, each under control of a promoter sequence.

The disclosure provides methods for treating GvHD in a subject, comprising administering engineered T cells comprising an insertion into the cell of a heterologous sequence encoding dmTGFB1 under control of a promoter sequence; a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding a regulatory T cell promoting molecule under control of a promoter sequence. The disclosure provides methods for treating GvHD in a subject, comprising administering engineered T cells comprising an insertion into the cell of a heterologous sequence encoding dmTGFB1 under control of a promoter sequence; a modification, e.g., knockdown, of an endogenous nucleic acid sequence encoding a TNFA; a modification, e.g., knockdown, of an endogenous nucleic acid sequence encoding an INFG or an IL17A and insertion into the cell of heterologous sequence(s) encoding IL10 under control of a promoter sequence. The disclosure provides methods for treating GvHD in a subject, comprising administering engineered T cells comprising an insertion into the cell of a heterologous sequence encoding dmTGFB1 under control of a promoter sequence; a modification, e.g., knockdown, of an endogenous nucleic acid sequence encoding a TNFA; a modification, e.g., knockdown, of an endogenous nucleic acid sequence encoding an IFNG or an IL17A; and insertion into the cell of heterologous sequence(s) encoding CTLA4 under control of a promoter sequence. The disclosure provides methods for treating GvHD in a subject, comprising administering engineered T cells comprising an insertion into the cell of a heterologous sequence encoding dmTGFB1 under control of a promoter sequence; a modification, e.g., knockdown, of an endogenous nucleic acid sequence encoding a TNFA; a modification, e.g., knockdown, of an endogenous nucleic acid sequence encoding an INFG or an IL17A; and insertion into the cell of heterologous sequences encoding IL10 and CTLA4, each under control of a promoter sequence.

The disclosure provides methods for suppressing an immune response in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding a regulatory T cell promoting molecule under control of a promoter sequence. The disclosure provides methods for suppressing an immune response in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of a heterologous sequence encoding IL10 under control of a promoter sequence. The disclosure provides methods for suppressing an immune response in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of a heterologous sequence encoding CTLA4 under control of a promoter sequence. The disclosure provides methods for suppressing an immune response in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of heterologous sequences encoding IL10 and CTLA4, each under control of a promoter sequence. In certain embodiments, the methods do not comprise administering engineered T cells comprising a modification comprising knockdown, of an endogenous nucleic acid sequence encoding an IFNG.

The disclosure provides methods for treating GvHD in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding a regulatory T cell promoting molecule under control of a promoter sequence. The disclosure provides methods for treating GvHD in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A and insertion into the cell of heterologous sequence(s) encoding IL10 under control of a promoter sequence. The disclosure provides methods for treating GvHD in a subject, comprising administering engineered T cells comprising a modification, e.g., knockdown, of an endogenous nucleic acid sequence encoding a TNFA; a modification, e.g., knockdown, of an endogenous nucleic acid sequence encoding an IL17A; and insertion into the cell of heterologous sequence(s) encoding CTLA4 under control of a promoter sequence. The disclosure provides methods for treating GvHD in a subject, comprising administering engineered T cells comprising an insertion into the cell of a modification, e.g., knockdown, of an endogenous nucleic acid sequence encoding a TNFA; a modification, e.g., knockdown, of an endogenous nucleic acid sequence encoding an IL17A; and insertion into the cell of heterologous sequences encoding IL10 and CTLA4, each under control of a promoter sequence. In certain embodiments, the methods do not comprise administering engineered T cells comprising a modification comprising knockdown, of an endogenous nucleic acid sequence encoding an IFNG.

In some embodiments, the methods comprise administering engineered T cells comprising a modification, e.g., knockdown, of an endogenous nucleic acid sequence encoding a TNFA; a modification, e.g., knockdown, of an endogenous nucleic acid sequence encoding an INFG or an IL17A; insertion of sequence(s) encoding a regulatory T cell promoting molecule, and further comprising a modification, e.g., knockdown, of TCR sequence(s).

In some embodiments, the methods comprise administering engineered T cells comprising a modification, e.g., knockdown, of an endogenous nucleic acid sequence encoding a TNFA; a modification, e.g., knockdown, of an endogenous nucleic acid sequence encoding an IL17A; insertion of sequence(s) encoding a regulatory T cell promoting molecule, and further comprising a modification, e.g., knockdown, of TCR sequence(s). In certain embodiments, the methods do not comprise administering engineered T cells comprising a modification comprising knockdown, of an endogenous nucleic acid sequence encoding an IFNG.

In some embodiments, the methods comprise administering engineered T cells comprising a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding a regulatory T cell promoting molecule, and further comprising insertion of sequence(s) encoding a targeting receptor, e.g., a CAR.

In some embodiments, the methods comprise administering engineered T cells comprising a modification, e.g., knockdown, of an endogenous nucleic acid sequence encoding a TNFA; a modification, e.g., knockdown, of an endogenous nucleic acid sequence encoding an IL17A; insertion of sequence(s) encoding a regulatory T cell promoting molecule, and further comprising insertion of sequence(s) encoding a targeting receptor, e.g., a CAR. In certain embodiments, the methods do not comprise administering engineered T cells comprising a modification comprising knockdown, of an endogenous nucleic acid sequence encoding an IFNG.

In some embodiments, the targeting receptor, e.g., a CAR, is capable of targeting engineered T cells to the gastrointestinal system, e.g., the targeting receptor is a CAR targeting MAdCAM-1, e.g., for suppressing immune responses, including inflammation, in disorders such as inflammatory bowel disease, ulcerative colitis, or Crohn's disease. In some embodiments, the targeting receptor, e.g., a CAR, is capable of targeting engineered T cells to tissues comprising endothelial cells, e.g., the targeting receptor is a CAR targeting VCAM-1, e.g., for suppressing immune responses, including inflammation, in disorders such as Crohn's disease and multiple sclerosis. In some embodiments, the targeting receptor, e.g., a CAR, is capable of targeting engineered T cells to endothelial cells, e.g., the targeting receptor is a CAR targeting CEACAM6, e.g., for suppressing immune responses in disorders such as Crohn's disease. In some embodiments, the targeting receptor, e.g., a CAR, is capable of targeting engineered T cells to B cells, e.g., the targeting receptor is a CAR targeting CD19, e.g., for suppressing immune responses in disorders such as in multiple sclerosis and systemic lupus erythematosus. In some embodiments, the targeting receptor, e.g., a CAR, is capable of targeting engineered T cells to B cells, e.g., the targeting receptor is a CAR targeting CD20, e.g., for suppressing immune responses in disorders such as in multiple sclerosis and systemic lupus erythematosus. In some embodiments, the targeting receptor, e.g., a CAR, is capable of targeting engineered T cells to an inflammatory tissue, e.g., the targeting receptor is a CAR targeting TNFA, e.g., for suppressing immune responses in disorders such as inflammatory bowel disease, ulcerative colitis, or Crohn's disease. In some embodiments, the targeting receptor, e.g., a CAR is capable of targeting engineered T cells to a neurological tissue, e.g., the targeting receptor is a CAR targeting MBP, MOG, or PLP, e.g., for suppressing immune responses in disorders such as multiple sclerosis. In some embodiments, the targeting receptor, e.g., a CAR, is capable of targeting engineered T cells to tissues comprising mature B lymphocytes, e.g., the targeting receptor is a CAR targeting TNFRSF17, e.g., for suppressing immune responses in disorders such as systemic lupus erythematosus. In some embodiments, the targeting receptor, e.g., a CAR, is capable of targeting engineered T cells to synovial tissue, e.g., the targeting receptor is a CAR targeting citrullinated vimentin e.g., for suppressing immune responses in disorders such as rheumatoid arthritis.

In some embodiments, the targeting receptor is a CAR targeting DPP6, SCL2A2, glutamate decarboxylase (GAD2), demoglein 3 (DSG3), and MHC class I HLA-A (HLA-A*02).

In some embodiments, the methods comprise administering engineered T cells comprising a modification, e.g., knockdown, of an endogenous nucleic acid sequence encoding a TNFA; a modification, e.g., knockdown, of an endogenous nucleic acid sequence encoding an IFNG or an IL17A; insertion of sequence(s) encoding a regulatory T cell promoting molecule, an insertion of sequence(s) encoding a targeting receptor, e.g., a CAR, and further comprising a modification, e.g., knockdown, of TCR sequence(s).

In some embodiments, the methods comprise administering engineered T cells comprising a modification, e.g., knockdown, of an endogenous nucleic acid sequence encoding a TNFA; a modification, e.g., knockdown, of an endogenous nucleic acid sequence encoding an IL17A; insertion of sequence(s) encoding a regulatory T cell promoting molecule, an insertion of sequence(s) encoding a targeting receptor, e.g., a CAR, and further comprising a modification, e.g., knockdown, of TCR sequence(s). In certain embodiments, the methods do not comprise administering engineered T cells comprising a modification comprising knockdown, of an endogenous nucleic acid sequence encoding an IFNG.

In some embodiments, the sequence(s) to be inserted are inserted into the sequence(s) to be modified, e.g., knocked down, e.g., a CAR sequence is inserted into a TNFA genomic sequence, thereby modifying, e.g., knocking down, the TNFA sequence.

In some embodiments, the methods comprise administering a population of T cells comprising T cells that are engineered as described above. In some embodiments, at least 40%, 45%, preferably at least 50%, 55%, 60%, 65%, 70%, 75%, 80%, 85%, 90%, 95%, or 100% of the T cells in the population of T cells are engineered, e.g., as assessed by sequencing, e.g., NGS.

In some embodiments, the autoimmune disorder is selected from ulcerative colitis, Crohn's disease, rheumatoid arthritis, psoriasis, multiple sclerosis, systemic lupus erythematosus, type 1 diabetes, and graft versus host disease (GvHD). In some embodiments, the engineered T cells have autologous or allogenic use.

In some embodiments, the effectiveness of treatment using the engineered T cell described above can be assessed in an animal model, e.g., mouse model, of graft versus host disease by measuring the animal's weight or survival (wherein the animals are sacrificed after loss of a substantial portion of body weight, e.g., 20% of starting body weight) following administration of the engineered T cell. In some embodiments, effective treatment results in a statistically significant increase in survival rate as compared to a suitable control, e.g., an animal treated with PBMC.

### EXAMPLES

The following examples are provided to illustrate certain disclosed embodiments and are not to be construed as limiting the scope of this disclosure in any way.

### Example 1. General Methods

### 1.1. Preparation of Lipid Nanoparticles

In general, the lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

Unless otherwise specified, the lipid nucleic acid assemblies contained ionizable Lipid A ((9Z,12Z)-3-((4,4-bis(octyloxy butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 35 Lipid A: 47.5 cholesterol: 15 DSPC: 2.5 PEG-DMG molar ratio, respectively. The lipid nucleic acid assemblies were formulated with a lipid amine to RNA phosphate (N:P) molar ratio of about 6, and a ratio of gRNA to mRNA of 1:1 by weight, unless otherwise specified.

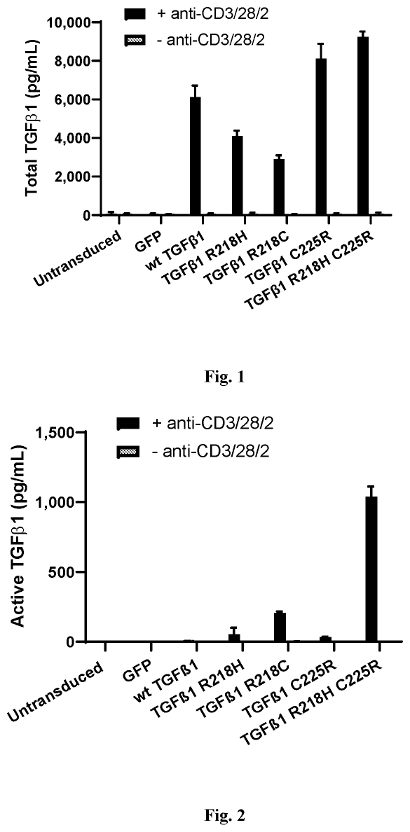

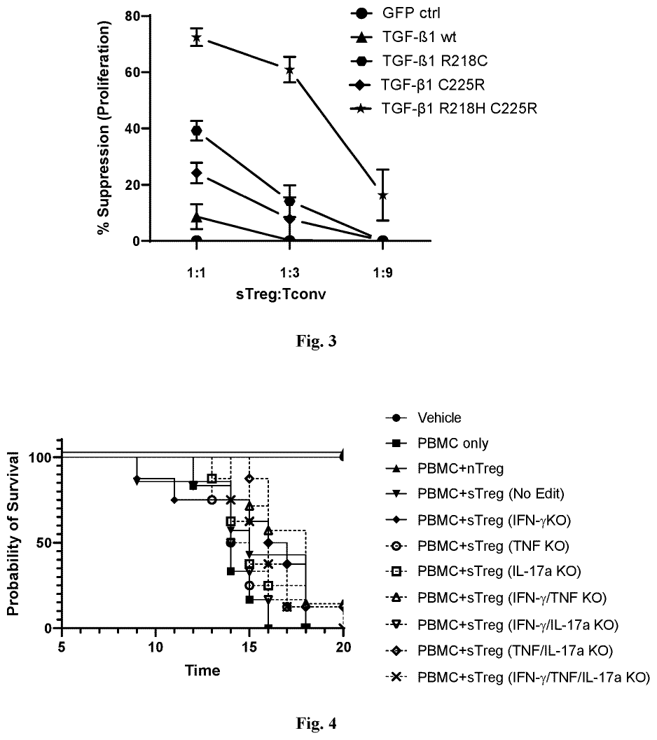

LNPs were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). LNPs were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNPs were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.2. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA encoding an open reading frame (SEQ ID NO: 17) was generated from plasmid DNA according to the nucleic acid sequences described herein. For the mRNA nucleic acid sequences below, it is understood that Ts should be replaced with Us (which were N1-methyl pseudouridines as described above). Messenger RNAs used in the Examples include a 5′ cap and a 3′ poly-A tail, e.g., up to 100 nts. Guide RNAs were chemically synthesized by methods known in the art.

### 1.3. Next-Generation Sequencing (“NGS”) and Analysis for On-Target Editing Efficiency

Genomic DNA was extracted using QuickExtract™ DNA Extraction Solution (Lucigen, Cat. QE09050) according to the manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq or NextSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an indel was calculated. The editing percentage (e.g., the “editing efficiency” or “percent editing” or “percent indels”) is defined as the total number of sequence reads with indels over the total number of sequence reads, including wild type.

### Example 2. Suppressive Activity of Engineered T Cells Expressing TGFB1 Alleles

The suppressive phenotype of T cells was assessed with lentivirus overexpression of constitutively active TGFB1 mutants that decouple TGFB1 from LAP. TGFB1 is a well known suppressive cytokine that is generated in a pro-form consisting of TGFB1 and the latency associated peptide (LAP). The activation of TGFB1 requires the cleavage of LAP ancillary proteins.

### Example 2.1. T Cell Preparation

Human CD3+CD4+ T cells were prepared from a fresh leukopak (AllCells, Donor #32015). CD3+CD4+ T cells were isolated by negative selection using the human CD4+ T cell isolation kit (Miltenyi; Cat no. 130-096-533) following the manufacturer's protocol. The CD3+CD4+ T cells were plated at a density of 1×10\hat{ }⁶ cells/mL in a total of 5 ml of OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) supplemented with 200 U/mL of recombinant human interleukin-2 (Miltenyi Biotec; Cat No. 130-097-746)), 5 ng/ml recombinant human interleukin 7 (StemCell technologies, Cat No. 78053.1), and 5 ng/ml recombinant human interleukin 15 (StemCell technologies, Cat No. 78031.1). Cells were activated with by addition of 25 uL/mL ImmunoCult Human CD3/CD28 T cell Activator (Stemcell Technologies, Cat. 10991) and cultured at 37° C. for 48 hours prior to lentiviral transduction.

### Example 2.2. T Cell Transduction and Cell Sorting

To overexpress wild-type or mutant TGFB1, activated CD3+CD4+ T cells were transduced with lentiviral constructs. Forty-eight hours after activation, CD3+CD4+ T cells were harvested, washed and resuspended at a density of 1×10\hat{ }⁶ cells/100 uL OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512). Concentrated viral supernatant was added to the CD3+CD4+ T cells and centrifuged at 1000×g for 60 mins at 37° C. Following transduction, the CD3+CD4+ T cells were resuspended in the cell/viral supernatant mixture and transferred to a single well of a 6-well G-rex (Wilson Wolf; Cat. 80240M) containing 20 mL OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) supplemented with 500 U/mL IL-2, 5 ng/mL IL-7, and 5 ng/ml IL-17. The transduced CD3+CD4+ T cells were cultured for 3-4 days and sorted using a BD FACSAria™ Fusion Cell Sorter (BD Biosciences) to isolate cells expressing the target of interest. Following sorting, the CD3+CD4+ T cells were cultured in 6-well Grex plates with 30 mL OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) supplemented with 500 U/mL IL-2, 5 ng/ml IL7, and 5 ng/ml IL17.

Natural regulatory T cells (nTregs) were prepared using methods known in the art. Briefly, CD25+ cells were isolated fromCD4+ T cells autologous to the PBMCs, isolated in example 2.1, using CD25 microbeads (Miltenyi, Cat. 130-092-983) according to manufacturer's instructions. Isolated CD25+CD4+ T cells were plated at 5 million per well in a 6-well Grex containing 30 ml of OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) supplemented with 1000 U/mL of recombinant human interleukin-2 (Miltenyi Biotec; Cat No. 130-097-746)). Cells were activated by addition of 25 uL/mL ImmunoCult Human CD3/CD28 T cell Activator (Stemcell Technologies, Cat. 10991) and cultured at 37° C. for 5 days. Activated CD3+CD4+CD25+ T cells were harvested and labelled with 50 ug/mL biotinylated anti-LAP antibody (Miltenyi; Cat. 130-095-213) for 15 min at 4 C. The labelled cells were washed twice and further labelled with anti-biotin microbeads (Miltenyi; Cat. 130-090-485) according to manufacturer's instructions. The cells were isolated using LS columns (Miltenyi; Cat. 130-042-401). The isolated natural Tregs were cultured in 6-well Grex plates with 30 mL OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) supplemented with 1000 U/mL IL-2.

### Example 2.3 Validating TGFB1 Protein Expression

TGFB1 protein expression from the various lentiviral constructs was validated by ELISA. One-hundred thousand sorted, transduced T cells were placed in a U-bottom plate containing OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512). Some wells were also received 25 uL/mL ImmunoCult Human CD3/CD28/2 T cell Activator (Stemcell Technologies, Cat. 10990). Wells that did not receive anti-CD3/CD28 stimulation were used to assess background cytokine release. The cells were cultured at 37° C. for 48 hours. Following 48 hours of culture, the culture plate was centrifuged, the supernatants collected and frozen for subsequent cytokine quantification using a LEGEND MAX Total TGF-β1 ELISA kit (BioLegend, Cat. 436707) or a LEGEND MAX Free Active TGF-β1 ELISA kit according to manufacturer's instructions. Total TGFB1 in culture supernatants are shown in Table 1 and FIG. 1. Active TGFB1 protein in culture supernatant is shown in Table 2 and FIG. 2. CD3+CD4+ T cells transduced with a lentiviral expression vector with a sequence encoding TGFB1 R218H C225R secreted elevated level of active TGF-β1.

TABLE 1
Total TGF-β1 quantified in culture supernatants in pg/ml

| | +anti-CD3/28/2 | | | −Anti CD3/28/2 | | |
|---|---|---|---|---|---|---|
| Groups | Mean | SD | n | Mean | SD | n |
| Untransduced | 68.59 | 93.77 | 3 | 10.35 | 73.63 | 2 |
| GFP | 44.42 | 40.94 | 3 | 13.38 | 37.39 | 2 |
| wt TGFβ1 | 6117.43 | 600.12 | 3 | 58.76 | 32.45 | 2 |
| TGFβ1 R218H | 4104.32 | 286.59 | 3 | 55.86 | 73.07 | 2 |
| TGFβ1 R218C | 2909.45 | 190.93 | 3 | 2.01 | 55.81 | 2 |
| TGFβ1 C225R | 8119.93 | 769.31 | 3 | 17.19 | 78.93 | 2 |
| TGFβ1 R218H | 9246.18 | 274.43 | 3 | 19.76 | 120.83 | 2 |
| C225R | | | | | | |

TABLE 2
Active TGF-β1 quantified in culture supernatants in pg/ml

| | +anti-CD3/28/2 | | | −anti-CD3/28/2 | | |
|---|---|---|---|---|---|---|
| Groups | Mean | SD | n | Mean | SD | n |
| Untransduced | 0 | 0 | 3 | 0 | 0 | 2 |
| GFP | 0 | 0 | 3 | 0 | 0 | 2 |
| wt TGFβ1 | 3.17 | 1.23 | 3 | 0.56 | 0.14 | 2 |
| TGFβ1 R218H | 53.13 | 45.99 | 3 | 0.28 | 0.07 | 2 |
| TGFβ1 R218C | 205.64 | 10.90 | 3 | 1.07 | 0.92 | 2 |
| TGFβ1 C225R | 32.72 | 2.59 | 3 | 0.10 | 0.14 | 2 |
| TGFβ1 R218H | 1038.34 | 73.99 | 3 | 0.11 | 0.16 | 2 |
| C225R | | | | | | |

### Example 2.4 Suppressive Capacity of Engineered T Cells Expressing Mutant TGF-β1

A mixed lymphocyte reaction (MLR) assay was used to assay the suppressive function of sorted transduced CD3+CD4+ T cells. MLR is an inflammatory reaction caused by T cells recognizing leukocytes of another (allogenic leukocytes) as foreign. Tregs suppress the MLR inflammatory reaction by suppressing proliferation of and production of inflammatory cytokines by inflammatory T cells. Cell proliferation is measured by labeling the initial population with an intracellular fluorescent dye that becomes diluted as it is passed as part of the cell contents to each successive generation.

The MLR assay was conducted in a 96-well U-bottom plate using OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512). Untransduced CD3+CD4+ T cells from the same donor as the transduced T cells (autologous T cells) were used as the responding cells. These untransduced cells were labelled with CellTrace Violet (CTV) (Thermofisher Scientific; Cat No. C34557) according to manufacturer's instructions. CD3-depleted PBMC from an allogenic donor relative to the transduced T cells were used to stimulate the responding, untransduced T cells. Cultures were prepared by combining 50,000 CTV-labelled autologous T cells, 50,000 CD3-depleted allogenic PBMC, and approximately 50,000 (1 to 1), 16,666 (3 to 1) or 5,555 (9 to 1) sorted transduced CD3+CD4+ T cells per well. Following 5 days of culture at 37° C., the culture plate was centrifuged, and culture supernatants were harvested for cytokine quantification. The cell pellet was resuspended in FACS buffer containing APC/Fire 750 anti-CD4 and placed at 4° C. for 30 mins. The cells were subsequently washed, processed on a CytoFlex flow cytometer (Beckman Coulter), and analyzed using the FlowJo software package. Cells were first gated by positive CD4 expression, followed by CTV signal and finally on the undiluted CTV population. Suppression of CTV-dilution was calculated using the following formula:

$$( log 2 ( y ) of CTV T cells - log 2 ( y ) of CTV T cells with Treg ) ) / log 2 ( y ) of CTV T cells * 100$$

Where y=mean fluorescent intensity of the entire CTV-labelled population/mean fluorescent intensity of the undiluted portion of the CTV-labelled population. Data are shown in FIG. 3 and Table 3.

TABLE 3
Percent suppression of autologous T cell proliferation
by T cells overexpressing wild-type or mutant
TGF-β1 as measured by CTV dilution

| | 1:1 | | | 1:3 | | | 1:9 | | |
|---|---|---|---|---|---|---|---|---|---|
| Groups | Mean | SD | n | Mean | SD | n | Mean | SD | n |
| GFP | 0 | 0 | 3 | 0 | 0.00 | 3 | 0 | 0 | 3 |
| wt TGFβ1 | 8.63 | 7.63 | 3 | 0.22 | 0.39 | 3 | 0 | 0 | 3 |
| TGFβ1 | 39.20 | 6.03 | 3 | 14.14 | 9.84 | 3 | 0 | 0 | 3 |
| R218C | | | | | | | | | |
| TGFβ1 | 24.18 | 6.15 | 3 | 7.74 | 13.41 | 3 | 0 | 0 | 3 |
| C225R | | | | | | | | | |
| TGFβ1 | 72.45 | 5.45 | 3 | 60.91 | 7.96 | 3 | 16.32 | 15.70 | 3 |
| R218H | | | | | | | | | |
| C225R | | | | | | | | | |

### Example 3. Suppressive Activity of Engineered T Cells Expressing an Active TGFB1 Mutant with Disruption of Cytokine Genes

### Example 3.1. T Cell Engineering

The in vivo suppressive function of the T cells expressing the TGFβ1 R218H C225R mutant was assessed using a graft versus host disease (GvHD) mouse model. Transduced T cells expressing mutant TGFB1 were assessed with the additional disruption of one or more of the following cytokine genes: Tumor necrosis factor-alpha (TNFa), interferon gamma (IFNg), or interleukin 17a (IL17a).

T cells were isolated and transduced with lentivirus as described in Example 2 and further engineered with lipid nanoparticle (LNP) delivery of Cas9 mRNA and guide RNA. LNPs were generated as described in Example 1 using a molar ratio of 35 Lipid A/47.5 cholesterol/15 DSPC/2.5 PEG2k-DMG. The LNPs were formulated with a lipid amine to RNA phosphate (N:P) molar ratio of about 6. LNPs were prepared with a ratio of 1:1 by weight of gRNA to Cas9 mRNA cargo. To disrupt the target gene, LNPs were prepared with G019753 (IFNg), G019757 (TNFa), or G027259 (IL17a).

Each LNP preparation was incubated in OpTmizer base media including CTS OpTmizer T Cell Expansion SFM (Gibco, Cat. A3705001), 1% Penicillin-Streptomycin, 1× Glutamax, 10 mM HEPES, 5% human AB serum (Gemini, Cat. 100-512), 200 U/mL recombinant human interleukin-2, 5 ng/ml recombinant human interleukin 7, and 5 ng/ml recombinant human interleukin-15 supplemented with 5 ug/ml recombinant human ApoE3 (Peprotech, Cat. 350-02) for 15 minutes at 37° C. Day 3 post-activation and transduction, the transduced T cells were washed and suspended in Serum Free OpTmizer media with 200 U/mL recombinant human interleukin-2, 5 ng/ml recombinant human interleukin 7, and 5 ng/ml recombinant human interleukin-15 at concentration of 1 million T cells/mL of media and plated in T-25 flask with 5 million cells per group.

Pre-incubated LNP mix was added to each T-25 flask to yield a final concentration of 2.5 ug/ml total RNA. Groups with multiple knockouts used 2.5 ug/ml of each respective LNP. Twenty-four hours after LNP treatment, T cells were collected, washed, and cultured in Optimizer media with 5% Human Serum with cytokines as described in Example 2, for expansion until day of injection (Day 15 post-activation). Cells were sorted about five to six days post activation as described in Example 2.

### Example 3.2 In Vivo Assessment of Immunosuppression in a Model of GvHD

Sorted CD3+CD4+ T cells for in vivo injections were harvested 15 days post-activation. PBMCs autologous to the engineered T cells were thawed as described above in the Examples. PBMCs were added to each assay population at a 1:1 ratio and cells resuspended in RPMI supplemented with 2% FBS and 10 mM HEPES to 8×10\hat{ }⁶/100 μL. The PBMC only group was resuspended at 4×10\hat{ }⁶/100 μL.

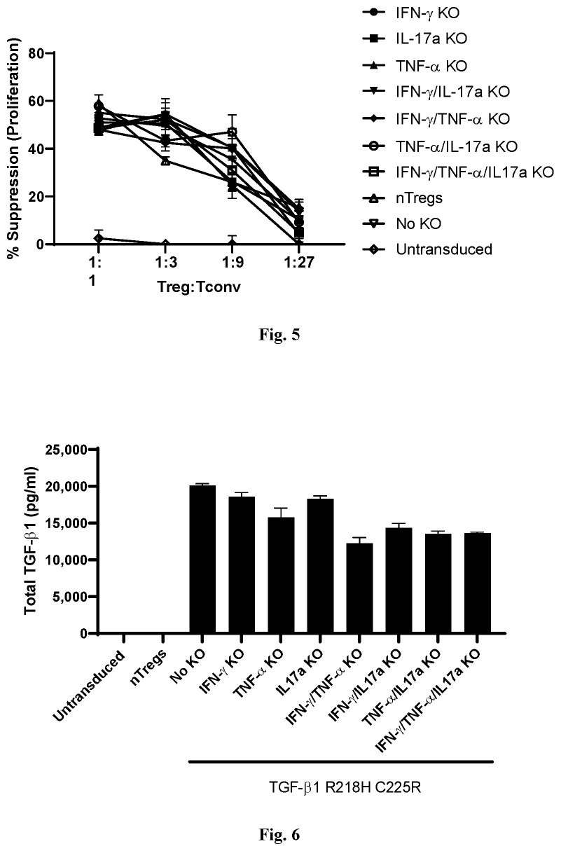

Female NOG mice (NOD.Cg-Prkdc^{scid} Il2rg^{tm1Sug}/TicTac; Taconic, Cat No. NOG-F) were conditioned for cellular transplant by sublethal irradiation (200 rads) using X-rays (RS-2000 irradiator; Rad Source Technologies) one day before injection. Cohorts of irradiated NOG mice were injected intravenously with 100 μL of each test cell population described in the paragraph above. Eight irradiated mice injected with 100 uL RPMI supplemented with 2% FBS and 10 mM HEPES were used as the irradiation only control. Body weight was monitored daily. Upon 20% weight loss, mice were sacrificed. Results are shown in FIG. 4 and Table 4.

TABLE 4
Percent survival days after injection of engineered CD3+CD4+ cells

| | MOCK | IFNG | TNF | IL17a | IFNG/TNF | IFNG/IL17a | TNF/IL17a | IFNG/TNF/IL17a | | | Irradiated |
|---|---|---|---|---|---|---|---|---|---|---|---|
| Days post | KO | KO | KO | KO | KO | KO | KO | KO | nTreg | PBMC | Only |
| injection | (n = 7) | (n = 8) | (n = 8) | (n = 8) | (n = 7) | (n = 6) | (n = 8) | (n = 8) | (n = 7) | (n = 6) | (n = 8) |
| P- value | 0.166 | 0.079 | 0.40 | 0.21 | 0.0064 | 0.43 | 0.0027 | 0.037 | 0.0002 | N/A | N/A |
| vs. PBMC | | | | | | | | | | | |
| 9 | 86 | 88 | 100 | 100 | 100 | 100 | 100 | 100 | 100 | 100 | 100 |
| 11 | 86 | 75 | 100 | 100 | 100 | 100 | 100 | 100 | 100 | 100 | 100 |
| 12 | 86 | 75 | 100 | 100 | 100 | 83 | 100 | 100 | 100 | 83 | 100 |
| 13 | 86 | 75 | 75 | 88 | 100 | 83 | 100 | 100 | 100 | 83 | 100 |
| 14 | 57 | 75 | 50 | 63 | 100 | 50 | 100 | 75 | 100 | 33 | 100 |
| 15 | 42 | 63 | 33 | 38 | 71 | 33 | 88 | 63 | 100 | 16 | 100 |
| 16 | 42 | 50 | 33 | 33 | 57 | 17 | 50 | 38 | 100 | 0 | 100 |
| 17 | 42 | 38 | 17 | 33 | 57 | 17 | 38 | 13 | 100 | | 100 |
| 18 | 0 | 0 | 0 | 0 | 14 | 0 | 13 | 0 | 100 | | 100 |
| 20 | | | | | 14 | | 13 | | 100 | | 100 |

### Example 3.3. Mixed Lymphocyte Reaction Assay of Suppressive Function

Engineered T cells from the population used for injection were assessed for suppressive function in vitro using a mixed lymphocyte reaction (MLR) as described in Example 2 with the ratios of CTV-labelled T cells to engineered T cells described in FIG. 5 and Table 5.

TABLE 5
Percent suppression of autologous T cell proliferation
by engineered T cells as measured by CTV dilution

| | 1 to 1 | | 1 to 3 | | 1 to 9 | | 1 to 27 | |
|---|---|---|---|---|---|---|---|---|
| Sample | Mean | SEM | Mean | SEM | Mean | SEM | Mean | SEM |
| Untransduced | 2.53 | 3.46 | −7.95 | 0.39 | −1.25 | 4.84 | −7.70 | 6.89 |
| T cells | | | | | | | | |
| nTreg | 58.83 | 3.72 | 34.93 | 1.71 | 26.00 | 3.31 | 15.42 | 3.41 |
| MOCK KO | 47.93 | 2.15 | 42.47 | 3.37 | 40.07 | 4.06 | 13.70 | 3.92 |
| IFNG KO | 48.83 | 3.11 | 54.50 | 6.48 | 40.37 | 5.07 | 4.32 | 1.92 |
| TNFA KO | 47.70 | 1.66 | 54.4 | 2.58 | 24.70 | 5.43 | −5.04 | 3.52 |
| IL17A KO | 51.00 | 3.00 | 50.6 | 4.93 | 26.20 | 3.95 | 10.34 | 4.53 |
| IFNG/ | 52.77 | 1.01 | 49.5 | 5.69 | 35.53 | 3.48 | 10.18 | 4.61 |
| IL17A KO | | | | | | | | |
| IFNG/ | 55.03 | 2.28 | 52.3 | 6.90 | 40.53 | 7.88 | 14.85 | 3.84 |
| TNFA KO | | | | | | | | |
| TNFA/ | 57.90 | 4.66 | 43.3 | 2.59 | 47.00 | 7.19 | 9.21 | 2.26 |
| IL17A KO | | | | | | | | |
| IFNG/TNFA/ | 48.57 | 1.93 | 52.1 | 2.78 | 30.9 | 5.27 | 4.83 | 3.81 |
| IL17A KO | | | | | | | | |

### Example 3.4. TGFB1 Expression for Transduced CD3+CD4+ T Cells

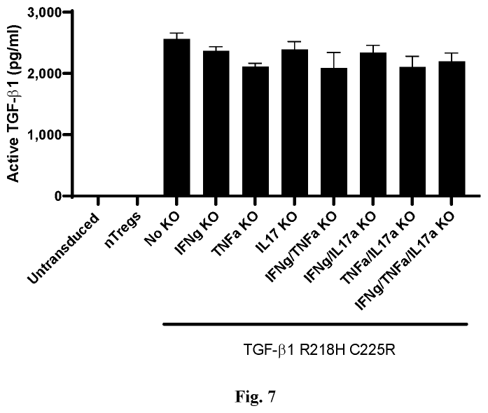

The engineered T cells from the population used for injection were assessed for levels of total TGFB1 and active TGFB1 by ELISA. Sorted transduced CD3+CD4+ T cells were stimulated to assess their cytokine profile. Sorted transduced CD3+CD4+ T cells were plated at 1×10\hat{ }5 T cells/well, in a U-bottom culture plate, in a total of 200 μL OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) with 25 uL/mL ImmunoCult Human CD3/CD28 T cell Activator (Stemcell Technologies, Cat. 10991) and cultured at 37° C. for 48 hours. Following 48 hours of culture, the culture plate was centrifuged, the supernatants collected and frozen for subsequent cytokine quantification using a LEGEND MAX Total TGF-β1 ELISA kit (BioLegend, Cat. 436707), and a LEGEND MAX Free Active TGF-β1 ELISA kit, according to manufacturer's instructions. Total TGFB1 in culture supernatants are shown in Table 6 and FIG. 6. Active TGFB1 in culture supernatants are shown in Table 7 and FIG. 7.

TABLE 6
Production of total TGF-β1 (pg/ml) by
transduced cells upon cell stimulation

| | Total TGF-β1 | | |
|---|---|---|---|
| Sample | Mean | SEM | n |
| Untransduced | 0.00 | 0.00 | 3 |
| CD3+CD4+ T cells | | | |
| nTregs | 0.00 | 0.00 | 3 |
| Mock KO | 20121.77 | 232.63 | 3 |
| IFNG KO | 18580.00 | 572.36 | 3 |
| TNFA KO | 15766.42 | 1289.79 | 3 |
| IL17A KO | 18326.28 | 394.94 | 3 |
| IFNG/TNFA KO | 12249.21 | 804.79 | 3 |
| IFNG/IL 17A KO | 14340.06 | 620.01 | 3 |
| TNFA/IL 17A KO | 13537.05 | 380.72 | 3 |
| IFNG/TNFA/IL17A KO | 13662.47 | 110.17 | 3 |

TABLE 7
Production of active TGF-β1 (pg/ml) by
transduced cells upon cell stimulation

| | Active TGF-β1 | | |
|---|---|---|---|
| Sample | Mean | SEM | n |
| Untransduced | 0.00 | 0.00 | 3 |
| CD3+ CD4+ T cells | | | |
| n Tregs | 0.00 | 0.00 | 3 |
| Mock KO | 2564.97 | 93.24 | 3 |
| IFNG KO | 2369.79 | 65.27 | 3 |
| TNFA KO | 2113.71 | 51.59 | 3 |
| IL17A KO | 2387.58 | 132.81 | 2 |
| IFNG/TNFA KO | 2092.25 | 250.90 | 2 |
| IFNG/IL 17A KO | 2338.87 | 119.41 | 2 |
| TNFA/IL 17A KO | 2105.15 | 176.12 | 2 |
| IFNG/TNFA/IL17A KO | 2197.58 | 136.73 | 2 |

### Example 3.5 Cytokine Profile Analysis of Transduced CD3+CD4+ T Cells

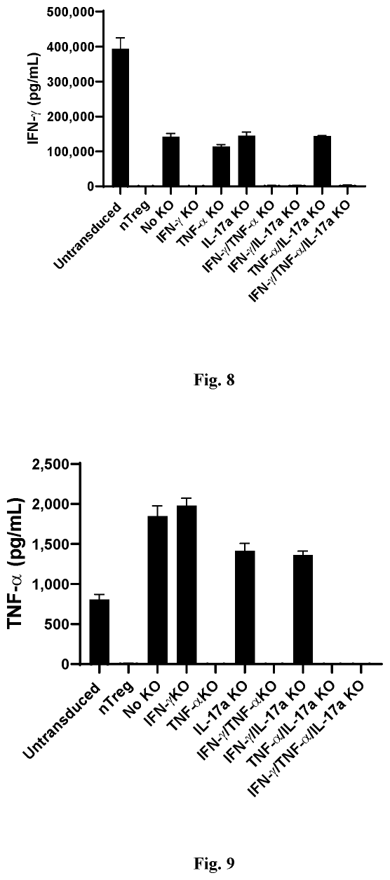

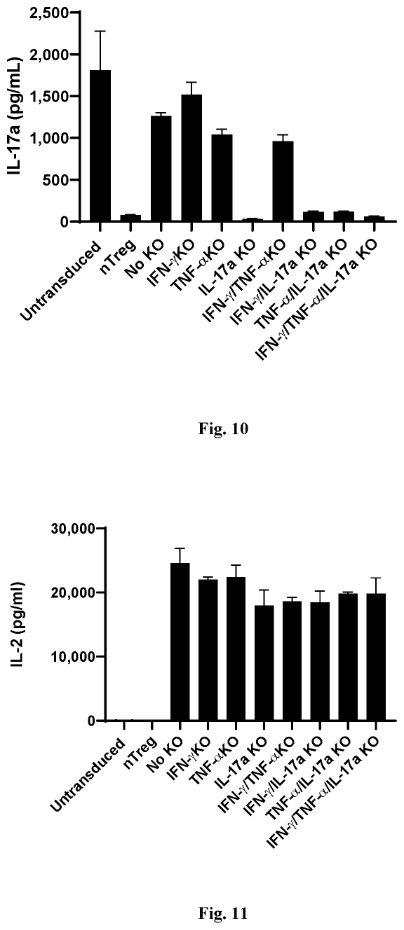

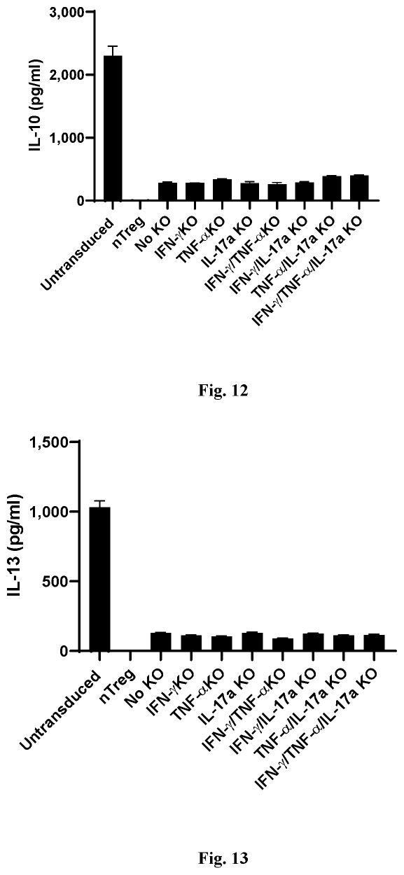

Engineered T cells from the population used for injection were assessed for levels of INFg, TNFa, IL17a, IL10, IL2, and IL13 by ELISA. Sorted transduced CD3+CD4+ T cells were stimulated to assess their cytokine profile. Sorted transduced CD3+CD4+ T cells were plated at 1×10\hat{ }5 T cells/well, in a U-bottom culture plate, in a total of 200 μL OpTmizer base media including CTS OpTmizer T Cell Expansion SFM (Gibco, Cat. A3705001), 1% Penicillin-Streptomycin, 1× Glutamax, 10 mM HEPES, 2.5% human AB serum (Gemini, Cat. 100-512) with 25 uL/mL ImmunoCult Human CD3/CD28 T cell Activator (Stemcell Technologies, Cat. 10991) and cultured at 37° C. for 48 hours. Following 48 hours of culture, the culture plate was centrifuged, the supernatants collected and frozen for subsequent cytokine quantification using a custom U-PLEX Biomarker kit (Meso Scale Diagnostics, Cat. K15067L-2), according to manufacturer's instructions. Specifically, the U-PLEX Biomarker kit was used to quantify the following human cytokines: IFNG, TNFA, IL17A, IL2, IL13, and IL10. The U-PLEX Biomarker plates were read using the Meso Quickplex SQ120 instrument (Meso Scale Discovery) and the data were analyzed with the Discovery Workbench 4.0 software package (Meso Scale Discovery). Results are shown in Table 8 and FIGS. 8-13.

TABLE 8
In vitro cytokine production (pg/ml) of transduced cells upon cell stimulation

| | IFNG | | IL10 | | IL13 | | IL2 | | IL17A | | TNFA | | |
|---|---|---|---|---|---|---|---|---|---|---|---|---|---|
| Sample | Mean | SEM | Mean | SEM | Mean | SEM | Mean | SEM | Mean | SEM | Mean | SEM | n |
| Untransduced | 393476 | 31324 | 2303 | 153 | 1031 | 46 | 138 | 1 | 1810 | 467 | 806 | 67 | 3 |
| CD3+CD4+ | | | | | | | | | | | | | |
| T cells | | | | | | | | | | | | | |
| nTregs | 1327 | 8 | 3 | 1 | 0 | 0 | 2 | 1 | 76 | 5 | 12 | 1 | 3 |
| Mock KO | 141979 | 9342 | 284 | 12 | 130 | 4 | 24569 | 2313 | 1262 | 41 | 1850 | 128 | 3 |
| IFNG KO | 976 | 72 | 282 | 2 | 112 | 5 | 21999 | 443 | 1518 | 147 | 1980 | 92 | 3 |
| TNFA KO | 113875 | 5580 | 342 | 6 | 104 | 4 | 22401 | 1874 | 1041 | 65 | 6 | 0 | 3 |
| IL17A KO | 145000 | 10222 | 275 | 30 | 131 | 4 | 17972 | 2422 | 32 | 6 | 1413 | 93 | 3 |
| IFNG/TNFA | 2474 | 55 | 261 | 28 | 88 | 4 | 18627 | 643 | 960 | 78 | 8 | 0 | 3 |
| KO | | | | | | | | | | | | | |
| IFNG/IL17A | 3337 | 240 | 288 | 12 | 126 | 2 | 18473 | 1776 | 114 | 10 | 1361 | 50 | 3 |
| KO | | | | | | | | | | | | | |
| TNFA/IL17A | 143780 | 1520 | 386 | 12 | 112 | 3 | 19810 | 285 | 117 | 6 | 8 | 1 | 3 |
| KO | | | | | | | | | | | | | |
| IFNG/TNFA/ | 3748 | 108 | 399 | 13 | 116 | 5 | 19830 | 2453 | 61 | 6 | 5 | 0 | 3 |
| IL17A KO | | | | | | | | | | | | | |

### Example 4: In Vivo Assessment of Mutant IL-10, CD25 Expression and IL-2 Knockout on Suppressive T Cells

### Example 4.1. T Cell Engineering

To generate engineered suppressive T cells (supT cells), CD4+ T cells were treated according to Table 9.

Isolated CD4+ T cells were activated by placing 5×10\hat{ }6 cells/well in a 6-well plate in 5 mL of OpTmizer containing 200 U/mL IL-2, 5 ng/ml IL-7, 5 ng/ml IL-15 and 25 uL/mL Immunocult (Stemcell Technologies, 10991). Two days after activation (Day 2), cells were transduced according to Table 9 with a lentivirus species encoding CTLA4 (SEQ ID NO: 130), IL-10 (SEQ ID NO: 124), and mScarlett with a lentivirus encoding CTLA4 (SEQ ID NO: 130), IL-10DE (SEQ ID NO: 229) and mScarlet. Concentrated viral supernatant was added to T cells and centrifuged at 1000×g for 60 mins at 37° C. T cells were resuspended in the cell/viral supernatant mixture and transferred to a single well of a 6-well G-rex (Wilson Wolf; Cat. 80240M) containing T cell base media supplemented with 500 U/mL IL-2, 5 ng/ml IL-7 and 5 ng/ml IL-15. Cells were split and media or cytokines replenished regularly. On Day 3, T cells transduced with CTLA4, IL-10 and mScarlet were further transduced with lentivirus encoding CD25 (NCBI Reference Sequence: NP_000408.1) in the same manner as the Day 2 transductions.

TABLE 9
T cell editing treatment groups. “LV” is for lentivirus.

| | | | | | LNP | |
|---|---|---|---|---|---|---|
| | | LV | LV | | IFNg | |
| | Starting | CTLA4 | CTLA4 & | LV | LNP | LNP |
| Group | Cell | & IL-10 | IL-10DE | CD25 | TNFa | IL-2 |
| nTreg | CD4+ CD25+ | − | − | − | − | − |
| Group 1 | CD4+ | + | − | − | + | − |
| Group 2 | CD4+ | + | − | + | + | − |
| Group 3 | CD4+ | + | − | + | + | + |
| Group 4 | CD4+ | − | + | − | + | − |

On Day 4, T cells were edited according to Table 9 with LNPs to deliver Cas9 mRNA and a guide RNA targeting INF-gamma (G019753), LNPs to deliver Cas9 mRNA and a guide RNA targeting TNF-alpha (G019757) and in some groups, also LNPs to deliver Cas9 mRNA and a guide RNA targeting IL-2 (G021925). LNPs were generated as described in Example 1 using a molar ratio of 35 Lipid A/47.5 cholesterol/15 DSPC/2.5 PEG2k-DMG. The LNPs were formulated with a lipid amine to RNA phosphate (N:P) molar ratio of about 6. LNPs were prepared with a ratio of 1:1 by weight of gRNA to Cas9 mRNA cargo.

Each LNP preparation was incubated in T cell base media supplemented with 5 ug/ml recombinant human ApoE3 (Peprotech, Cat. 350-02) for 15 minutes at 37° C. One day after transduction, T cells were washed and suspended in Serum Free T cell base media with 1000 U/mL recombinant human interleukin-2, 10 ng/ml recombinant human interleukin 7, and 10 ng/ml recombinant human interleukin-15 at concentration of 1 million T cells/mL of media and plated in T-25 flask with 5 million cells per group.

Pre-incubated LNP mix was added to each T-25 flask to yield a final concentration of 1.25 ug/ml total RNA as described in Table 9. Groups with multiple knockouts used 1.25 ug/ml of each respective LNP. Twenty-four hours after LNP treatment, T cells were collected, washed, and cultured in OpTmizer media with 5% Human Serum with cytokines as described in Example 2, for expansion. On Day 5, transduced samples were sorted by flow cytometry to obtain enriched mScarlet+ populations.

CD4+CD25+ T cell populations are known to be enriched for natural Tregs (nTregs). CD4+CD25+ cells were isolated by methods known in the art. The CD25+ cells were activated on Day 0 by placing 5×10\hat{ }6 cells/well in a 6-well Grex containing 30 mL T cell base media supplemented with 1000 U/mL IL-2 and 25 uL/mL ImmunoCult (Stemcell Technologies, 10991). On Day 5, CD4+CD25+ samples were stained with antibodies and sorted by flow cytometry to obtain purified LAP+ populations for use as untranduced “nTreg” group in in vivo mouse studies. Latency-associated peptide (LAP) is a marker of activated Treg cells.

### Example 4.2. In Vivo Functional Characterization of Suppressive T Cells

T cells for in vivo injections were harvested 14 days post-activation. PBMCs autologous to the T cells that were engineered were thawed as described above in the Examples. PBMCs were added to each assay population at a 1:1 ratio and cells resuspended in RPMI supplemented with 2% FBS and 10 mM HEPES to 1.5×10\hat{ }7/mL. The PBMC only group was resuspended at 3×10\hat{ }6/mL.

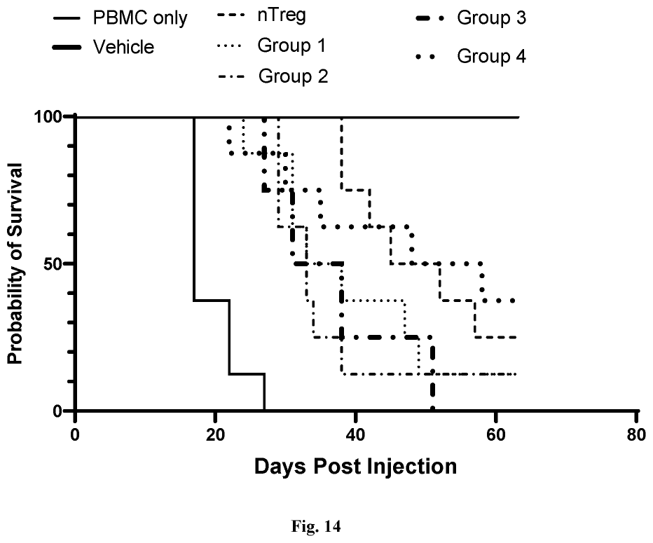

Female NOG mice (NOD.Cg-Prkdcscid Il2rgtm1Sug/JicTac; Taconic, Cat No. NOG-F) were conditioned for cellular transplant by sublethal irradiation (150 rads) using X-rays (RS-2000 irradiator; Rad Source Technologies) one day before injection. Cohorts of irradiated NOG mice were injected intravenously with 100 μL of each test cell population described in the paragraph above. Ten irradiated mice injected with 100 uL RPMI supplemented with 2% FBS and 10 mM HEPES were used as the irradiation only control. Body weight was monitored daily. Upon 20% weight loss, mice were sacrificed. Results are shown in FIG. 14 and Table 10.

TABLE 10
Percent survival of mice in each cohort surviving
at each timepoint after injection.

| | | PBMC | | | | | |
|---|---|---|---|---|---|---|---|
| | Vehicle | only | nTreg | Group 1 | Group 2 | Group 3 | Group 4 |
| Day | n = 8 | n = 8 | n = 8 | n = 8 | n = 8 | n = 4 | n = 8 |
| 1 | 100 | 100 | 100 | 100 | 100 | 100 | 100 |
| 17 | 100 | 37.5 | 100 | 100 | 100 | 100 | 100 |
| 22 | 100 | 12.5 | 100 | 100 | 100 | 100 | 87.5 |
| 24 | 100 | 12.5 | 100 | 87.5 | 100 | 100 | 87.5 |
| 27 | 100 | 0 | 100 | 87.5 | 100 | 75 | 87.5 |
| 29 | 100 | 0 | 100 | 87.5 | 62.5 | 75 | 87.5 |
| 30 | 100 | 0 | 100 | 87.5 | 62.5 | 75 | 75 |
| 31 | 100 | 0 | 100 | 62.5 | 62.5 | 50 | 75 |
| 33 | 100 | 0 | 100 | 50 | 37.5 | 50 | 75 |
| 34 | 100 | 0 | 100 | 50 | 25 | 50 | 75 |
| 35 | 100 | 0 | 100 | 50 | 25 | 50 | 62.5 |
| 38 | 100 | 0 | 75 | 37.5 | 12.5 | 25 | 62.5 |
| 42 | 100 | 0 | 62.5 | 37.5 | 12.5 | 25 | 62.5 |
| 45 | 100 | 0 | 50 | 37.5 | 12.5 | 25 | 62.5 |
| 47 | 100 | 0 | 50 | 25 | 12.5 | 25 | 62.5 |
| 48 | 100 | 0 | 50 | 25 | 12.5 | 25 | 50 |
| 49 | 100 | 0 | 50 | 12.5 | 12.5 | 25 | 50 |
| 51 | 100 | 0 | 50 | 12.5 | 12.5 | 0 | 50 |
| 52 | 100 | 0 | 37.5 | 12.5 | 12.5 | 0 | 50 |
| 57 | 100 | 0 | 25 | 12.5 | 12.5 | 0 | 50 |
| 58 | 100 | 0 | 25 | 12.5 | 12.5 | 0 | 37.5 |
| 63 | 100 | 0 | 25 | 12.5 | 12.5 | 0 | 37.5 |

### Example 5. In Vivo Assessment of Mutant IL-10 Expression in Suppressive T Cells

### Example 5.1. T Cell Engineering

To generate engineered suppressive T cells (supT cells), CD4+ T cells were engineered according to Table 11.

Isolated CD4+ T cells were activated by placing 5×10\hat{ }6 cells/well in a 6-well plate in 5 mL of OpTmizer containing 200 U/mL IL-2, 5 ng/ml IL-7, 5 ng/mL IL-15 and 25 uL/mL Immunocult (Stemcell Technologies, 10991). Two days after activation (Day 2), cells were transduced according to Table 11 with a lentivirus species encoding CTLA4 (SEQ ID NO: 130), IL-10 (SEQ ID NO: 124), and mScarlet, with a lentivirus encoding CTLA4 (SEQ ID NO: 130), IL-10DE (SEQ ID NO: 229), and mScarlet, or with a lentivirus encoding GFP. Concentrated viral supernatant was added to T cells and centrifuged at 1000×g for 60 mins at 37° C. T cells were resuspended in the cell/viral supernatant mixture and transferred to a single well of a 6-well G-rex (Wilson Wolf; Cat. 80240M) containing OpTmizer supplemented with 500 U/mL IL-2, 5 ng/ml IL-7 and 5 ng/ml IL-15. Cells were split and media or cytokines replenished regularly.

TABLE 11
T cell editing treatment groups. ″LV″ is for lentivirus.

| | | | | | LNP |
|---|---|---|---|---|---|
| | | LV | LV | | IFNg |
| | | CTLA4 | CTLA4 & | LV | LNP |
| Group | Starting Cell | & IL-10 | IL-10DE | GFP | TNFa |
| LAP nTreg | CD4+ CD25+ | - | - | - | - |
| CD45RA | CD4+ CD25+ | - | - | - | - |
| nTreg | CD127− | | | | |
| | CD45RA+ | | | | |
| Group 1 | CD4+ | + | - | - | + |
| Group 2 | CD4+ | | + | | + |
| Group 3 | CD4+ | - | - | + | - |

On Day 3, T cells were edited according to Table 11 with LNPs to deliver Cas9 mRNA and a guide RNA targeting INF-gamma (G019753) and LNPs to deliver Cas9 mRNA and a guide RNA targeting TNF-alpha (G019757). LNPs were generated as described in Example 1 using a molar ratio of 35 Lipid A/47.5 cholesterol/15 DSPC/2.5 PEG2k-DMG. The LNPs were formulated with a lipid amine to RNA phosphate (N:P) molar ratio of about 6. LNPs were prepared with a ratio of 1:1 by weight of gRNA to Cas9 mRNA cargo.

Each LNP preparation was incubated in T cell base media supplemented with 5 ug/ml recombinant human ApoE3 (Peprotech, Cat. 350-02) for 15 minutes at 37° C. One day after transduction, T cells were washed and suspended in Serum Free OpTmizer media with 1000 U/mL recombinant human interleukin-2, 10 ng/ml recombinant human interleukin 7, and 10 ng/ml recombinant human interleukin-15 at concentration of 1 million T cells/mL of media and plated in T-25 flask with 5 million cells per group.

Pre-incubated LNP mix was added to each T-25 flask to yield a final concentration of 1.25 ug/ml total RNA as described in Table 11. Groups with multiple knockouts used 1.25 ug/ml of each respective LNP. Twenty-four hours after LNP treatment, T cells were collected, washed, and cultured in OpTmizer media with 5% Human Serum with cytokines as described in Example 2, for expansion. On Day 5, transduced samples were sorted by flow cytometry to obtain enriched mScarlet+ populations.

CD4+CD25+ T cell populations are known to be enriched for natural Tregs (nTregs). CD4+CD25+ cells and CD4+CD25+CD127−CD45RA+ were isolated by methods known in the art. The isolated cells were activated on Day 0 by placing 5×10\hat{ }6 cells/well in a 6-well Grex containing 30 mL OpTmizer supplemented with 1000 U/mL IL-2 and 25 uL/mL ImmunoCult (Stemcell Technologies, 10991). On Day 5, samples were stained with antibodies and sorted by flow cytometry to obtain purified LAP+ populations for use as “LAP nTreg” group and to obtain enriched CD45+ populations for us as “CD45RA nTreg” group for use in in vivo mouse studies. Latency-associated peptide (LAP) is a marker of activated Treg cells.

### Example 5.2. In Vivo Functional Characterization of Suppressive T Cells

T cells for in vivo injections were harvested 13 days post-activation. PBMCs autologous to the T cells that were engineered were thawed as described above in the Examples. PBMCs were added to each assay population at a 1:1 ratio and cells resuspended in RPMI supplemented with 2% FBS and 10 mM HEPES to 5×10\hat{ }7/mL. The PBMC only group was resuspended at 2.5×10\hat{ }7/mL.

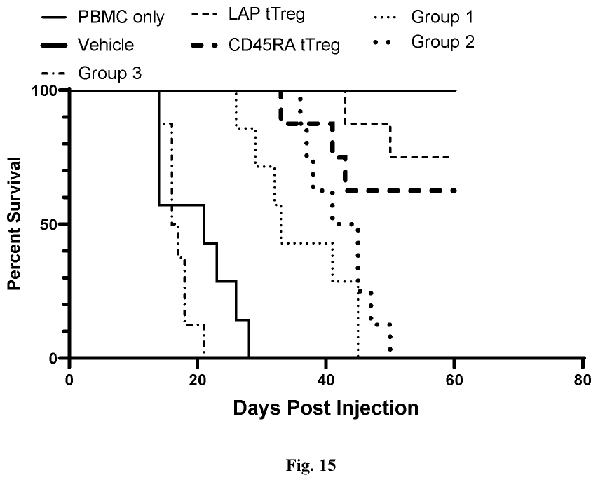

Female NOG mice (NOD.Cg-Prkdcscid Il2rgtm1Sug/JicTac; Taconic, Cat No. NOG-F) were conditioned for cellular transplant by sublethal irradiation (150 rads) using X-rays (RS-2000 irradiator; Rad Source Technologies) one day before injection. Cohorts of irradiated NOG mice were injected intravenously with 100 μL of each test cell population described in the paragraph above. Ten irradiated mice injected with 100 uL RPMI supplemented with 2% FBS and 10 mM HEPES were used as the irradiation only control. Body weight was monitored daily. Upon 20% weight loss, mice were sacrificed. Results are shown in FIG. 15 and Table 12.

TABLE 12
Percent survival of mice in each cohort surviving
at each timepoint after injection.

| | | PBMC | LAP | CD45RA | | | |
|---|---|---|---|---|---|---|---|
| | Vehicle | only | nTreg | nTreg | Group 1 | Group 2 | Group 3 |
| Day | n = 3 | n = 7 | n = 8 | n = 8 | n = 7 | n = 8 | n = 8 |
| 1 | 100 | 100 | 100 | 100 | 100 | 100 | 100 |
| 14 | 100 | 57 | 100 | 100 | 100 | 100 | 88 |
| 16 | 100 | 57 | 100 | 100 | 100 | 100 | 50 |
| 17 | 100 | 57 | 100 | 100 | 100 | 100 | 38 |
| 18 | 100 | 57 | 100 | 100 | 100 | 100 | 13 |
| 21 | 100 | 43 | 100 | 100 | 100 | 100 | 0 |
| 23 | 100 | 29 | 100 | 100 | 100 | 100 | 0 |
| 26 | 100 | 14 | 100 | 100 | 86 | 100 | 0 |
| 28 | 100 | 0 | 100 | 100 | 86 | 100 | 0 |
| 29 | 100 | 0 | 100 | 100 | 71 | 100 | 0 |
| 32 | 100 | 0 | 100 | 100 | 57 | 100 | 0 |
| 33 | 100 | 0 | 100 | 88 | 43 | 100 | 0 |
| 36 | 100 | 0 | 100 | 88 | 43 | 88 | 0 |
| 37 | 100 | 0 | 100 | 88 | 43 | 75 | 0 |
| 38 | 100 | 0 | 100 | 88 | 43 | 63 | 0 |
| 41 | 100 | 0 | 100 | 75 | 29 | 50 | 0 |
| 43 | 100 | 0 | 88 | 63 | 29 | 50 | 0 |
| 45 | 100 | 0 | 88 | 63 | 0 | 25 | 0 |
| 47 | 100 | 0 | 88 | 63 | 0 | 13 | 0 |
| 50 | 100 | 0 | 75 | 63 | 0 | 0 | 0 |
| 60 | 100 | 0 | 75 | 63 | 0 | 0 | 0 |

### Example 6: Suppressive Ability of Engineered T Cells in Inflammatory Bowel Disease Model

### Example 6.1 Assessment of Efficacy of nTregs in a Humanized Mouse Model of Colitis

Tregs are known to suppress the induction of colitis in pre-clinical models (see, e.g., Goettel et al., Low-Dose Interleukin-2 Ameliorates Colitis in a Preclinical Humanized Mouse Model. Cell Mol Gastroenterol Hepatol. 2019; 8 (2): 193-195), or in the CD45RBhi transfer model of IBD (see, e.g., Asseman et al., An Essential Role for Interleukin 10 in the Function of Regulatory T Cells That Inhibit Intestinal Inflammation. J Expt Med. 190(7): 995-1003).

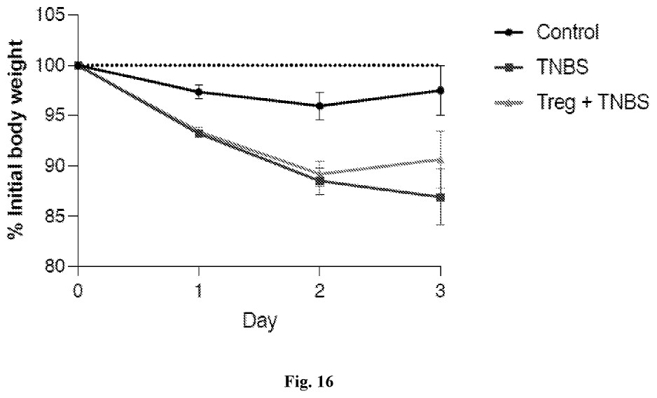

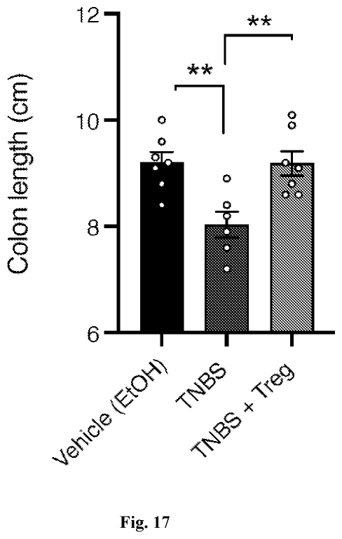

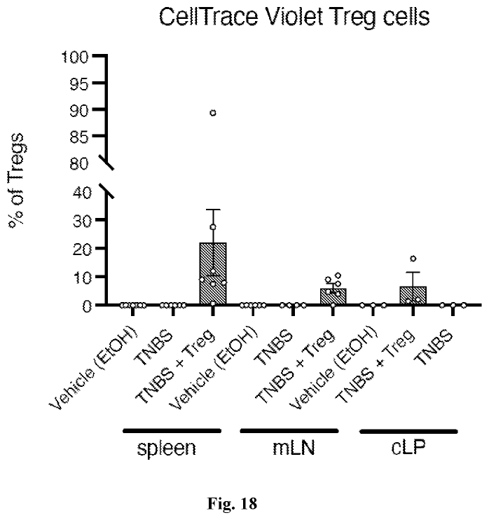

Natural Tregs were isolated using methods known in the art and administered to a humanized mouse model of IBD wherein the application of trinitrobenzene sulfonic acid (TNBS) to the colons of TNBS-sensitized mice induces colitis. Briefly, NSG mice were reconstituted with Human CD34+ cells were obtained from a commercial source (Jackson Laboratory). Following a 1-week acclimation period, mice were injected subcutaneously (s.c.) with TNBS or vehicle, and received an intraperitoneal (i.p.) injection of vehicle or natural Tregs (nTregs) [1×10\hat{ }⁶]. Three days later, the mice received an enema of vehicle (50% ethanol) or TNBS in 50% ethanol as described in Table 13. Body weight was recorded daily and results are shown in FIG. 16 (+/−SEM) and Table 14.

TABLE 13
Experimental design

| | Sensitization | Treg Treatment | Enema | |
|---|---|---|---|---|
| | Phase | (1 × 10⁶; | Phase | Number |
| Group | (Day −7) | Day −7) | (Day 0) | of mice |
| 1 (Control) | Vehicle | Vehicle | Vehicle | 7 |
| 2 | TNBS | Vehicle | TNBS | 7 |
| 3 | TNBS | Treg | TNBS | 7 |

TABLE 14
Percent of initial body weight individual mice in treatment groups.

| | DAY | DAY | DAY | DAY |
|---|---|---|---|---|
| Group | 0 | 1 | 2 | 3 |
| Control | 100.0 | 99.5 | 97.1 | 99.5 |
| | 100.0 | 95.8 | 96.6 | 99.2 |
| | 100.0 | 95.1 | 89.3 | 83.5 |
| | 100.0 | 98.8 | 93.8 | 101.7 |
| | 100.0 | 99.6 | 100.9 | 102.6 |
| | 100.0 | 96.4 | 98.2 | 99.1 |
| | 100.0 | 96.3 | 95.9 | 97.0 |
| TNBS | 100.0 | 94.7 | 95.1 | 99.6 |
| | 100.0 | 92.9 | 85.8 | 82.0 |
| | 100.0 | 93.2 | 85.0 | 80.0 |
| | 100.0 | 94.4 | 90.1 | 86.1 |
| | 100.0 | 91.7 | 86.5 | 82.9 |
| | 100.0 | 92.2 | 89.8 | 94.9 |
| | 100.0 | 93.5 | 87.3 | 82.9 |
| Treg + | 100.0 | 93.2 | 86.0 | 82.1 |
| TNBS | 100.0 | 91.5 | 84.3 | 80.6 |
| | 100.0 | 93.9 | 91.9 | 92.7 |
| | 100.0 | 93.0 | 90.4 | 96.1 |
| | 100.0 | 94.6 | 93.3 | 98.7 |
| | 100.0 | 94.6 | 88.0 | 86.5 |
| | 100.0 | 93.2 | 90.5 | 97.7 |

At day 3 post-enema, mice were sacrificed. Colon lengths were determined to assess protection against colitis. Spleen, mesenteric lamina propria (mLN), and colonic lamina propria (cLP) were harvested and stained with CellTrace Violet to determine persistence of Tregs in the various tissues for the duration of the model. Results are shown in FIGS. 17 (+/−SEM) and 18 (+/−SEM) and Tables 15 and 16 respectively.

TABLE 15
Colon length in cm.

| | Colon |
|---|---|
| | Length |
| Group | (cm) |
| Control | 9.3 |
| | 8.4 |
| | 9.2 |
| | 10 |
| | 9.6 |
| | 8.8 |
| | 9.1 |
| TNBS | 8.4 |
| | 7.2 |
| | 8.9 |
| | 7.6 |
| | 7.9 |
| | 8.2 |
| TNBS + | 8.6 |
| Treg | 8.6 |
| | 9.1 |
| | 10.1 |
| | 9.2 |
| | 8.8 |
| | 9.9 |

TABLE 16
Percent of Tregs stained with Cell Trace Violet
recovered from each tissue in individual mice.

| | Vehicle | | TNBS + |
|---|---|---|---|
| Source | (EtOH) | TNBS | Treg |
| Spleen | 0 | 0 | 89.3 |
| | 0 | 0 | 9 |
| | 0 | 0 | 7.9 |
| | 0 | 0 | 7.5 |
| | 0 | 0 | 0.7 |
| | 0 | 0 | 27.5 |
| | 0 | | 12 |
| mLN | 0 | 0 | 0 |
| | 0 | 0 | 7.6 |
| | 0 | 0 | 4.8 |
| | 0 | 0 | 9.1 |
| | 0 | | |
| | 0 | | 10.5 |
| | | | 4 |
| CLP | 0 | 0 | 16.4 |
| | 0 | 0 | 1.4 |
| | 0 | 0 | 2 |

These data demonstrate that nTregs are protective against colitis in the humanized mouse model based on the rebound of loss of body weight and longer colon length in nTreg treated TNBS treated mice as compared vehicle control treated TNBS treated mice. CellTrace Violet staining demonstrate the persistence of nTregs in all of the assessed compartments throughout the duration of the experiment.

### Example 6.2: Comparison of Efficacy of nTregs and Engineered T Cells in a Humanized Mouse Model of Colitis

Based on this demonstration of the efficacy of nTregs in providing protection against colitis in the humanized mouse model, engineered T cells are assessed for protection against colitis in the same mouse model. The engineered T cells are prepared generally as described in Example 3.1 and are engineered to disrupt TNFA and IFNG, and to provide expression of TGFB1 R218H C225R, CTLA4, and IL-10 D25A/E96A. The study further includes a time course to assess outcomes. As above, NSG mice reconstituted with Human CD34+ cells are obtained from a commercial source (Jackson Laboratory). Following a 1-week acclimation period, mice are injected s.c. with TNBS or vehicle, and receive an i.p. injection of vehicle, nTregs or engineered Tregs, as outlined in the Table 17. One week later, the mice receive an enema of vehicle (50% ethanol) or TNBS in 50% ethanol. Body weight is recorded daily. At day 3, day 5 and day 7 post-enema, 6 mice from group 1 and 8 mice from groups 2, 3, 4 are sacrificed.

TABLE 17
Experimental treatment groups.

| | | Treg | | |
|---|---|---|---|---|
| | Sensitization | Treatment | Enema | |
| | Phase | (1 × 10\hat{ }⁶; | Phase | Number |
| Group | (Day −7) | Day −7) | (Day 0) | of mice |
| 1 | Vehicle | Vehicle | Vehicle | 18 |
| 2 | TNBS | Vehicle | TNBS | 24 |
| 3 | TNBS | n Tregs | TNBS | 24 |
| 4 | TNBS | Engineered | TNBS | 24 |
| | | T cells | | |

Body weight is monitored throughout the study. At sacrifice, colons are harvested and measured to determine length. Colon sections are analyzed using 5-parameter blinded histology following H&E staining. The study confirms the results from the single time point study demonstrating the protection provided by nTregs in the model of colitis and demonstrates the effect of engineered T cells to provide protection against colitis in the same model. The study is used to select the best time post-enema to assess study outcomes in the model.

### Example 6.3: Assessment of Efficacy of Targeted Engineered T Cells in a Humanized Mouse Model of Colitis

A further study is performed to determine the effect of targeting of the engineered T cells using chimeric antigen receptors (CARs) that bind mouse MadCAM, a surface protein present on various tissues in the gastrointestinal tract. As in the prior experiment, T cells are engineered generally as described in Example 4. The T cells are further edited using SpyCas9 and known methods to insert a construct for expression of the MadCAM CAR into the TRAC locus. To allow delivered engineered cells to be differentiated from the endogenous mouse T cells, the engineered T cells are also transduced with a lentivirus expressing either green fluorescent protein (GFP) or mScarlet. Time points for use in the study are selected based on the time course study above. The experimental groups are described in Table 18.

TABLE 18
Experimental Design

| | | Treg | | |
|---|---|---|---|---|
| | Sensitization | Treatment | Enema | |
| | Phase | (1 × 10⁶; | Phase | Number |
| Group | (Day −7) | Day −7) | (Day 0) | of mice |
| 1 | Vehicle | Vehicle | Vehicle | 10 |
| 2 | TNBS | Vehicle | TNBS | 10 |
| 3 | TNBS | Engineered T cell | TNBS | 10 |
| 4 | TNBS | MadCAM-CAR | TNBS | 10 |

Body weight is monitored throughout the study. At sacrifice, colons are harvested and measured to determine length. Colon sections are analyzed using 5-parameter blinded histology following H&E staining. Spleen, mesenteric lamina propria, and colonic lamina propria are harvested for analysis using Bulk RNA-seq using routine methods of the transferred engineered T cells and endogenous T effector cells. Flow cytometry is also performed on T cells from the spleen, mesenteric lamina propria, and colonic lamina propria by staining for CD3, CD4, CD8, hCD45, mCD45, and live/dead with the delivered engineered T cells identifiable by GFP or mScarlet.

The study confirms the results from the prior study demonstrating the protection provided by nTregs and engineered T cells in the model of colitis. The study demonstrates targeted engineered T cells are also provide protection in the model of colitis.

1. An engineered T cell, comprising:
a heterologous nucleic acid sequence encoding a dual mutant transforming growth factor beta 1 (dmTGFB1) under control of a promoter sequence.

2. The engineered T cell of claim 1, further comprising:
i) a modification of an endogenous a nucleic acid sequence encoding a tumor necrosis factor alpha (TNFA) wherein the modification knocks down expression of TNFA; and
ii) a modification of an endogenous nucleic acid sequence encoding an interferon-gamma (IFNG) wherein the modification knocks down expression of the IFNG or a modification of an endogenous a nucleic acid sequence encoding an interleukin17A (IL17A) wherein the modification knocks down expression of IL17A.

3. The engineered T cell of claim 1 or 2, comprising a modification of an endogenous nucleic acid sequence encoding an IFNG wherein the modification knocks down expression of IFNG and a modification of an endogenous nucleic acid sequence encoding an IL17A wherein the modification knocks down expression of the IL17A.

4. An engineered T cell, comprising:
i) a heterologous nucleic acid sequence encoding a regulatory T cell promoting molecule under control of a promoter sequence;
ii) a modification of an endogenous nucleic acid sequence encoding an interleukin17A (IL17A) wherein the modification knocks down expression of the IL17A; and
iii) a modification of an endogenous a nucleic acid sequence encoding a tumor necrosis factor alpha (TNFA) wherein the modification knocks down expression of TNFA.

5. The engineered T cell of claim 4, wherein the regulatory T cell promoting molecule is a selected from interleukin-10 (IL10), cytotoxic T-lymphocyte associated protein 4 (CTLA4), indoleamine 2,3-dioxygenase 1 (IDO1), ectonucleoside triphosphate diphosphohydrolase 1 (ENTPD1), 5′-nucleotidase ecto (NT5E), interleulin-22 (IL-22), amphiregulin (AREG), interleukin-35 (IL35), GARP, CD274 molecule (CD274), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), BTB domain and CNC homolog 2 (BACH2), and interleukin 2 receptor subunit alpha (IL2RA).

6. The engineered T cell of claim 5, wherein the regulatory T cell promoting molecule is IL10.

7. The engineered T cell of claim 5, wherein the regulatory T cell promoting molecule is CTLA4.

8. The engineered T cell of any of claims 5-7, wherein the regulatory T cell promoting molecule is a first regulatory T cell promoting molecule, and further comprising a heterologous nucleic acid sequence encoding a second regulatory T cell promoting molecule under control of a promoter sequence.

9. The engineered T cell of claim 8, wherein the first and the second regulatory T cell promoting molecules are IL10 and CTLA4.

10. The engineered T cell of any one of claims 1-9, further comprising a modification of an endogenous nucleic acid sequence encoding an interleukin 17A (IL17A), an interleukin-2 (IL2), an interleukin 6 (IL6), a perforin 1 (PRF1), a granzyme A (GZMA), a granzyme B (GZMB), a Fas ligand (FASL, NF superfamily, member 6), a ryanodine receptor 2 (RYR2), and colony stimulating factor 2 (CSF2) wherein the modification knocks down expression of the IL17A, the IL2, the IL6, the PRF1, the GZMA, the GZMB, the FASL, the RYR2, or the CSF2 respectively.

11. The engineered T cell of any one of claims 1-10, further comprising a modification of an endogenous nucleic acid sequence encoding an endogenous T cell receptor (TCR), wherein the modification knocks down expression of the endogenous TCR.

12. The engineered T cell of any one of claims 1-11, further comprising a heterologous coding sequence for a targeting receptor under control of a promoter sequence.

13. The engineered T cell of claim 12, wherein the targeting receptor is targeted to a ligand selected from mucosal vascular addressin cell adhesion molecule 1 (MADCAM1), tumor necrosis factor alpha (TNFA), CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), desmoglein 3 (DSG3), and MHC class I HLA-A (HLA-A*02),

14. The engineered T cell of claim 12, wherein the targeting receptor is targeted to MADCAM1.

15. The engineered T cell of any of claims 12-14, wherein the targeting receptor comprises a chimeric antigen receptor (CAR) or a T cell receptor (TCR).

16. The engineered T cell of any one of claims 12-15, wherein the heterologous nucleic acid sequence encoding the targeting receptor is incorporated into an expression construct.

17. The engineered T cell of any one of claims 12-16, wherein the heterologous nucleic acid sequence encoding a targeting receptor is in an expression construct that does not comprise a nucleic acid sequence encoding a regulatory T cell promoting molecule.

18. The engineered T cell of any one of claims 8-17, wherein the heterologous nucleic acid sequence encoding the first regulatory T cell promoting molecule is incorporated into an expression construct and the heterologous nucleic acid sequence encoding the second regulatory T cell promoting molecule is incorporated in an expression construct.

19. The engineered T cell of any one of claims 8-18, wherein the heterologous nucleic acid sequence encoding the first regulatory T cell promoting molecule and the heterologous nucleic acid sequence encoding the second regulatory T cell promoting molecule are incorporated into separate expression constructs.

20. The engineered T cell of claim 17 or 18, wherein the heterologous nucleic acid sequence encoding the first regulatory T cell promoting molecule and the heterologous nucleic acid sequence encoding the second regulatory T cell promoting molecule are incorporated into a single expression construct.

21. The engineered T cell of claim 16 or 18-20, wherein the expression construct further comprises a nucleic acid sequence encoding the targeting receptor.

22. The engineered T cell of any one of claims 15-21, wherein at least one heterologous coding sequence is in an episomal expression construct.

23. The engineered T cell of any one of claims 1-20, wherein at least one heterologous nucleic acid sequence is inserted into the genome.

24. The engineered T cell of claim 23, wherein the insertion into the genome is an untargeted insertion.

25. The engineered T cell of claim 23, wherein the insertion is a targeted insertion.

26. The engineered T cell of claim 25, wherein the targeted insertion is into a site selected from a TCR gene locus, a TNF gene locus, an IFNG gene locus, IL17A gene locus, IL6 gene locus, IL2 gene locus, an adeno-associated virus integration site 1 (AAVS1) locus.

27. The engineered T cell of claim 26, wherein the TCR gene locus is a T cell receptor alpha constant (TRAC) locus.

28. The engineered T cell of any one of claims 2-27, wherein the modification that knocks down expression of a gene comprises one or more of an insertion, a deletion, or a substitution.

29. A population of cells comprising the engineered T cell of any of claims 1-28.

30. A population of engineered T cells comprising the engineered T cells of any of claims 1-28, wherein at least 30%, preferably at least 40%, of cells of the population comprise a heterologous nucleic acid sequence encoding a dmTGFB1 under control of a promoter sequence.

31. The population of engineered T cells of claim 30, wherein
at least 50%, preferably at least 70%, of cells of the population comprise a modification of an endogenous nucleic acid sequence encoding a TNFA; and
at least 50%, preferably at least 70%, of cells of the population comprise a modification of an endogenous nucleic acid sequence encoding an IFNG; or
at least 50%, preferably at least 70%, of cells of the population comprise a modification of an endogenous nucleic acid sequence encoding an IL17A.

32. The population of engineered T cells of claim 30 or 31, wherein at least 50%, preferably at least 70%, of cells of the population comprise a modification of an endogenous nucleic acid sequence encoding an IFNG; and
wherein at least 50%, preferably at least 70%, of cells of the population comprise a modification of an endogenous nucleic acid sequence encoding an interleukin (IL17A) wherein the modification knocks down expression of the IL17A.

33. The population of engineered T cells of claim 31 or 32, wherein at least 50%, preferably at least 70%, of cells of the population comprise a modification of an endogenous nucleic acid sequence encoding a TNFA; and
at least 50%, preferably at least 70%, of cells of the population comprise a modification of an endogenous nucleic acid sequence encoding an interleukin (IL17A) wherein the modification knocks down expression of the IL17A.

34. The population of engineered T cells of any of claims 31-33, wherein at least 30%, preferably at least 40%, of cells of the population comprise a heterologous nucleic acid sequence encoding a regulatory T cell promoting molecule under control of a promoter sequence.

35. The population of engineered T cells of claim 29, wherein
at least 30%, preferably at least 40%, of cells of the population comprise a heterologous nucleic acid sequence encoding a regulatory T cell promoting molecule under control of a promoter sequence;
at least 50%, preferably at least 70%, of cells of the population comprise a modification of an endogenous nucleic acid sequence encoding a TNFA; and
at least 50%, preferably at least 70%, of cells of the population comprise a modification of an endogenous nucleic acid sequence encoding an IL17A.

36. The population of engineered cells of claim 35, wherein at least 50%, preferably at least 70%, of cells of the population comprise a modification of an endogenous nucleic acid sequence encoding an IFNG.

37. The population of engineered T cells of any of claims 29-36, wherein the percent of cells comprising an insertion or a modification is determined by the percent of reads by next generation sequencing (NGS).

38. The population of engineered T cells of claim 34, wherein the regulatory T cell promoting molecule is a selected from interleukin-10 (IL10), cytotoxic T-lymphocyte associated protein 4 (CTLA4), indoleamine 2,3-dioxygenase 1 (IDO1), ectonucleoside triphosphate diphosphohydrolase 1 (ENTPD1), 5′-nucleotidase ecto (NT5E), interleulin-22 (IL22), amphiregulin (AREG), forkhead box P3 (FOXP3), IKAROS family zinc finger 2 (IKZF2), eosinophilia familial (EOS), interferon regulatory factor 4 (IRF4), lymphoid enhancer binding factor 1 (LEF1), BTB domain and CNC homolog 2 (BACH2), and interleukin 2 receptor subunit alpha (IL2RA).

39. The population of engineered T cells of any of claims 34-38, wherein the regulatory T cell promoting molecule is IL10.

40. The population of engineered T cell of any of claims 34-39, wherein the regulatory T cell promoting molecule is CTLA4.

41. The population of engineered T cells of any of claims 34-40, wherein the regulatory T cell promoting molecule is a first regulatory T cell promoting molecule, and further comprising a heterologous nucleic acid sequence encoding a second regulatory T cell promoting molecule under control of a promoter sequence.

42. The engineered T cell of claim 41, wherein the first and the second regulatory T cell promoting molecules are IL10 and CTLA4.

43. The population of engineered T cell of any one of claims 29-42, further comprising a modification of at least one endogenous nucleic acid sequence encoding an interleukin 17A (IL17A), an interleukin 6 (IL6), an interleukin 2 (IL2), a perforin 1 (PRF1), a granzyme A (GZMA), a granzyme B (GZMB), a Fas ligand (FASL, NF superfamily, member 6), a ryanodine receptor 2 (RYR2), and colony stimulating factor 2 (CSF2), wherein the population of cells comprises a modification in the at least one of the IL17A, the IL6, the IL2, the PRF1, the GZMA, the GZMB, the FASL, the RYR2, or the CSF2 respectively, in at least 70% of the population of cells, preferably at least 80% of the population of cells, wherein the modification knocks down expression of the at least one of the IL17A, the IL6, the IL2, the PRF1, the GZMA, the GZMB, the FASL, the RYR2, or the CSF2 respectively.

44. The population of engineered T cell of claim 43, wherein the modification of at least one endogenous nucleic acid sequence encoding interleukin 2 (IL2), wherein the population of cells comprises a modification in the IL2 in at least 70% of the population of cells, preferably at least 80% of the population of cells, wherein the modification knocks down expression of the IL2.

45. The population of engineered T cells of any one of claims 29-44, wherein at least 50%, preferably at least 70%, of the cells include a knockdown of a TCR.

46. The population of engineered T cells of any one of claims 29-45, wherein at least 30%, preferably at least 40%, of the cells include an insertion of a nucleic acid coding sequence of a targeting receptor.

47. The population of engineered T cells of claim 46, wherein the targeting receptor binds specifically to a ligand selected from mucosal vascular addressin cell adhesion molecule 1 (MADCAM1), tumor necrosis factor alpha (TNFA), CEA cell adhesion molecule 6 (CEACAM6), vascular cell adhesion molecule 1 (VCAM1), citrullinated vimentin, myelin basic protein (MBP), MOG (myelin oligodendrocyte glycoprotein), proteolipid protein 1 (PLP1), CD19 molecule (CD19), CD20 molecule (CD20), TNF receptor superfamily member 17 (TNFRSF17), dipeptidyl peptidase like 6 (DPP6), solute carrier family 2 member 2 (SCL2A2), glutamate decarboxylase (GAD2), desmoglein 3 (DSG3), and MHC class I HLA-A (HLA-A*02).

48. The population of engineered T cells of claim 46 or 47, wherein the targeting receptor comprises a chimeric antigen receptor (CAR) or a T cell receptor (TCR).

49. The population of engineered T cells of any one of claims 46-48, wherein the heterologous nucleic acid sequence encoding the targeting receptor is incorporated into an expression construct.

50. The population of engineered T cells of claim 49, wherein the heterologous nucleic acid sequence encoding a targeting receptor is in an expression construct that does not comprise a nucleic acid sequence encoding a regulatory T cell promoting molecule.

51. The population of engineered T cells of any one of claims 29-50, wherein the heterologous nucleic acid sequence encoding a first of the at least one regulatory T cell promoting molecule is incorporated into an expression construct and the heterologous nucleic acid sequence encoding a second of the at least one regulatory T cell promoting molecule is incorporated in an expression construct.

52. The population of engineered T cells of claim 51, wherein the heterologous nucleic acid sequence encoding the first regulatory T cell promoting molecule and the heterologous nucleic acid sequence encoding the second regulatory T cell promoting molecule are incorporated into separate expression constructs.

53. The population of engineered T cells of claim 51, wherein the heterologous nucleic acid sequence encoding the first regulatory T cell promoting molecule and the heterologous nucleic acid sequence encoding the second regulatory T cell promoting molecule are incorporated into a single expression construct.

54. The population of engineered T cells of claim 49-53, wherein the expression construct further comprises a nucleic acid sequence encoding a targeting receptor.

55. The population of engineered T cells of any one of claims 29-54, wherein at least one heterologous coding sequence is in an episomal expression construct.

56. The population of engineered T cells of any one of claims 29-55, wherein at least one heterologous coding sequence is inserted into the genome.

57. The population of engineered T cells of claim 56, wherein the insertion into the genome is an untargeted insertion.

58. The population of engineered T cells of claim 56, wherein the insertion is a targeted insertion.

59. The population of engineered T cells of claim 58, wherein the targeted insertion is into a site selected from a TCR gene locus, a TNF gene locus, an IL2 gene locus, a IL6 gene locus, a IL17A gene locus, an IFNG gene locus, an adeno-associated virus integration site 1 (AAVS1) locus.

60. The population of engineered T cells of claim 59, wherein the TCR gene locus is a T cell receptor alpha constant (TRAC) locus.

61. The population of engineered T cells of claims 29-60, wherein the modification that knocks down expression of a gene comprises one or more of an insertion, a deletion, or a substitution.

62. A pharmaceutical composition comprising any of the engineered T cells of claims 1-28 or population of engineered T cells of claims 29-61.

63. A method or use of administering a cell of any one of claims 1-28 or a population of cells of any one of claims 29-61, or the pharmaceutical composition of claim 62 to a subject.

64. The method or use of claim 63, wherein the subject is in need of immunosuppression.

65. The method or use of claim 63 or 64, for treatment of an immune disorder.

66. The method of use of any one of claims 63-65, for treatment of an autoimmune disease.

67. The method or use of claim 66, wherein the autoimmune disease is selected from ulcerative colitis, Crohn's disease, rheumatoid arthritis, psoriasis, multiple sclerosis, systemic lupus erythematosus, and type 1 diabetes.

68. The method or use of any one of claims 63-65, for treatment of graft versus host disease (GvHD).

69. A polypeptide comprising a dmTGFB1.

70. The polypeptide of claim 69, wherein the dmTGFB1 is a human dmTGFB1.

71. The polypeptide of claim 70, wherein the dmTGFB1 comprises a mutation at two or more amino acids selected from F198, D199, V200, L208, F217, L219, R218, H222, C223, and C225 relative to a wild type human TGFB1.

72. The polypeptide of claim 70 or 71, wherein the dmTGFB1 comprises a mutation at two or more amino acids selected from R218, H222, C223, and C225 relative to a wild type human TGFB1.

73. The polypeptide of any one of claims 70-72, wherein the dmTGFB1 comprises two or more amino acid mutations selected from R218C/H, H222D, C223S/R/G, and C225R relative to a wild type human TGFB1.

74. The polypeptide of any one of claims 70-72, wherein the dmTGFB1 comprises amino acid mutations R218C/H and C225R.

75. A nucleic acid sequence encoding the polypeptide of one of claims 69-74.
